(19) United States
(12) Patent Application Publication
(10) Pub. No.: US 2011/0165147 A1
(43) Pub. Date: Jul. 7, 2011

(54) NOVEL 18636, 2466, 43238, 1983, 52881, 2398, 45449, 50289, 52872 AND 26908 MOLECULES AND USES THEREFOR

(75) Inventors: Maria A. Glucksmann, Lexington, MA (US); Inmaculada Silos-Santiago, Del Mar, CA (US); Joseph M. Carroll, Boulder, CO (US); Katherine M. Galvin, Jamaica Plain, MA (US)

(73) Assignee: Millennium Pharmaceuticals, Inc., Cambridge, MA (US)

(21) Appl. No.: 13/037,699

(22) Filed: Mar. 1, 2011

Related U.S. Application Data
(63) Continuation of application No. 12/387,287, filed on Apr. 28, 2009, now abandoned.
Application No. 12/387,287 is a continuation of application No. 11/522,789, filed on Sep. 18, 2006, now abandoned.
Application No. 11/522,789 is a continuation of application No. 10/407,079, filed on Apr. 3, 2003, now abandoned.
Application No. 10/407,079 is a continuation-in-part of application No. 10/226,102, filed on Aug. 22, 2002, now abandoned.
Application No. 10/407,079 is a continuation-in-part of application No. 10/225,094, filed on Aug. 21, 2002, now abandoned.
Application No. 10/407,079 is a continuation-in-part of application No. 10/272,417, filed on Oct. 15, 2002, now abandoned.
Application No. 10/272,417 is a continuation of application No. 09/715,790, filed on Nov. 17, 2000, now abandoned.
Application No. 10/407,079 is a continuation-in-part of application No. 10/282,837, filed on Oct. 29, 2002, now abandoned.
Application No. 10/282,837 is a continuation of application No. 09/796,338, filed on Feb. 28, 2001, now abandoned.
Application No. 10/407,079 is a continuation-in-part of application No. 09/863,200, filed on May 22, 2001, now abandoned.
(60) Provisional application No. 60/314,041, filed on Aug. 22, 2001.
(60) Provisional application No. 60/314,185, filed on Aug. 22, 2001.
(60) Provisional application No. 60/191,845, filed on Mar. 24, 2000.
(60) Provisional application No. 60/186,059, filed on Feb. 29, 2000.
(60) Provisional application No. 60/206,019, filed on May 22, 2000.

(51) Int. Cl. A61K 39/395; C07H 21/04; C12N 5/10; C07K 14/00; C07K 16/00; C12P 21/02; C12Q 1/68; G01N 33/00; G01N 33/53; A61K 38/00; A61K 38/16; A61K 31/711; A61P 43/00

(52) U.S. Cl. 424/130.1; 536/23.1; 435/325; 530/350; 530/324; 530/387.9; 435/69.1; 435/6.11; 436/86; 435/7.1; 514/1.1; 514/21.2; 514/21.3; 514/44 A

(57) ABSTRACT

The invention provides isolated nucleic acids molecules, designated 18636, 2466, 43238, 1983, 52881, 2398, 45449, 50289, 52872 and 26908 nucleic acid molecules. The invention also provides antisense nucleic acid molecules, recombinant expression vectors containing 18636, 2466, 43238, 1983, 52881, 2398, 45449, 50289, 52872 and 26908 nucleic acid molecules, host cells into which the expression vectors have been introduced, and nonhuman transgenic animals in which a 18636, 2466, 43238, 1983, 52881, 2398, 45449, 50289, 52872 or 26908 gene has been introduced or disrupted. The invention still further provides isolated 18636, 2466, 43238, 1983, 52881, 2398, 45449, 50289, 52872 or 26908 proteins, fusion proteins, antigenic peptides and anti-18636, 2466, 43238, 1983, 52881, 2398, 45449, 50289, 52872 or 26908 antibodies. Diagnostic and therapeutic methods utilizing compositions of the invention are also provided.

### RELATED APPLICATIONS

The present application is a continuation of U.S. patent application Ser. No. 12/387,287, filed Apr. 28, 2009 (pending) which is a continuation of U.S. patent application Ser. No. 11/522,789, filed Sep. 18, 2006 (abandoned), which is a continuation of U.S. Patent Application Ser. No. 10/407,079, filed Apr. 3, 2003 (abandoned), which is a continuation-in-part of U.S. patent application Ser. No. 10/226,102, filed Aug. 22, 2002 (abandoned), which claims the benefit of U.S. Provisional Application Ser. No. 60/314,041, filed Aug. 22, 2001 (abandoned). U.S. patent application Ser. No. 10/407,079 is also a continuation-in-part of U.S. patent application Ser. No. 10/225,094, filed Aug. 21, 2002 (abandoned), which claims the benefit of U.S. Provisional Application Ser. No. 60/314,185, filed Aug. 22, 2001 (abandoned). U.S. patent application Ser. No. 10/407,079 is also a continuation-in-part of U.S. patent application Ser. No. 10/272,417, filed Oct. 15, 2002 (abandoned), which is a continuation of U.S. patent application Ser. No. 09/715,790, filed Nov. 17, 2000 (abandoned), which claims the benefit of U.S. Provisional Application Ser. No. 60/191,845, filed Mar. 24, 2000 (abandoned). U.S. patent application Ser. No. 10/407,079 is also a continuation-in-part of U.S. patent application Ser. No. 10/282,837, filed Oct. 29, 2002 (abandoned), which is a continuation of U.S. patent application Ser. No. 09/796,338, filed Feb. 28, 2001 (abandoned), which claims the benefit of U.S. Provisional Application Ser. No. 60/186,059, filed Feb. 29, 2000 (abandoned). U.S. patent application Ser. No. 10/407,079 is also a continuation-in-part of U.S. patent application Ser. No. 09/863,200, filed May 22, 2001 (abandoned), which claims the benefit of U.S. Provisional Application Ser. No. 60/206,019, filed May 22, 2000 (abandoned). The entire contents of each of the above-referenced patent applications are incorporated herein by this reference.

### BACKGROUND OF THE INVENTION

### G-Protein Coupled Receptors

G-protein coupled receptors (GPCRs) constitute a major class of proteins responsible for transducing a signal within a cell. GPCRs have three structural domains: an amino terminal extracellular domain, a transmembrane domain containing seven transmembrane segments, three extracellular loops, and three intracellular loops, and a carboxy terminal intracellular domain. Upon binding of a ligand to an extracellular portion of a GPCR, a signal is transduced within the cell that results in a change in a biological or physiological property of the cell. GPCRs, along with G-proteins and effectors (intracellular enzymes and channels modulated by G-proteins), are the components of a modular signaling system that connects the state of intracellular second messengers to extracellular inputs.

GPCR genes and gene-products are potential causative agents of disease (Spiegel et al., J. Clin. Invest. 92:1119-1125 (1993); McKusick et al., J. Med. Genet. 30:1-26 (1993)). Specific defects in the rhodopsin gene and the V2 vasopressin receptor gene have been shown to cause various forms of retinitis pigmentosum (Nathans et al., Annu. Rev. Genet. 26:403-424 (1992)), and nephrogenic diabetes insipidus (Holtzman et al., Hum. Mol. Genet. 2:1201-1204 (1993)). These receptors are of critical importance to both the central nervous system and peripheral physiological processes. Evolutionary analyses suggest that the ancestor of these proteins originally developed in concert with complex body plans and nervous systems.

The GPCR protein superfamily can be divided into five families: Family I, receptors typified by rhodopsin and the β2-adrenergic receptor and currently represented by over 200 unique members (Dohlman et al., Annu. Rev. Biochem. 60:653-688 (1991)); Family II, the parathyroid hormone/calcitonin/secretin receptor family (Juppner et al., Science 254:1024-1026 (1991); Lin et al., Science 254:1022-1024 (1991)); Family III, the metabotropic glutamate receptor family (Nakanishi, Science 258 597:603 (1992)); Family IV, the cAMP receptor family, important in the chemotaxis and development of D. discoideum (Klein et al., Science 241:1467-1472 (1988)); and Family V, the fungal mating pheromone receptors such as STE2 (Kurjan, Annu. Rev. Biochem. 61:1097-1129 (1992)).

There are also a small number of other proteins which present seven putative hydrophobic segments and appear to be unrelated to GPCRs; they have not been shown to couple to G-proteins. Drosophila expresses a photoreceptor-specific protein, bride of sevenless (boss), a seven-transmembrane-segment protein which has been extensively studied and does not show evidence of being a GPCR (Hart et al., Proc. Natl. Acad Sci. USA 90:5047-5051 (1993)). The gene frizzled (fz) in Drosophila is also thought to be a protein with seven transmembrane segments. Like boss, fz has not been shown to couple to G-proteins (Vinson et al., Nature 338:263-264 (1989)).

G proteins represent a family of heterotrimeric proteins composed of α, β, and γ subunits, that bind guanine nucleotides. These proteins are usually linked to cell surface receptors, e.g., receptors containing seven transmembrane segments. Following ligand binding to the GPCR, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cAMP (e.g., by activation of adenyl cyclase), diacylglycerol or inositol phosphates. Greater than 20 different types of α-subunits are known in humans. These subunits associate with a smaller pool of β and γ subunits. Examples of mammalian G proteins include Gi, Go, Gq, Gs and Gt. G proteins are described extensively in Lodish et al., Molecular Cell Biology, (Scientific American Books Inc., New York, N.Y., 1995), the contents of which are incorporated herein by reference. GPCRs, G proteins and G protein-linked effector and second messenger systems have been reviewed in The G-Protein Linked Receptor Fact Book, Watson et al., eds., Academic Press (1994).

GPCRs are a major target for drug action and development. Accordingly, it is valuable to the field of pharmaceutical development to identify and characterize previously unknown GPCRs. The present invention advances the state of the art by providing a previously unidentified human GPCR.

GPCRs have the ability to, for example: to interact or associate with a G protein; to bind a ligand such as a peptide, neuropeptide (e.g., FF, H2, glanin), platelet-activating factor; to be phosphorylated or dephosphorylated; the ability to affect cGMP or cAMP concentrations in the cell; to regulate, sense and/or transmit an extracellular signal into a cell, (for example, a heart cell, a bone cell (e.g., an osteoclast or an osteoblast), a hematopoietic cell, a neural cell); to interact with (e.g., binding to) an extracellular signal or a cell surface receptor; to mobilize an intracellular molecule that participates in a signal transduction pathway (e.g., adenylate cyclase or phosphatidylinositol 4,5-bisphosphate (PIP₂), inositol 1,4,5-triphosphate (IP₃)); to regulate polarization of the plasma membrane; to control production or secretion of molecules; to alter the structure of a cellular component; to modulate cell proliferation, e.g., synthesis of DNA; to modulate cell migration, cell differentiation; and cell survival; as well as many others. Accordingly, there exists a need to identify additional human GPCRs, for example, for use as disease markers and as targets for identifying various therapeutic modulators.

### SUMMARY OF THE INVENTION

The present invention is based, at least in part, on the discovery of novel nucleic acid molecules and proteins encoded by such nucleic acid molecules, referred to herein as “18636, 2466, 43238, 1983, 52881, 2398, 45449, 50289, 52872 or 26908”. The 18636, 2466, 43238, 1983, 52881, 2398, 45449, 50289, 52872 or 26908 nucleic acid and protein molecules of the present invention are useful as modulating agents in regulating a variety of cellular processes, e.g., including cell signalling. In particular, these nucleic acid molecules will be advantageous in the regulation of any cellular function, uncontrolled proliferation and differentiation, such as in cases of pain. Accordingly, in one aspect, this invention provides isolated nucleic acid molecules encoding 18636, 2466, 43238, 1983, 52881, 2398, 45449, 50289, 52872 or 26908 proteins or biologically active portions thereof, as well as nucleic acid fragments suitable as primers or hybridization probes for the detection of 18636, 2466, 43238, 1983, 52881, 2398, 45449, 50289, 52872 or 26908-encoding nucleic acids.

The nucleotide sequence of the cDNA encoding 18636, 2466, 43238, 1983, 52881, 2398, 45449, 50289, 52872 or 26908, and the amino acid sequence of 18636, 2466, 43238, 1983, 52881, 2398, 45449, 50289, 52872 or 26908 polypeptides are depicted in Table 1.

TABLE 1
Sequences of the invention

| Gene Name | cDNA | Protein | Coding Region |
|---|---|---|---|
| 18636 | SEQ ID NO: 1 | SEQ ID NO: 2 | SEQ ID NO: 3 |
| 2466 | SEQ ID NO: 29 | SEQ ID NO: 30 | SEQ ID NO: 31 |
| 43238 | SEQ ID NO: 69 | SEQ ID NO: 70 | SEQ ID NO: 71 |
| 1983 | SEQ ID NO: 77 | SEQ ID NO: 78 | SEQ ID NO: 79 |
| 52881 | SEQ ID NO: 80 | SEQ ID NO: 81 | SEQ ID NO: 82 |
| 2398 | SEQ ID NO: 83 | SEQ ID NO: 84 | SEQ ID NO: 85 |
| 45449 | SEQ ID NO: 86 | SEQ ID NO: 87 | SEQ ID NO: 88 |
| 50289 | SEQ ID NO: 89 | SEQ ID NO: 90 | SEQ ID NO: 91 |
| 52872 | SEQ ID NO: 92 | SEQ ID NO: 93 | SEQ ID NO: 94 |
| 26908 | SEQ ID NO: 103 | SEQ ID NO: 104 | SEQ ID NO: 105 |

Accordingly, in one aspect, the invention features a nucleic acid molecule which encodes a 18636, 2466, 43238, 1983, 52881, 2398, 45449, 50289, 52872 or 26908 protein or polypeptide, e.g., a biologically active portion of the 18636, 2466, 43238, 1983, 52881, 2398, 45449, 50289, 52872 or 26908 protein. In a preferred embodiment, the isolated nucleic acid molecule encodes a polypeptide having the amino acid sequence of SEQ ID NO:2, 30, 70, 78, 81, 84, 87, 90, 93 or 104. In other embodiments, the invention provides isolated 18636, 2466, 43238, 1983, 52881, 2398, 45449, 50289, 52872 or 26908 nucleic acid molecules having the nucleotide sequence shown in SEQ ID NO:1, 3, 29, 31, 69, 71, 77, 79, 80, 82, 83, 85, 86, 88, 89, 91, 92, 94, 103 or 105. In still other embodiments, the invention provides nucleic acid molecules that are substantially identical (e.g., naturally occurring allelic variants) to the nucleotide sequence shown in SEQ ID NO:1, 3, 29, 31, 69, 71, 77, 79, 80, 82, 83, 85, 86, 88, 89, 91, 92, 94, 103 or 105. In other embodiments, the invention provides a nucleic acid molecule which hybridizes under a stringent hybridization condition as described herein to a nucleic acid molecule comprising the nucleotide sequence of SEQ ID NO:1, 3, 29, 31, 69, 71, 77, 79, 80, 82, 83, 85, 86, 88, 89, 91, 92, 94, 103 or 105, wherein the nucleic acid encodes a full length 18636, 2466, 43238, 1983, 52881, 2398, 45449, 50289, 52872 or 26908 protein or an active fragment thereof.

In a related aspect, the invention further provides nucleic acid constructs which include a 18636, 2466, 43238, 1983, 52881, 2398, 45449, 50289, 52872 or 26908 nucleic acid molecule described herein. In certain embodiments, the nucleic acid molecules of the invention are operatively linked to native or heterologous regulatory sequences. Also included are vectors and host cells containing the 18636, 2466, 43238, 1983, 52881, 2398, 45449, 50289, 52872 or 26908 nucleic acid molecules of the invention e.g., vectors and host cells suitable for producing polypeptides.

In another related aspect, the invention provides nucleic acid fragments suitable as primers or hybridization probes for the detection of 18636, 2466, 43238, 1983, 52881, 2398, 45449, 50289, 52872 or 26908-encoding nucleic acids.

In still another related aspect, isolated nucleic acid molecules that are antisense to a 18636, 2466, 43238, 1983, 52881, 2398, 45449, 50289, 52872 or 26908 encoding nucleic acid molecule are provided.

In another aspect, the invention features 18636, 2466, 43238, 1983, 52881, 2398, 45449, 50289, 52872 or 26908 polypeptides, and biologically active or antigenic fragments thereof that are useful, e.g., as reagents or targets in assays applicable to treatment and diagnosis of 18636, 2466, 43238, 1983, 52881, 2398, 45449, 50289, 52872 or 26908-associated disorders. In another embodiment, the invention provides 18636, 2466, 43238, 1983, 52881, 2398, 45449, 50289, 52872 or 26908 polypeptides having a 18636, 2466, 43238, 1983, 52881, 2398, 45449, 50289, 52872 or 26908 activity.

In other embodiments, the invention provides 18636, 2466, 43238, 1983, 52881, 2398, 45449, 50289, 52872 or 26908 polypeptides, e.g., a 18636, 2466, 43238, 1983, 52881, 2398, 45449, 50289, 52872 or 26908 polypeptide having the amino acid sequence shown in SEQ ID NO:2, 30, 70, 78, 81, 84, 87, 90, 93 or 104; an amino acid sequence that is substantially identical to the amino acid sequence shown in SEQ ID NO:2, 30, 70, 78, 81, 84, 87, 90, 93 or 104; or an amino acid sequence encoded by a nucleic acid molecule having a nucleotide sequence which hybridizes under a stringent hybridization condition as described herein to a nucleic acid molecule comprising the nucleotide sequence of SEQ ID NO:1, 3, 29, 31, 69, 71, 77, 79, 80, 82, 83, 85, 86, 88, 89, 91, 92, 94, 103 or 105, wherein the nucleic acid encodes a full length 18636, 2466, 43238, 1983, 52881, 2398, 45449, 50289, 52872 or 26908 protein or an active fragment thereof.

In a related aspect, the invention further provides nucleic acid constructs which include a 18636, 2466, 43238, 1983, 52881, 2398, 45449, 50289, 52872 or 26908 nucleic acid molecule described herein.

In a related aspect, the invention provides 18636, 2466, 43238, 1983, 52881, 2398, 45449, 50289, 52872 or 26908 polypeptides or fragments operatively linked to non-18636, 2466, 43238, 1983, 52881, 2398, 45449, 50289, 52872 or 26908 polypeptides to form fusion proteins.

In another aspect, the invention features antibodies and antigen-binding fragments thereof, that react with, or more preferably specifically or selectively bind 18636, 2466, 43238, 1983, 52881, 2398, 45449, 50289, 52872 or 26908 polypeptides.

In another aspect, the invention provides methods of screening for compounds that modulate the expression or activity of the 18636, 2466, 43238, 1983, 52881, 2398, 45449, 50289, 52872 or 26908 polypeptides or nucleic acids.

In still another aspect, the invention provides a process for modulating 18636, 2466, 43238, 1983, 52881, 2398, 45449, 50289, 52872 or 26908 polypeptide or nucleic acid expression or activity, e.g., using the compounds identified in the screens described herein. In certain embodiments, the methods involve treatment of conditions related to aberrant activity or expression of the 18636, 2466, 43238, 1983, 52881, 2398, 45449, 50289, 52872 or 26908 polypeptides or nucleic acids, such as conditions or disorders involving aberrant or deficient 18636, 2466, 43238, 1983, 52881, 2398, 45449, 50289, 52872 or 26908 expression. Examples of such disorders include, but are not limited to cellular proliferative and/or differentiative disorders, brain disorders, blood vessel disorders, platelet disorders, kidney or renal disorders, hematopoeitic disorders, prostate disorders, testicular disorders, skin disorders, eye or opthalmological disorders, disorders associated with bone metabolism, immune e.g., inflammatory disorders, cardiovascular disorders, endothelial cell disorders, liver disorders, viral diseases, pain, metabolic disorders, hormonal disorders, neurological or CNS disorders, hematological disorders, intestinal disorders, respiratory disorders, fibrotic disorders or angiogenic disorders.

The invention also provides assays for determining the activity of or the presence or absence of 18636, 2466, 43238, 1983, 52881, 2398, 45449, 50289, 52872 or 26908 polypeptides or nucleic acid molecules in a biological sample, including for disease diagnosis.

In a further aspect, the invention provides assays for determining the presence or absence of a genetic alteration in a 18636, 2466, 43238, 1983, 52881, 2398, 45449, 50289, 52872 or 26908 polypeptide or nucleic acid molecule, including for disease diagnosis.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 18636, 2466, 43238, 1983, 52881, 2398, 45449, 50289, 52872 or 26908 molecule. In one embodiment, the capture probe is a nucleic acid, e.g., a probe complementary to a 18636, 2466, 43238, 1983, 52881, 2398, 45449, 50289, 52872 or 26908 nucleic acid sequence. In another embodiment, the capture probe is a polypeptide, e.g., an antibody specific for 18636, 2466, 43238, 1983, 52881, 2398, 45449, 50289, 52872 or 26908 polypeptides. Also featured is a method of analyzing a sample by contacting the sample to the aforementioned array and detecting binding of the sample to the array.

Other features and advantages of the invention will be apparent from the following detailed description, and from the claims.

### DETAILED DESCRIPTION OF THE INVENTION

### Human 18636

The present invention is based, in part, on the discovery of a novel G-protein-coupled receptor, referred to herein as “18636”.

The human 18636 receptor protein sequence (SEQ ID NO:1), which is approximately 1797 nucleotides long including untranslated regions, contains a predicted methionine-initiated coding sequence of about 1068 nucleotides, not including the termination codon (nucleotides 437-1504 of SEQ ID NO:1; 1-1068 of SEQ ID NO:3). The coding sequence encodes a 356 amino acid protein (SEQ ID NO:2).

The human 18636 protein of SEQ ID NO:2 includes an amino-terminal hydrophobic amino acid sequence, consistent with a signal sequence, of about 14 amino acids (from amino acid 1 to about amino acid 14 of SEQ ID NO:2, PSORT, Nakai and Kanehisa (1992) Genomics 14:897-911), which upon cleavage results in the production of a mature protein form.). This mature protein form is approximately 342 amino acid residues in length (from about amino acid 15 to amino acid 356 of SEQ ID NO:2). The sequence of the mature form of 18636 is shown in SEQ ID NO:4.

Human 18636 receptor protein contains the following regions or other structural features (for general information regarding PFAM identifiers, PS prefix and PF prefix domain identification numbers, refer to Sonnhammer et al. (1997) Protein 28:405-420).

Amino acids 1 to 45 or 15 to 45 of 18636 (SEQ ID NO:2) are predicted to be the amino terminal extracellular domain, amino acids 45-318 of SEQ ID NO:2 the transmembrane domain, and amino acids 319-356 of SEQ ID NO:2 the carboxy terminal intracellular domain. The transmembrane domain contains seven transmembrane segments, three extracellular loops and three intracellular loops. The transmembrane segments are found from about amino acid 45 to about amino acid 69, from about amino acid 79 to about amino acid 103, from about amino acid 116 to about amino acid 137, from about amino acid 160 to about amino acid 177, from about amino acid 215 to about amino acid 239, from about amino acid 257 to about amino acid 281, and from about 294 to about amino acid 318 of SEQ ID NO:2. Within the region spanning the entire transmembrane domain are three intracellular and three extracellular loops. The three intracellular loops are found from about amino acid 69 to about amino acid 79, from about amino acid 137 to about amino acid 160, and from about amino acid 239 to about amino acid 257 of SEQ ID NO:2. The three extracellular loops are found from about amino acid 103 to about amino acid 116, from about amino acid 177 to about amino acid 215, and from about amino acid 281 to about amino acid 294 of SEQ ID NO:2.

A comparison of the 18636 receptor protein against the Prosite database of protein patterns identifies amino acids corresponding to specific functional sites: N-glycosylation sites are found at about amino acids 20-33 (NSSG; SEQ ID NO:18), 199-202 (NETA; SEQ ID NO:19), and 308-311 (NSSL; SEQ ID NO:20) of SEQ ID NO:2. Four protein kinase C (PKC) phosphorylation site (Prosite PS00005) are found at about amino acids 12-14 (SDR), 26-28 (SRK), 41-43 (SLR), and 181-183 (TTR) of SEQ ID NO:2. A casein kinase II phosphorylation (Prosite PS00006) is found at about amino acids 197-200 (SDNE; SEQ ID NO:21) of SEQ ID NO:2. Five N-myristoylation sites (Prosite PS00008) are found at about amino acids 3-8 (GVSEGT; SEQ ID NO:22), 57-62 (GVLGNG; SEQ ID NO:23), 187-192 (GCTHCY; SEQ ID NO:24), 208-213 (GVVEGH; SEQ ID NO:25), and 305-310 (GCVNSS; SEQ ID NO:26) of SEQ ID NO:2. In addition, a G protein coupled receptor signature sequence (PS00237) is found at about amino acids 127-143 (ASNCLLVFISVDRCISV; SEQ ID NO:27) of SEQ ID NO:2.

Based on a search of complete domains in PFAM, the 18636 receptor protein is a member of the rhodopsin family of seven transmembrane receptor proteins (7tm_{—}1). A search of the ProDom database reveals that the 18636 receptor protein is very similar to GPR32, a G protein coupled receptor. In a BLAST alignment, the human 18636 G protein coupled receptor domain aligns with a consensus amino acid sequence of a domain derived from the ProDomain database (“Receptor protein-coupled G-protein GPR 32 glycoprotein transmembrane;” No. PD164880, PD169336, PD164664; ProDomain Release 2001.1). Amino acid residues 1 to 60 and 61 to 78 of the 78 amino acid PD164880 consensus sequence (SEQ ID NO:6) aligns with amino acid residues 279 to 338 and 339 to 356 of the receptor protein-coupled G-protein GPR glycoprotein transmembrane domain of human 18636 (SEQ ID NO:2). Amino acid residues 1 to 73 of the 73 amino acid PD169336 consensus sequence (SEQ ID NO:7) aligns with amino acid residues 1 to 73 of the receptor protein-coupled G-protein GPR glycoprotein transmembrane domain of human 18636 (SEQ ID NO:2). Amino acid residues 1 to 42 of the 42 amino acid PD164664 consensus sequence (SEQ ID NO:8) aligns with amino acid residues 181 to 222 of the receptor protein-coupled G-protein GPR glycoprotein transmembrane domain of human 18636 (SEQ ID NO:2).

Finally, searches performed using the “ProDom protein domain database” identified the following homologous domains within 18636: 1) a “receptor coupled G-protein transmembrane glycoprotein phosphorylation lipoprotein palmitate family multigene” domain (SEQ ID NO:9) is 31% identical over amino acids 74 to 179 of SEQ ID NO:2; 2) a “receptor LTB4” domain (SEQ ID NO:10) is 29% identical over amino acids 224 to 351 of SEQ ID NO:2; 3) a “receptor coupled G-protein transmembrane glycoprotein chemokine type lipoprotein palmitate” domain (SEQ ID NO:11) is 37% identical over amino acids 215 to 278 of SEQ ID NO:2; 4) a “receptor GPR25 protein-coupled G-protein transmembrane” domain (SEQ ID NO:12) is 31% identical over amino acids 222 to 337 of SEQ ID NO:2; 5) a “T14B1.2” domain (SEQ ID NO:13-14) is 26% and 30% identical over amino acids 90 to 166 and 289 to 317 of SEQ ID NO:2 in two HSPs, respectively; 6) a “receptor coupled G-protein homolog protein-coupled transmembrane” domain (SEQ ID NO:15) is 25% identical over amino acids 81 to 206 of SEQ ID NO:2; and 7) a “CG12610” domain (SEQ ID NO:16) is 22% identical over amino acids 71 to 148 of SEQ ID NO:2.

In a GAP alignment of human 18636 with human probable G protein-coupled receptor GPR32 (Accession number in Genbank 075388), amino acids 437 to 1504 of human 18636 (SEQ ID NO:2) aligns with amino acids 1 to 356 of 075388 (SEQ ID NO:17). GAP alignments use a matrix made by matblas from blosum62.iij.

A hydropathy plot of human 18636 receptor protein was performed. Polypeptides of the invention include fragments which include: all or part of a hydrophobic sequence, e.g., the sequence from about amino acid 43 to 95, from about 110 to 145, from about 155 to 175, from about 195 to 240, from about 250 to 275, from about 290 to 305, and from about 310 to 320 of SEQ ID NO:2; all or part of a hydrophilic sequence, e.g., the sequence from about amino acid 1 to 35, from about 145 to 155, from about 170 to 185, from about 187 to 198, from about 245 to 255, and from about 310 to 320 of SEQ ID NO:2; a sequence which includes a Cys, or a glycosylation site.

The 18636 receptor protein contains a significant number of structural characteristics in common with members of the G-protein coupled receptor family, for example, an amino terminal extracellular domain, a transmembrane domain, and a carboxy terminal intracellular domain as well as a G protein coupled receptor signature sequence.

As used herein, the term “G protein coupled receptor” includes a protein or polypeptide which is capable of binding a ligand such as a chemokine, transducing the signal through a membrane, and interacting with a G protein. The G protein coupled receptor may be a member of a subfamily of G protein coupled receptors. Particularly interesting subfamilies include the rhodopsin subfamily and the chemokine R family.

Members of the G protein coupled receptor family of proteins are characterized by seven transmembrane domains, an N-terminal extracellular domain, three extracellular loops, three intracellular loops, and a C-terminal intracellular domain which may interact with a G protein. An alignment of the 18636 protein with G protein coupled receptor GPR32 demonstrates about 100% sequence identity between the two sequences (as calculated in matblas from the blosum62.iij matrix).

A 18636 polypeptide can include a “seven transmembrane domain” or regions homologous with a “seven transmembrane domain”. A 18636 polypeptide can further include a “cytoplasmic domain” or regions homologous with a “cytoplasmin domain,” and can further include an “extracellular domain” or regions homologous with an “extracellular domain”. A 18636 polypeptide can include a domain which binds a ligand such as a chemokine and/or may include a domain which interacts with a G protein.

As used herein, the term “seven transmembrane domain” includes an amino acid sequence of about 60 to 316 amino acid residues in length and having a bit score for the alignment of the sequence to the seven transmembrane domain (HMM) of at least 100. Preferably a seven transmembrane domain mediates a signal transduction function such as transducing a signal of ligand binding through the membrane to a G protein. Preferably, a seven transmembrane domain includes at least about 100 to 300 amino acids, more preferably about 200 to 300 amino acid residues, or about 225 to 275 amino acids and has a bit score for the alignment of the sequence to the seven transmembrane domain (HMM) of at least 100, 110, 120, 130, or greater. The seven transmembrane domain can include G-protein coupled receptor signature sequence PS00237 ([GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R—[FYWCSH]-x(2)-[LIVM]; SEQ ID NO:28), or sequences homologous thereto. In the above conserved signature sequence, and other motifs or signature sequences described herein, the standard IUPAC one-letter code for the amino acids is used. Each element in the pattern is separated by a dash (-); square brackets ([ ]) indicate the particular residues that are accepted at that position; x indicates that any residue is accepted at that position; and numbers in parentheses (( )) indicate the number of residues represented by the accompanying amino acid. The signature sequence is located as part of the third transmembrane domain extending into an intracellular loop of human 18636 polypeptide and which corresponds to about amino acids 127 to 143 of SEQ ID NO:2. The seven transmembrane receptor (rhodopsin family) domain (HMM) has been assigned the PFAM Accession Number PFAM 7tm_{—}1. Additionally, as used herein, the “G protein coupled receptor domain” is a portion of the human 18636 protein which is homologous, e.g., at least about 79%, 80%, 81%, 82%, 83%, 84%, 85%, 86%, 87%, 88%, 89%, 90%, 91%, 92%, 93%, 94%, 95% or 98% identical to either or both of the following ProDom family “receptor protein-coupled G-protein GPR32 glycoprotein transmembrane” domain (ProDomain Release 2001.1). An alignment of the G protein coupled receptor domain (amino acids 1 to 73, 181 to 222, 279 to 338, and 339 to 356 of SEQ ID NO:2) of human 18636 with PD169336, PD164664, and PD164880, SEQ ID NO:6-8, derived from a BLAST search model shows 95% identity (as calculated in ProDomain from the blosum62 matrix). The seven transmembrane receptor (rhodopsin family) domain (amino acids 60 to 316 of SEQ ID NO:2) of human 18636 aligns with the Pfam 7tm_{—}1 consensus amino acid sequence (SEQ ID NO:5) derived from a hidden Markov model.

In a preferred embodiment, a 18636 polypeptide or protein has a “seven transmembrane domain” or a region which includes at least about 100 to 400 more preferably about 200 to 400 or 250 to 400 amino acid residues and has at least about 60%, 70% 80% 90% 95%, 99%, or 100% homology with a “seven transmembrane domain,” e.g., the seven transmembrane domain of human 18636 (e.g., residues 60 to 316 of SEQ ID NO:2).

To identify the presence of a “seven transmembrane receptor (rhodopsin family)” domain in a 18636 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “7 transmembrane receptor (rhodopsin family) domain” domain in the amino acid sequence of human 18636 at about residues 60 to 316 of SEQ ID NO:2.

For further identification of the presence of a “G protein coupled receptor” domain in a 18636 protein sequence, and make the determination that a polypeptide or protein of interest has a particular profile, the amino acid sequence of the protein can be searched against a database of domains, e.g., the ProDom database (Corpet et al. (1999), Nucl. Acids Res. 27:263-267). The ProDom protein domain database consists of an automatic compilation of homologous domains. Current versions of ProDom are built using recursive PSI-BLAST searches (Altschul et al. (1997) Nucleic Acids Res. 25:3389-3402; Gouzy et al. (1999) Computers and Chemistry 23:333-340) of the SWISS-PROT 38 and TREMBL protein databases. The database automatically generates a consensus sequence for each domain. A BLAST search was performed against the HMM database resulting in the identification of a “receptor protein-coupled G-protein GPR32 glycoprotein transmembrane” domain in the amino acid sequence of human 18636 at about residues 1 to 73, 181 to 222, and 279 to 356 of SEQ ID NO:2.

A 18636 polypeptide can include at least one, two, three, four, five, six, preferably seven “transmembrane domains” or regions homologous with a “transmembrane domain”. As used herein, the term “transmembrane domain” includes an amino acid sequence of about 10 to 40 amino acid residues in length and spans the plasma membrane. Transmembrane domains are rich in hydrophobic residues, e.g., at least 50%, 60%, 70%, 80%, 90%, 95% or more of the amino acids of a transmembrane domain are hydrophobic, e.g., leucines, isoleucines, tyrosines, or tryptophans. Transmembrane domains typically have alpha-helical structures and are described in, for example, Zagotta et al., (1996) Annual Rev. Neurosci. 19:235-263, the contents of which are incorporated herein by reference. The transmembrane domains of human 18636 is located at about residues 45 to 69, 79 to 103, 116 to 137, 160 to 177, 215 to 239, 257 to 281, and 294 to 318 of SEQ ID NO:2.

In a preferred embodiment, a 18636 polypeptide or protein has at least one, two, three, four, five, six, preferably seven “transmembrane domains” or regions which include at least about 12 to 35 more preferably about 14 to 30 or 15 to 25 amino acid residues and has at least about 60%, 70% 80% 90% 95%, 99%, or 100% homology with a “transmembrane domain,” e.g., the transmembrane domains of human 18636 (e.g., residues 45 to 69, 79 to 103, 116 to 137, 160 to 177, 215 to 239, 257 to 281, and 294 to 318 of SEQ ID NO:2). The transmembrane domain of human 18636 can be visualized in a hydropathy plot as regions of about 15 to 25 amino acids where the hydropathy trace is mostly above the horizontal line.

To identify the presence of a “transmembrane” domain in a 18636 protein sequence, and make the determination that a polypeptide or protein of interest has a particular profile, the amino acid sequence of the protein can be analyzed by a transmembrane prediction method that predicts the secondary structure and topology of integral membrane proteins based on the recognition of topological models (MEMSAT, Jones et al., (1994) Biochemistry 33:3038-3049).

A 18636 polypeptide can include at least one, two, three, four, five, six, seven, preferably eight “non-transmembrane regions.” As used herein, the term “non-transmembrane region” includes an amino acid sequence not identified as a transmembrane domain. The non-transmembrane regions in 18636 are located at about amino acids 1 to 45, 69 to 79, 103 to 116, 137 to 160, 177 to 215, 239 to 257, 281 to 294, and 318 to 356 of SEQ ID NO:2. Non-transmembrane regions can be extracellular, intracellular, cytoplasmic, or lumenal, and the non-transmembrane regions may play a role in signal transduction such as binding a ligand such as a chemokine, interaction with a G-protein, etc.

The non-transmembrane regions of 18636 receptor protein include at least one, two, three, preferably four extra-cellular regions. When located at the N-terminus, the extracellular region is referred to herein as the “N-terminal extracellular domain.” As used herein, an “N-terminal cytoplasmic domain” includes an amino acid sequence having about 1 to 100, preferably about 1 to 75, more preferably about 1 to 50, or even more preferably about 1 to 45 amino acid residues in length, is located outside of a cell and may function to aid in binding of the receptor's ligand. The C-terminal amino acid residue of an “N-terminal cytoplasmic domain” is adjacent to an N-terminal amino acid residue of a transmembrane domain in a 18636 protein. For example, an N-terminal cytoplasmic domain is located at about amino acid residues 1 to 45 of SEQ ID NO:2.

In a preferred embodiment, a 18636 polypeptide or protein has an N-terminal extracellular domain or a region which includes about 1 to 100, preferably about 1 to 75, and more preferably about 1 to 50 amino acid residues and has at least about 60%, 70% 80% 90% 95%, 99%, or 100% homology with an “N-terminal extracellular domain,” e.g., the N-terminal extracellular domain of human 18636 (e.g., residues 1 to 45 of SEQ ID NO:2) with or without the N-terminal signal sequence removed.

In another embodiment, a 18636 cytoplasmic region includes at least one, two, preferably three cytoplasmic loops. As used herein, the term “loop” includes an amino acid sequence which is not included within a phospholipid membrane, having a length of at least about 4, preferably about 5 to 50, more preferably about 6 to 40 amino acid residues, and has an amino acid sequence that connects two transmembrane domains within a protein or polypeptide. Accordingly, the N-terminal amino acid of a loop is adjacent to a C-terminal amino acid of a transmembrane domain in a 18636 molecule, and the C-terminal amino acid of a loop is adjacent to an N-terminal amino acid of a transmembrane domain in a 18636 molecule. As used herein, a “cytoplasmic loop” includes a loop located inside of a cell or within the cytoplasm of a cell. For example, a “cytoplasmic loop” can be found at about amino acid residues 69 to 79, 137 to 160, and 239 to 257 of SEQ ID NO:2.

In a preferred embodiment, a 18636 polypeptide or protein has a cytoplasmic loop or a region which includes at least about 4, preferably about 5 to 20, and more preferably about 6 to 18 amino acid residues and has at least about 60%, 70% 80% 90% 95%,99%, or 100% homology with a “cytoplasmic loop,” e.g., a cytoplasmic loop of human 18636 (e.g., residues 69 to 79, 137 to 160, and 239 to 257 of SEQ ID NO:2).

In another embodiment, a 18636 non-transmembrane region includes at least one, two, preferably three non-cytoplasmic loops. As used herein, a “non-cytoplasmic loop” includes a loop located outside of a cell or within an intracellular organelle. Non-cytoplasmic loops include extracellular domains (i.e., outside of the cell) and intracellular domains (i.e., within the cell). When referring to membrane-bound proteins found in intracellular organelles (e.g., mitochondria, endoplasmic reticulum, peroxisomes microsomes, vesicles, endosomes, and lysosomes), non-cytoplasmic loops include those domains of the protein that reside in the lumen of the organelle or the matrix or the intermembrane space. For example, a “non-cytoplasmic loop” can be found at about amino acid residues 103 to 116, 177 to 215, and 281 to 294 of SEQ ID NO:2.

In a preferred embodiment, a 18636 polypeptide or protein has at least one non-cytoplasmic loop or a region which includes at least about 4, preferably about 5 to 50, more preferably about 6 to 40 amino acid residues and has at least about 60%, 70% 80% 90% 95%, 99%, or 100% homology with a “non-cytoplasmic loop,” e.g., at least one non-cytoplasmic loop of human 18636 (e.g., residues 103 to 116, 177 to 215, and 281 to 294 of SEQ ID NO:2).

In another embodiment, a cytoplasmic region of a 18636 protein can include the C-terminus and can be a “C-terminal cytoplasmic domain,” also referred to herein as a “C-terminal cytoplasmic tail.” As used herein, a “C-terminal cytoplasmic domain” includes an amino acid sequence having a length of at least about 10, preferably about 10 to 100, more preferably about 20 to 50 amino acid residues, is located inside of a cell or within the cytoplasm of a cell and has the function of interacting with intracellular proteins such as G proteins in order to transduce a signal from the extracellular milieu to the intracellular space. The N-terminal amino acid residue of a “C-terminal cytoplasmic domain” is adjacent to a C-terminal amino acid residue of a transmembrane domain in a 18636 protein. For example, a C-terminal cytoplasmic domain is located at about amino acid residues 318 to 356 of SEQ ID NO:2.

In a preferred embodiment, a 18636 polypeptide or protein has a C-terminal cytoplasmic domain or a region which includes at least about 10, preferably about 10 to 100, and more preferably about 20 to 50 amino acid residues and has at least about 60%, 70% 80% 90% 95%, 99%, or 100% homology with a “C-terminal cytoplasmic domain,” e.g., the C-terminal cytoplasmic domain of human 18636 (e.g., residues 318 to 356 of SEQ ID NO:2).

A 18636 family member can include at least one seven transmembrane domain. A 18636 family member can include at least one G-protein coupled receptor signature sequence. Furthermore, a 18636 family member can include at least one, two, three, preferably four protein kinase C phosphorylation sites (Prosite PS00005); at least one casein kinase II phosphorylation sites (Prosite PS00006); at least one, two, preferably three N-glycosylation site (Prosite PS00001); and at least one, two, three, four, preferably five N-myristoylation sites (Prosite PS00008).

As the 18636 polypeptides of the invention can modulate 18636-mediated activities, they can be useful for developing novel diagnostic and therapeutic agents for G protein coupled receptor-associated or other 18636-associated disorders, as described below.

The 18636 G protein coupled receptor is in the chemokine R family of GPCR. By agonizing this receptor protein, one can maintain a growth factor signaling pathway and cause cell's expressing this receptor to differentiate and/or proliferate. Agonist of 18636 therefore may be useful in the treatment of diseases in which cellular proliferation is needed such as, but not limited to, anemia, wound healing, immunodeficiency syndromes, etc. As used herein, a “G protein coupled receptor-associated activity” includes an activity which involves cellular proliferation and/or differentiation. Members of the family can play a role in neoplastic diseases, physiological differentiation, physiological growth, etc.

As used herein, a “18636 activity”, “biological activity of 18636” or “functional activity of 18636”, refers to an activity exerted by a 18636 protein, polypeptide or nucleic acid molecule on e.g., a 18636-responsive cell or on a 18636 substrate, e.g., a protein substrate, as determined in vivo or in vitro. In one embodiment, a 18636 activity is a direct activity, such as an association with a 18636 target molecule. A “target molecule” or “binding partner” is a molecule with which a 18636 protein binds or interacts in nature. In an exemplary embodiment, 18636 is a receptor, e.g., G-protein coupled receptor, and thus binds to or interacts in nature with a molecule, e.g., ligand, peptide, chemokine.

A 18636 activity can also be an indirect activity, e.g., a cellular signaling activity mediated by interaction of the 18636 protein with a 18636 receptor. Based on the above-described sequence structures and similarities to molecules of known function, the 18636 molecules of the present invention can have similar biological activities as G protein coupled receptor family members. For example, the 18636 proteins of the present invention can have one or more of the following activities: (1) the ability to bind a ligand such as a peptide, small molecule, chemokine, etc.; (2) the ability to transduce a signal through a membrane; (3) the ability to interaction with cellular proteins such as G proteins; (4) the ability to modulate second messenger levels such as cAMP and cGMP; and (5) the ability to cause a cell to divide and/or differentiate.

The 18636 molecules of the invention can modulate the activities of cells in tissues where they are expressed. For example, TaqMan analysis shows 18636 mRNA is expressed in bone marrow, eythrocyte precursors, platelet precursors, neutrophil precursors, myeloid tissues, erythroid tissues, proerythroblasts, hematopoietic cells, brain, fetal liver, peripheral blood, smooth muscle cells, stem cells, pluripotent stem cells, and progenitor cells. Accordingly, the 18636 molecules of the invention can act as therapeutic or diagnostic agents for hematological, cardiovascular, or neurological disorders.

The 18636 molecules can be used to treat hematological disorders in part because the 18636 mRNA is expressed in the bone marrow and other hematopoietic tissues. The 18636 mRNA has been found to be expressed in early progenitor cells (CD34+) and committed erythroid cells. Thus, agonizing the 18636 receptor is useful in the treatment of anemia and other diseases associated with decreased proliferation of hematopoietic cells.

Thus, the 18636 molecules can act as novel diagnostic targets and therapeutic agents for controlling one or more hematological disorders or other G protein coupled receptor disorders. As used herein, “G protein coupled receptor disorders” are diseases or disorders whose pathogenesis is caused by, is related to, or is associated with aberrant or deficient G protein coupled receptor protein function or expression. G protein coupled receptor-associated or other 18636-associated disorders, include but are not limited to, cancers, neoplasms, hematological disorder including cancers of the blood, anemias, disorders of hematopoiesis, cellular proliferative and/or differentiative disorders, disorders associated with bone metabolism, immune e.g., inflammatory, disorders, cardiovascular disorders, endothelial cell disorders, liver disorders, viral diseases, pain or metabolic disorders.

The 18636 receptor protein molecules can be used to treat hematological disorders in part because G protein coupled receptor family members are found in the hematopoietic tissues. These disorders may be proliferative disorders resulting from increased hematopoiesis, or may be anemias or immunodeficiency disorders resulting from decreased hematopoiesis.

The 18636 receptor protein molecules of the invention can be used to monitor, treat and/or diagnose a variety of hematological disorders.

The 18636 receptor protein molecules of the invention can be used to monitor, treat and/or diagnose a variety of proliferative disorders. Such disorders include hematopoietic neoplastic disorders.

Aberrant expression and/or activity of 18636 receptor protein molecules can mediate disorders associated with bone metabolism.

The 18636 nucleic acid and protein of the invention can be used to treat and/or diagnose a variety of immune, e.g., inflammatory, (e.g. respiratory inflammatory) disorders.

Additionally, 18636 molecules can play an important role in the etiology of certain viral diseases, including but not limited to Hepatitis B, Hepatitis C and Herpes Simplex Virus (HSV). Modulators of 18636 activity could be used to control/viral diseases. The modulators can be used in the treatment and/or diagnosis of viral infected tissue or virus-associated tissue fibrosis, especially liver and liver fibrosis. Also, 18636 modulators can be used in the treatment and/or diagnosis of virus-associated carcinoma, especially hepatocellular cancer.

Additionally, 18636 can play an important role in the regulation of metabolism or pain disorders.

### Gene Expression Analysis of 18636

Human 18636 expression was measured by TaqMan® quantitative PCR (Perkin Elmer Applied Biosystems) in cDNA prepared from a variety of normal and diseased (e.g., cancerous) human tissues or cell lines. The results indicate significant 18636 expression in hematopoietic cells, brain, fetal liver, progenitor cells (CD34+), cord blood, erythrocytes, bone marrow, erythroid cell lines, and coronary smooth muscle cells (see Tables 2-5).

TABLE 2
18636 Heme Taqman-Ph1 Expression Data

| Tissue | Expression |
|---|---|
| Heart PT-262 | 0.0 |
| Brain Md 400 | 0.4 |
| Liver NDR 379 | 0.0 |
| Lung Chl 330 | 0.1 |
| Spleen 380 | 0.0 |
| Fetal Liver Bwh 054 | 0.5 |
| Kidney 27 | 0.0 |
| CD3 4 hr resting LF 137 | 0.0 |
| CD3 4 hr activated LF 137 | 0.0 |
| CD3 24 hr resting LF 137 | 0.0 |
| CD3 24 hr activated FL 137 | 0.0 |
| CD4 24 hr resting LF141 | 0.0 |
| CD4 24 hr activated LF137 | 0.0 |
| CD8 4 hr resting LF137 | 0.0 |
| CD8 4 hr activated LF137 | 0.0 |
| CD8 24 hr activating LF137 | 0.1 |
| CD19LF 131 | 0.8 |
| CD14 LF 129 | 0.0 |
| Grans A LF138 | 0.0 |
| mBM MNC LP7 | 0.0 |
| mBM CD34+ LP92 | 4.8 |
| mPB CD34+ LP 162 | 1.0 |
| BM CD34+ LF 89 | 0.3 |
| Cord Blood CD34+ LF 101 | 1.5 |
| BM GPA high LF85 | 0.2 |
| Pooled Neut D6 LF78, 71, 141 | 0.0 |
| Pooled Neut D12 LF137, 141 | 0.0 |
| Pooled Eryth D12 LF23- | 0.5 |
| 8, 139, 170 | |
| Pooled Mega LF 140, 170, LP | 0.0 |
| 125, 128, 127 | |
| BM CD15+/CD14− LP32 | 0.0 |
| mBMCD15+/11b−CD14−LF120 | 0.0 |
| K502 | 0.2 |
| HL60 | 0.1 |
| Mock Dt PBL | 0.0 |
| Mock D3 PBL | 0.0 |
| Mock D5 PBL | 0.0 |
| HIV-1 Dt PBL | 0.0 |
| HIV-1 D3 PBL | 0.0 |
| HIV-1 D5 PBL | 0.0 |

TABLE 3
18636 Heme Taqman-Ph2a Expression Data

| Tissue | Expression |
|---|---|
| Lung CHT330 | 0.01 |
| Heart PT262 | 0.01 |
| Spleen 38 | 0.01 |
| Kidney 27 | 0.00 |
| Liver NDR 379 | 0.00 |
| Fetal Liver BWH 054 | 0.02 |
| Brain Mcl 400 | 0.11 |
| mBM MNO LP7 | 0.00 |
| mBM CD34+ LP92 | 2.34 |
| mPB CD34+ LP350 | 0.14 |
| mPB 412 LP162 | 0.17 |
| BM CD34− LF154 | 0.16 |
| BM CD34+ LF81 | 0.04 |
| Cord Blood CD34+ MF | 0.09 |
| Cord Blood CD34+ LF101 | 0.42 |
| BM GPA High LF 149 | 0.47 |
| BM GPA High LF 69 | 0.35 |
| BM GPA High LF 85 | 0.06 |
| BM GPA LP 151 | 0.03 |
| BM GPA LoLF149 | 0.12 |
| mPB CD41+/CD4− LF148 | 0.01 |
| mPB CD41+/CD4− LF134 | 0.04 |
| mPB CD41+/CD4− LF150 | 0.01 |
| BM CD41+/CD4− LF132 | 0.20 |
| BM CD41+/CD4− LF146 | 0.09 |
| mBM CD15+ | 0.00 |
| mBM CD14/11b−/15+ LF120 | 0.02 |
| mBM CD15+/11b LF 120 | 0.00 |
| BM CD15+/CD1b− LF 128 | 0.02 |
| BM CD14−/11b−/15+ LF145 | 0.05 |
| BM CD15+/CD1b+ LF128 | 0.00 |
| BM-1 CD15 ench LP4 | 0.01 |

TABLE 4
18636 Heme Taqman-Ph2b Expression Data

| Tissue | Expression |
|---|---|
| Eryth D 0 LF143 | 1.00 |
| Eryth D 0 LF139 | 0.44 |
| Eryth D 0 LF127 | 0.21 |
| Eryth 24 hr LF127 | 0.33 |
| Eryth 24 hr ILF139 | 0.32 |
| Eryth 24 hr LF143 | 0.65 |
| Eryth 48 hr LF127 | 0.27 |
| Eryth 48 hr LF139 | 0.13 |
| Eryth 48 hr LF143 | 0.46 |
| Eryth D 6 LF127 | 0.08 |
| Eryth D 6 LF139 | 0.04 |
| Eryth D 6 LF143 | 0.49 |
| Eryth D 8 LF127 | 0.32 |
| Eryth D 9 LF143 | 0.06 |
| Eryth D 10 LP170 | 0.06 |
| Eryth D 12 LF139 | 0.17 |
| Eryth D 12 LF23-8 | 0.11 |
| Eryth D 12 LF143 | 0.37 |
| Meg D LF140 | 0.50 |
| Meg 24 hr LF102 | 0.47 |
| Meg 24 hr LD110 | 0.31 |
| Meg 24 hr LF140 | 0.76 |
| Meg D 10 LF110 | 0.01 |
| Meg D 10 LF112 | 0.00 |
| Meg D 10 LP126 | 0.01 |
| Meg D 12 LF140 | 0.00 |
| Meg D 12 LF26 | 0.00 |
| Meg D 14 LP31-5 | 0.00 |
| Platelets LP57 | 0.01 |
| Neut D 0 LF141 | 0.35 |
| Neut D 0 LF144 | 0.10 |
| Neut 48 hr LF141 | 0.02 |
| Neut 48 hr LF144 | 0.04 |
| Neut D 4 LF141 | 0.02 |
| Neut D 4 LF144 | 0.03 |
| Neut D 6 LF141 | 0.02 |
| Neut D 6 LF144 | 0.02 |
| Neut D 6 LF71 | 0.03 |
| Neut D 8 LF144 | 0.01 |
| Neut D 11 LF137 | 0.00 |
| Neut D 12 LF141 | 0.00 |
| Neut D 12 LF144 | 0.00 |
| BFU-Eryth D 7 LP79 | 0.06 |
| BFU-Eryth D 7 LP95 | 0.00 |
| BFU-Eryth D 7+3epo LP8 | 0.01 |
| BFU-Eryth D 7+3epo LP104 | 0.08 |
| Mast Cell LP118 | 0.04 |

TABLE 5
Organ Recital-18636-Taqman ExpressionData

| Tissue | Expression |
|---|---|
| Artery, normal | 0 |
| Aorta, diseased | 0 |
| Vein, normal | 0 |
| Coronary SMC | 1.3526 |
| HUVEC | 0 |
| Hemangioma | 0 |
| Heart, normal | 0 |
| Heart, CHF | 0 |
| Kidney | 0 |
| Skeletal muscle | 0 |
| Adipose, normal | 0 |
| Pancreas | 0 |
| Primary osteoblasts | 0 |
| Osteoclasts (diff.) | 0 |
| Skin, normal | 0 |
| Spinal cord, normal | 0 |
| Brain cortex normal | 0 |
| Brain hypothalamus, normal | 0 |
| Nerve | 0 |
| DRG (Dorsal root ganglion) | 0 |
| Breast, normal | 0 |
| Breast, tumor | 0 |
| Ovary, normal | 0 |
| Ovary, tumor | 0 |
| Prostate, normal | 0 |
| Prostate, tumor | 0 |
| Salivary glands | 0 |
| Colon, normal | 0 |
| Colon, tumor | 0 |
| Lung, normal | 0 |
| Lung, tumor | 0 |
| Lung, COPD | 0 |
| Colon, IBD | 0 |
| Liver, normal | 0 |
| Liver, fibrosis | 0 |
| Spleen, normal | 0 |
| Tonsil, normal | 0 |
| Lymph node, normal | 0 |
| Small intestine, normal | 0 |
| Skin decubitus | 0 |
| Synovium | 0 |
| BM-MNO | 0 |
| Activated PBMC | 0 |
| Neutrophils | 0 |
| Megakaryocytes | 0 |
| Erythroid | 1.0467 |

### Human 2466

The present invention is based, in part, on the discovery of a novel G protein coupled receptor family member, referred to herein as “2466”.

The human 2466 sequence (SEQ ID NO:29), which is approximately 1801 nucleotides long including untranslated regions, contains a predicted methionine-initiated coding sequence of about 1482 nucleotides, not including the termination codon (nucleotides 53-1534 of SEQ ID NO:29; 1-1482 of SEQ ID NO:31). The coding sequence encodes a 494 amino acid protein (SEQ ID NO:30).

The human 2466 protein of SEQ ID NO:30 includes an amino-terminal hydrophobic amino acid sequence, consistent with a signal sequence, of about 53 amino acids (from amino acid 1 to about amino acid 53 of SEQ ID NO:30, PSORT, Nakai and Kanehisa (1992) Genomics 14:897-911), which upon cleavage results in the production of a mature protein form. This mature protein form (SEQ ID NO:32) is approximately 441 amino acid residues in length (from about amino acid 54 to amino acid 494 of SEQ ID NO:30).

Human 2466 contains the following regions or other structural features (for general information regarding PFAM identifiers, PS prefix and PF prefix domain identification numbers, refer to Sonnhammer et al. (1997) Protein 28:405-420): A seven transmembrane receptor domain (PFAM Accession Number PF00001) located at about amino acid residues 120 to 387 of SEQ ID NO:30; a GTP binding rhodopsin domain (ProDom No. PD047622) located at about amino acid residues 13 to 330 of SEQ ID NO:30; a receptor striatum-specific G protein-coupled protein-coupled GPR88 domain (ProDom No. PD340346) located at about amino acid residues 121 to 424 of SEQ ID NO:30; a CG12290 domain (ProDom No. PD326817) located at about amino acid residues 197 to 457 of SEQ ID NO:30; a RE2 receptor coupled G-protein domain (ProDom No. PD180341) located at about amino acid residues 285 to 399 of SEQ ID NO:30; seven transmembrane domain (predicted by MEMSAT, Jones et al. (1994) Biochemistry 33:3038-3049) at about amino acids 36 to 296 of SEQ ID NO:30; a leucine zipper pattern (Prosite PS00029) located at about amino acids 143 to 164 of SEQ ID NO:30 (LSLSLSDLLTALLCLPAAFLDL; SEQ ID NO:49); a G protein coupled receptor signature sequence (Prosite PS00237) located at about amino acids 197 to 213 of SEQ ID NO:30 (VSTLSVALISLDRYCAI; SEQ ID NO:50); a protein kinase C phosphorylation sites (Prosite PS00005) located at about amino acids 303 to 305 (TVR) of SEQ ID NO:30; two casein kinase II phosphorylation sites (Prosite PS00006) located at about amino acids 146 to 149 (SLSD; SEQ ID NO:51), and 266 to 269 (TSPD; SEQ ID NO:52) of SEQ ID NO:30; a glycosaminoglycan attachment site (Prosite PS00002) located at about amino acids 59 to 62 (SGGG; SEQ ID NO:53) of SEQ ID NO:30; a tyrosine kinase phosphorylated site (Prosite PS00007) located at about amino acids 400 to 406 (RNREEGY; SEQ ID NO:54) of SEQ ID NO:30; an amidation site (Prosite PS00009) located at about amino acids 221 to 224 (IGRR; SEQ ID NO:55) of SEQ ID NO:30; two ASN N-glycosylation sites (Prosite PS00001) located at about amino acids 54 to 57 (NLSD; SEQ ID NO:56), and 393 to 396 (NISM; SEQ ID NO:57) of SEQ ID NO:30; and ten N-myristoylation sites (Prosite PS00008) located at about amino acids 17 to 22 (GSQHSG; SEQ ID NO:58), 31 to 36 (GGTSSA; SEQ ID NO:59), 60 to 65 (GGGTAA; SEQ ID NO:60), 69 to 74 (GGGLGG; SEQ ID NO:61), 100 to 105 (GAAVAA; SEQ ID NO:62), 170 to 175 (GSAPAA; SEQ ID NO:63), 195 to 200 (GIVSTL; SEQ ID NO:64), 232 to 237 (GAWLTA; SEQ ID NO:65), 274 to 279 (GAAFSV; SEQ ID NO:66), and 280 to 285 (GLVVAC; SEQ ID NO:67) of SEQ ID NO:30.

Finally, searches performed using the “ProDom protein domain database” identified the following homologous domains within 2466: 1) a “RE2 receptor coupled G-protein” domain (SEQ ID NO:35-36) is 37% and 27% identical over amino acids 285 to 311 and 315 to 399 of SEQ ID NO:30 in two HSPs, respectively; 2) a “CG12290” domain (SEQ ID NO:37-38) is 22% and 25% identical over amino acids 197 to 312 and 306 to 457 of SEQ ID NO:30 in two HSPs, respectively; and 3) a “receptor striatum-specific G protein-coupled protein-coupled GPR88” domain (SEQ ID NO:39-42) is 40%, 33%, 47% and 27% identical over amino acids 121 to 142, 197 to 315, 372 to 391 and 373 to 424 of SEQ ID NO:30 in four HSPs, respectively.

A hydropathy plot of human 2466 was performed. Polypeptides of the invention include fragments which include: all or part of a hydrophobic sequence, e.g., the sequence from about amino acid 25 to 50, from about 85 to 123, from about 133 to 158, from about 180 to 200, from about 215 to 230, from about 265 to 290, from about 321 to 345, and from about 355 to 370 of SEQ ID NO:30; all or part of a hydrophilic sequence, e.g., the sequence from about amino acid 125 to 132, from about 210 to 223, from about 395 to 405, and from about 410 to 430 of SEQ ID NO:30; a sequence which includes a Cys, or a glycosylation site.

In a BLAST alignment of the human 2466 seven transmembrane domain with a consensus amino acid sequence of a domain derived from the ProDomain database (“GTP-binding rhodopsin” No. PD047622; ProDomain Release 2001.1), amino acid residues 1 to 315 of the 322 amino acid PD047622 consensus sequence (SEQ ID NO:34) aligns with amino acid residues 13 to 330 of the seven transmembrane domain of human 2466 (SEQ ID NO:30).

In a GAP alignment of 2466 with Homo sapiens leukocyte platelet-activating factor receptor mRNA (HUMNPIIY20, Genbank M76676), base pairs 1 to 650, 643 to 693, 695 to 1019, 1019 to 1442, 1432 to 1469, and 1469 to 1487 of human 2466 (SEQ ID NO:29) aligns with base pairs 1 to 650, 641 to 691, 692 to 1016, 1015 to 1438, 1427 to 1464, and 1463 to 1481 of M76676 (SEQ ID NO:43-48). GAP alignments use a matrix made by matblas from blosum62.iij.

The 2466 protein contains a significant number of structural characteristics in common with members of the G protein coupled receptor family. As used herein, the term “G protein coupled receptor” includes a protein or polypeptide which is capable of binding a ligand such as platelet-activating factor, interacting with a G protein, and/or transducing a signal from the extracellular milieu to the intracellular space of a cell.

Members of a G protein coupled receptor family of proteins are characterized by an N-terminal extraceullar domain, three extracellular loops, seven transbrane domains, three intracellular loops, and a C-terminal intracellular domain. Member of this family are known to bind a ligand, and transduce a signal from the extracellular space to the intracellular space. Members of this family are known to interact with G proteins in a G protein signaling cascade. Other members of this family include rhodopsin, beta-adrenergic receptor, serotonin receptor, muscarinic receptor, etc. An alignment of the 2466 gene with Homo sapiens leukocyte platelet-activating factor receptor mRNA demonstrates about 97% sequence identity between the two sequences (as calculated in matblas from the blosum62.iij matrix).

A 2466 polypeptide can include a “transmembrane domain”, a “extracellular domain”, a “intracellular domain”, or regions homologous with such domains. A 2466 polypeptide can further include a “N-terminal extracellular domain” or regions homologous with a “N-terminal extracellular domain,” and a “C-terminal intracellular domain” or regions homologous with a “C-terminal intracellular domain”.

As used herein, the term “seven transmembrane receptor (rhodopsin family) domain” includes an amino acid sequence of about 200 to 300 amino acid residues in length and having a bit score for the alignment of the sequence to the seven transmembrane receptor (rhodopsin family) domain (HMM) of at least 120. Preferably a seven transmembrane receptor (rhodopsin family) domain mediates transduction of an extracellular signal to the intracellular space. Preferably, a seven transmembrane receptor domain includes at least about 200 to 300 amino acids, more preferably about 230 to 300 amino acid residues, or about 250 to 300 amino acids and has a bit score for the alignment of the sequence to the seven transmembrane receptor domain (HMM) of at least about 100, more preferably at least about 120, and most preferably at least about 130 or greater. The seven transmembrane receptor domain can include a Prosite G protein coupled receptor signature sequence PS00237 ([GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R-[FYWCSH]-x(2)-[LIVM]; SEQ ID NO:68) such as amino acids 197 to 213 (VSTLSVALISLDRYCAI; SEQ ID NO:50) of SEQ ID NO:30, or sequences homologous thereto. In the above conserved signature sequence, and other motifs or signature sequences described herein, the standard IUPAC one-letter code for the amino acids is used. Each element in the pattern is separated by a dash (-); square brackets ([ ]) indicate the particular residues that are accepted at that position; x indicates that any residue is accepted at that position; and numbers in parentheses (( )) indicate the number of residues represented by the accompanying amino acid. The signature sequence of 2466 is located approximately between the fourth and fifth transmembrane domains of human 2466 polypeptide and which corresponds to about amino acids 197 to 213 of SEQ ID NO:30.

The seven transmembrane receptor domain (HMM) has been assigned the PFAM Accession Number PF00001. As used herein, the “seven transmembrane domain” is a portion of the human 2466 protein which is homologous, e.g., at least about 46%, 47%, 48%, 49%, 50%, 51%, 52%, 53%, 54%, 55%, 56%, 57%, 58%, 59%, 60%, or 61% identical to either or both of the following ProDom family “GTP-Binding Rhodopsin” domain (ProDomain Release 2001.1). An alignment of the seven transmembrane domain (amino acids 13 to 330 of SEQ ID NO:30) of human 2466 with PD047622, SEQ ID NO:34, derived from a BLAST search model shows 52% identity (as calculated in ProDomain from the blosum62 matrix). The seven transmembrane receptor domain (amino acids 120 to 387 of SEQ ID NO:30) of human 2466 aligns with the Pfam 7 transmembrane receptor (rhodopsin family) (7tm_{—}1; PF00001)) consensus amino acid sequence (SEQ ID NO:33) derived from a hidden Markov model.

In a preferred embodiment, a 2466 polypeptide or protein has a “seven transmembrane receptor domain” or a region which has at least about 60%, 70%, 80%, 90%, 95%, 99%, or 100% homology with a “seven transmembrane receptor domain,” e.g., the seven transmembrane receptor domain of human 2466 (e.g., residues 120 to 387 of SEQ ID NO:30).

To identify the presence of a “seven transmembrane receptor” domain in a 2466 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “seven transmembrane receptor” domain in the amino acid sequence of human 2466 at about residues 120 to 387 of SEQ ID NO:30.

To identify the presence of a “G protein coupled receptor” domain in a 2466 protein sequence, and make the determination that a polypeptide or protein of interest has a particular profile, the amino acid sequence of the protein can be searched against a database of domains, e.g., the ProDom database (Corpet et al. (1999), Nucl. Acids Res. 27:263-267). The ProDom protein domain database consists of an automatic compilation of homologous domains. Current versions of ProDom are built using recursive PSI-BLAST searches (Altschul et al. (1997) Nucleic Acids Res. 25:3389-3402; Gouzy et al. (1999) Computers and Chemistry 23:333-340) of the SWISS-PROT 38 and TREMBL protein databases. The database automatically generates a consensus sequence for each domain. A BLAST search was performed against the HMM database resulting in the identification of a “G protein coupled receptor” domain in the amino acid sequence of human 2466 at about residues 13 to 330, 121 to 424, 197 to 457, and 285 to 399 of SEQ ID NO:30.

A 2466 polypeptide can include at least one, two, three, four, five, six, or preferably seven “transmembrane domains” or regions homologous with a “transmembrane domain”. As used herein, the term “transmembrane domain” includes an amino acid sequence of about 10 to 40 amino acid residues in length and spans the plasma membrane. Transmembrane domains are rich in hydrophobic residues, e.g., at least 50%, 60%, 70%, 80%, 90%, 95%, or more of the amino acids of a transmembrane domain are hydrophobic, e.g., leucines, isoleucines, tyrosines, or tryptophans. Transmembrane domains typically have alpha-helical structures and are described in, for example, Zagotta et al., (1996) Annual Rev. Neurosci. 19:235-263, the contents of which are incorporated herein by reference. The transmembrane domains of human 2466 are located at about residues 54 to 76, 87 to 109, 136 to 154, 173 to 189, 220 to 243, 280 to 303, and 314 to 336 of mature peptide of SEQ ID NO:32.

In a preferred embodiment, a 2466 polypeptide or protein has at least one, two, three, four, five, six, or preferably seven “transmembrane domains” or a region which includes at least about 12 to 35 more preferably about 14 to 30 or 15 to 25 amino acid residues and has at least about 60%, 70% 80% 90% 95%, 99%, or 100% homology with a “transmembrane domain,” e.g., the transmembrane domains of human 2466 (e.g., residues 54 to 76, 87 to 109, 136 to 154, 173 to 189, 220 to 243, 280 to 303, and 314 to 336 of SEQ ID NO:32). The transmembrane domain of human 2466 can be visualized in a hydropathy plot as regions of about 15 to 25 amino acids where the hydropathy trace is mostly above the horizontal line.

To identify the presence of a “transmembrane” domain in a 2466 protein sequence, and make the determination that a polypeptide or protein of interest has a particular profile, the amino acid sequence of the protein can be analyzed by a transmembrane prediction method that predicts the secondary structure and topology of integral membrane proteins based on the recognition of topological models (MEMSAT, Jones et al., (1994) Biochemistry 33:3038-3049).

A 2466 polypeptide can include at least one, two, three, four, five, six, seven, or preferably eight “non-transmembrane regions.” As used herein, the term “non-transmembrane region” includes an amino acid sequence not identified as a transmembrane domain. The non-transmembrane regions in 2466 are located at about amino acids 1 to 53, 77 to 86, 110 to 135, 155 to 172, 190 to 119, 244 to 279, 304 to 313, and 337 to 441 of SEQ ID NO:32.

The non-transmembrane regions of 2466 include at least one, two, three, or preferably four cytoplasmic regions. When located at the C-terminus, the cytoplasmic region is referred to herein as the “C-terminal cytoplasmic domain.” As used herein, an “C-terminal cytoplasmic domain” includes an amino acid sequence having about 1 to 200, preferably about 1 to 175, more preferably about 1 to 150, or even more preferably about 1 to 125 amino acid residues in length, is located inside of a cell or within the cytoplasm of a cell and has the function of interacting with or controlling the interaction with a G protein. The N-terminal amino acid residue of an “C-terminal cytoplasmic domain” is adjacent to a C-terminal amino acid residue of a transmembrane domain in a 2466 protein. For example, a C-terminal cytoplasmic domain is located at about amino acid residues 337 to 441 of SEQ ID NO:32.

In a preferred embodiment, a 2466 polypeptide or protein has a C-terminal cytoplasmic domain or a region which includes about 1 to 200, preferably about 1 to 175, and more preferably about 1 to 150 amino acid residues and has at least about 60%, 70% 80% 90% 95%, 99%, or 100% homology with a “C-terminal cytoplasmic domain,” e.g., the C-terminal cytoplasmic domain of human 2466 (e.g., residues 337 to 441 of SEQ ID NO:32).

In another embodiment, a 2466 cytoplasmic region includes at least one, two, or preferably three cytoplasmic loops. As used herein, the term “loop” includes an amino acid sequence which is not included within a phospholipid membrane, having a length of at least about 4, preferably about 5 to 15, more preferably about 6 to 37 amino acid residues, and has an amino acid sequence that connects two transmembrane domains within a protein or polypeptide. Accordingly, the N-terminal amino acid of a loop is adjacent to a C-terminal amino acid of a transmembrane domain in a 2466 molecule, and the C-terminal amino acid of a loop is adjacent to an N-terminal amino acid of a transmembrane domain in a 2466 molecule. As used herein, a “cytoplasmic loop” includes a loop located inside of a cell or within the cytoplasm of a cell. For example, a “cytoplasmic loop” can be found at about amino acid residues 77 to 86, 155 to 172, and 244 to 279 of SEQ ID NO:32.

In a preferred embodiment, a 2466 polypeptide or protein has a cytoplasmic loop or a region which includes at least about 4, preferably about 5 to 15, and more preferably about 6 to 37 amino acid residues and has at least about 60%, 70% 80% 90% 95%, 99%, or 100% homology with a “cytoplasmic loop,” e.g., a cytoplasmic loop of human 2466 (e.g., residues 77 to 86, 155 to 172, and 244 to 279 of SEQ ID NO:32).

In another embodiment, a 2466 non-transmembrane region includes at least one, two, or preferably three non-cytoplasmic loops. As used herein, a “non-cytoplasmic loop” includes a loop located outside of a cell or within an intracellular organelle. Non-cytoplasmic loops include extracellular domains (i.e., outside of the cell) and intracellular domains (i.e., within the cell). When referring to membrane-bound proteins found in intracellular organelles (e.g., mitochondria, endoplasmic reticulum, peroxisomes microsomes, vesicles, endosomes, and lysosomes), non-cytoplasmic loops include those domains of the protein that reside in the lumen of the organelle or the matrix or the intermembrane space. For example, a “non-cytoplasmic loop” can be found at about amino acid residues 110 to 135, 190 to 219, and 304 to 313 of SEQ ID NO:32.

In a preferred embodiment, a 2466 polypeptide or protein has at least one non-cytoplasmic loop or a region which includes at least about 4, preferably about 5 to 15, more preferably about 6 to 37 amino acid residues and has at least about 60%, 70% 80% 90% 95%, 99%, or 100% homology with a “non-cytoplasmic loop,” e.g., at least one non-cytoplasmic loop of human 2466 (e.g., residues 110 to 135, 190 to 219, and 304 to 313 of SEQ ID NO:32).

In another embodiment, a non-cytoplasmic region of a 2466 protein can include the N-terminus and can be a “N-terminal non-cytoplasmic domain.” As used herein, an “N-terminal non-cytoplasmic domain” includes an amino acid sequence having a length of at least about 10, preferably about 10 to 100, more preferably about 10 to 60 amino acid residues, is located outside of a cell or within the lumen of the organelle or the matrix or the intermembrane space and may function to bind a ligand of the receptor. The C-terminal amino acid residue of an “N-terminal non-cytoplasmic domain” is adjacent to an N-terminal amino acid residue of a transmembrane domain in a 2466 protein. For example, an N-terminal non-cytoplasmic domain is located at about amino acid residues 1 to 53 of SEQ ID NO:32.

In a preferred embodiment, a 2466 polypeptide or protein has an N-terminal non-cytoplasmic domain or a region which includes at least about 10, preferably about 10 to 100, and more preferably about 10 to 60 amino acid residues and has at least about 60%, 70% 80% 90% 95%, 99%, or 100% homology with an N-terminal non-cytoplasmic domain,” e.g., the N-terminal non-cytoplasmic domain of human 2466 (e.g., residues 1 to 53 of SEQ ID NO:32).

A human 2466 protein can further include a leucine zipper pattern (Prosite PS00029) such as the one found at about amino acids 143 to 164 of SEQ ID NO:30.

A 2466 family member can include at least one leucine zipper pattern. Furthermore, a 2466 family member can include at least one protein kinase C phosphorylation site (Prosite PS00005); at least one, preferably two casein kinase II phosphorylation sites (Prosite PS00006); at least one tyrosine kinase phosphorylation site (Prosite PS00007); at least one, two, three, four, five, six, seven, preferably eight N-myristoylation sites (Prosite PS00008); at least one amidation site (Prosite PS00009); at least one, preferably two, N-glycosylation sites (Prosite PS00001); and at least one glycosaminoglycan attachment site (PS00002).

As the 2466 polypeptides of the invention can modulate 2466-mediated activities, they can be useful for developing novel diagnostic and therapeutic agents for G protein coupled receptor-associated or other 2466-associated disorders, as described below.

As used herein, a “G protein coupled receptor-associated activity” includes an activity which involves transduction of a signal across a membrane including binding a ligand such a platelet-activating factor or a neuropeptide and interacting with a G protein. Members of the family can play a role in neurological, psychiatric, or pain disease.

As used herein, a “2466 activity”, “biological activity of 2466” or “functional activity of 2466”, refers to an activity exerted by a 2466 protein, polypeptide or nucleic acid molecule on e.g., a 2466-responsive cell or on a 2466 substrate, e.g., a protein substrate, as determined in vivo or in vitro. In one embodiment, a 2466 activity is a direct activity, such as an association with a 2466 target molecule. A “target molecule” or “binding partner” is a molecule with which a 2466 protein binds or interacts in nature. In an exemplary embodiment, 2466 is a receptor, e.g., leukocyte platelet-activating factor receptor, and thus binds to or interacts in nature with a ligand, e.g., platelet-activating factor (PAF).

A 2466 activity can also be an indirect activity, e.g., a cellular signaling activity mediated by interaction of the 2466 protein with a 2466 receptor. Based on the above-described sequence structures and similarities to molecules of known function, the 2466 molecules of the present invention can have similar biological activities as G protein coupled receptor family members. For example, the 2466 proteins of the present invention can have one or more of the following activities: (1) the ability to interact or associate with a G protein; (2) the ability to bind a ligand such as a peptide, neuropeptide (e.g., FF, H2, glanin), platelet-activating factor; (3) the ability to transduce a signal through a membrane; (4) the ability to be phosphorylated or dephosphorylated; (5) the ability to transduce a pain signal; and (6) the ability to affect cGMP or cAMP concentrations in the cell.

The 2466 molecules of the invention can modulate the activities of cells in tissues where they are expressed. For example, TaqMan analysis shows 2466 mRNA is expressed in brain, spinal cord, dorsal root ganglia (DRG), optic nerve, testes, kidney, and thyroid. Accordingly, the 2466 molecules of the invention can act as therapeutic or diagnostic agents for neurological, psychiatric, opthalmological, renal, reproductive, sexual, and endocrinolgocial disorders.

The 2466 molecules can be used to treat pain disorders in part because the 2466 mRNA is expressed in the brain, spinal cord, and dorsal root ganglia. 2466 may play an important role in the regulation of metabolism or pain disorders.

Thus, the 2466 molecules can act as novel diagnostic targets and therapeutic agents for controlling one or more neurological or pain-related disorders or other G protein coupled receptor disorders.

As used herein, “G protein coupled receptor disorders” are diseases or disorders whose pathogenesis is caused by, is related to, or is associated with aberrant or deficient G protein coupled receptor protein function or expression. Examples of such disorders, e.g., G protein coupled receptor-associated or other 2466-associated disorders, include but are not limited to, cellular proliferative and/or differentiative disorders, disorders associated with bone metabolism, immune e.g., inflammatory, disorders, cardiovascular disorders, endothelial cell disorders, liver disorders, viral diseases, pain, or metabolic disorders.

The 2466 molecules can be used to treat neurological disorders in part because G protein coupled receptor family members are found in the brain, spinal cord, and dorsal root ganglia. Additionally, 2466 can play an important role in the regulation of metabolism or pain disorders.

Examples of cellular proliferative and/or differentiative disorders include cancer, e.g., carcinoma, sarcoma, metastatic disorders, or hematopoietic neoplastic disorders, e.g., leukemias. A metastatic tumor can arise from a multitude of primary tumor types, including but not limited to those of prostate, colon, lung, breast, and liver origin.

The 2466 molecules of the invention can be used to monitor, treat and/or diagnose a variety of proliferative disorders. Such disorders include hematopoietic neoplastic disorders.

Aberrant expression and/or activity of 2466 molecules can mediate disorders associated with bone metabolism.

The 2466 nucleic acid and protein of the invention can be used to treat and/or diagnose a variety of immune, e.g., inflammatory, (e.g. respiratory inflammatory) disorders.

Additionally, 2466 molecules can play an important role in the etiology of certain viral diseases, including but not limited to Hepatitis B, Hepatitis C, and Herpes Simplex Virus (HSV). Modulators of 2466 activity could be used to control viral diseases. The modulators can be used in the treatment and/or diagnosis of viral infected tissue or virus-associated tissue fibrosis, especially liver and liver fibrosis. Also, 2466 modulators can be used in the treatment and/or diagnosis of virus-associated carcinoma, especially hepatocellular cancer.

### Gene Expression Analysis of 2466

Human 2466 expression was measured by TaqMan® quantitative PCR (Perkin Elmer Applied Biosystems) in cDNA prepared from a variety of normal and diseased (e.g., cancerous) human tissues or cell lines.

The results indicate significant 2466 expression in brain, spinal cord, dorsal root ganglia, testes, ovary, kidney, aorta, and thyroid (see tables 6-14).

TABLE 6
2466 Human Panel Phase I

| Tissue | Expression |
|---|---|
| Adrenal Gland | 0.17 |
| Brain | 1.12 |
| Heart | 0.09 |
| Kidney | 0.16 |
| Liver | 0.03 |
| Lung | 0.01 |
| Mammary Gland | 0.08 |
| Pancreas | 0.31 |
| Placenta | 0.09 |
| Prostate | 0.25 |
| Salivary Gland | 0.01 |
| Muscle | 0.43 |
| Sm. Intestine | 0.08 |
| Spleen | 0.03 |
| Stomach | 0.16 |
| Teste | 31.58 |
| Thymus | 0.06 |
| Trachea | 0.47 |
| Uterus | 0.42 |
| Spinal Cord | 0.64 |
| DRG | 0.12 |
| Skin | 0.02 |

TABLE 7
m45493 Rat Panel Phase 1.1.4

| Tissue | Expression |
|---|---|
| Brain | 0.003 |
| Spinal Cord | 0.001 |
| DRG | 0.002 |
| SCG | 0.004 |
| Optic Nerve | 0.001 |
| Hairy Skin | 0.000 |
| Gastro Muscle | 0.000 |
| Heart | 0.000 |
| Kidney | 0.000 |
| Liver | 0.000 |
| Lung | 0.000 |
| Spleen | 0.000 |
| Aorta | 0.000 |
| Adrenal Gland | 0.000 |
| Salivary Gland | 0.000 |
| Thyroid | 0.002 |
| Prostrate | 0.000 |
| Thymus | 0.000 |
| Trachea | 0.000 |
| Esophagus | 0.000 |
| Duodenum | 0.000 |
| Diaphragm | 0.000 |
| Colon | 0.000 |

TABLE 8
m45493 Mouse Panel Phase 1.1.1

| Tissue | Expression |
|---|---|
| Brain | 0.07 |
| DRG | 0.01 |
| Spinal Cord | 0.10 |
| Sk Muscle | 0.01 |
| Skin | 0.00 |
| Ventricle | 0.00 |
| Atrium | 0.00 |
| Aorta | 0.03 |
| Small Intestine | 0.00 |
| Spleen | 0.01 |
| Esophagus | 0.00 |
| Stomach | 0.00 |
| Colon | 0.00 |
| Sub Maxillary | 0.00 |
| Thymus | 0.00 |
| Kidney | 0.01 |
| Liver | 0.00 |
| Lung | 0.00 |
| Teste | 0.04 |
| Trachea | 0.01 |
| Adrenal | 0.01 |
| Prostrate | 0.01 |

TABLE 9
Phase 1.3.4 Expression of 2466 w/ [symbol] 2

| Tissue | Expression |
|---|---|
| Artery normal | 0 |
| Vein normal | 0 |
| Aortic SMC EARLY | 1.1493 |
| Coronary SMC | 1.2107 |
| Static HUVEC | 0.337 |
| Shear HUVEC | 0.7274 |
| Heart normal | 3.2734 |
| Heart CHF | 0.3441 |
| Kidney | 1.603 |
| Skeletal Muscle | 0.5727 |
| Adipose normal | 0 |
| Pancreas | 0.214 |
| Primary osteoblasts | 0.2581 |
| Osteoclasts (diff) | 0 |
| Skin normal | 0 |
| Spinal cord normal | 1.4598 |
| Brain Cortex normal | 29.977 |
| Brain Hypothalamus normal | 9.6517 |
| Nerve | 0 |
| DRG (Dorsal Root Ganglion) | 0.9466 |
| Glial Cells (Astrocytes) | 16.6885 |
| Glioblastoma | 0.4447 |
| Breast normal | 0 |
| Breast tumor | 0.187 |
| Ovary normal | 5.2082 |
| Ovary Tumor | 0.1044 |
| Prostate Normal | 0.8771 |
| Prostate Tumor | 0.2975 |
| Epithelial Cells (Prostate) | 0.5251 |
| Colon normal | 0.0257 |
| Colon Tumor | 0.5055 |
| Lung normal | 0 |
| Lung tumor | 0.1645 |
| Lung COPD | 0.0189 |
| Colon IBD | 0 |
| Liver normal | 0 |
| Liver fibrosis | 0.4588 |
| Dermal Cells - fibroblasts | 0.2617 |
| Spleen normal | 0.0389 |
| Tonsil normal | 0.0187 |
| Lymph node | 0.0396 |
| Small Intestine | 0 |
| Skin-Decubitus | 0.1578 |
| Synovium | 0 |
| BM-MNC (Bone marrow monor) | 0.0043 |
| Activated PBMC | 0 |

TABLE 10
m45493 Rat Phase II Standard Error
N = 3

| Tissue | Expression |
|---|---|
| Naïve DRG | 0.0017 |
| I DRG CCI 3 | 0.0022 |
| I DRG CCI 7 | 0.0013 |
| I DRG CCI 10 | 0.0018 |
| I DRG CCI 28 | 0.0014 |
| Naïve DRG | 0.0008 |
| I DRG CFA 1 | 0.0009 |
| I DRG CFA 3 | 0.0006 |
| I DRG CFA 7 | 0.0012 |
| I DRG CFA 10 | 0.0007 |
| I DRG CFA 14 | 0.0003 |
| I DRG CFA 28 | 0.0005 |
| Naïve DRG | 0.0004 |
| I DRG AXT 1 | 0.0003 |
| I DRG AXT 3 | 0.0005 |
| I DRG AXT 7 | 0.0003 |
| I DRG AXT 14 | 0.0002 |

TABLE 11
m45493 Rat Phase III TPx Standard Error
N = 3

| Tissue | Expression |
|---|---|
| Naïve SC | 0.0001 |
| I SC CCI 3 | 0.0002 |
| I SC CCI 7 | 0.0003 |
| I SC CCI 10 | 0.0007 |
| I SC CCI 14 | 0.0003 |
| I SC CCI 28 | 0.0002 |
| Naïve SC | 0.0001 |
| I SC CFA 1 | 0.0001 |
| I SC CFA 3 | 0.0002 |
| I SC CFA 7 | 0.0003 |
| I SC CFA 10 | 0.0004 |
| I SC CFA 14 | 0.0002 |
| I SC CFA 28 | 0.0001 |
| Naïve SC | 0.0000 |
| I SC AXT 1 | 0.0003 |
| I SC AXT 3 | 0.0005 |
| I SC AXT 7 | 0.0006 |
| I SC AXT 14 | 0.0006 |

TABLE 12
m45493 Rat Panels Phase II & III

| Tissue | Expression |
|---|---|
| Naïve DRG | 0.001 |
| I DRG CCI 3 | 0.004 |
| I DRG CCI 7 | 0.002 |
| I DRG CCI 10 | 0.003 |
| I DRG CCI 14 | 0.022 |
| I DRG CCI 28 | 0.001 |
| Naïve DRG | 0.001 |
| I DRG CFA 1 | 0.002 |
| I DRG CFA 3 | 0.001 |
| I DRG CFA 7 | 0.002 |
| I DRG CFA 10 | 0.001 |
| I DRG CFA 14 | 0.001 |
| I DRG CFA 28 | 0.000 |
| Naïve DRG | 0.000 |
| I DRG AXT 1 | 0.000 |
| 1 DRG AXT 3 | 0.001 |
| 1 DRG AXT 7 | 0.000 |
| I DRG AXT 14 | 0.000 |
| Naïve SC | 0.001 |
| I SC CCI 3 | 0.005 |
| I SC CCI 7 | 0.005 |
| I SC CCI 10 | 0.009 |
| I SC CCI 14 | 0.003 |
| I SC CCI 28 | 0.002 |
| Naïve SC | 0.001 |
| I SC CFA 1 | 0.004 |
| I SC CFA 3 | 0.003 |
| I SC CFA 7 | 0.002 |
| I SC CFA 10 | 0.001 |
| I SC CFA 14 | 0.001 |
| I SC CFA 28 | 0.001 |
| Naïve SC | 0.001 |
| I SC AXT 1 | 0.002 |
| I SC AXT 3 | 0.001 |
| I SC AXT 7 | 0.001 |
| I SC AXT 14 | 0.000 |

TABLE 13
Phase 1.3.4 Expression of 2466 w/β2

| Tissue | Expression |
|---|---|
| Artery normal | 0 |
| Vein normal | 0 |
| Aortic SMC EARLY | 1.1493 |
| Coronary SMC | 1.2107 |
| Static HUVEC | 0.337 |
| Shear HUVEC | 0.7274 |
| Heart normal | 3.2734 |
| Heart CHF | 0.3441 |
| Kidney | 1.603 |
| Skeletal Muscle | 0.5727 |
| Adipose normal | 0 |
| Pancreas | 0.214 |
| primary osteoblasts | 0.2581 |
| Osteoclasts (diff) | 0 |
| Skin normal | 0 |
| Spinal cord normal | 1.4598 |
| Brain Cortex normal | 29.977 |
| Brain Hypothalamus normal | 9.6517 |
| Nerve | 0 |
| DRG (Dorsal Root Ganglion) | 0.9466 |
| Glial Cells (Astrocytes) | 16.6885 |
| Glioblastoma | 0.4447 |
| Breast normal | 0 |
| Breast tumor | 0.187 |
| Ovary normal | 5.2082 |
| Ovary tumor | 0.1044 |
| Prostate Normal | 0.8771 |
| Prostate Tumor | 0.2975 |
| Epithelial Cells (Prostate) | 0.5251 |
| Colon normal | 0.0257 |
| Colon Tumor | 0.5055 |
| Lung normal | 0 |
| Lung tumor | 0.1645 |
| Lung COPD | 0.0189 |
| Colon IBD | 0 |
| Liver normal | 0 |
| Liver fibrosis | 0.4588 |
| Dermal Cells-fibroblasts | 0.2617 |
| Spleen normal | 0.0389 |
| Tonsil normal | 0.0187 |
| Lymph node | 0.0396 |
| Small Intestine | 0 |
| Skin-Decubitus | 0.1578 |
| Synovium | 0 |
| BM-MNC (Bone marrow) | 0.0043 |
| Activated PBMC | 0 |

TABLE 14
m45493 Rat Panel Phase III

| Tissue | Expression |
|---|---|
| Naïve SC | 0.001 |
| I SC CCI 3 | 0.005 |
| I SC CCI 7 | 0.005 |
| I SC CCI 10 | 0.009 |
| I SC CCI 14 | 0.003 |
| I SC CCI 28 | 0.002 |
| Naïve SC | 0.001 |
| I SC CFA 1 | 0.004 |
| I SC CFA 3 | 0.003 |
| I SC CFA 7 | 0.002 |
| I SC CFA 10 | 0.001 |
| I SC CFA 14 | 0.001 |
| I SC CFA 28 | 0.001 |
| Naïve SC | 0.001 |
| I SC AXT 1 | 0.002 |
| I SC AXT 3 | 0.001 |
| I SC AXT 7 | 0.001 |
| I SC AXT 14 | 0.000 |

### Human 43238

The present invention is based, in part, on the discovery of novel G-protein coupled receptors, with similarities to known olfactory receptors, and nucleic acids encoding these receptors, referred to herein collectively as “GPCRs,” or by the individual clone name “43238.”

The human 43238 nucleotide sequence (SEQ ID NO:69), which is approximately 1970 nucleotides long including untranslated regions, contains a predicted methionine-initiated coding sequence of about 936 nucleotides (166-1101 of SEQ ID NO:69; 1-936 of SEQ ID NO:71). The coding sequence encodes a 312 amino acid protein (SEQ ID NO:70).

Human 43238 protein of SEQ ID NO:70 includes an amino-terminal hydrophobic amino acid sequence, consistent with a signal sequence, of about 42 amino acids (from amino acid 1 to about amino acid 42 of SEQ ID NO:70), which upon protease removal results in the production of the mature protein. The mature protein is approximately 270 amino acid residues in length (from about amino acid 43 to amino acid 312 of SEQ ID NO:70).

The mature human 43238 protein contains the following structural features: a predicted seven transmembrane (7™) domain located at about amino acids 43 to 312 of SEQ ID NO:70. The seven transmembrane domain shows homology to members of the rhodopsin family. Predicted transmembrane domains of the mature protein extend from about amino acid 58 (extracellular end) to about amino acid 77 (cytoplasmic end) of SEQ ID NO:70; from about amino acid 90 (cytoplasmic end) to about amino acid 114 (extracellular end) of SEQ ID NO:70; from about amino acid 143 (extracellular end) to about amino acid 165 (cytoplasmic end) of SEQ ID NO:70; from about amino acid 176 (cytoplasmic end) to about amino acid 194 (extracellular end) of SEQ ID NO:70; from about amino acid 204 (extracellular end) to about amino acid 225 (cytoplasmic end) of SEQ ID NO:70; from about amino acid 236 (cytoplasmic end) to about amino acid 259 (extracellular end) of SEQ ID NO:70; and from about amino acid 272 (extracellular end) to about amino acid 291 (cytoplasmic end); three cytoplasmic loops found at about amino acids 78-89, 166-175 and 226-235 of SEQ ID NO:70; three extracellular loops found at about amino acid 113-142, 195-203 and 260-271 of SEQ ID NO:70; and a C-terminal cytoplasmic domain is found at about amino acid residues 292-312 of SEQ ID NO:70.

The 43238 receptor protein additionally contains two predicted N-glycosylation sites (PS00001) from about amino acids 5-8 and 20-23 of SEQ ID NO:70; a predicted protein kinase C phosphorylation sites (PS00005) from about amino acids 290-292 of SEQ ID NO:70; three predicted casein kinase II phosphorylation sites (PS00006) from about amino acids 7-10, 66-69 and 266-269 of SEQ ID NO:70; four predicted N-myristoylation sites (PS00008) from about amino acids 111-116, 145-150, 151-156 and 212-217 of SEQ ID NO:70; one predicted amidation site (PS00009) from about amino acid 231-234 of SEQ ID NO:70; two predicted prokaryotic membrane lipoprotein lipid attachment sites (PS00013) located at about amino acids 98-108 and 207-217 of SEQ ID NO:70; and one G-protein coupled receptors signature site (PS00237) located at about amino acid 109-125 of SEQ ID NO:70.

A hydropathy plot of the human 43238 receptor was performed, thereby demonstrating the relative hydrophobic residues, the relative hydrophilic residues, the location of the transmembrane domains, the location of the extracellular domains and the location of the intracellular loops.

PFAM search results depict an alignment of the seven transmembrane (7tm) domain of human 43238 with a consensus amino acid sequence derived from a hidden Markov model. The consensus amino acid sequence (SEQ ID NO:72) aligns with amino acids 40 to 289 of SEQ ID NO:70. Additionally, searches performed using the “ProDom protein domain database” identified the following homologous domains within 43238: 1) an “olfactory receptor protein receptor-like G-protein coupled transmembrane glycoprotein multigene family” domain (SEQ ID NO:73) aligns with amino acids 245 to 305 of SEQ ID NO:70; 2) a “receptor olfactory protein receptor-like G-protein coupled transmembrane glycoprotein multigene family” domain (SEQ ID NO:74) aligns with amino acids 165 to 244 of SEQ ID NO:70; 3) a “receptor coupled G-protein transmembrane glycoprotein phosphorylation lipoprotein palmitate protein family” domain (SEQ ID NO:75) aligns with amino acids 58 to 161 of SEQ ID NO:70; and 4) a “putative G-protein coupled receptor RAIC” domain (SEQ ID NO:76) aligns with amino acids 246 to 306 of SEQ ID NO:70.

For general information regarding PFAM identifiers, PS prefix and PF prefix domain identification numbers, refer to Sonnhammer et al. (1997) Protein 28:405-420.

The primary and secondary protein structure of human 43238 can be plotted. The following plots can be used: Garnier-Robson plots providing the predicted location of alpha-, beta-, and turn regions (Garnier et al. (1978) J. Mol. Biol. 120:97); Chou-Fasman plots providing the predicted location of alpha-, beta-, turn and coil regions (Chou and Fasman (1978) Adv. In Enzymol. Mol. 47:45-148); Kyte-Doolittle hydrophilicity/hydrophobicity plots (Kyte and Doolittle (1982) J. Mol. Biol. 157:105-132); Eisenberg plots providing the predicted location of alpha- and beta-amphipathic regions (Eisenberg et al. (1982) Nature 299:371-374); a Karplus-Schultz plot providing the predicted location of flexible regions (Karplus and Schulz (1985) Naturwissens-Chafen 72:212-213); a plot of the antigenic index (Jameson-Wolf) (Jameson and Wolf (1988) CABIOS 4:121-136); and a surface probability plot (Emini algorithm) (Emini et al. (1985) J. Virol. 55:836-839).

The 43238 receptors of the present invention contain a significant number of structural characteristics in common with members of the G-protein coupled receptor family. As used herein, the term “G protein-coupled receptor” or “GPCR” refers to a family of proteins that preferably comprise an N-terminal extracellular domain, seven transmembrane domains (also referred to as membrane-spanning domains), three extracellular domains (also referred to as extracellular loops), three cytoplasmic domains (also referred to as cytoplasmic loops), and a C-terminal cytoplasmic domain (also referred to as a cytoplasmic tail). Members of the GPCR family also share certain conserved amino acid residues, some of which have been determined to be critical to receptor function and/or G protein signaling. For example, GPCRs usually contain the following features including a conserved asparagine residue in the first transmembrane domain.

Based on structural similarities, members of the GPCR family have been classified into various subfamilies, including: Subfamily I which comprises receptors typified by rhodopsin and the beta2-adrenergic receptor and currently contains over 200 unique members (reviewed by Dohlman et al. (1991) Annu. Rev. Biochem. 60:653-688); Subfamily II, which includes the parathyroid hormone/calcitonin/secretin receptor family (Juppner et al. (1991) Science 254:1024-1026; Lin et al. (1991) Science 254:1022-1024); Subfamily III, which includes the metabotropic glutamate receptor family in mammals, such as the GABA receptors (Nakanishi et al. (1992) Science 258: 597-603); Subfamily IV, which includes the cAMP receptor family that is known to mediate the chemotaxis and development of D. discoideum (Klein et al. (1988) Science 241:1467-1472); and Subfamily V, which includes the fungal mating pheromone receptors such as STE2 (reviewed by Kurjan I et al. (1992) Annu. Rev. Biochem. 61:1097-1129). Within each family, distinct, highly conserved motifs have been identified. These motifs have been suggested to be critical for the structural integrity of the receptor, as well as for coupling to G proteins.

Based on the results from the HMM analysis (HMMER Version 2.1.1), the 43238 polypeptide appears to belong to the rhodopsin subfamily of GPCRs (family 1).

In one embodiment, a 43238 protein includes at least one “7 transmembrane receptor profile” or regions homologous with a “7 transmembrane receptor profile”. As used herein, the term “7 transmembrane receptor profile” includes an amino acid sequence having at least about 10-500, preferably about 100-300, more preferably about 200-300 amino acid residues, or at least about 225-275 amino acids in length and having a bit score for the alignment of the sequence to the 7tm_{—}1 family Hidden Markov Model (HMM) of at least 10, preferably 20-30, more preferably 30-40, more preferably 40-50, 50-75, 75-100, 100-200 or greater. The 7tm_{—}1 family HMM has been assigned the PFAM Accession PF00001.

To identify the presence of a 7 transmembrane receptor profile in a 43238 receptor, the amino acid sequence of the protein is searched against a database of HMMs (e.g., the Pfam database, release 2.1) using the default parameters. For example, the hmmsf program, which is available as part of the HMMER package of search programs, is a family specific default program for PF00001 and score of 15 is the default threshold score for determining a hit. Alternatively, the seven transmembrane domain can be predicted based on stretches of hydrophobic amino acids forming α-helices (SOUSI server). For example, using a SOUSI server, a 7 TM receptor profile was identified in the amino acid sequence of SEQ ID NO:70 (e.g., amino acids 40-289 of SEQ ID NO:70). Accordingly, 43238 proteins having at least 50-60% homology, preferably about 60-70%, more preferably about 70-80%, or about 80-90% homology with the 7 transmembrane receptor profile of human 43238 are within the scope of the invention.

In one embodiment, a 43238 protein includes at least one extracellular domain. When located at the N-terminal domain the extracellular domain is referred to herein as an “N-terminal extracellular domain”, or as an N-terminal extracellular loop in the amino acid sequence of the protein. As used herein, an “N-terminal extracellular domain” includes an amino acid sequence having about 1-600, preferably about 1-500, preferably about 1-400, preferably about 1-300, preferably about 1-100, more preferably about 1-70, more preferably about 1-60, more preferably about 1-50, or even more preferably about 1-30 amino acid residues in length and is located outside of a cell or extracellularly. The C-terminal amino acid residue of a “N-terminal extracellular domain” is adjacent to an N-terminal amino acid residue of a transmembrane domain in a naturally-occurring 43238, or 43238-like protein.

In another embodiment, a 43238 protein includes at least one, two, three, four, five, six, or preferably, seven transmembrane domains. As used herein, the term “transmembrane domain” includes an amino acid sequence of about 15 amino acid residues in length which spans the plasma membrane. More preferably, a transmembrane domain includes about at least 16, 18, 20, 25, 30, 35 or 40 amino acid residues and spans the plasma membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al, (1996) Annual Rev. Neurosci. 19: 235-63, the contents of which are incorporated herein by reference.

In a preferred embodiment, a 43238 polypeptide or protein has at least one transmembrane domain or a region which includes at least 16, 18, 20, 25 30, 35 or 40 amino acid residues and has at least about 60%, 70% 80% 90% 95%, 99%, or 100% homology with a “transmembrane domain,” e.g., at least one transmembrane domain of human 43238. Preferably, the transmembrane domain transduces a signal, e.g., an extracellular signal across a cell membrane, and/or activates a signal transduction pathway.

In another embodiment, a 43238 protein includes at least one extracellular loop. As defined herein, the term “loop” includes an amino acid sequence having a length of at least about 4, preferably about 5-10, and more preferably about 10-20 amino acid residues, and has an amino acid sequence that connects two transmembrane domains within a protein or polypeptide. Accordingly, the N-terminal amino acid of a loop is adjacent to a C-terminal amino acid of a transmembrane domain in a naturally-occurring a 43238, or a 43238-like molecule, and the C-terminal amino acid of a loop is adjacent to an N-terminal amino acid of a transmembrane domain in a naturally-occurring 43238, or a 43238-like molecule. As used herein, an “extracellular loop” includes an amino acid sequence located outside of a cell, or extracellularly. For example, extracellular loops can be found at about amino acids 115-142, 195-203 and 260-271 of SEQ ID NO:70.

In a preferred embodiment, a 43238 polypeptide or protein has at least one extracellular loop or a region which includes at least about 4, preferably about 5-10, preferably about 10-20, and more preferably about 20-30 amino acid residues and has at least about 60%, 70% 80% 90% 95%, 99%, or 100% homology with an “extracellular loop,” e.g., at least one extracellular loop of human 43238 (e.g., residues 115-142, 195-203 and 260-271 of SEQ ID NO:70.

In another embodiment, a 43238 protein includes at least one cytoplasmic loop, also referred to herein as a cytoplasmic domain. As used herein, a “cytoplasmic loop” includes an amino acid sequence having a length of at least about 5, preferably about 5-10, and more preferably about 10-20 amino acid residues located within a cell or within the cytoplasm of a cell. For example, a cytoplasmic loop is found at about amino acids 77-90, 165-176 and 225-236 of SEQ ID NO:70.

In a preferred embodiment, a 43238 polypeptide or protein has at least one cytoplasmic loop or a region which includes at least about 5, preferably about 5-10, and more preferably about 10-20 amino acid residues and has at least about 60%, 70% 80% 90% 95%, 99%, or 100% homology with an “cytoplasmic loop,” e.g., at least one cytoplasmic loop of human 43238 (e.g., residues 76-89, 166-175 and 226-235 of SEQ ID NO:70.

In another embodiment, a 43238 protein includes a “C-terminal cytoplasmic domain”, also referred to herein as a C-terminal cytoplasmic tail, in the sequence of the protein. As used herein, a “C-terminal cytoplasmic domain” includes an amino acid sequence having a length of at least about 20, more preferably at least about 50, preferably about 50-100, more preferably about 70-93 amino acid residues and is located within a cell or within the cytoplasm of a cell. Accordingly, the N-terminal amino acid residue of a “C-terminal cytoplasmic domain” is adjacent to a C-terminal amino acid residue of a transmembrane domain in a naturally-occurring 43238 or 43238-like protein. For example, a C-terminal cytoplasmic domain is found at about amino acid residues 292-312 of SEQ ID NO:70.

In a preferred embodiment, a 43238 polypeptide or protein has a C-terminal cytoplasmic domain or a region which includes at least about 5, preferably about 5-20, more preferably about 5-22 amino acid residues and has at least about 60%, 70% 80% 90% 95%, 99%, or 100% homology with an “C-terminal cytoplasmic domain,” e.g., the C-terminal cytoplasmic domain of human 43238 (e.g., residues 292-312 of SEQ ID NO:70).

In yet another embodiment, a 43238 molecule can further include a signal sequence. As used herein, a “signal sequence” refers to a peptide of about 20-30 amino acid residues in length which occurs at the N-terminus of secretory and integral membrane proteins and which contains a majority of hydrophobic amino acid residues. For example, a signal sequence contains at least about 15-45 amino acid residues, preferably about 20-45 amino acid residues, more preferably about 20-45 amino acid residues, and more preferably about 40-43 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 43238 protein contains a signal sequence of about amino acids 1-42 of SEQ ID NO:70. The “signal sequence” is cleaved during processing of the mature protein. The mature 43238 proteins correspond to amino acids 43 to 312 of SEQ ID NO:70.

As the 43238 polypeptides of the invention may modulate 43238-mediated activities, they may be useful as of for developing novel diagnostic and therapeutic agents for 43238-mediated or related disorders, as described below.

As used herein, a “43238 activity”, “biological activity of 43238” or “functional activity of 43238”, refers to an activity exerted by a 43238 protein, polypeptide or nucleic acid molecule on e.g., a 43238-responsive cell or on a 43238 substrate, e.g., a protein substrate, as determined in vivo or in vitro. In one embodiment, a 43238 activity is a direct activity, such as an association with a 43238 target molecule. A “target molecule” or “binding partner” is a molecule with which a 43238 protein binds or interacts in nature. A 43238 activity can also be an indirect activity, e.g., a cellular signaling activity mediated by interaction of the 43238 protein with a 43238 receptor.

The 43238 molecules of the present invention are predicted to have similar biological activities as G-protein coupled receptor family members. For example, the 43238 proteins of the present invention can have one or more of the following activities: (1) regulating, sensing and/or transmitting an extracellular signal into a cell, (for example, a heart cell, a bone cell (e.g., an osteoclast or an osteoblast), a hematopoietic cell, a neural cell); (2) interacting with (e.g., binding to) an extracellular signal or a cell surface receptor; (3) mobilizing an intracellular molecule that participates in a signal transduction pathway (e.g., adenylate cyclase or phosphatidylinositol 4,5-bisphosphate (PIP₂), inositol 1,4,5-triphosphate (IP₃)); (4) regulating polarization of the plasma membrane; (5) controlling production or secretion of molecules; (6) altering the structure of a cellular component; (7) modulating cell proliferation, e.g., synthesis of DNA; (8) modulating cell migration, cell differentiation; and cell survival; and (9) providing targets for analgesic agents (agonists and antagonists) and agents which may interact with other disorders. Thus, the 43238 molecules can act as novel diagnostic targets and therapeutic agents for controlling G-protein coupled receptor-related disorders. Other activities, as described below, include the ability to modulate function, survival, morphology, proliferation and/or differentiation of cells of tissues in which 43238 molecules are expressed.

The response mediated by a 43238 receptor protein depends on the type of cell. For example, in some cells, binding of a ligand to the receptor protein may stimulate an activity such as release of compounds, gating of a channel, cellular adhesion, migration, differentiation, etc., through phosphatidylinositol or cyclic AMP metabolism and turnover while in other cells, the binding of the ligand will produce a different result. Regardless of the cellular activity/response modulated by the receptor protein, it is universal that the protein is a GPCR and interacts with G proteins to produce one or more secondary signals, in a variety of intracellular signal transduction pathways, e.g., through phosphatidylinositol or cyclic AMP metabolism and turnover, in a cell. As used herein, a “signaling transduction pathway” refers to the modulation (e.g., stimulation or inhibition) of a cellular function/activity upon the binding of a ligand to the GPCR (43238 protein). Examples of such functions include mobilization of intracellular molecules that participate in a signal transduction pathway, e.g., phosphatidylinositol 4,5-bisphosphate (PIP₂), inositol 1,4,5-triphosphate (IP₃) and adenylate cyclase.

As used herein, “phosphatidylinositol turnover and metabolism” refers to the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of ligand to the receptor activates, in some cells, the plasma-membrane enzyme phospholipase C that in turn can hydrolyze PIP₂ to produce 1,2-diacylglycerol (DAG) and inositol 1,4,5-triphosphate (IP₃). Once formed IP₃ can diffuse to the endoplasmic reticulum surface where it can bind an IP₃ receptor, e.g., a calcium channel protein containing an IP₃ binding site. IP₃ binding can induce opening of the channel, allowing calcium ions to be released into the cytoplasm. IP₃ can also be phosphorylated by a specific kinase to form inositol 1,3,4,5-tetraphosphate (IP₄), a molecule which can cause calcium entry into the cytoplasm from the extracellular medium. IP₃ and IP₄ can subsequently be hydrolyzed very rapidly to the inactive products inositol 1,4-biphosphate (IP₂) and inositol 1,3,4-triphosphate, respectively. These inactive products can be recycled by the cell to synthesize PIP₂. The other second messenger produced by the hydrolysis of PIP₂, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, refers to an activity of PIP₂ or one of its metabolites.

Another signaling pathway in which the receptor may participate is the cAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand-induced stimulation of certain G protein coupled receptors. In the cAMP signaling pathway, binding of a ligand to a GPCR can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

Based on the above-described sequence similarities, the 43238 molecules of the present invention are predicted to have similar biological activities as G-protein coupled receptor family members. Thus, the 43238 molecules can act as novel diagnostic targets and therapeutic agents for controlling one or more of cellular proliferative and/or differentiative disorders, disorders associated with bone metabolism, immune disorders, hematopoietic disorders, cardiovascular disorders, liver disorders, viral diseases, pain or metabolic disorders.

Aberrant expression and/or activity of 43238 molecules may mediate disorders associated with bone metabolism

The 43238 nucleic acid and protein of the invention can be used to treat and/or diagnose a variety of immune disorders. Exemplary immune disorders include hematopoietic neoplastic disorders.

Additionally, 43238 molecules may play an important role in the etiology of certain viral diseases, including but not limited to Hepatitis B, Hepatitis C and Herpes Simplex Virus (HSV). Modulators of 43238 activity could be used to control viral diseases. The modulators can be used in the treatment and/or diagnosis of viral infected tissue or virus-associated tissue fibrosis, especially liver and liver fibrosis. Also, 43238 modulators can be used in the treatment and/or diagnosis of virus-associated carcinoma, especially hepatocellular cancer.

Additionally, 43238 may play an important role in the regulation of metabolism or, in a preferred embodiment, pain disorders.

Expression and Tissue Distribution of 43238 mRNA

Relative expression levels of the 43238 was assessed in using TaqMan PCR and high expression was found in brain, spinal cord and testis, as well as DRG.

A molecular pathology panel indicates very low levels of expression overall and shows highest 43238 expression in brain tissue followed by dorsal root ganglion (DRG) and spinal cord.

A pain panel indicates very low levels of expression overall and shows highest 43238 expression in brain followed by testis and spinal cord.

Another pain panel indicates very low levels of expression overall and shows highest 43238 expression in human brain followed by human spinal cord.

Expression profiling results using in situ hybridization techniques have been repeatedly attempted with three different probes that cover the entire gene in both the pain group and molecular pathology and none of these results have shown positive results.

### Human 1983, 52881, 2398, 45449, 50289, and 52872

The present invention is based, in part, on the discovery of novel G-protein coupled receptors and nucleic acids encoding these receptors, referred to herein collectively as “GPCRs,” or by the individual clone name “1983, 52881, 2398, 45449, 50289, and 52872.”

### Human 1983

The human 1983 nucleotide sequence (SEQ ID NO:77), which is approximately 3127 nucleotides long including untranslated regions, contains a predicted methionine-initiated coding sequence of about 2070 nucleotides, not including the termination codon (nucleotides 171-2240 of SEQ ID NO:77; 1-2070 of SEQ ID NO:79). The coding sequence encodes a 690 amino acid protein (SEQ ID NO:78).

Human 1983 protein of SEQ ID NO:78 includes an amino-terminal hydrophobic amino acid sequence, consistent with a signal sequence, of about 19 amino acids (from amino acid 1 to about amino acid 19 of SEQ ID NO:78), which upon protease removal results in the production of the mature protein. The mature protein is approximately 671 amino acid residues in length (from about amino acid 20 to amino acid 690 of SEQ ID NO:78).

The human 1983 protein contains a predicted seven transmembrane (7™) domain (SEQ ID NO:95) located at about amino acids 424 to 671 of SEQ ID NO:78. Human 1983 additionally includes a predicted extracellular domain which extends from about amino acid 20 to about amino acid 432 of SEQ ID NO:78. The extracellular domain of the 1983 protein includes two EGF-like domains (SEQ ID NO:96) located at about amino acids 21-57 and 61-108 of SEQ ID NO:78. The extracellular domain of the 1983 protein additionally includes a latrophilin CL-1-like GPS domain (SEQ ID NO:97) located at about amino acids 366-418 of SEQ ID NO:78.

The seven transmembrane domain of the 1983 protein shows homology to members of the secretin family. Predicted transmembrane domains extend from about amino acid 433 (extracellular end) to about amino acid 452 (cytoplasmic end) of SEQ ID NO:78; from about amino acid 465 (cytoplasmic end) to about amino acid 481 (extracellular end) of SEQ ID NO:78; from about amino acid 500 (extracellular end) to about amino acid 524 (cytoplasmic end) of SEQ ID NO:78; from about amino acid 533 (cytoplasmic end) to about amino acid 553 (extracellular end) of SEQ ID NO:78; from about amino acid 570 (extracellular end) to about amino acid 594 (cytoplasmic end) of SEQ ID NO:78; from about amino acid 619 (cytoplasmic end) to about amino acid 636 (extracellular end) of SEQ ID NO:78; and from about amino acid 643 (extracellular end) to about amino acid 667 (cytoplasmic end) of SEQ ID NO:78; three cytoplasmic loops are located at about amino acids 453-464, 525-532 and 595-618 of SEQ ID NO:78; three extracellular loops are located at about amino acid 482-499, 554-569 and 637-642 of SEQ ID NO:78; and a C-terminal cytoplasmic domain is located at about amino acid residues 668-690 of SEQ ID NO:78.

The 1983 receptor protein additionally contains one predicted EF-hand calcium binding domain (PS00018) from about amino acids 153-165 of SEQ ID NO:78; ten predicted protein kinase C phosphorylation sites (PS00005) from about amino acids 135-137, 181-183, 233-235, 358-360, 363-365, 400-402, 457-459, 485-487, 558-560, and 668-670 of SEQ ID NO:78; fifteen predicted casein kinase II phosphorylation sites (PS00006) from about amino acids 54-57, 68-71, 76-79, 94-97, 135-138, 150-153, 155-158, 161-164, 181-184, 190-193, 244-247, 310-313, 325-328, 346-349, and 608-611 of SEQ ID NO:78; eight predicted N-myristoylation sites (PS00008) from about amino acids 38-43, 50-55, 80-85, 382-387, 388-393, 434-439, 480-485, and 521-526 of SEQ ID NO:78; ten predicted N-glycosylation sites (PS00001) from about amino acids 15-18, 21-24, 64-67, 74-77, 127-130, 177-180, 188-191, 249-252, 381-384, and 395-398 of SEQ ID NO:78; one predicted glycosaminoglycan attachment site (PS00002) from about amino acid 49-52 of SEQ ID NO:78; one predicted cAMP/cGMP phosphorylation site (PS00004) located at about amino acid 360-363 of SEQ ID NO:78; two tyrosine kinase phosphorylation sites (PS00007) located at about amino acid 36-43 and 669-676 of SEQ ID NO:78; and one aspartic acid and asparagine hydroxylation site (PS00010) located at about amino acid 75-86 of SEQ ID NO:78.

For general information regarding PFAM identifiers, PS prefix and PF prefix domain identification numbers, refer to Sonnhammer et al. (1997) Protein 28:405-420.

A hydropathy plot of human 1983 receptor was performed. Polypeptides of the invention include fragments which include: all or part of a hydrophobic sequence, e.g., the sequence from about amino acid 255-265, from about 335-345, and from about 410-420 of SEQ ID NO:78; all or part of a hydrophilic sequence, e.g., the sequence of from about amino acid 60-80, from about 240-250, and from about 320-330 of SEQ ID NO:78; a sequence which includes a Cys, or a glycosylation site.

### Human 52881

The human 52881 sequence (SEQ ID NO:80), which is approximately 4238 nucleotides long including untranslated regions, contains a predicted methionine-initiated coding sequence of about 1827 nucleotides, not including the termination codon (nucleotides 403-2229 of SEQ ID NO:80; 1-1827 of SEQ ID NO:82). The coding sequence encodes a 609 amino acid protein (SEQ ID NO:81).

The 52881 protein contains a predicted seven transmembrane (7™) domain (SEQ ID NO:98) located at about amino acids 80 to 154 of SEQ ID NO:81. The seven transmembrane domain shows homology to members of the rhodopsin family. Predicted transmembrane domains extend from about amino acids 11-34, 44-67, 85-106, 127-149, 172-196, and 245-269 of SEQ ID NO:81. Predicted non-transmembrane domains extend from about amino acids 1-10, 35-43, 68-84, 107-126, 150-171, 197-244, and 270-609 of SEQ ID NO:81.

The 52881 protein additionally contains: four predicted cAMP/cGMP phosphorylation sites (PS00004) located at about amino acids 225-228, 393-396, 436-439, and 453-456 of SEQ ID NO:81; six predicted protein kinase C phosphorylation sites (PS00005) located at about amino acids 153-155, 268-270, 392-394, 462-464, 482-484, and 560-562 of SEQ ID NO:81; ten predicted casein kinase II phosphorylation sites (PS00006) located at about amino acids 228-231, 324-327, 328-331, 364-367, 396-399, 417-420, 466-469, 506-509, 568-571, and 590-593 of SEQ ID NO:81; one predicted tyrosine kinase phosphorylation site (PS00007) located at about amino acids 342-348 of SEQ ID NO:81; ten predicted N-myristoylation sites (PS00008) located at about amino acids 9-14, 169-174, 181-186, 187-192, 232-237, 244-249, 531-536, 564-569, 573-578 and 579-584 of SEQ ID NO:81; and one predicted amidation site (PS00009) from about amino acids 223-226 of SEQ ID NO:81.

A hydropathy plot of human 52881 was performed. Polypeptides of the invention include fragments which include: all or part of a hydrophobic sequence, e.g., the sequence from about amino acid 280-300, from about 420-430, and from about 495-505 of SEQ ID NO:81; all or part of a hydrophilic sequence, e.g., the sequence of from about amino acid 225-240, from about 475-490, and from about 540-555 of SEQ ID NO:81; a sequence which includes a Cys, or a glycosylation site.

### Human 2398

The human 2398 nucleotide sequence (SEQ ID NO:83), which is approximately 1113 nucleotides long including untranslated regions, contains a predicted methionine-initiated coding sequence of about 1050 nucleotides, not including the termination codon (nucleotides 1-1050 of SEQ ID NO:83; 1-1050 of SEQ ID NO:85). The coding sequence encodes a 350 amino acid protein (SEQ ID NO:84).

The 2398 protein contains a G-protein receptor signature (PS00237) located at about amino acids 125-141 of SEQ ID NO:84. The 2398 protein also includes a predicted seven transmembrane (7™) domain (SEQ ID NO:99) located at about amino acids 58 to 303 of SEQ ID NO:84. The seven transmembrane domain shows homology to members of the rhodopsin family. An extracellular domain extends from about amino acids 1-41 of SEQ ID NO:84. Predicted transmembrane domains extend from about amino acid 42 (extracellular end) to about amino acid 66 (cytoplasmic end) of SEQ ID NO:84; from about amino acid 78 (cytoplasmic end) to about amino acid 99 (extracellular end) of SEQ ID NO: 84; from about amino acid 114 (extracellular end) to about amino acid 135 (cytoplasmic end) of SEQ ID NO:84; from about amino acid 154 (cytoplasmic end) to about amino acid 176 (extracellular end) of SEQ ID NO:84; from about amino acid 202 (extracellular end) to about amino acid 224 (cytoplasmic end) of SEQ ID NO:84; from about amino acid 241 (cytoplasmic end) to about amino acid 259 (extracellular end) of SEQ ID NO:84; and from about amino acid 291 (extracellular end) to about amino acid 310 (cytoplasmic end) of SEQ ID NO:84; three cytoplasmic loops are located at about amino acids 67-77, 136-153, and 225-240 of SEQ ID NO:84; three extracellular loops are located at about amino acid 100-113, 177-201, and 260-290 of SEQ ID NO:84; and a C-terminal cytoplasmic domain is located at about amino acid residues 311-350 of SEQ ID NO:84.

The 2398 receptor protein additionally contains five predicted protein kinase C phosphorylation sites (PS00005) from about amino acids 195-197, 223-225, 278-280, 309-311 and 323-325 of SEQ ID NO:84; four predicted casein kinase II phosphorylation sites (PS00006) from about amino acids 25-28, 74-77, 177-180, and 330-333 of SEQ ID NO:84; one predicted glycosaminoglycan attachment site (PS00002) located at about amino acids 148-151 of SEQ ID NO:84; one predicted N-myristoylation site (PS00008) from about amino acids 55-60 of SEQ ID NO:84; and one tyrosine kinase phosphorylation site (PS00007) located at about amino acid 263-269 of SEQ ID NO:84.

A hydropathy plot of human 2398 was performed. Polypeptides of the invention include fragments which include: all or part of a hydrophobic sequence, e.g., the sequence from about amino acid 265-275 and from about 285-295 of SEQ ID NO:84; all or part of a hydrophilic sequence, e.g., the sequence of from about amino acid 1-25, from about 70-80, and from about 320-330 of SEQ ID NO:84; a sequence which includes a Cys, or a glycosylation site.

### Human 45449

The human 45449 nucleotide sequence (SEQ ID NO:86), which is approximately 1109 nucleotides long including untranslated regions, contains a predicted methionine-initiated coding sequence of about 669 nucleotides, not including the termination codon (nucleotides 326-994 of SEQ ID NO:86; 1-669 of SEQ ID NO:88). The coding sequence encodes a 223 amino acid protein (SEQ ID NO:87).

The 45449 protein contains a predicted seven transmembrane (7™) domain (SEQ ID NO:100) located at about amino acids 1 to 176 of SEQ ID NO:87. The seven transmembrane domain shows homology to members of the rhodopsin family.

An N-terminal domain extends from about amino acids 1-11 of SEQ ID NO:87. Predicted transmembrane domains extend from about amino acid 12-33, 68-90, and 123-147 of SEQ ID NO:87. Predicted non-transmembrane domains extend from about amino acids 91-122 and 34-67 of SEQ ID NO:87. A C-terminal domain is located at about amino acid residues 148-324 of SEQ ID NO:87.

The 45449 receptor protein additionally contains: one predicted opsin retinal binding site located at about amino acids 165-183 of SEQ ID NO:87; three predicted protein kinase C phosphorylation sites (PS00005) from about amino acids 99-101, 194-196, and 209-211 of SEQ ID NO:87; one predicted casein kinase II phosphorylation sites (PS00006) from about amino acid 99-102 of SEQ ID NO:87; two predicted N-myristoylation sites (PS00008) from about amino acids 50-55 and 189-194 of SEQ ID NO:87; and two predicted cAMP/cGMP dependent protein kinase phosphorylation site located at about amino acids 195-198 and 211-214 of SEQ ID NO:87.

A hydropathy plot of human 45449 was performed. Polypeptides of the invention include fragments which include: all or part of a hydrophobic sequence, e.g., the sequence from about amino acid 160-170 of SEQ ID NO:87; all or part of a hydrophilic sequence, e.g., the sequence of from about amino acid 100-110 and from about 195-205 of SEQ ID NO:87; a sequence which includes a Cys, or a glycosylation site.

### Human 50289

The human 50289 nucleotide sequence (SEQ ID NO:89), which is approximately 3489 nucleotides long including untranslated regions, contains a predicted methionine-initiated coding sequence of about 2556 nucleotides, including the termination codon (nucleotides 52-2607 of SEQ ID NO:89; 1-2556 of SEQ ID NO:91). The coding sequence encodes a 852 amino acid protein (SEQ ID NO:90).

Human 50289 protein of SEQ ID NO:90 includes an amino-terminal hydrophobic amino acid sequence, consistent with a signal sequence, of about 20 amino acids (from amino acid 1 to about amino acid 20 of SEQ ID NO:90), which upon protease removal results in the production of the mature protein. The mature protein is approximately 832 amino acid residues in length (from about amino acid 21 to amino acid 852 of SEQ ID NO:90).

The mature 50289 protein contains a natriuretic peptide (ANF) ligand binding domain (SEQ ID NO:101) located at about amino acids 61 to 470 of SEQ ID NO:90. The ANF domain is located at the extracellular domain of the human 50289, which extends from about amino acid 1-546 of SEQ ID NO:90. Predicted transmembrane domains extend from about amino acid 567 (extracellular end) to about amino acid 590 (cytoplasmic end) of SEQ ID NO:90; from about amino acid 600 (cytoplasmic end) to about amino acid 623 (extracellular end) of SEQ ID NO:90; from about amino acid 641 (extracellular end) to about amino acid 659 (cytoplasmic end) of SEQ ID NO:90; from about amino acid 679 (cytoplasmic end) to about amino acid 702 (extracellular end) of SEQ ID NO:90; from about amino acid 726 (extracellular end) to about amino acid 750 (cytoplasmic end) of SEQ ID NO:90; from about amino acid 762 (cytoplasmic end) to about amino acid 782 (extracellular end) of SEQ ID NO:90; and from about amino acid 799 (extracellular end) to about amino acid 810 (cytoplasmic end) of SEQ ID NO:90; three extracellular loops located at about amino acids 624-640, 703-678, and 751-761 of SEQ ID NO:90; three cytoplasmic loops located at about amino acid 591-599, 660-678, and 703-725 of SEQ ID NO:90; and a C-terminal cytoplasmic domain is found at about amino acid residues 811-851 of SEQ ID NO:90.

The 50289 receptor protein additionally contains: one GPCR family 3 signature 2 domain located at about amino acids 516-540 of SEQ ID NO:90; nine predicted glycosylation sites located at about amino acids 85-88, 130-133, 264-267, 285-288, 380-383, 411-414, 432-435, 474-478, and 736-739 of SEQ ID NO:90; nine predicted protein kinase C phosphorylation sites (PS00005) from about amino acids 153-155, 175-177, 189-191, 289-291, 293-295, 477-479, 480-482, 527-529 and 550-552 of SEQ ID NO:90; three predicted casein kinase II phosphorylation sites (PS00006) from about amino acid 102-105, 175-178, and 214-217 of SEQ ID NO:90; fourteen predicted N-myristoylation sites (PS00008) from about amino acids 20-25, 69-74, 92-97, 234-239, 319-324, 476-481, 580-585, 602-607, 645-650, 730-735, 762-767, 803-808, 830-835, and 838-843 of SEQ ID NO:90; and one predicted cAMP/cGMP dependent protein kinase phosphorylation site located at about amino acids 555-558 of SEQ ID NO:90.

A hydropathy plot of human 50289 was performed. Polypeptides of the invention include fragments which include: all or part of a hydrophobic sequence, e.g., the sequence from about amino acid 70-80, from about 150-165, and from about 220-240 of SEQ ID NO:90; all or part of a hydrophilic sequence, e.g., the sequence of from about amino acid 50-60, from about 480-510, and from about 545-560 of SEQ ID NO:90; a sequence which includes a Cys, or a glycosylation site.

### Human 52872

The human 52872 sequence (SEQ ID NO:92), which is approximately 1609 nucleotides long including untranslated regions, contains a predicted methionine-initiated coding sequence of about 1194 nucleotides, not including the termination codon (nucleotides 176-1369 of SEQ ID NO:92; 1-1194 of SEQ ID NO:94). The coding sequence encodes a 398 amino acid protein (SEQ ID NO:93).

The 52872 protein contains a predicted seven transmembrane (7™) domain (PFAM Accession Number PF00001; SEQ ID NO:99) located at about amino acids 59 to 323 of SEQ ID NO:93. The seven transmembrane domain shows homology to members of the rhodopsin family. An extracellular domain extends from about amino acids 1-42 of SEQ ID NO:93. Predicted transmembrane domains extend from about amino acid 43 (extracellular end) to about amino acid 67 (cytoplasmic end) of SEQ ID NO:93; from about amino acid 76 (cytoplasmic end) to about amino acid 110 (extracellular end) of SEQ ID NO:93; from about amino acid 117 (extracellular end) to about amino acid 136 (cytoplasmic end) of SEQ ID NO:93; from about amino acid 158 (cytoplasmic end) to about amino acid 180 (extracellular end) of SEQ ID NO:93; from about amino acid 204 (extracellular end) to about amino acid 228 (cytoplasmic end) of SEQ ID NO:93; from about amino acid 264 (cytoplasmic end) to about amino acid 285 (extracellular end) of SEQ ID NO:93; and from about amino acid 310 (extracellular end) to about amino acid 326 (cytoplasmic end) of SEQ ID NO:93; three cytoplasmic loops at about amino acids 68-75, 137-157, and 229-263 of SEQ ID NO:93; three extracellular loops at about amino acid 111-116, 181-203, and 286-309 of SEQ ID NO:93; and a C-terminal cytoplasmic domain at about amino acid residues 327-398 of SEQ ID NO:93.

The 52872 receptor protein additionally contains: three predicted N-glycosylation sites (PS00001) from about amino acids 10-13, 18-21, and 28-31 of SEQ ID NO:93; two predicted Protein Kinase C phosphorylation sites (PS00005) at about amino acids 36-38 and 155-157 of SEQ ID NO:93; and five predicted N-myristylation sites (PS00008) from about 14-19, 21-26, 56-61, 247-252, and 255-260 of SEQ ID NO:93.

A hydropathy plot of human 52872 was performed. Polypeptides of the invention include fragments which include: all or part of a hydrophobic sequence, e.g., the sequence from about amino acid 45-65, from about 165-180, and from about 210-225 of SEQ ID NO:93; all or part of a hydrophilic sequence, e.g., the sequence of from about amino acid 295-300, from about 345-360, and from about 370-380 of SEQ ID NO:93; a sequence which includes a Cys, or a glycosylation site.

The 1983, 52881, 2398, 45449, 50289, and 52872 proteins contain a significant number of structural characteristics in common with members of the G protein-coupled receptor family. The G-protein coupled receptor family of proteins is an extensive group of proteins, which transduce extracellular signals triggered by, e.g., hormones, neurotransmitters, odorants and light, by interaction with guanine nucleotide-binding (G) proteins. G-protein coupled receptors typically have seven hydrophobic membrane spanning regions. The N-terminus of a G-protein coupled receptor is typically located on the extracellular side of the membrane and is often glycosylated, while the C-terminus is cytoplasmic and generally phosphorylated. Three extracellular loops alternate with three intracellular loops to link the seven transmembrane regions. Some G-protein coupled receptors possess a signal peptide. Generally, the most conserved portions of G-protein coupled receptors are the transmembrane regions and the first two cytoplasmic loops. A conserved acidic-arginine-aromatic triplet is present in the N-terminal extremity of the second cytoplasmic loop and may be implicated in the interaction with G proteins.

Based on structural similarities, members of the GPCR family have been classified into various subfamilies, including: Subfamily I, which comprises receptors typified by rhodopsin and the beta2-adrenergic receptor and currently contains over 200 unique members (reviewed by Dohlman et al. (1991) Annu. Rev. Biochem. 60:653-688); Subfamily II, which includes the parathyroid hormone/calcitonin/secretin receptor family (Juppner et al. (1991) Science 254:1024-1026; Lin et al. (1991) Science 254:1022-1024); Subfamily III, which includes the metabotropic glutamate receptor family in mammals, such as the GABA receptors (Nakanishi et al. (1992) Science 258: 597-603); Subfamily IV, which includes the cAMP receptor family that is known to mediate the chemotaxis and development of D. discoideum (Klein et al. (1988) Science 241:1467-1472); and Subfamily V, which includes the fungal mating pheromone receptors such as STE2 (reviewed by Kurjan I et al. (1992) Annu. Rev. Biochem. 61:1097-1129). Within each family, distinct, highly conserved motifs have been identified. These motifs have been suggested to be critical for the structural integrity of the receptor, as well as for coupling to G proteins.

Based upon the results of the HMM analysis (HMMER Version 2.1.1), the 52881, 2398, 45449, and 52872 polypeptides appear to belong to the rhodopsin subfamily of GPCRs (Subfamily I). 1983 appears to belong to the secretin subfamily of GPCRs (Subfamily II).

A 52881, 2398, 45449 or 52872 polypeptide can include a “rhodopsin-related seven transmembrane receptor domain” or regions homologous with a “rhodopsin-related seven transmembrane receptor domain”.

As used herein, the term “rhodopsin-related seven transmembrane receptor domain” includes an amino acid sequence of about 40-300 amino acid residues in length and having a bit score for the alignment of the sequence to the rhodopsin-related seven transmembrane receptor domain (HMM) of at least 15 or greater. Preferably, the rhodopsin-related seven transmembrane receptor domain includes an amino acid sequence which is about 50-280 amino acids, more preferably about 70-270 amino acids in length, and has a bit score for the alignment of the sequence to the rhodopsin-related seven transmembrane receptor domain (HMM) of at least 20 or greater, preferably 30 or greater. A 52881 protein preferably contains an amino acid sequence of about 75 amino acid residues in length, having a bit score for the alignment of the sequence to the rhodopsin-related seven transmembrane receptor domain at least 30. A 2398 protein preferably contains an amino acid sequence of about 246 amino acid residues in length, having a bit score for the alignment of the sequence to the rhodopsin-related seven transmembrane receptor domain at least 260. A 45449 protein preferably contains an amino acid sequence of about 176 amino acid residues in length, having a bit score for the alignment of the sequence to the rhodopsin-related seven transmembrane receptor domain at least 50. A 52872 protein preferably contains an amino acid sequence of about 265 amino acid residues in length, having a bit score for the alignment of the sequence to the rhodopsin-related seven transmembrane receptor domain at least 220.

The rhodopsin-related seven transmembrane receptor domain (HMM) has been assigned the PFAM Accession Number PF00001 The rhodopsin-related seven transmembrane receptor domain (amino acids 80 to 154 of SEQ ID NO:81) of human 52881 aligns with a consensus amino acid sequence (SEQ ID NO:98) derived from a hidden Markov model. The rhodopsin-related seven transmembrane receptor domain (amino acids 58 to 303 of SEQ ID NO:84) of human 2398 aligns with a consensus amino acid sequence (SEQ ID NO:99) derived from a hidden Markov model. The rhodopsin-related seven transmembrane receptor domain (amino acids 1 to 176 of SEQ ID NO:87) of human 45449 aligns with a consensus amino acid sequence (SEQ ID NO:100) derived from a hidden Markov model. The rhodopsin-related seven transmembrane receptor domain (amino acids 59 to 323 of SEQ ID NO:93) of human 52872 aligns with a consensus amino acid sequence (SEQ ID NO:99) derived from a hidden Markov model.

In a preferred embodiment, a 52881, 2398, 45449 or 52872 polypeptide or protein has a “rhodopsin-related seven transmembrane receptor domain” or a region which includes at least about 40-300 amino acid residues in length, preferably about 50-280 amino acids, more preferably about 70-270 amino acids and has at least about 50%, 60%, 70% 80% 90% 95%, 99%, or 100% homology with a “rhodopsin-related seven transmembrane receptor domain,” e.g., the rhodopsin-related seven transmembrane receptor domain of human 52881, 2398, 45449 or 52872 (e.g., amino acids 80 to 154 of SEQ ID NO:81, amino acids 58 to 303 of SEQ ID NO:84, amino acids 1 to 176 of SEQ ID NO:87, or amino acids 59 to 323 of SEQ ID NO:93).

To identify the presence of a “rhodopsin-related seven transmembrane receptor domain” in a 52881, 2398, 45449 or 52872 protein sequence, and make the determination that a polypeptide or protein of interest has a particular profile, the amino acid sequence of the protein can be searched against a database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “rhodopsin-related seven transmembrane receptor domain” domain in the amino acid sequence of human 52881, 2398, 45449 and 52872 (at about amino acids 80 to 154 of SEQ ID NO:81, amino acids 58 to 303 of SEQ ID NO:84, amino acids 1 to 176 of SEQ ID NO:87, and amino acids 59 to 323 of SEQ ID NO:93).

A 1983 polypeptide can include a “secretin-related seven transmembrane receptor domain” or regions homologous with a “rhodopsin-related seven transmembrane receptor domain”.

As used herein, the term “secretin-related seven transmembrane receptor domain” includes an amino acid sequence of about 50-300 amino acid residues in length, preferably about 100-280 amino acids, preferably about 150-260 amino acids, more preferably about 248 amino acids and having a bit score for the alignment of the sequence to the secretin-related seven transmembrane receptor domain (HMM) of at least 200 or greater, preferably 250 or greater.

The secretin-related seven transmembrane receptor domain (HMM) has been assigned the PFAM Accession Number PF00002. The secretin-related seven transmembrane receptor domain (amino acids 424-671 of SEQ ID NO:78) of human 1983 aligns with a consensus amino acid sequence (SEQ ID NO:95) derived from a hidden Markov model.

In a preferred embodiment, a 1983 polypeptide or protein has a “secretin-related seven transmembrane receptor domain” or a region which includes at least about 40-300 amino acid residues, preferably about 50-300 amino acids, preferably about 100-280 amino acids, preferably about 150-260 amino acids and has at least about 50%, 60%, 70% 80% 90% 95%, 99%, or 100% homology with a “secretin-related seven transmembrane receptor domain,” e.g., the secretin-related seven transmembrane receptor domain of human 1983 (e.g., amino acids 424-671 of SEQ ID NO:78).

To identify the presence of a “secretin-related seven transmembrane receptor domain” in a 1983 protein sequence, and make the determination that a polypeptide or protein of interest has a particular profile, the amino acid sequence of the protein can be searched against a database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “secretin-related seven transmembrane receptor domain” domain in the amino acid sequence of human 1983 (at about amino acids 424-671 of SEQ ID NO:78).

In one embodiment, a 1983 protein includes at least one at least one EGF-like domains. Preferably, the EGF-like domain is found in the extracellular domain of a 1983 protein. As used herein, an “EGF-like domain” refers to an amino acid sequence of about 25 to 50, preferably about 30 to 45, and more preferably 30 to 40 amino acid residues in length. An EGF domain further contains at least about 2 to 10, preferably, 3 to 9, 4 to 8, or 6 to 7 conserved cysteine residues. A consensus EGF-like domain sequence includes six cysteines, all of which are thought to be involved in disulfide bonds having the following amino acid sequence: Xaa(4)-Cys-Xaa(0, 48)-Cys-Xaa(3, 12)-Cys-Xaa(1, 70)-Cys-Xaa(1, 6)-Cys-Xaa(2)-Gly-Aro-Xaa(0, 21)-Gly-Xaa(2)-Cys-Xaa (SEQ ID NO:102), where Xaa is any amino acid and Aro is any aromatic amino acid. The region between the fifth and the sixth cysteine typically contains two conserved glycines of which at least one is present in most EGF-like domains. Proteins having such domains may play a role in mediating protein-protein interactions, and thus can influence a wide variety of biological processes, including cell surface recognition; modulation of cell-cell contact; modulation of cell fate determination; and modulation of wound healing and tissue repair. The EGF-like domain (HMM) has been assigned the PFAM Accession Number PF00008.

In a preferred embodiment, a 1983 polypeptide or protein has at least one, preferably two EGF-like domains of about 25 to 50, preferably about 30 to 45, and more preferably 30 to 40 amino acid residues in length, and has at least about 60%, 70% 80% 90% 95%, 99%, or 100% homology with an “EGF-like domain,” e.g., at least two EGF-like domains of human 1983 (e.g., residues 21-57 and 61-108 of SEQ ID NO:78).

In another embodiment, a 1983 protein includes at least one latrophilin CL-1-like GPS domain. As used herein, a “latrophilin CL-1-like GPS” domain refers to an amino acid sequence of about 25-120 amino acids, preferably about 40-80, and most preferably, about 50 amino acids which is capable of binding alpha-Latrotoxin, a potent excitatory neurotoxin. The latrophilin CL-1-like GPS domain (HMM) has been assigned the PFAM Accession Number PF01825.

In a preferred embodiment, a 1983 polypeptide or protein has at least one latrophilin CL-1-like GPS domain of about 25-120 amino acids, preferably about 40-80, and most preferably, about 50 amino acid residues in length, and has at least about 60%, 70% 80% 90% 95%, 99%, or 100% homology with an “latrophilin CL-1-like GPS domain,” e.g., at least one latrophilin CL-1-like GPS domain of human 1983 (e.g., residues 366 to 418 of SEQ ID NO:78).

In one embodiment, a 50289 protein includes at least one at least one ANF ligand binding domain. Preferably, the ANF ligand binding domain is found in the extracellular domain of a 50289 protein. As used herein, an “ANF ligand binding domain” refers to an amino acid sequence of about 100 to 600, preferably, 200-500, more preferably, 300-450, and most preferably, about 409 amino acids which is preferably located outside a cell or extracellularly. Preferably, the ANF ligand binding domain interacts (e.g., binds to) a natriuretic peptide (i.e., a hormone involved in the regulation of fluid and electrolyte homeostasis). Preferred, ANF ligand binding domains mediate the intracellular production of a second messenger, e.g., cGMP, thereby transducing an extracellular signal. Preferred ANF ligand binding domains are involved in modulating a cellular activity, e.g., the regulation of fluid and electrolyte homeostasis. The ANF ligand binding domain (HMM) has been assigned the PFAM Accession Number PF01094.

In a preferred embodiment, a 50289 polypeptide or protein has at least one ANF ligand binding domain of about 100 to 600, preferably, 200-500, more preferably, 300-450, and most preferably, about 409 amino acid residues in length, and has at least about 60%, 70% 80% 90% 95%, 99%, or 100% homology with an “ANF ligand binding domain,” e.g., at least one ANF ligand binding domain of human 50289 (e.g., residues 61 to 470 of SEQ ID NO:90).

In one embodiment, a 1983, 52881, 2398, 45449, 50289 or 52872 protein includes at least one, two, three, four, five, six, or preferably, seven transmembrane domains. As used herein, the term “transmembrane domain” includes an amino acid sequence of about 15 amino acid residues in length which spans the plasma membrane. More preferably, a transmembrane domain includes about at least 16, 18, 20, 25, 30, 35 or 40 amino acid residues and spans the plasma membrane. Transmembrane domains are rich in hydrophobic residues, and typically have a α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al, (1996) Annual Rev. Neuronsci. 19: 235-63, the contents of which are incorporated herein by reference.

In a preferred embodiment, a 1983, 52881, 2398, 45449, 50289 or 52872 polypeptide or protein has at least one transmembrane domain or a region which includes at least 16, 18, 20, 25 30, 35 or 40 amino acid residues and has at least about 60%, 70% 80% 90% 95%, 99%, or 100% homology with a “transmembrane domain,” e.g., at least one transmembrane domain of human 1983, 52881, 2398, 45449, 50289 or 52872 (e.g., amino acid residues 433-452, 465-482, 500-524, 533-553, 570-594, 619-636, and 643-667 of SEQ ID NO:78; amino acid residues 11-34, 44-67, 85-106, 127-149, 172-196, and 245-269 of SEQ ID NO:81; amino acid residues 42-66, 78-99, 114-135, 154-176, 202-224, 241-259, 291-310 of SEQ ID NO:84; amino acid residues 12-33, 68-90, and 123-147 of SEQ ID NO:87; amino acid residues 567-590, 600-623, 641-659, 679-702, 726-750, 762-782, and 799-810 of SEQ ID NO:90; and amino acid residues 43-67, 76-110, 117-136, 158-180, 204-228, 264-285, and 310-326 of SEQ ID NO:93). Preferably, the transmembrane domain transduces a signal, e.g., an extracellular signal across a cell membrane, and/or activates a signal transduction pathway.

In another embodiment, a 1983, 2398, 50289, or 52872 protein includes at least one extracellular domain. When located at the N-terminal domain the extracellular domain is referred to herein as an “N-terminal extracellular domain”, or as an N-terminal extracellular loop in the amino acid sequence of the protein. As used herein, an “N-terminal extracellular domain” includes an amino acid sequence having about 1-600, preferably about 1-500, preferably about 1-400, preferably about 1-300, preferably about 1-100, more preferably about 1-70, more preferably about 1-60, more preferably about 1-50, or even more preferably about 1-45 amino acid residues in length and is located outside of a cell or extracellularly. The C-terminal amino acid residue of a “N-terminal extracellular domain” is adjacent to an N-terminal amino acid residue of a transmembrane domain in a naturally-occurring 1983, 2398, 50289, or 52872, or 1983, 2398, 50289, or 52872-like protein. For example, an N-terminal cytoplasmic domain is located at about amino acid residues 20-432 of SEQ ID NO:78, 1-41 of SEQ ID NO:84, 1-546 of SEQ ID NO:90, and 1-42 of SEQ ID NO:93.

In a preferred embodiment, a 1983, 2398, 50289, or 52872 polypeptide or protein has an “N-terminal extracellular domain” or a region which includes at least about 1-600, preferably about 1-500, preferably about 1-400, preferably about 1-300, preferably about 1-100, more preferably about 1-70, more preferably about 1-60, more preferably about 1-50, or even more preferably about 1-45 amino acid residues and has at least about 60%, 70% 80% 90% 95%, 99%, or 100% homology with an “N-terminal extracellular domain,” e.g., the N-terminal extracellular domain of human 1983, 2398, 50289, or 52872 (e.g., residues 20-432 of SEQ ID NO:78, 1-41 of SEQ ID NO:84, 1-546 of SEQ ID NO:90, and 1-42 of SEQ ID NO:93). Preferably, the N-terminal extracellular domain is capable of interacting (e.g., binding to) with an extracellular signal, for example, a ligand or a cell surface receptor. Most preferably, the N-terminal extracellular domain mediates protein-protein interactions, signal transduction and/or cell adhesion. For example, an EGF-like domain of a 1983 polypeptide may mediate protein-protein interactions. Similarly, an ANF binding domain of a 50289 receptor may mediate ligand binding and/or transduction of an extracellular signal.

In another embodiment, a 1983, 2398, 50289 or 52872 protein include at least one extracellular loop. As defined herein, the term “loop” includes an amino acid sequence having a length of at least about 4, preferably about 5-10, and more preferably about 10-20 amino acid residues, and has an amino acid sequence that connects two transmembrane domains within a protein or polypeptide. Accordingly, the N-terminal amino acid of a loop is adjacent to a C-terminal amino acid of a transmembrane domain in a naturally-occurring a 1983, 2398, 50289 or 52872, or a 1983, 2398, 50289 or 52872-like molecule, and the C-terminal amino acid of a loop is adjacent to an N-terminal amino acid of a transmembrane domain in a naturally-occurring 1983, 2398, 50289 or 52872, or a 1983, 2398, 50289 or 52872-like molecule. As used herein, an “extracellular loop” includes an amino acid sequence located outside of a cell, or extracellularly. For example, an extracellular loop can be found at about amino acids 482-499, 554-569 and 637-642 of SEQ ID NO:78; at about amino acids 100-113, 177-201, and 260-290 of SEQ ID NO:84; at about amino acids 624-640, 703-678, and 751-761 of SEQ ID NO:90; and at about amino acids 111-116, 181-203, and 286-309 of SEQ ID NO:93.

In a preferred embodiment, a 1983, 2398, 50289 or 52872 polypeptide or protein has at least one extracellular loop or a region which includes at least about 4, preferably about 5-10, preferably about 10-20, and more preferably about 20-30 amino acid residues and has at least about 60%,70% 80% 90% 95%, 99%, or 100% homology with an “extracellular loop,” e.g., at least one extracellular loop of human 1983, 2398, 50289 or 52872 (e.g., residues 482-499, 554-569 and 637-642 of SEQ ID NO:78; residues 100-113, 177-201, and 260-290 of SEQ ID NO:84; residues 624-640, 703-678, and 751-761 of SEQ ID NO:90; and residues 111-116, 181-203, and 286-309 of SEQ ID NO:93).

In another embodiment, a 1983, 2398, 50289 or 52872 protein includes at least one cytoplasmic loop, also referred to herein as a cytoplasmic domain. As used herein, a “cytoplasmic loop” includes an amino acid sequence having a length of at least about 5, preferably about 5-10, and more preferably about 10-20 amino acid residues located within a cell or within the cytoplasm of a cell. For example, a cytoplasmic loop is found at about amino acids 453-464, 525-532 and 595-618 of SEQ ID NO:78; at about amino acids 67-77, 136-153, and 225-240 of SEQ ID NO:84; at about amino acids 591-599, 660-678, and 703-725 of SEQ ID NO:90; and at about amino acids 68-75, 137-157, and 229-263 of SEQ ID NO:93.

In a preferred embodiment, a 1983, 2398, 50289 or 52872 polypeptide or protein has at least one cytoplasmic loop or a region which includes at least about 5, preferably about 5-10, and more preferably about 10-20 amino acid residues and has at least about 60%, 70% 80% 90% 95%, 99%, or 100% homology with an “cytoplasmic loop,” e.g., at least one cytoplasmic loop of human 1983, 2398, 50289 or 52872 (e.g., residues 453-464, 525-532 and 595-618 of SEQ ID NO:78; residues 67-77, 136-153, and 225-240 of SEQ ID NO:84; residues 591-599, 660-678, and 703-725 of SEQ ID NO:90; or residues 68-75, 137-157, and 229-263 of SEQ ID NO:93).

In another embodiment, a 1983, 2398, 50289 or 52872 protein includes a “C-terminal cytoplasmic domain”, also referred to herein as a C-terminal cytoplasmic tail, in the sequence of the protein. As used herein, a “C-terminal cytoplasmic domain” includes an amino acid sequence having a length of at least about 50, preferably about 50-100, more preferably about 70-93 amino acid residues and is located within a cell or within the cytoplasm of a cell. Accordingly, the N-terminal amino acid residue of a “C-terminal cytoplasmic domain” is adjacent to a C-terminal amino acid residue of a transmembrane domain in a naturally-occurring 1983, 2398, 50289 or 52872 or 1983, 2398, 50289 or 52872-like protein. For example, a C-terminal cytoplasmic domain is found at about amino acid residues 668-690 of SEQ ID NO:78; at about amino acid residues 311-350 of SEQ ID NO:84; at about amino acid residues 811-851 of SEQ ID NO:90; and at about amino acid residues 327-398 of SEQ ID NO:93.

In a preferred embodiment, a 1983, 2398, 50289 or 52872 polypeptide or protein has a C-terminal cytoplasmic domain or a region which includes at least about 50, preferably about 50-100, more preferably about 70-93 amino acid residues and has at least about 60%, 70% 80% 90% 95%, 99%, or 100% homology with an “C-terminal cytoplasmic domain,” e.g., the C-terminal cytoplasmic domain of human 1983, 2398, 50289 or 52872 (e.g., residues 668-690 of SEQ ID NO:78; residues 311-350 of SEQ ID NO:84; residues 811-851 of SEQ ID NO:90; or residues 327-398 of SEQ ID NO:93).

In one embodiment, a 52881 or 45449 protein includes at least one N-terminal domain. As used herein, an “N-terminal domain” includes an amino acid sequence having about 1-50 or more preferably about 1-10 amino acids, located at the N-terminus of the protein. The C-terminal amino acid residue of a “N-terminal domain” is adjacent to an N-terminal amino acid residue of a transmembrane domain in a naturally-occurring 52881 or 45449-like protein. For example, an N-terminal domain is located at about amino acid residues 1-10 of SEQ ID NO:81 and amino acid residues 1-11 of SEQ ID NO:87.

In a preferred embodiment, a 52881 or 45449 polypeptide or protein has an “N-terminal domain” or a region which includes at least about 1-50, or 1-10 amino acid residues and has at least about 60%, 70% 80% 90% 95%, 99%, or 100% homology with an “N-terminal domain,” e.g., the N-terminal domain of human 52881 or 45449 (e.g., residues 1-10 of SEQ ID NO:81 or residues 1-11 of SEQ ID NO:87).

In another embodiment, a 52881 or 45449 protein includes a “C-terminal domain”, also referred to herein as a C-terminal tail, in the sequence of the protein. As used herein, a “C-terminal domain” includes an amino acid sequence having a length of at least about 50, preferably about 100-500, more preferably about 200-450, most preferably about 403 amino acid residues. Accordingly, the N-terminal amino acid residue of a “C-terminal domain” is adjacent to a C-terminal amino acid residue of a transmembrane domain in a naturally-occurring 52881 or 45449-like protein. For example, a C-terminal domain is found at about amino acid residues 270-609 of SEQ ID NO:81 and 148-324 of SEQ ID NO:87.

In a preferred embodiment, a 52881 or 45449 polypeptide or protein has a C-terminal domain or a region which includes at least about 50, preferably about 100-500, more preferably about 200-450 amino acid residues and has at least about 60%, 70% 80% 90% 95%, 99%, or 100% homology with an “C-terminal domain,” e.g., the C-terminal domain of human 52881 or 45449 (e.g., residues 270-609 of SEQ ID NO:81 or residues 148-324 of SEQ ID NO:87).

In another embodiment, a 52881 or 45449 protein include at least one non-transmembrane loop. As defined herein, the term “loop” includes an amino acid sequence having a length of at least about 4, preferably about 5-100, and more preferably about 9-50 amino acid residues, and has an amino acid sequence that connects two transmembrane domains within a protein or polypeptide.

In a preferred embodiment, a 52881 or 45449 polypeptide or protein has at least one non-transmembrane loop or a region which includes at least about 4, preferably about 5-100, preferably about 9-50, and has at least about 60%, 70% 80% 90% 95%, 99%, or 100% homology with a “non-transmembrane loop,” e.g., at least one non-transmembrane loop of human 52881 or 45449 (e.g., residues 35-43, 68-84, 107-126, 150-171, or 197-244 of SEQ ID NO:81 or residues 91-122 or 34-67 of SEQ ID NO:87).

In one embodiment of the invention, a 1983 polypeptide includes at least one, and preferably six or seven, transmembrane domains and/or at least one cytoplasmic loop, and/or at least one extracellular loop. In another embodiment, a 1983 polypeptide further includes an N-terminal extracellular domain and/or a C-terminal cytoplasmic domain. In another embodiment, a 1983 polypeptide can include seven transmembrane domains, three cytoplasmic loops, three extracellular loops and can further include an N-terminal extracellular domain and/or a C-terminal cytoplasmic domain.

In one embodiment of the invention, a 52881 protein includes at least one, and preferably two, three, four, five, or six transmembrane domains and/or at least one, and preferably two, three, four, or five non-transmembrane loops. In another embodiment, the 52881 protein further includes an N-terminal domain and/or a C-terminal domain. The 52881 molecules of the present invention can further include at least one, two, three, and preferably four cAMP/cGMP phosphorylation sites. The 52881 molecules can additionally include at least one, two, three, four, five, and preferably six protein kinase C phosphorylation sites. The 52881 molecules can additionally include at least one, two, three, four, five, six, seven, eight, nine, and preferably 10 casein kinase II phosphorylation sites. The 52881 molecules can additionally include at least one tyrosine kinase phosphorylation site. The 52881 molecules can additionally include at least one, two, three, four, five, six, seven, eight, nine, and preferably 10 N-myristoylation sites. The 52881 molecules can further include at least one amidation site.

In one embodiment of the invention, a 2398 polypeptide includes at least one, and preferably six or seven, transmembrane domains and/or at least one cytoplasmic loop, and/or at least one extracellular loop. In another embodiment, a 2398 polypeptide further includes an N-terminal extracellular domain and/or a C-terminal cytoplasmic domain. In another embodiment, a 2398 polypeptide can include seven transmembrane domains, three cytoplasmic loops, three extracellular loops and can further include an N-terminal extracellular domain and/or a C-terminal cytoplasmic domain.

In one embodiment of the invention, a 45449 protein includes at least one, and preferably two, or three transmembrane domains and/or at least one, and preferably two non-transmembrane loops. In another embodiment, the 45449 protein further includes an N-terminal domain and/or a C-terminal domain.

In one embodiment of the invention, a 50289 polypeptide includes at least one, and preferably six or seven, transmembrane domains and/or at least one cytoplasmic loop, and/or at least one extracellular loop. In another embodiment, a 50289 polypeptide further includes an N-terminal extracellular domain and/or a C-terminal cytoplasmic domain. In another embodiment, a 50289 polypeptide can include seven transmembrane domains, three cytoplasmic loops, three extracellular loops and can further include an N-terminal extracellular domain and/or a C-terminal cytoplasmic domain.

In one embodiment of the invention, a 52872 polypeptide includes at least one, and preferably six or seven, transmembrane domains and/or at least one cytoplasmic loop, and/or at least one extracellular loop. In another embodiment, a 52872 polypeptide further includes an N-terminal extracellular domain and/or a C-terminal cytoplasmic domain. In another embodiment, a 52872 polypeptide can include seven transmembrane domains, three cytoplasmic loops, three extracellular loops and can further include an N-terminal extracellular domain and/or a C-terminal cytoplasmic domain. The 52872 molecules of the present invention can further include at least one, two, and preferably three N-glycosylation sites. The 52872 molecules can additionally include at least one, preferably two protein kinase C phosphorylation sites. The 52872 molecules can further include at least one, two, three, four and preferably five N-myristylation sites.

Based on the above-described sequence similarities, the 1983, 52881, 2398, 45449, 50289, and 52872 molecules of the present invention are predicted to have similar biological activities as members of the GPCR family. The response mediated by a 1983, 52881, 2398, 45449, 50289, or 52872 receptor protein can depend on the type of cell. For example, in some cells, binding of a ligand to the receptor protein may stimulate an activity such as release of compounds, gating of a channel, cellular adhesion, migration, differentiation, etc., through phosphatidylinositol or cyclic AMP metabolism and turnover while in other cells, the binding of the ligand can produce a different result. Regardless of the cellular activity/response modulated by the receptor protein, it is universal that the protein is a GPCR and interacts with G proteins to produce one or more secondary signals, in a variety of intracellular signal transduction pathways, e.g., through phosphatidylinositol or cyclic AMP metabolism and turnover, in a cell. As used herein, a “signaling transduction pathway” refers to the modulation (e.g., stimulation or inhibition) of a cellular function/activity upon the binding of a ligand to the GPCR (52872 protein). Examples of such functions include mobilization of intracellular molecules that participate in a signal transduction pathway, e.g., phosphatidylinositol 4,5-bisphosphate (PIP₂), inositol 1,4,5-triphosphate (IP₃) and adenylate cyclase.

As used herein, “phosphatidylinositol turnover and metabolism” refers to the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of ligand to the receptor activates, in some cells, the plasma-membrane enzyme phospholipase C that in turn can hydrolyze PIP₂ to produce 1,2-diacylglycerol (DAG) and inositol 1,4,5-triphosphate (IP₃). Once formed IP₃ can diffuse to the endoplasmic reticulum surface where it can bind an IP₃ receptor, e.g., a calcium channel protein containing an IP₃ binding site. IP₃ binding can induce opening of the channel, allowing calcium ions to be released into the cytoplasm. IP₃ can also be phosphorylated by a specific kinase to form inositol 1,3,4,5-tetraphosphate (IP₄), a molecule which can cause calcium entry into the cytoplasm from the extracellular medium. IP₃ and IP₄ can subsequently be hydrolyzed very rapidly to the inactive products inositol 1,4-biphosphate (IP₂) and inositol 1,3,4-triphosphate, respectively. These inactive products can be recycled by the cell and used to synthesize PIP₂. The other second messenger produced by the hydrolysis of PIP₂, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it may be activated by DAG. The activation of protein kinase C in different cells results in various cellular responses such as the phosphorylation of glycogen synthase, or the phosphorylation of various transcription factors, e.g., NF-κB. The language “phosphatidylinositol activity”, as used herein, refers to an activity of PIP₂ or one of its metabolites.

Another signaling pathway in which the receptor may participate is the cAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand-induced stimulation of certain G protein coupled receptors. In the cAMP signaling pathway, binding of a ligand to a GPCR can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

TaqMan analysis shows 52872 is highly expressed in the central and peripheral nervous system. 52872 mRNA is expressed at high levels, relative to other tissues tested, in the human brain and spinal cord. Expression was also detected in placenta, testes, thymus, and dorsal root ganglion (DRG). In the monkey, high level 52872 expression was detected in the cortex and the spinal cord. In situ hybridization showed expression of 52872 in the brain cortex, striatum, thalamus, spinal cord, and dorsal horni. Low levels of expression were detected in a small population of medium size DRG neurons.

Animal models of pain response include, but are not limited to: axotomy, the cutting or severing of an axon (Gustafsson et al. (2000) Neuroreport 11:3345-48); chronic constriction injury (CCI), also known as the Bennett model, a model of neuropathic pain which involves ligation of the sciatic nerve in rodents, e.g., rats (Eaton et al. (2000) Cell Transplant. 9:637-56); or intraplantar complete Freund's adjuvant (CFA) injection as a model of arthritic pain (Fraser et al. (2000) Br. J. Pharmacol. 129:1668-72). Other animal models of pain response are described in, e.g., ILAR Journal (1999) Volume 40, Number 3 (entire issue).

52872 expression was shown to be regulated in three different pain response models. Specifically, the upregulation of 52872 expression was detected in DRG following CFA injection (28 days), axotomy (7 days), and CCI (7 days). The upregulation of 52872 expression was also detected in the spinal cord following CFA injection (28 days), axotomy (1-7 days), and CCI (1-14 days).

52872 shows homology to the human galanin receptor type 2 (GAL2-R) (GenBank3 Accession No. 043603). GAL2-R is expressed abundantly within the central nervous system in both the hypothalamus and hippocampus. GAL2-R is a receptor for the hormone galanin, a 29 amino acid neoropeptide that is present in sensory and spinal dorsal horn neurons. Conditions associated with chronic pain such as peripheral nerve injury and inflammation are associated with upregulated synthesis of galanin, e.g., in sensory neurons and spinal cord neurons. Endogenous galanin has been proposed to function as a modulator of nociceptive input, e.g., at the spinal level. The administration of exogenous galanin exerts complex effects on spinal nociceptive transmission, although inhibitory action appears to predominate (Xu et al. (2000) Neuropeptides 34:137-47). Despite these observations, the precise role of galanin in pain processing remains a subject of debate (liu et al. (2000) Brain Res. 886:67-72). Galanin may participate in nociceptive processing by mediating interrelated inhibitory and excitatory effects (Kerr et al. (2000) Eur. J. Neurosci 12:793-802).

Based upon the expression patterns of 52872, the regulated expression in pain models, and its homology to the galanin receptor type 2, 52872 is likely a receptor for a neuropeptide, e.g., a neuropeptide involved in nociception.

52872 associated disorders can detrimentally affect regulation and modulation of the pain response, vasoconstriction, inflammatory response and pain therefrom. Examples of disorders in which the 52872 molecules of the invention may be directly or indirectly involved include pain, pain syndromes, and inflammatory disorders, including inflammatory pain as described in more detail below.

52881 mRNA is expressed in cultured endothelial cells and its expression is downregulated during the formation of vascular tube-like structures. This regulation of 52881 expression suggests that the 52881 protein may inhibit vascular tube formation, a process thought to be similar to angiogenesis. This observation also suggests that 52881 may participate in atherosclerosis and/or the control of vascular tone, as endothelial cell phenotype plays an important role in both of these processes. For example, the expression of cyclooxygenase-2 and endothelin-1, two genes with established relevance to atherosclerosis and the control of vascular tone, have been shown to be regulated in models. Based upon the regulated endothelial cell expression of 52881, the polypeptides of the invention may be useful for developing novel diagnostic and therapeutic agents for 52881-mediated or related disorders, e.g., cardiovascular disorders and angiogenesis-related disorders.

Based upon the 1983, 2398, 45449 expression in cardiovascular tissues (e.g., the heart and endothelial cells), it is likely that these molecules are involved in cardiovascular disorders, including hyperproliferative vascular diseases (e.g., hypertension, vascular restenosis and atherosclerosis, ischaemia reperfusion injury, cardiac hypertrophy, coronary artery disease, myocardial infarction, arrythmia, cardiomyopathies, and congestive heart failure), as described in more detail below.

The 1983 molecules of the invention may be involved in skin disorders. Similarly, 1983 molecules are expressed liver cells, e.g., hemangiomas, and thus may be involved in mediating liver disorders (as described in more detail below). Accordingly, 1983 molecules can act as novel diagnostic targets and therapeutic agents for controlling disorders involving aberrant activities of these cells.

Similarly, expression of 52872, 1983, 2398, 45449 and 50289 is detected in the neural tissues, e.g., the brain. Accordingly, these molecules can act as novel diagnostic targets and therapeutic agents for controlling neurological disorders. 50289 mRNA expression is also detected in the testis, small intestine and the pituitary. 45449 mRNA expression is also detected in granulocytes and liver cells. Thus, it is likely that 50289 and 45449 molecules are involved in disorders involving aberrant activities of these cells.

As the 1983, 52881, 2398, 45449, 50289, or 52872 polypeptides of the invention may modulate 1983, 52881, 2398, 45449, 50289, or 52872-mediated activities, they may be useful as of for developing novel diagnostic and therapeutic agents for 1983, 52881, 2398, 45449, 50289, or 52872-mediated or related disorders, as described below.

As used herein, a “1983, 52881, 2398, 45449, 50289, or 52872 activity”, “biological activity of 1983, 52881, 2398, 45449, 50289, or 52872” or “functional activity of 1983, 52881, 2398, 45449, 50289, or 52872”, refers to an activity exerted by a 1983, 52881, 2398, 45449, 50289, or 52872 protein, polypeptide or nucleic acid molecule on e.g., a 1983, 52881, 2398, 45449, 50289, or 52872-responsive cell or on a 1983, 52881, 2398, 45449, 50289, or 52872 substrate, e.g., a protein substrate, as determined in vivo or in vitro. In one embodiment, a 1983, 52881, 2398, 45449, 50289, or 52872 activity is a direct activity, such as an association with a 52872 target molecule. A “target molecule” or “binding partner” is a molecule with which a 1983, 52881, 2398, 45449, 50289, or 52872 protein binds or interacts in nature. In an exemplary embodiment, 1983, 52881, 2398, 45449, 50289, or 52872 is a receptor, e.g., a receptor for a neuropeptide.

A 1983, 52881, 2398, 45449, 50289, or 52872 activity can also be an indirect activity, e.g., a cellular signaling activity mediated by interaction of the 1983, 52881, 2398, 45449, 50289, or 52872 protein with a 1983, 52881, 2398, 45449, 50289, or 52872 receptor. Based on the above-described sequence similarities, the 1983, 52881, 2398, 45449, 50289, or 52872 molecules of the present invention are predicted to have similar biological activities as G protein-coupled receptor family members, e.g., neuropeptide receptors. For example, the 1983, 52881, 2398, 45449, 50289, or 52872 proteins of the present invention can have one or more of the following activities: (1) regulating, sensing and/or transmitting an extracellular signal into a cell, for example, transmitting a pain related signal from a neuropeptide; (2) signaling to G proteins; (3) modulating a pain or inflammation response; (4) modulating angiogenesis and/or the control of vascular tone; (5) interacting with (e.g., binding to) an extracellular signal, e.g., a neuropeptide, or a cell surface receptor; (6) mobilizing an intracellular molecule that participates in a signal transduction pathway (e.g., adenylate cyclase or phosphatidylinositol 4,5-bisphosphate (PIP₂), inositol 1,4,5-triphosphate (IP₃)); (7) controlling production or secretion of molecules; (8) altering the structure of a cellular component; (9) modulating cell proliferation, e.g., synthesis of DNA; or (10) modulating cell migration, cell differentiation; and cell survival

As the 1983, 52881, 2398, 45449, 50289, or 52872 polypeptides of the invention may modulate 1983, 52881, 2398, 45449, 50289, or 52872-mediated activities, they may be useful for developing novel diagnostic and therapeutic agents for 1983, 52881, 2398, 45449, 50289, or 52872-mediated or related disorders. For example, the 1983, 52881, 2398, 45449, 50289, or 52872 molecules can act as novel diagnostic targets and therapeutic agents controlling cardiovascular disorders.

Preferred examples of cardiovascular disorders or diseases include a cardiovascular condition associated with interventional procedures (“procedural vascular trauma”), such as restenosis following angioplasty, placement of a shunt, stet, stent, synthetic or natural excision grafts, indwelling catheter, valve or other implantable devices.

In some embodiments, the therapeutic and prophylactic uses of the compositions of the invention, further include the administration of cholesterol lowering agents as a combination drug therapies. The term “combination therapy” as used herein refers to the administration to a subject (concurrently or sequentially) of two or more cholesterol lowering agents. Current combination therapy therapies using combinations of niacin and statins are being used with positive results to treat hyperlipidemia (Guyton, J R. (1999) Curr Cardiol Rep. 1(3):244-250; Otto, C. et al. (1999) Internist (Berl) 40(12):1338-45). Other useful drug combinations include those derived by addition of fish oil, bile acid binding resins, or stanol esters, as well as nonstatin combinations such as niacin-resin or fibrate-niacin (Guyton, J R. (1999) supra). For examples of dosages and administration schedules of the cholesterol lowering agents, the teachings of Guyton, J R. (1999) supra, Otto, C. et al. (1999) supra, Guyton, J R et al. (1998) Am J Cardiol 82(12A):82U-86U; Guyton, J R et al. (1998) Am J Cardiol. 82(6):737-43; Vega, G L et al. (1998) Am J. Cardiol. 81(4A):36B-42B; Schectman, G. (1996) Ann Intern Med. 125(12):990-1000; Nakamura, H. et al. (1993) Nippon Rinsho 51(8):2101-7; Goldberg, A. et al. (2000) Am J Cardiol 85(9):1100-5; Morgan, J M et al. (1996) J Cardiovasc. Pharmac. Ther. 1(3):195-202; Stein, E A et al. (1996) J Cardiovasc Pharmacol Ther 1(2): 107-116; and Goldberg, A C (1998) Am Cardiol 82(12A):35U-41U, are expressly incorporated by reference.

As used herein, “cholesterol lowering agents” include agents which are useful for lowering serum cholesterol such as for example bile acid sequestering resins (e.g. colestipol hydrochloride or cholestyramine), fish oil, stanol esters, an ApoAII-lowering agent, a VLDL lowering agent, an ApoAI-stimulating agent, fibric acid derivatives (e.g. clofibrate, fenofibrate, or gemfibrozil), thiazolidenediones (e.g. troglitazone), or HMG-CoA reductase inhibitors (e.g. statins, such as fluvastatin sodium, lovastatin, pravastatin sodium, or simvastatin), as well as nicotinic acid, niacin, or probucol.

“VLDL-lowering agent” includes an agent which decreases the hepatic synthesis of triglyceride-rich lipoproteins or increases the catabolism of triglyceride-rich lipoproteins, e.g., fibrates such as gemfibrozil, or the statins, increases the expression of the apoE-mediated clearance pathway, or improves insulin sensitivity in diabetics, e.g., the thiazolidene diones.

As the 1983, 52881, 2398, 45449, 50289, or 52872 polypeptides of the invention may modulate 1983, 52881, 2398, 45449, 50289, or 52872-mediated activities, they may be useful for developing novel diagnostic and therapeutic agents for 1983, 52881, 2398, 45449, 50289, or 52872-mediated or related disorders. For example, the 1983, 52881, 2398, 45449, 50289, or 52872 molecules can act as novel diagnostic targets and therapeutic agents controlling pain, pain disorders, and inflammatory disorders. For example, a 1983, 52881, 2398, 45449, 50289, or 52872 inhibitor can be useful in the treatment of pain, as 1983, 52881, 2398, 45449, 50289, or 52872 inhibition could increase the endogenous levels of enkephalins and thereby increase the associated analgesic response.

Agents that modulate 1983, 52881, 2398, 45449, 50289, or 52872 polypeptide or nucleic acid activity or expression can be used to treat pain elicited by any medical condition. A subject receiving the treatment can be additionally treated with a second agent, e.g., an anti-inflammatory agent, an antibiotic, or a chemotherapeutic agent, to further ameliorate the condition.

The 1983, 52881, 2398, 45449, 50289, or 52872 molecules can also act as novel diagnostic targets and therapeutic agents controlling pain caused by other disorders, e.g., cancer, e.g., prostate cancer.

Tissue Distribution of 52872 mRNA

Endogenous human 52872 gene expression was determined using the Perkin-Elmer/ABI 7700 Sequence Detection System which employs TaqMan technology. 52872 mRNA levels were analyzed in a variety of samples of human tissues, and in rodent models of pain response. The highest 52872 mRNA expression was observed in brain, placenta, testes, thymus, spinal cord, and DRG.

In another TaqMan panel, the highest 52872 mRNA expression was observed in human brain and spinal cord.

In situ hybridization showed expression of 52872 in the brain cortex, striatum, thalamus, spinal cord, and dorsal horni. Low level expression was detected in a small population of medium size DRG neurons.

Further analysis was performed in mRNA derived from monkey and human tissue samples. Highest expression in monkey tissues was detected in cortex and spinal cord. Highest expression in human tissues was detected in brain.

Taqman experiments in rodent models of pain response showed that the 52872 gene is regulated in three different pain response models. There was upregulation of 52872 expression in DRG following CFA injection (28 days), axotomy (7 days), and CCI (7 days). There was also upregulation of 52872 expression in the spinal cord following CFA injection (28 days), axotomy (1-7 days), and CCI (1-14 days).

### Expression of 52881 in Endothelial Cells

Human umbilical vein endothelial cells (HUVEC) were gown under a variety of conditions and the levels of 52881 expression were determined by microarray hybridization.

2881 is expressed in cultured endothelial cells and is down-regulated during the formation of vascular tube-like structures that are induced by plating on Matrigel.

Tissue Distribution of 1983, 2398, 45449, and 50289 mRNA

Human 1983, 2398, 45449 and 50289 gene expression were evaluated using TaqMan technology as described herein.

1983 mRNA was expressed in the heart, e.g., the diseased heart (e.g., heart tissue from humans with cardiac myopathy, or congestive heart failure). 1983 mRNA was also expressed in blood vessels, e.g., aorta, veins, human umbilical cord vein-derived endothelial cells (HUVEC), human microvascular endothelial cells (HMVEC), and endothelial cells, as well as in the skin. 1983 mRNA was also found at relatively high levels in the brain and the kidney and in hemangioma. 1983 mRNA was also expressed in mouse hindlimb.

2398 mRNA was expressed in vessels, e.g., static HUVEC, shear HUVEC, as well as in the brain and dermal cells. 2398 mRNA levels were also detected in tissues and cell samples rich in vascular cells.

45449 mRNA was expressed in heart and brain, as well as in HepG2-A cells.

50289 mRNA was expressed at elevated levels in, e.g., testes, small intestine, and the pituitary gland.

### Human 26908

The present invention is based, in part, on the discovery of novel G-protein coupled receptors and nucleic acids encoding these receptors, referred to herein collectively as “GPCRs,” or by the individual clone name “26908.”

The human 26908 nucleotide sequence (SEQ ID NO:103), which is approximately 1829 nucleotides long including untranslated regions, contains a predicted coding sequence of about 1260 nucleotides (142-1401 of SEQ ID NO:103; 1-1260 of SEQ ID NO: 105), not including the terminal codon. The coding sequence encodes a 420 amino acid protein (SEQ ID NO:104). The 26908 protein has homology with a seven transmembrane (7™) domain.

Human 26908 contains the following regions or other structural features (for general information regarding PFAM identifiers, PS prefix and PF prefix domain identification numbers, refer to Sonnhammer et al. (1997) Protein 28:405-420: five dileucine motifs in the tail (LL) (predicted by PSORT, Nakai, K. and Kanehisa, M. (1992) Genomics 14:897-911) at about amino acids 121 to 122, 245 to 246, 332 to 333, 351 to 352, and 352 to 353 of SEQ ID NO:104; one leucine zipper pattern site (Prosite PS00029) located at about amino acids 238 to 259 of SEQ ID NO:104; four transmembrane domains (predicted by MEMSAT, Jones et al. (1994) Biochemistry 33:3038-3049) at about amino acids 63 to 79, 118 to 135, 338 to 354 and 388 to 406 of SEQ ID NO:104; six protein kinase C phosphorylation sites (Prosite PS00005) at about amino acids 3 to 5, 87 to 89, 95 to 97, 208 to 210, 255 to 257, and 311 to 313 of SEQ ID NO:104; five casein kinase II phosphorylation sites (Prosite PS00006) located at about amino acids 87 to 90, 161 to 164, 247 to 250, 255 to 258, and 390 to 393 of SEQ ID NO:104; one tyrosine kinase phosphorylation site (Prosite PS00007) located at about amino acids 383 to 389 of SEQ ID NO:104; and five N-myristoylation sites (Prosite PS00008) located at about amino acids 16 to 21, 47 to 52, 63 to 68, 233 to 238, and 338 to 343 of SEQ ID NO:104.

A BLAST alignment of human 26908 with a consensus amino acid sequence derived from a ProDomain No. PD138974 “receptor transmembrane adriamycin G sensitivity modifying testis-specific lantibiotic p40 enzyme” (ProDomain Release 2000.1) shows amino acid residues 6 to 294 of the amino acid consensus sequence (SEQ ID NOs: 106-108) aligns with the “receptor transmembrane adriamycin G sensitivity modifying testis-specific lantibiotic p40 enzyme” of human 26908, amino acid residues 120 to 145, 202-410, and 245 to 363 of SEQ ID NO:104. The BLAST algorithm identifies multiple local alignments between the consensus amino acid sequence and human 26908.

A BLAST alignment of human 26908 with a consensus amino acid sequence derived from a ProDomain No. PD250635 “receptor transmembrane G p40 protein-coupled sensitivity testis-specific adriamycin BcDNA:LD28247 seven-transmembrane domain” (ProDomain Release 2000.1) shows amino acid residues 53 to 200 of the amino acid consensus sequence (SEQ ID NO:109) aligns with the “receptor transmembrane G p40 protein-coupled sensitivity testis-specific adriamycin BcDNA:LD28247 seven-transmembrane domain” of human 26908, amino acid residues 87 to 231 of SEQ ID NO: 104.

A hydropathy plot of human 26908 was performed. Polypeptides of the invention include fragments which include: all or part of a hydrophobic sequence, e.g., the sequence from about amino acid 120 to 140, from about 150 to 165, and from about 230 to 245 of SEQ ID NO:104; all or part of a hydrophilic sequence, e.g., the sequence from about amino acid 90 to 100, from about 210 to 220, and from about 270 to 290 of SEQ ID NO: 104; a sequence which includes a Cys, or a glycosylation site.

TaqMan analysis shows 26908 mRNA is highly expressed in fetal liver, erythroid cells, megakaryocytes, and K562 cells. 26908 mRNA is also expressed in skin, bone marrow and mobilized peripheral blood CD34+ cells and platelets. Based on 26908 expression, it is likely that 26908 molecules of the present invention may be involved in disorders characterized by aberrant activity of these cells. Similarly, the 26908 molecules of the invention may be involved in skin disorders. Accordingly, 26908 molecules can act as novel diagnostic targets and therapeutic agents for controlling disorders involving aberrant activities of these cells.

Additionally, and without being bound by theory, 26908 molecules may be expressed in hematopoietic cells in a lineage restricted manner to play a role in regulating the development of the lineage cells, erythrocytes, neutrophils or megakaryocytes/platelets and/or their function. Low but significant levels of mRNA were detectable in hematopoietic progenitor CD34+ cells and expression increased upon restriction to the megakaryocyte lineage. Expression also increased as bone marrow/blood cell differentiation proceeded, suggesting a role in platelet function and thrombosis. As such, 26908 molecules may serve as specific and novel identifiers of such hematopoietic cells. Further, the 26908 molecules are also useful for the treatment of hematological disorders.

The 26908 receptors of the present invention contains a significant number of structural characteristics in common with members of the G-protein coupled receptor family. Based on structural similarities, members of the GPCR family have been classified into various subfamilies, including: Subfamily I which comprises receptors typified by rhodopsin and the beta2-adrenergic receptor and currently contains over 200 unique members (reviewed by Dohlman et al. (1991) Annu. Rev. Biochem. 60:653-688); Subfamily II, which includes the parathyroid hormone/calcitonin/secretin receptor family (Juppner et al. (1991) Science 254:1024-1026; Lin et al. (1991) Science 254:1022-1024); Subfamily III, which includes the metabotropic glutamate receptor family in mammals, such as the GABA receptors (Nakanishi et al. (1992) Science 258: 597-603); Subfamily IV, which includes the CAMP receptor family that is known to mediate the chemotaxis and development of D. discoideum (Klein et al. (1988) Science 241:1467-1472); and Subfamily V, which includes the fungal mating pheromone receptors such as STE2 (reviewed by Kurjan I et al. (1992) Annu. Rev. Biochem. 61:1097-1129). Within each family, distinct, highly conserved motifs have been identified. These motifs have been suggested to be critical for the structural integrity of the receptor, as well as for coupling to G proteins.

A 26908 polypeptide can include at least one, two, three, preferably four “transmembrane domains” or regions homologous with a “transmembrane domain”. As used herein, the term “transmembrane domain” includes an amino acid sequence of about 10 to 40 amino acid residues in length and spans the plasma membrane. Transmembrane domains are rich in hydrophobic residues, e.g., at least 50%, 60%, 70%, 80%, 90%, 95% or more of the amino acids of a transmembrane domain are hydrophobic, e.g., leucines, isoleucines, tyrosines, or tryptophans. Transmembrane domains typically have alpha-helical structures and are described in, for example, Zagotta, W. N. et al., (1996) Annual Rev. Neurosci. 19:235-263, the contents of which are incorporated herein by reference.

In a preferred embodiment, a 26908 polypeptide or protein has at least one, gradually increase preferably actual “transmembrane domain” or a region which includes at least about 12 to 35 more preferably about 14 to 30 or 15 to 25 amino acid residues and has at least about 60%, 70% 80% 90% 95%, 99%, or 100% homology with a “transmembrane domain,” e.g., the transmembrane domains of human 26908 (e.g., residues 63 to 79, 118 to 135, 338 to 354, and 388 to 406 of SEQ ID NO:104). The transmembrane domain of human 26908 can be visualized in a hydropathy plot as regions of about 15 to 25 amino acids where the hydropathy trace is mostly above the horizontal line.

To identify the presence of a “transmembrane” domain in a 26908 protein sequence, and make the determination that a polypeptide or protein of interest has a particular profile, the amino acid sequence of the protein can be analyzed by a transmembrane prediction method that predicts the secondary structure and topology of integral membrane proteins based on the recognition of topological models (MEMSAT, Jones et al., (1994) Biochemistry 33:3038-3049).

A 26908 polypeptide can include at least one, two, three, four preferably, five “non-transmembrane regions.” As used herein, the term “non-transmembrane region” includes an amino acid sequence not identified as a transmembrane domain. The non-transmembrane regions in 26908 are located at about amino acids 1 to 62, 80 to 117, 136 to 337, and 355 to 387 of SEQ ID NO:104.

The non-transmembrane regions of 26908 include at least one, and preferably two cytoplasmic regions. In one embodiment, a 26908 protein includes at least one, preferably two cytoplasmic loops. As used herein, the term “loop” includes an amino acid sequence that resides outside of a phospholipid membrane, having a length of at least about 4, preferably about 5 to 30, more preferably about 6 to 38 amino acid residues, and has an amino acid sequence that connects two transmembrane domains within a protein or polypeptide. Accordingly, the N-terminal amino acid of a loop is adjacent to a C-terminal amino acid of a transmembrane domain in a 26908 molecule, and the C-terminal amino acid of a loop is adjacent to an N-terminal amino acid of a transmembrane domain in a 26908 molecule. As used herein, a “cytoplasmic loop” includes a loop located inside of a cell or within the cytoplasm of a cell. For example, a “cytoplasmic loop” can be found at about amino acid residues 80 to 117 and 355 to 387 of SEQ ID NO:104.

In a preferred embodiment, a 26908 polypeptide or protein has a cytoplasmic loop or a region which includes at least about 4, preferably about 5 to 30, more preferably about 6 to 38 amino acid residues and has at least about 60%, 70% 80% 90% 95%, 99%, or 100% homology with a cytoplasmic loop,” e.g., a cytoplasmic loop of human 26908 (e.g., residues 80 to 117 and 355 to 387 of SEQ ID NO:104).

In another embodiment, a 26908 protein includes at least one non-cytoplasmic loop. As used herein, a “non-cytoplasmic loop” includes an amino acid sequence located outside of a cell or within an intracellular organelle. Non-cytoplasmic loops include extracellular domains (i.e., outside of the cell) and intracellular domains (i.e., within the cell). When referring to membrane-bound proteins found in intracellular organelles (e.g., mitochondria, endoplasmic reticulum, peroxisomes microsomes, vesicles, endosomes, and lysosomes), non-cytoplasmic loops include those domains of the protein that reside in the lumen of the organelle or the matrix or the intermembrane space. For example, a “non-cytoplasmic loop” can be found at about amino acid residues 136 to 337 of SEQ ID NO:104.

In a preferred embodiment, a 26908 polypeptide or protein has at least one non-cytoplasmic loop or a region which includes at least about 4, preferably about 5 to 200, more preferably about 6 to 202 amino acid residues and has at least about 60%, 70% 80% 90% 95%, 99%, or 100% homology with a “non-cytoplasmic loop,” e.g., at least one non-cytoplasmic loop of human 26908 (e.g., residues 136 to 337 of SEQ ID NO:104).

When located at the N-terminus, an extracellular region is referred to herein as the “N-terminal extracellular domain.” As used herein, an “N-terminal extracellular domain” includes an amino acid sequence having about 1 to 80, preferably about 1 to 70, more preferably about 1 to 65, or even more preferably about 1 to 62 amino acid residues in length and is located outside of a cell or outside the cytoplasm of a cell. The C-terminal amino acid residue of an “N-terminal extracellular domain” is adjacent to an N-terminal amino acid residue of a transmembrane domain in a 26908 protein. For example, an N-terminal extracellular domain is located at about amino acid residues 1 to 62 of SEQ ID NO:104.

In a preferred embodiment, a polypeptide or protein has an N-terminal extracellular domain or a region which includes at least about 5, preferably about 1 to 70, and more preferably about 1 to 62 amino acid residues and has at least about 60%, 70% 80% 90% 95%, 99%, or 100% homology with an “N-terminal extracellular domain,” e.g., the N-terminal extracellular domain of human 26908 (e.g., residues 1 to 62 of SEQ ID NO:104).

In another embodiment, an extracellular region of a 26908 protein can include the C-terminus and can be a “C-terminal extracellular domain,” also referred to herein as a “C-terminal extracellular tail.” As used herein, a “C-terminal extracellular domain” includes an amino acid sequence having a length of at least about 5, preferably about 5 to 10, more preferably about 5 to 14 amino acid residues and is located outside of a cell or outside the cytoplasm of a cell. The N-terminal amino acid residue of a “C-terminal extracellular domain” is adjacent to a C-terminal amino acid residue of a transmembrane domain in a 26908 protein. For example, a C-terminal extracellular domain is located at about amino acid residues 407 to 420 of SEQ ID NO:104.

In a preferred embodiment, a 26908 polypeptide or protein has a C-terminal extracellular domain or a region which includes at least about 5, preferably about 5 to 10, and more preferably about 5 to 14 amino acid residues and has at least about 60%, 70% 80% 90% 95%, 99%, or 100% homology with a C-terminal extracellular domain,” e.g., the C-terminal extracellular domain of human 26908 (e.g., residues 407 to 420 of SEQ ID NO:104).

As used herein, the term “leucine zipper domain” includes an amino acid sequence of about 2 to 10 amino acid residues in length. Preferably a leucine zipper domain has been proposed to explain how some eukaryotic gene regulatory proteins work. The leucine zipper consist of a periodic repetition of leucine residues at every seventh position over a distance covering eight helical turns. The segments containing these periodic arrays of leucine residues seem to exist in an alpha-helical conformation. The leucine side chains extending from one alpha-helix interact with those from a similar alpha helix of a second polypeptide, facilitating dimerization; the structure formed by cooperation of these two regions forms a coiled coil.

Preferably, a leucine zipper domain includes at least about 2 to 10 amino acids, more preferably about 2 to 6 amino acid residues, or about 2 to 4 amino acids. Preferably, the leucine zipper site includes the following amino acid consensus sequence: L-x(6)-L-x(6)-L-x(6)-L (SEQ ID NO:110) having Prosite signatures as PS00029, or sequences homologous thereto. In the above conserved motif, and other motifs described herein, the standard IUPAC one-letter code for the amino acids is used. Each element in the pattern is separated by a dash (-); square brackets ([ ]) indicate the particular residues that are accepted at that position; x indicates that any residue is accepted at that position; and numbers in parentheses (( )) indicate the number of residues represented by the accompanying amino acid. The leucine zipper domain is located between the second and third transmembrane domains of human 26908 polypeptide and which corresponds to about amino acids 238 to 259 of SEQ ID NO:104.

To identify the presence of a GPCR domain in a 26908 protein sequence, and make the determination that a polypeptide or protein of interest has a particular profile, the amino acid sequence of the protein can be searched against a database of domains, e.g., the ProDom database (Corpet et al. (1999), Nucl. Acids Res. 27:263-267). The ProDom protein domain database consists of an automatic compilation of homologous domains. Current versions of ProDom are built using recursive PSI-BLAST searches (Altschul S F et al. (1997) Nucleic Acids Res. 25:3389-3402; Gouzy et al. (1999) Computers and Chemistry 23:333-340) of the SWISS-PROT 38 and TREMBL protein databases. The database automatically generates a consensus sequence for each domain. A BLAST search was performed against the HMM database resulting in the identification of a “GPCR” domain in the amino acid sequence of human 26908 at about residues 120 to 410 of SEQ ID NO:104.

The GPCR domain is homologous to ProDom family PD138974 (“receptor transmembrane adriamycin G sensitivity modifying testis-specific lantibiotic p40 enzyme” SEQ ID NOs: 106-108, ProDomain Release 2000.1). The GPCR domain (amino acids 120 to 410 of SEQ ID NO:104) of human 26908 aligns with a consensus amino acid sequence (SEQ ID NOs: 106-108) derived from a hidden Markov model. The consensus sequence for SEQ ID NO:106 is 39% identical over amino acids 202 to 410 of SEQ ID NO:104. The consensus sequence for SEQ ID NO:107 is 25% identical over amino acids 245 to 363 of SEQ ID NO:104. The consensus sequence for SEQ ID NO:108 is 34% identical over amino acids 120 to 145 of SEQ ID NO:104.

The GPCR domain is also homologous to ProDom family PD250635 (“receptor transmembrane G p40 protein-coupled sensitivity testis-specific adriamycin BcDNA:LD28247 seven-transmembrane domain” SEQ ID NO:109, ProDomain Release 2000.1). The GPCR domain (amino acids 87 to 231 of SEQ ID NO:104) of human 26908 aligns with a consensus amino acid sequence (SEQ ID NO:109) derived from a hidden Markov model. The consensus sequence for SEQ ID NO:109 is 31% identical over amino acids 87 to 231 of SEQ ID NO:104.

To identify the presence of a “GPCR” domain in a 26908 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

A 26908 family member can include at least one, two, three, four, five, six, seven, eight, or preferably nine transmembrane or non-transmembrane domains, or at least one, two, three, four, preferably five dileucine motifs. Furthermore, a 26908 family member can include at least one, two, three, four, five, and preferably six protein kinase C phosphorylation sites (PS00005); at least one, two, three, four, and preferably five casein kinase II phosphorylation sites (Prosite PS00006); at least one tyrosine kinase phosphorylation site (Prosite PS00007); at least one, two, three, four, and preferably five N-myristoylation sites (PS00008); and at least one leucine zipper pattern site (Prosite PS00029).

As the 26908 polypeptides of the invention may modulate 26908-mediated activities, they may be useful as of for developing novel diagnostic and therapeutic agents for 26908-mediated or related disorders, as described below.

As used herein, a “26908 activity”, “biological activity of 26908” or “functional activity of 26908”, refers to an activity exerted by a 26908 protein, polypeptide or nucleic acid molecule on e.g., a 26908-responsive cell or on a 26908 substrate, e.g., a protein substrate, as determined in vivo or in vitro. In one embodiment, a 26908 activity is a direct activity, such as an association with a 26908 target molecule. A “target molecule” or “binding partner” is a molecule with which a 26908 protein binds or interacts in nature. In an exemplary embodiment, is a 26908 receptor. A 26908 activity can also be an indirect activity, e.g., a cellular signaling activity mediated by interaction of the 26908 protein with a 26908 receptor.

The 26908 molecules of the present invention are predicted to have similar biological activities as G-protein coupled receptor family members. For example, the 26908 proteins of the present invention can have one or more of the following activities: (1) regulating, sensing and/or transmitting an extracellular signal into a cell, (for example, a heart cell, a bone cell (e.g., an osteoclast or an-osteoblast), a hematopoietic cell, a neural cell); (2) interacting with (e.g., binding to) an extracellular signal or a cell surface receptor; (3) mobilizing an intracellular molecule that participates in a signal transduction pathway (e.g., adenylate cyclase or phosphatidylinositol 4,5-bisphosphate (PIP2), inositol 1,4,5-triphosphate (IP3)); (4) regulating polarization of the plasma membrane; (5) controlling production or secretion of molecules; (6) altering the structure of a cellular component; (7) modulating cell proliferation, e.g., synthesis of DNA; and (8) modulating cell migration, cell differentiation; and cell survival. Thus, the 26908 molecules can act as novel diagnostic targets and therapeutic agents for controlling G-protein coupled receptor-related disorders. Other activities, as described below, include the ability to modulate function, survival, morphology, proliferation and/or differentiation of cells of tissues in which 26908 molecules are expressed.

The response mediated by a 26908 receptor protein depends on the type of cell. For example, in some cells, binding of a ligand to the receptor protein may stimulate an activity such as release of compounds, gating of a channel, cellular adhesion, migration, differentiation, etc., through phosphatidylinositol or cyclic AMP metabolism and turnover while in other cells, the binding of the ligand will produce a different result. Regardless of the cellular activity/response modulated by the receptor protein, it is universal that the protein is a GPCR and interacts with G proteins to produce one or more secondary signals, in a variety of intracellular signal transduction pathways, e.g., through phosphatidylinositol or cyclic AMP metabolism and turnover, in a cell. As used herein, a “signaling transduction pathway” refers to the modulation (e.g., stimulation or inhibition) of a cellular function/activity upon the binding of a ligand to the GPCR (26908 protein). Examples of such functions include mobilization of intracellular molecules that participate in a signal transduction pathway, e.g., phosphatidylinositol 4,5-bisphosphate (PIP2), inositol 1,4,5-triphosphate (IP3) and adenylate cyclase.

As used herein, “phosphatidylinositol turnover and metabolism” refers to the molecules involved in the turnover and metabolism of phosphatidylinositol 4,5-bisphosphate (PIP2) as well as to the activities of these molecules. PIP2 is a phospholipid found in the cytosolic leaflet of the plasma membrane. Binding of ligand to the receptor activates, in some cells, the plasma-membrane enzyme phospholipase C that in turn can hydrolyze PIP2 to produce 1,2-diacylglycerol (DAG) and inositol 1,4,5-triphosphate (IP3). Once formed IP3 can diffuse to the endoplasmic reticulum surface where it can bind an IP3 receptor, e.g., a calcium channel protein containing an IP3 binding site. IP3 binding can induce opening of the channel, allowing calcium ions to be released into the cytoplasm. IP3 can also be phosphorylated by a specific kinase to form inositol 1,3,4,5-tetraphosphate (IP4), a molecule which can cause calcium entry into the cytoplasm from the extracellular medium. IP3 and IP4 can subsequently be hydrolyzed very rapidly to the inactive products inositol 1,4-biphosphate (IP2) and inositol 1,3,4-triphosphate, respectively. These inactive products can be recycled by the cell to synthesize PIP2. The other second messenger produced by the hydrolysis of PIP2, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, refers to an activity of PIP2 or one of its metabolites.

Another signaling pathway in which the receptor may participate is the CAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand-induced stimulation of certain G protein coupled receptors. In the cAMP signaling pathway, binding of a ligand to a GPCR can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

Based on the above-described sequence similarities, the 26908 molecules of the present invention are predicted to have similar biological activities as G-protein coupled receptor family members. Thus, the 26908 molecules can act as novel diagnostic targets and therapeutic agents for controlling one or more of cellular proliferative and/or differentiative disorders, disorders associated with bone metabolism, immune disorders, hematopoietic disorders, cardiovascular disorders, liver disorders, viral diseases, pain or metabolic disorders.

Additionally, 26908 molecules may play an important role in the etiology of certain viral diseases, including but not limited to Hepatitis B, Heptitis C and Herpes Simplex Virus (HSV). Modulators of 26908 activity could be used to control viral diseases. The modulators can be used in the treatment and/or diagnosis of viral infected tissue or virus-associated tissue fibrosis, especially liver and liver fibrosis. Also, 26908 modulators can be used in the treatment and/or diagnosis of virus-associated carcinoma, especially hepatocellular cancer.

Additionally, 26908 may play an important role in the regulation of metabolism or pain disorders.

Expression and Tissue Distribution of 26908 mRNA

The relative expression levels of 26908 in various tissues was assessed using TaqMan PCR, and significant expression is found in normal fetal liver, erythroid, DRG and megakaryocytes.

### DEFINITIONS

The 18636, 2466, 43238, 1983, 52881, 2398, 45449, 50289, 52872 or 26908 protein, fragments thereof, and derivatives and other variants of the sequence in SEQ ID NO:2, 30, 70, 78, 81, 84, 87, 90, 93 or 104 thereof are collectively referred to as “polypeptides or proteins of the invention” or “18636, 2466, 43238, 1983, 52881, 2398, 45449, 50289, 52872 or 26908 polypeptides or proteins”. Nucleic acid molecules encoding such polypeptides or proteins are collectively referred to as “nucleic acids of the invention” or “18636, 2466, 43238, 1983, 52881, 2398, 45449, 50289, 52872 or 26908 nucleic acids.”

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology (1989) John Wiley & Sons, N.Y., 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a 18636, 2466, 43238, 1983, 52881, 2398, 45449, 50289, 52872 or 26908 protein, preferably a mammalian 18636, 2466, 43238, 1983, 52881, 2398, 45449, 50289, 52872 or 26908 protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of 18636, 2466, 43238, 1983, 52881, 2398, 45449, 50289, 52872 or 26908 protein having less than about 30%, 20%, 10% and more preferably 5% (by dry weight), of non-18636, 2466, 43238, 1983, 52881, 2398, 45449, 50289, 52872 or 26908 protein (also referred to herein as a “contaminating protein”), or of chemical precursors or non-18636, 2466, 43238, 1983, 52881, 2398, 45449, 50289, 52872 or 26908 chemicals. When the 18636, 2466, 43238, 1983, 52881, 2398, 45449, 50289, 52872 or 26908 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 18636, 2466, 43238, 1983, 52881, 2398, 45449, 50289, 52872 or 26908 (e.g., the sequence of SEQ ID NO:1, 3, 29, 31, 69; 71, 77, 79, 80, 82, 83, 85, 86, 88, 89, 91, 92, 94, 103 or 105) without abolishing or more preferably, without substantially altering a biological activity, whereas an “essential” amino acid residue results in such a change. For example, amino acid residues that are conserved among the polypeptides of the present invention, e.g., those present in the conserved domains, are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 18636, 2466, 43238, 1983, 52881, 2398, 45449, 50289, 52872 or 26908 protein is preferably replaced with another amino acid residue from the same side chain family. Alternatively, in another embodiment, mutations can be introduced randomly along all or part of a 18636, 2466, 43238, 1983, 52881, 2398, 45449, 50289, 52872 or 26908 coding sequence, such as by saturation mutagenesis, and the resultant mutants can be screened for 18636, 2466, 43238, 1983, 52881, 2398, 45449, 50289, 52872 or 26908 biological activity to identify mutants that retain activity. Following mutagenesis of SEQ ID NO:1, 3, 29, 31, 69, 71, 77, 79, 80, 82, 83, 85, 86, 88, 89, 91, 92, 94, 103 or 105, the encoded protein can be expressed recombinantly and the activity of the protein can be determined.

As used herein, a “biologically active portion” of a 18636, 2466, 43238, 1983, 52881, 2398, 45449, 50289, 52872 or 26908 protein includes a fragment of a 18636, 2466, 43238, 1983, 52881, 2398, 45449, 50289, 52872 or 26908 protein which participates in an interaction between a 18636, 2466, 43238, 1983, 52881, 2398, 45449, 50289, 52872 or 26908 molecule and a non-18636, 2466, 43238, 1983, 52881, 2398, 45449, 50289, 52872 or 26908 molecule. Biologically active portions of a 18636, 2466, 43238, 1983, 52881, 2398, 45449, 50289, 52872 or 26908 protein include peptides comprising amino acid sequences sufficiently homologous to or derived from the amino acid sequence of the 18636, 2466, 43238, 1983, 52881, 2398, 45449, 50289, 52872 or 26908 protein, e.g., the amino acid sequence shown in SEQ ID NO:2, 30, 70, 78, 81, 84, 87, 90, 93 or 104, which include fewer amino acids than the full length 18636, 2466, 43238, 1983, 52881, 2398, 45449, 50289, 52872 or 26908 protein, and exhibit at least one activity of a 18636, 2466, 43238, 1983, 52881, 2398, 45449, 50289, 52872 or 26908 protein. Typically, biologically active portions comprise a domain or motif with at least one activity of the 18636, 2466, 43238, 1983, 52881, 2398, 45449, 50289, 52872 or 26908 protein. A biologically active portion of a 18636, 2466, 43238, 1983, 52881, 2398, 45449, 50289, 52872 or 26908 protein can be a polypeptide which is, for example, 10, 25, 50, 100, 200 or more amino acids in length. Biologically active portions of a 18636, 2466, 43238, 1983, 52881, 2398, 45449, 50289, 52872 or 26908 protein can be used as targets for developing agents which modulate a 18636, 2466, 43238, 1983, 52881, 2398, 45449, 50289, 52872 or 26908 mediated activity.

Calculations of homology or sequence identity (the terms “homology” and “identity” are used interchangeably herein) between sequences are performed as follows:

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to 18636, 2466, 43238, 1983, 52881, 2398, 45449, 50289, 52872 or 26908 nucleic acid molecules of the invention. BLAST protein searches can be performed with the XBLAST program, score=50, wordlength=3 to obtain amino acid sequences homologous to 18636, 2466, 43238, 1983, 52881, 2398, 45449, 50289, 52872 or 26908 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

Particular 18636, 2466, 43238, 1983, 52881, 2398, 45449, 50289, 52872 or 26908 polypeptides of the present invention have an amino acid sequence substantially identical to the amino acid sequence of SEQ ID NO:2, 30, 70, 78, 81, 84, 87, 90, 93 or 104. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:2, 30, 70, 78, 81, 84, 87, 90, 93 or 104 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to SEQ ID NO:1, 3, 29, 31, 69, 71, 77, 79, 80, 82, 83, 85, 86, 88, 89, 91, 92, 94, 103 or 105 are termed substantially identical.

“Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject”, as used herein, can refer to a mammal, e.g., a human, or to an experimental or animal or disease model. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

As used herein, cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Examples of cellular proliferative and/or differentiative disorders of the lung include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders involving the colon include, but are not limited to, tumors of the colon, such as non-neoplastic polyps, adenomas, familial syndromes, colorectal carcinogenesis, colorectal carcinoma, and carcinoid tumors.

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As used herein, disorders involving the kidney (renal disorders) include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schönlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypernephroma, adenocarcinoma of kidney), which includes urothelial carcinomas of renal pelvis.

As used herein, hormonal disorders and diseases include type I and type II diabetes mellitus, pituitary disorders (e.g., growth disorders), thyroid disorders (e.g., hypothyroidism or hyperthyroidism), and reproductive or fertility disorders (e.g., disorders which affect the organs of the reproductive system, e.g., the prostate gland, the uterus, or the vagina; disorders which involve an imbalance in the levels of a reproductive hormone in a subject; disorders affecting the ability of a subject to reproduce; and disorders affecting secondary sex characteristic development, e.g., adrenal hyperplasia).

Aberrant expression and/or activity of the molecules of the invention can mediate disorders associated with bone metabolism. “Bone metabolism” refers to direct or indirect effects in the formation or degeneration of bone structures, e.g., bone formation, bone resorption, etc., which can ultimately affect the concentrations in serum of calcium and phosphate. This term also includes activities mediated by the molecules of the invention in bone cells, e.g. osteoclasts and osteoblasts, that can in turn result in bone formation and degeneration. For example, molecules of the invention can support different activities of bone resorbing osteoclasts such as the stimulation of differentiation of monocytes and mononuclear phagocytes into osteoclasts. Accordingly, molecules of the invention that modulate the production of bone cells can influence bone formation and degeneration, and thus can be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

As used herein, “a prostate disorder” refers to an abnormal condition occurring in the male pelvic region characterized by, e.g., male sexual dysfunction and/or urinary symptoms. This disorder may be manifested in the form of genitourinary inflammation (e.g., inflammation of smooth muscle cells) as in several common diseases of the prostate including prostatitis, benign prostatic hyperplasia and cancer, e.g., adenocarcinoma or carcinoma, of the prostate.

Examples of immune, e.g., inflammatory, (e.g. respiratory inflammatory) disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, inflammatory bowel disease, e.g. Crohn's disease and ulcerative colitis, aphthous ulcer, iritis, conjunctivitis, keratoconjunctivitis, asthma, allergic asthma, chronic obstructive pulmonary disease, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

As used herein, disorders involving the heart, or “cardiovascular disease” or a “cardiovascular disorder” includes a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovascular disease or disorder also can include an endothelial cell disorder.

As used herein, the term “atherosclerosis” is intended to have its clinical meaning. This term refers to a cardiovascular condition occurring as a result of narrowing down of the arterial walls. The narrowing is due to the formation of plaques (raised patches) or streaks in the inner lining of the arteries. These plaques consist of foam cells of low-density lipoproteins, oxidized-LDL, decaying muscle cells, fibrous tissue, clumps of blood platelets, cholesterol, and sometimes calcium. They tend to form in regions of turbulent blood flow and are found most often in people with high concentrations of cholesterol in the bloodstream. The number and thickness of plaques increase with age, causing loss of the smooth lining of the blood vessels and encouraging the formation of thrombi (blood clots). Sometimes fragments of thrombi break off and form emboli, which travel through the bloodstream and block smaller vessels. The blood supply is restricted to the heart, eventually forming a blood clot leading to death. The major causes of atherosclerosis are hypercholesterolemia (and low HDL), hypoalphoproteinemia, and hyperlipidemia marked by high circulating cholesterol and high lipids like LDL-cholesterol and triglycerides in the blood. These lipids are deposited in the arterial walls, obstructing the blood flow and forming atherosclerotic plaques leading to death.

As used herein the term “hypercholesterolemia” is a condition with elevated levels of circulating total cholesterol, LDL-cholesterol and VLDL-cholesterol as per the guidelines of the Expert Panel Report of the National Cholesterol Educational Program (NCEP) of Detection, Evaluation of Treatment of high cholesterol in adults (see, Arch. Int. Med. (1988) 148, 36-39).

As used herein the term “hyperlipidemia” or “hyperlipemia” is a condition where the blood lipid parameters are elevated in the blood. This condition manifests an abnormally high concentration of fats. The lipid fractions in the circulating blood are, total cholesterol, low density lipoproteins, very low density lipoproteins and triglycerides.

As used herein the term “lipoprotein” such as VLDL, LDL and HDL, refers to a group of proteins found in the serum, plasma and lymph and are important for lipid transport. The chemical composition of each lipoprotein differs in that the HDL has a higher proportion of protein versus lipid, whereas the VLDL has a lower proportion of protein versus lipid.

As used herein, the term “triglyceride” means a lipid or neutral fat consisting of glycerol combined with three fatty acid molecules.

As used herein the term “xanthomatosis” is a disease evidenced by a yellowish swelling or plaques in the skin resulting from deposits of fat. The presence of xanthomas are usually accompanied by raised blood cholesterol levels.

As used herein the term “apolipoprotein B” or “apoprotein B” or “Apo B” refers to the protein component of the LDL cholesterol transport proteins. Cholesterol synthesized de novo is transported from the liver and intestine to peripheral tissues in the form of lipoproteins. Most of the apolipoprotein B is secreted into the circulatory system as VLDL.

As used herein the term “apolipoprotein A” or “apoprotein A” or “Apo A” refers to the protein component of the HDL cholesterol transport proteins.

“Procedural vascular trauma” includes the effects of surgical/medical-mechanical interventions into mammalian vasculature, but does not include vascular trauma due to the organic vascular pathologies listed hereinabove, or to unintended traumas, such as due to an accident. Thus, procedural vascular traumas within the scope of the present treatment method include (1) organ grafting or transplantation, such as transplantation and grafting of heart, kidney, liver and the like, e.g., involving vessel anastomosis; (2) vascular surgery, such as coronary bypass surgery, biopsy, heart valve replacement, atheroectomy, thrombectomy, and the like; (3) transcatheter vascular therapies (TVT) including angioplasty, e.g., laser angioplasty and PTCA procedures discussed hereinbelow, employing balloon catheters, or indwelling catheters; (4) vascular grafting using natural or synthetic materials, such as in saphenous vein coronary bypass grafts, dacron and venous grafts used for peripheral arterial reconstruction, etc.; (5) placement of a mechanical shunt, such as a PTFE hemodialysis shunt used for arteriovenous communications; and (6) placement of an intravascular stent, which may be metallic, plastic or a biodegradable polymer. See U.S. patent application Ser. No. 08/389,712, filed Feb. 15, 1995, which is incorporated by reference herein. For a general discussion of implantable devices and biomaterials from which they can be formed, see H. Kambic et al., “Biomaterials in Artificial Organs”, Chem. Eng. News, 30 (Apr. 14, 1986), the disclosure of which is incorporated by reference herein.

As used herein, disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicella-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

As used herein, disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

As used herein, disorders involving the testis and epididymis include, but are not limited to, congenital anomalies such as cryptorchidism, regressive changes such as atrophy, inflammations such as nonspecific epididymitis and orchitis, granulomatous (autoimmune) orchitis, and specific inflammations including, but not limited to, gonorrhea, mumps, tuberculosis, and syphilis, vascular disturbances including torsion, testicular tumors including germ cell tumors that include, but are not limited to, seminoma, spermatocytic seminoma, embryonal carcinoma, yolk sac tumor choriocarcinoma, teratoma, and mixed tumors, tumore of sex cord-gonadal stroma including, but not limited to, Leydig (interstitial) cell tumors and sertoli cell tumors (androblastoma), and testicular lymphoma, and miscellaneous lesions of tunica vaginalis.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, a₁-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

Disorders which can be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein can be used for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Additionally, the molecules of the invention can play an important role in the etiology of certain viral diseases, including but not limited to Hepatitis B, Hepatitis C and Herpes Simplex Virus (HSV). Modulators of the activity of the molecules of the invention could be used to control viral diseases. The modulators can be used in the treatment and/or diagnosis of viral infected tissue or virus-associated tissue fibrosis, especially liver and liver fibrosis. Also, such modulators can be used in the treatment and/or diagnosis of virus-associated carcinoma, especially hepatocellular cancer.

As used herein, “hematological disorders” include, but are not limited to, disorders which result from decreased hematopoiesis or increased destruction of blood cells: aplastic anemia, pure red cell aplasia, Diamond-Blackfan syndrome, anemia of chronic renal failure, anemia resulting from hemolysis, anemia resulting from bleeding, anemia of pituitary disease, anemia of thyroid disease, anemia of adrenal disease, anemia of gonadal disease, anemia of cancer, myelodysplastic syndromes, myelophthisis, megaloblastic anemia, anemia resulting from iron deficiency, hypochromic anemias, normocytic anemias, anemia resulting from vitamin or mineral deficiencies such as folate, vitamin B₁₂, iron, etc., sickle cell anemia, thalassemias, hemolytic anemia, immunohemolytic anemia, thrombocytopenia, and HIV infection.

Anemias include, but are not limited to, aplastic anemia, pure red cell aplasia, Diamond-Blackfan syndrome, anemia of chronic renal failure, anemia resulting from hemolysis, anemia resulting from bleeding, anemia of pituitary disease, anemia of thyroid disease, anemia of adrenal disease, anemia of gonadal disease, anemia of cancer, myelodysplastic syndromes, myelophthisis, megaloblastic anemia, anemia resulting from iron deficiency, hypochromic anemias, normocytic anemias, anemia resulting from vitamin or mineral deficiencies such as folate, vitamin B₁₂, iron, etc., sickle cell anemia, thalassemias, hemolytic anemia, and immunohemolytic anemia.

Disorders related to reduced platelet number, thrombocytopenia, include idiopathic thrombocytopenic purpura, including acute idiopathic thrombocytopenic purpura, drug-induced thrombocytopenia, HIV-associated thrombocytopenia, and thrombotic microangiopathies: thrombotic thrombocytopenic purpura and hemolytic-uremic syndrome.

As used herein, neurological disorders include disorders of the central nervous system (CNS) and the peripheral nervous system, e.g., cognitive, neurodegenerative and psychiatric disorders. Examples of neurological disorders include, but are not limited to, autonomic function disorders such as hypertension and sleep disorders, and neuropsychiatric disorders, such as depression, schizophrenia, schizoaffective disorder, Korsakoff's psychosis, alcoholism, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Such neurological disorders include, for example, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicella-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer's disease and Pick's disease, degenerative diseases of basal ganglia and brain stem, including Parkinsonism, idiopathic Parkinson's disease (paralysis agitans) and other Lewy diffuse body diseases, progressive supranuclear palsy, corticobasal degenration, multiple system atrophy, including striatonigral degenration, Shy-Drager syndrome, and olivopontocerebellar atrophy, and Huntington's disease, senile dementia, Gilles de la Tourette's syndrome, epilepsy, and Jakob-Creutzfieldt disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease. Further CNS-related disorders include, for example, those listed in the American Psychiatric Association's Diagnostic and Statistical manual of Mental Disorders (DSM), the most current version of which is incorporated herein by reference in its entirety.

As used herein, diseases of the skin, include but are not limited to, disorders of pigmentation and melanocytes, including but not limited to, vitiligo, freckle, melasma, lentigo, nevocellular nevus, dysplastic nevi, and malignant melanoma; benign epithelial tumors, including but not limited to, seborrheic keratoses, acanthosis nigricans, fibroepithelial polyp, epithelial cyst, keratoacanthoma, and adnexal (appendage) tumors; premalignant and malignant epidermal tumors, including but not limited to, actinic keratosis, squamous cell carcinoma, basal cell carcinoma, and merkel cell carcinoma; tumors of the dermis, including but not limited to, benign fibrous histiocytoma, dermatofibrosarcoma protuberans, xanthomas, and dermal vascular tumors; tumors of cellular immigrants to the skin, including but not limited to, histiocytosis X, mycosis fungoides (cutaneous T-cell lymphoma), and mastocytosis; disorders of epidermal maturation, including but not limited to, ichthyosis; acute inflammatory dermatoses, including but not limited to, urticaria, acute eczematous dermatitis, and erythema multiforme; chronic inflammatory dermatoses, including but not limited to, psoriasis, lichen planus, and lupus erythematosus; blistering (bullous) diseases, including but not limited to, pemphigus, bullous pemphigoid, dermatitis herpetiformis, and noninflammatory blistering diseases: epidermolysis bullosa and porphyria; disorders of epidermal appendages, including but not limited to, acne vulgaris; panniculitis, including but not limited to, erythema nodosum and erythema induratum; and infection and infestation, such as verrucae, molluscum contagiosum, impetigo, superficial fungal infections, and arthropod bites, stings, and infestations.

As used herein, disorders involving the eye and vision (opthalmological) include, but are not limited to, granulomatous uveitis, cataracts, trachoma, corneal dystrophies, e.g., granular dystrophy or lattice dystrophy, glaucomas, retrolental fibroplasia, diabetes mellitus, hypertensive and arteriosclerotic retinopathy, retinitis pigmentosa, macular degeneration, retinoblastoma, papillaedema, and optic neuritis.

Additionally, molecules of the invention can play an important role in the regulation of metabolism or pain disorders. Diseases of metabolic imbalance include, but are not limited to, obesity, anorexia nervosa, cachexia, lipid disorders, and diabetes. Examples of pain disorders include, but are not limited to, pain response elicited during various forms of tissue injury, e.g., inflammation, infection, and ischemia, usually referred to as hyperalgesia (described in, for example, Fields (1987) Pain, New York:McGraw-Hill); pain associated with musculoskeletal disorders, e.g., joint pain; tooth pain; headaches; pain associated with surgery; pain related to irritable bowel syndrome; or chest pain. Preferably the disorder is pain, neuropathic pain, inflammatory pain, chronic pain, post-operative pain, rheumatoid arthritic pain, neuropathies, neuralgia, algesia, neurodegeneration, nerve injury, stroke, ischaemia migraine, irritable bowl syndrome (IBS), respiratory disorder such as asthma or chronic obstructive pulmonary disease (COPD), a urological disorder such as diabetic neuropathy, incontinence and interstitial cystitis, or an inflammatory disorder.

Examples of intestinal (e.g., small intestinal) disorders include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors. Disorders involving the small intestine include the malabsorption syndromes such as, celiac sprue, tropical sprue (postinfectious sprue), whipple disease, disaccharidase (lactase) deficiency, abetalipoproteinemia, and tumors of the small intestine including adenomas and adenocarcinoma.

Respiratory disorders include, but are not limited to, apnea, asthma, particularly bronchial asthma, berillium disease, bronchiectasis, bronchitis, bronchopneumonia, cystic fibrosis, diphtheria, dyspnea, emphysema, chronic obstructive pulmonary disease, allergic bronchopulmonary aspergillosis, pneumonia, acute pulmonary edema, pertussis, pharyngitis, atelectasis, Wegener's granulomatosis, Legionnaires disease, pleurisy, rheumatic fever, and sinusitis.

Fibrotic disorders or diseases include fibrosis in general, e.g., chronic pulonary obstructive disease; ideopathic pulmonary fibrosis; crescentic glomerulofibrosis; sarcoidosis; cystic fibrosis; fibrosis/cirrhosis, including cirrhosis secondary to chronic alcoholism, cirrhosis secondary to hepatitis type B or hepatitis type C, and primary biliary cirrhosis; liver disorders, particularly liver fibrosis; and other fibrotic diseases; as well as in the treatment of burns and scarring.

As used herein, an “angiogenesis disorder” includes a disease or disorder which affects or is caused by aberrant or deficient angiogenesis. Disorders involving angiogenesis include, but are not limited to, aberrant or excess angiogenesis in tumors such as hemangiomas and Kaposi's sarcoma, von Hippel-Lindau disease, as well as the angiogenesis associated with tumor growth; aberrant or excess angiogenesis in diseases such as a Castleman's disease or fibrodysplasia ossificans progressiva; aberrant or deficient angiogenesis associated with aging, complications of healing certain wounds and complications of diseases such as diabetes and rheumatoid arthritis; or aberrant or deficient angiogenesis associated with hereditary hemorrhagic telangiectasia, autosomal dominant polycystic kidney disease, myelodysplastic syndrome or Klippel-Trenaunay-Weber syndrome.

Various aspects of the invention are described in further detail below.

### Isolated Nucleic Acid Molecules

In one aspect, the invention provides, an isolated or purified, nucleic acid molecule that encodes a 18636, 2466, 43238, 1983, 52881, 2398, 45449, 50289, 52872 or 26908 polypeptide described herein, e.g., a full length 18636, 2466, 43238, 1983, 52881, 2398, 45449, 50289, 52872 or 26908 protein or a fragment thereof, e.g., a biologically active portion of 18636, 2466, 43238, 1983, 52881, 2398, 45449, 50289, 52872 or 26908 protein. Also included is a nucleic acid fragment suitable for use as a hybridization probe, which can be used, e.g., to identify a nucleic acid molecule encoding a polypeptide of the invention, 18636, 2466, 43238, 1983, 52881, 2398, 45449, 50289, 52872 or 26908 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:1, 3, 29, 31, 69, 71, 77, 79, 80, 82, 83, 85, 86, 88, 89, 91, 92, 94, 103 or 105, or a portion of any of this nucleotide sequence. In one embodiment, the nucleic acid molecule includes sequences encoding the human 18636, 2466, 43238, 1983, 52881, 2398, 45449, 50289, 52872 or 26908 protein (i.e., “the coding region” of SEQ ID NO:1, 29, 69, 77, 80, 83, 86, 89, 92 or 103, as shown in SEQ ID NO:3, 31, 71, 79, 82, 85, 88, 91, 94 or 105, respectively), as well as 5′ untranslated sequences and 3′ untranslated sequences. Alternatively, the nucleic acid molecule can include only the coding region of SEQ ID NO:1, 29, 69, 77, 80, 83, 86, 89, 92 or 103 (e.g., SEQ ID NO:3, 31, 71, 79, 82, 85, 88, 91, 94 or 105) and, e.g., no flanking sequences which normally accompany the subject sequence. In another embodiment, the nucleic acid molecule encodes a sequence corresponding to a fragment of the protein corresponding to domains within SEQ ID NO:2, 30, 70, 78, 81, 84, 87, 90, 93 or 104.

In another embodiment, an isolated nucleic acid molecule of the invention includes a nucleic acid molecule which is a complement of the nucleotide sequence shown in SEQ ID NO:1, 3, 29, 31, 69, 71, 77, 79, 80, 82, 83, 85, 86, 88, 89, 91, 92, 94, 103 or 105, or a portion of any of these nucleotide sequences. In other embodiments, the nucleic acid molecule of the invention is sufficiently complementary to the nucleotide sequence shown in SEQ ID NO:1, 3, 29, 31, 69, 71, 77, 79, 80, 82, 83, 85, 86, 88, 89, 91, 92, 94, 103 or 105 such that it can hybridize to the nucleotide sequence shown in SEQ ID NO:1, 3, 29, 31, 69, 71, 77, 79, 80, 82, 83, 85, 86, 88, 89, 91, 92, 94, 103 or 105, thereby forming a stable duplex.

In one embodiment, an isolated-nucleic acid molecule of the present invention includes a nucleotide sequence which is at least about: 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:1, 3, 29, 31, 69, 71, 77, 79, 80, 82, 83, 85, 86, 88, 89, 91, 92, 94, 103 or 105, or a portion, preferably of the same length, of any of these nucleotide sequences.

### 18636, 2466, 43238, 1983, 52881, 2398, 45449, 50289, 52872 or 26908 Nucleic Acid Fragments

A nucleic acid molecule of the invention can include only a portion of the nucleic acid sequence of SEQ ID NO:1, 3, 29, 31, 69, 71, 77, 79, 80, 82, 83, 85, 86, 88, 89, 91, 92, 94, 103 or 105. For example, such a nucleic acid molecule can include a fragment which can be used as a probe or primer or a fragment encoding a portion of a 18636, 2466, 43238, 1983, 52881, 2398, 45449, 50289, 52872 or 26908 protein, e.g., an immunogenic or biologically active portion of a 18636, 2466, 43238, 1983, 52881, 2398, 45449, 50289, 52872 or 26908 protein. A fragment can comprise those nucleotides of SEQ ID NO:1, 3, 29, 31, 69, 71, 77, 79, 80, 82, 83, 85, 86, 88, 89, 91, 92, 94, 103 or 105, which encode a domain of human 18636, 2466, 43238, 1983, 52881, 2398, 45449, 50289, 52872 or 26908. The nucleotide sequence determined from the cloning of the 18636, 2466, 43238, 1983, 52881, 2398, 45449, 50289, 52872 or 26908 gene allows for the generation of probes and primers designed for use in identifying and/or cloning other 18636, 2466, 43238, 1983, 52881, 2398, 45449, 50289, 52872 or 26908 family members, or fragments thereof, as well as 18636, 2466, 43238, 1983, 52881, 2398, 45449, 50289, 52872 or 26908 homologs, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 100 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 18636, 2466, 43238, 1983, 52881, 2398, 45449, 50289, 52872 or 26908 nucleic acid fragment can include a sequence corresponding to a domain, as described herein.

18636, 2466, 43238, 1983, 52881, 2398, 45449, 50289, 52872 or 26908 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO:1, 3, 29, 31, 69, 71, 77, 79, 80, 82, 83, 85, 86, 88, 89, 91, 92, 94, 103 or 105, or of a naturally occurring allelic variant or mutant of SEQ ID NO:1, 3, 29, 31, 69, 71, 77, 79, 80, 82, 83, 85, 86, 88, 89, 91, 92, 94, 103 or 105.

In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a domain identified in the 18636, 2466, 43238, 1983, 52881, 2398, 45449, 50289, 52872 or 26908 sequences.

In another embodiment a set of primers is provided, e.g., primers suitable for use in a PCR, which can be used to amplify a selected region of a 18636, 2466, 43238, 1983, 52881, 2398, 45449, 50289, 52872 or 26908 sequence, e.g., a domain, region, site or other sequence described herein. The primers should be at least 5, 10, or 50 base pairs in length and less than 100, or less than 200, base pairs in length. The primers should be identical, or differ by one base from a sequence disclosed herein or from a naturally occurring variant.

A nucleic acid fragment can encode an epitope bearing region of a polypeptide described herein.

A nucleic acid fragment encoding a “biologically active portion of a 18636, 2466, 43238, 1983, 52881, 2398, 45449, 50289, 52872 or 26908 polypeptide” can be prepared by isolating a portion of the nucleotide sequence of SEQ ID NO:1, 3, 29, 31, 69, 71, 77, 79, 80, 82, 83, 85, 86, 88, 89, 91, 92, 94, 103 or 105, which encodes a polypeptide having a 18636, 2466, 43238, 1983, 52881, 2398, 45449, 50289, 52872 or 26908 biological activity (e.g., the biological activities of the 18636, 2466, 43238, 1983, 52881, 2398, 45449, 50289, 52872 or 26908 proteins are described herein), expressing the encoded portion of the 18636, 2466, 43238, 1983, 52881, 2398, 45449, 50289, 52872 or 26908 protein (e.g., by recombinant expression in vitro) and assessing the activity of the encoded portion of the 18636, 2466, 43238, 1983, 52881, 2398, 45449, 50289, 52872 or 26908 protein. A nucleic acid fragment encoding a biologically active portion of a 18636, 2466, 43238, 1983, 52881, 2398, 45449, 50289, 52872 or 26908 polypeptide, can comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200 or more nucleotides in length and hybridizes under stringent hybridization conditions to a nucleic acid molecule of SEQ ID NO:1, 3, 29, 31, 69, 71, 77, 79, 80, 82, 83, 85, 86, 88, 89, 91, 92, 94, 103 or 105.

### 18636, 2466, 43238, 1983, 52881, 2398, 45449, 50289, 52872 or 26908 Nucleic Acid Variants

The invention further encompasses nucleic acid molecules that differ from the nucleotide sequence shown in SEQ ID NO:1, 3, 29, 31, 69, 71, 77, 79, 80, 82, 83, 85, 86, 88, 89, 91, 92, 94, 103 or 105. Such differences can be due to degeneracy of the genetic code (and result in a nucleic acid which encodes the same 18636, 2466, 43238, 1983, 52881, 2398, 45449, 50289, 52872 or 26908 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2, 30, 70, 78, 81, 84, 87, 90, 93 or 104. If alignment is needed for this comparison the sequences should be aligned for maximum homology. “Looped” out sequences from deletions or insertions, or mismatches, are considered differences.

Nucleic acids of the inventor can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:1, 3, 29, 31, 69, 71, 77, 79, 80, 82, 83, 85, 86, 88, 89, 91, 92, 94, 103 or 105, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:2, 30, 70, 78, 81, 84, 87, 90, 93 or 104 or a fragment of this sequence. Such nucleic acid molecules can readily be identified as being able to hybridize under stringent conditions, to the nucleotide sequence shown in SEQ ID NO:2, 30, 70, 78, 81, 84, 87, 90, 93 or 104 or a fragment of the sequence. Nucleic acid molecules corresponding to orthologs, homologs, and allelic variants of the 18636, 2466, 43238, 1983, 52881, 2398, 45449, 50289, 52872 or 26908 cDNAs of the invention can further be isolated by mapping to the same chromosome or locus as the 18636, 2466, 43238, 1983, 52881, 2398, 45449, 50289, 52872 or 26908 gene.

Preferred variants include those that are correlated with activities specific to the molecules of the invention, i.e. GPCR activity, or other.

Allelic variants of 18636, 2466, 43238, 1983, 52881, 2398, 45449, 50289, 52872 or 26908, e.g., human 18636, 2466, 43238, 1983, 52881, 2398, 45449, 50289, 52872 or 26908, include both functional and non-functional proteins. Functional allelic variants are naturally occurring amino acid sequence variants of the 18636, 2466, 43238, 1983, 52881, 2398, 45449, 50289, 52872 or 26908 protein within a population that maintain the ability to bind a 18636, 2466, 43238, 1983, 52881, 2398, 45449, 50289, 52872 or 26908 ligand or substrate and/or modulate cell signalling mechanisms. Functional allelic variants will typically contain only conservative substitution of one or more amino acids of SEQ ID NO:2, 30, 70, 78, 81, 84, 87, 90, 93 or 104, or substitution, deletion or insertion of non-critical residues in non-critical regions of the protein. Non-functional allelic variants are naturally-occurring amino acid sequence variants of the 18636, 2466, 43238, 1983, 52881, 2398, 45449, 50289, 52872 or 26908, e.g., human 18636, 2466, 43238, 1983, 52881, 2398, 45449, 50289, 52872 or 26908, protein within a population that do not have the ability to bind a 18636, 2466, 43238, 1983, 52881, 2398, 45449, 50289, 52872 or 26908 ligand or substrate and/or modulate cell signalling mechanisms. Non-functional allelic variants will typically contain a non-conservative substitution, a deletion, or insertion, or premature truncation of the amino acid sequence of SEQ ID NO:2, 30, 70, 78, 81, 84, 87, 90, 93 or 104, or a substitution, insertion, or deletion in critical residues or critical regions of the protein.

Moreover, nucleic acid molecules encoding other 18636, 2466, 43238, 1983, 52881, 2398, 45449, 50289, 52872 or 26908 family members and, thus, which have a nucleotide sequence which differs from the 18636, 2466, 43238, 1983, 52881, 2398, 45449, 50289, 52872 or 26908 sequences of SEQ ID NO:1, 3, 29, 31, 69, 71, 77, 79, 80, 82, 83, 85, 86, 88, 89, 91, 92, 94, 103 or 105 are intended to be within the scope of the invention.

Antisense Nucleic Acid Molecules, Ribozymes and Modified 18636, 2466, 43238, 1983, 52881, 2398, 45449, 50289, 52872 or 26908 nucleic Acid Molecules

In another aspect, the invention features, an isolated nucleic acid molecule which is antisense to 18636, 2466, 43238, 1983, 52881, 2398, 45449, 50289, 52872 or 26908. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 18636, 2466, 43238, 1983, 52881, 2398, 45449, 50289, 52872 or 26908 coding strand, or to only a portion thereof (e.g., the coding region of human 18636, 2466, 43238, 1983, 52881, 2398, 45449, 50289, 52872 or 26908 corresponding to SEQ ID NO:3, 31, 71, 79, 82, 85, 88, 91, 94 or 105, respectively). In another embodiment, the antisense nucleic acid molecule is antisense to a “noncoding region” of the coding strand of a nucleotide sequence encoding 18636, 2466, 43238, 1983, 52881, 2398, 45449, 50289, 52872 or 26908 (e.g., the 5′ and 3′ untranslated regions).

An antisense nucleic acid can be designed such that it is complementary to the entire coding region of 18636, 2466, 43238, 1983, 52881, 2398, 45449, 50289, 52872 or 26908 mRNA, but more preferably is an oligonucleotide which is antisense to only a portion of the coding or noncoding region of 18636, 2466, 43238, 1983, 52881, 2398, 45449, 50289, 52872 or 26908 mRNA. For example, the antisense oligonucleotide can be complementary to the region surrounding the translation start site of 18636, 2466, 43238, 1983, 52881, 2398, 45449, 50289, 52872 or 26908 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 18636, 2466, 43238, 1983, 52881, 2398, 45449, 50289, 52872 or 26908 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically or selectively bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 18636, 2466, 43238, 1983, 52881, 2398, 45449, 50289, 52872 or 26908-encoding nucleic acid can include one or more sequences complementary to the nucleotide sequence of a 18636, 2466, 43238, 1983, 52881, 2398, 45449, 50289, 52872 or 26908 cDNA disclosed herein (i.e., SEQ ID NO:1, 3, 29, 31, 69, 71, 77, 79, 80, 82, 83, 85, 86, 88, 89, 91, 92, 94, 103 or 105), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 18636, 2466, 43238, 1983, 52881, 2398, 45449, 50289, 52872 or 26908-encoding mRNA. See, e.g., Cech et al. U.S. Pat. No. 4,987,071; and Cech et al. U.S. Pat. No. 5,116,742. Alternatively, 18636, 2466, 43238, 1983, 52881, 2398, 45449, 50289, 52872 or 26908 mRNA can be used to select a catalytic RNA having a specific ribonuclease activity from a pool of RNA molecules. See, e.g., Bartel and Szostak (1993) Science 261:1411-1418.

18636, 2466, 43238, 1983, 52881, 2398, 45449, 50289, 52872 or 26908 gene expression can be inhibited by targeting nucleotide sequences complementary to the regulatory region of the 18636, 2466, 43238, 1983, 52881, 2398, 45449, 50289, 52872 or 26908 (e.g., the 18636, 2466, 43238, 1983, 52881, 2398, 45449, 50289, 52872 or 26908 promoter and/or enhancers) to form triple helical structures that prevent transcription of the 18636, 2466, 43238, 1983, 52881, 2398, 45449, 50289, 52872 or 26908 gene in target cells. See generally, Helene (1991) Anticancer Drug Des. 6:569-84; Helene (1992) Ann. N.Y. Acad. Sci. 660:27-36; and Maher (1992) Bioassays 14:807-15. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or calorimetric.

A 18636, 2466, 43238, 1983, 52881, 2398, 45449, 50289, 52872 or 26908 nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) Bioorganic & Medicinal Chemistry 4: 5-23).

As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. 93: 14670-675.

PNAs of 18636, 2466, 43238, 1983, 52881, 2398, 45449, 50289, 52872 or 26908 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 18636, 2466, 43238, 1983, 52881, 2398, 45449, 50289, 52872 or 26908 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup et al. (1996) supra)); or as probes or primers for DNA sequencing or hybridization (Hyrup et al. (1996) supra; Perry-O'Keefe supra).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide can be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 18636, 2466, 43238, 1983, 52881, 2398, 45449, 50289, 52872 or 26908 nucleic acid of the invention, two complementary regions one having a fluorophore and one a quencher such that the molecular beacon is useful for quantitating the presence of the 18636, 2466, 43238, 1983, 52881, 2398, 45449, 50289, 52872 or 26908 nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

### Isolated 18636, 2466, 43238, 1983, 52881, 2398, 45449, 50289, 52872 or 26908 Polypeptides

In another aspect, the invention features, an isolated 18636, 2466, 43238, 1983, 52881, 2398, 45449, 50289, 52872 or 26908 protein, or fragment, e.g., a biologically active portion, for use as immunogens or antigens to raise or test (or more generally to bind) anti-18636, 2466, 43238, 1983, 52881, 2398, 45449, 50289, 52872 or 26908 antibodies. 18636, 2466, 43238, 1983, 52881, 2398, 45449, 50289, 52872 or 26908 protein can be isolated from cells or tissue sources using standard protein purification techniques. 18636, 2466, 43238, 1983, 52881, 2398, 45449, 50289, 52872 or 26908 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when the polypeptide is expressed in a native cell, or in systems which result in the alteration or omission of post-translational modifications, e.g., glycosylation or cleavage, present in a native cell.

In a preferred embodiment, a 18636, 2466, 43238, 1983, 52881, 2398, 45449, 50289, 52872 or 26908 polypeptide has one or more of the following characteristics: it has the ability: to interact or associate with a G protein; to bind a ligand such as a peptide, neuropeptide (e.g., FF, H2, glanin), platelet-activating factor; to be phosphorylated or dephosphorylated; the ability to affect cGMP or cAMP concentrations in the cell; to regulate, sense and/or transmit an extracellular signal into a cell, (for example, a heart cell, a bone cell (e.g., an osteoclast or an osteoblast), a hematopoietic cell, a neural cell); to interact with (e.g., binding to) an extracellular signal or a cell surface receptor; to mobilize an intracellular molecule that participates in a signal transduction pathway (e.g., adenylate cyclase or phosphatidylinositol 4,5-bisphosphate (PIP₂), inositol 1,4,5-triphosphate (IP₃)); to regulate polarization of the plasma membrane; to control production or secretion of molecules; to alter the structure of a cellular component; to modulate cell proliferation, e.g., synthesis of DNA; to modulate cell migration, cell differentiation; and cell survival; it has a molecular weight, e.g., a deduced molecular weight, preferably ignoring any contribution of post translational modifications, amino acid composition or other physical characteristic of a 18636, 2466, 43238, 1983, 52881, 2398, 45449, 50289, 52872 or 26908 polypeptide, e.g., a polypeptide of SEQ ID NO:2, 30, 70, 78, 81, 84, 87, 90, 93 or 104; it has an overall sequence similarity of at least 60%, preferably at least 70%, more preferably at least 80, 90, or 95%, with a polypeptide of SEQ ID NO:2, 30, 70, 78, 81, 84, 87, 90, 93 or 104; it is expressed in a multitude of human tissues and cell lines (refer to section for each molecule of the invention); and it has specific domains which are preferably about 70%, 80%, 90% or 95% identical to the identified amino acid residues of SEQ ID NO:2, 30, 70, 78, 81, 84, 87, 90, 93 or 104 (refer to section for each molecule of the invention for domain names and locations within amino acid sequence).

In a preferred embodiment the 18636, 2466, 43238, 1983, 52881, 2398, 45449, 50289, 52872 or 26908 protein, or fragment thereof, differs from the corresponding sequence in SEQ ID NO:2, 30, 70, 78, 81, 84, 87, 90, 93 or 104. In one embodiment it differs by at least one but by less than 15, 10 or 5 amino acid residues. In another it differs from the corresponding sequence in SEQ ID NO:2, 30, 70, 78, 81, 84, 87, 90, 93 or 104 by at least one residue but less than 20%, 15%, 10% or 5% of the residues in it differ from the corresponding sequence in SEQ ID NO:2, 30, 70, 78, 81, 84, 87, 90, 93 or 104. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the identified or conserved domain(s) within SEQ ID NO:2, 30, 70, 78, 81, 84, 87, 90, 93 or 104. In another embodiment one or more differences are in the cidentified or conserved domain(s) within SEQ ID NO:2, 30, 70, 78, 81, 84, 87, 90, 93 or 104.

Other embodiments include a protein that contains one or more changes in amino acid sequence, e.g., a change in an amino acid residue which is not essential for activity. Such 18636, 2466, 43238, 1983, 52881, 2398, 45449, 50289, 52872 or 26908 proteins differ in amino acid sequence from SEQ ID NO:2, 30, 70, 78, 81, 84, 87, 90, 93 or 104, yet retain biological activity.

In one embodiment, the protein includes an amino acid sequence at least about 60%, 65%, 70%, 75%, 80%, 85%, 90%, 95%, 98% or more homologous to SEQ ID NO:2, 30, 70, 78, 81, 84, 87, 90, 93 or 104.

A 18636, 2466, 43238, 1983, 52881, 2398, 45449, 50289, 52872 or 26908 protein or fragment is provided which varies from the sequence of SEQ ID NO:2, 30, 70, 78, 81, 84, 87, 90, 93 or 104 in regions defined by amino acids that are not within identified or conserved domains or regions by at least one but by less than 15, 10 or 5 amino acid residues in the protein or fragment but which does not differ from SEQ ID NO:2, 30, 70, 78, 81, 84, 87, 90, 93 or 104 in regions defined by amino acids that are within identified or conserved domains or regions. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 18636, 2466, 43238, 1983, 52881, 2398, 45449, 50289, 52872 or 26908 protein includes an identified domain (refer to section for each molecule of the invention). Moreover, other biologically active portions, in which other regions of the protein are deleted, can be prepared by recombinant techniques and evaluated for one or more of the functional activities of a native 18636, 2466, 43238, 1983, 52881, 2398, 45449, 50289, 52872 or 26908 protein.

In a preferred embodiment, the 18636, 2466, 43238, 1983, 52881, 2398, 45449, 50289, 52872 or 26908 protein has an amino acid sequence shown in SEQ ID NO:2, 30, 70, 78, 81, 84, 87, 90, 93 or 104. In other embodiments, the 18636, 2466, 43238, 1983, 52881, 2398, 45449, 50289, 52872 or 26908 protein is sufficiently or substantially identical to SEQ ID NO:2, 30, 70, 78, 81, 84, 87, 90, 93 or 104. In yet another embodiment, the 18636, 2466, 43238, 1983, 52881, 2398, 45449, 50289, 52872 or 26908 protein is sufficiently or substantially identical to SEQ ID NO:2, 30, 70, 78, 81, 84, 87, 90, 93 or 104 and retains the functional activity of the protein of SEQ ID NO:2, 30, 70, 78, 81, 84, 87, 90, 93 or 104, as described in detail in the subsections above.

### 18636, 2466, 43238, 1983, 52881, 2398, 45449, 50289, 52872 or 26908 Chimeric or Fusion Proteins

In another aspect, the invention provides 18636, 2466, 43238, 1983, 52881, 2398, 45449, 50289, 52872 or 26908 chimeric or fusion proteins. As used herein, a 18636, 2466, 43238, 1983, 52881, 2398, 45449, 50289, 52872 or 26908“chimeric protein” or “fusion protein” includes a 18636, 2466, 43238, 1983, 52881, 2398, 45449, 50289, 52872 or 26908 polypeptide linked to a non-18636, 2466, 43238, 1983, 52881, 2398, 45449, 50289, 52872 or 26908 polypeptide. A “non-18636, 2466, 43238, 1983, 52881, 2398, 45449, 50289, 52872 or 26908 polypeptide” refers to a polypeptide having an amino acid sequence corresponding to a protein which is not substantially homologous to the 18636, 2466, 43238, 1983, 52881, 2398, 45449, 50289, 52872 or 26908 protein, e.g., a protein which is different from the 18636, 2466, 43238, 1983, 52881, 2398, 45449, 50289, 52872 or 26908 protein and which is derived from the same or a different organism. The 18636, 2466, 43238, 1983, 52881, 2398, 45449, 50289, 52872 or 26908 polypeptide of the fusion protein can correspond to all or a portion e.g., a fragment described herein of a 18636, 2466, 43238, 1983, 52881, 2398, 45449, 50289, 52872 or 26908 amino acid sequence. In a preferred embodiment, a 18636, 2466, 43238, 1983, 52881, 2398, 45449, 50289, 52872 or 26908 fusion protein includes at least one (or two) biologically active portion of a 18636, 2466, 43238, 1983, 52881, 2398, 45449, 50289, 52872 or 26908 protein. The non-18636, 2466, 43238, 1983, 52881, 2398, 45449, 50289, 52872 or 26908 polypeptide can be fused to the N-terminus or C-terminus of the 18636, 2466, 43238, 1983, 52881, 2398, 45449, 50289, 52872 or 26908 polypeptide.

The fusion protein can include a moiety which has a high affinity for a ligand. For example, the fusion protein can be a GST-18636, 2466, 43238, 1983, 52881, 2398, 45449, 50289, 52872 or 26908 fusion protein in which the 18636, 2466, 43238, 1983, 52881, 2398, 45449, 50289, 52872 or 26908 sequences are fused to the C-terminus of the GST sequences. Such fusion proteins can facilitate the purification of recombinant 18636, 2466, 43238, 1983, 52881, 2398, 45449, 50289, 52872 or 26908. Alternatively, the fusion protein can be a 18636, 2466, 43238, 1983, 52881, 2398, 45449, 50289, 52872 or 26908 protein containing a heterologous signal sequence at its N-terminus. In certain host cells (e.g., mammalian host cells), expression and/or secretion of 18636, 2466, 43238, 1983, 52881, 2398, 45449, 50289, 52872 or 26908 can be increased through use of a heterologous signal sequence.

Fusion proteins can include all or a part of a serum protein, e.g., a portion of an immunoglobulin (e.g., IgG, IgA, or IgE), e.g., an Fc region and/or the hinge C1 and C2 sequences of an immunoglobulin or human serum albumin.

The 18636, 2466, 43238, 1983, 52881, 2398, 45449, 50289, 52872 or 26908 fusion proteins of the invention can be incorporated into pharmaceutical compositions and administered to a subject in vivo. The 18636, 2466, 43238, 1983, 52881, 2398, 45449, 50289, 52872 or 26908 fusion proteins can be used to affect the bioavailability of a 18636, 2466, 43238, 1983, 52881, 2398, 45449, 50289, 52872 or 26908 substrate. 18636, 2466, 43238, 1983, 52881, 2398, 45449, 50289, 52872 or 26908 fusion proteins can be useful therapeutically for the treatment of disorders caused by, for example, (i) aberrant modification or mutation of a gene encoding a 18636, 2466, 43238, 1983, 52881, 2398, 45449, 50289, 52872 or 26908 protein; (ii) mis-regulation of the 18636, 2466, 43238, 1983, 52881, 2398, 45449, 50289, 52872 or 26908 gene; and (iii) aberrant post-translational modification of a 18636, 2466, 43238, 1983, 52881, 2398, 45449, 50289, 52872 or 26908 protein.

Moreover, the 18636, 2466, 43238, 1983, 52881, 2398, 45449, 50289, 52872 or 26908-fusion proteins of the invention can be used as immunogens to produce anti-18636, 2466, 43238, 1983, 52881, 2398, 45449, 50289, 52872 or 26908 antibodies in a subject, to purify 18636, 2466, 43238, 1983, 52881, 2398, 45449, 50289, 52872 or 26908 ligands and in screening assays to identify molecules which inhibit the interaction of 18636, 2466, 43238, 1983, 52881, 2398, 45449, 50289, 52872 or 26908 with a 18636, 2466, 43238, 1983, 52881, 2398, 45449, 50289, 52872 or 26908 substrate.

Expression vectors are commercially available that already encode a fusion moiety (e.g., a GST polypeptide). A 18636, 2466, 43238, 1983, 52881, 2398, 45449, 50289, 52872 or 26908-encoding nucleic acid can be cloned into such an expression vector such that the fusion moiety is linked in-frame to the 18636, 2466, 43238, 1983, 52881, 2398, 45449, 50289, 52872 or 26908 protein.

### Variants of 18636, 2466, 43238, 1983, 52881, 2398, 45449, 50289, 52872 or 26908 Proteins

In another aspect, the invention also features a variant of a 18636, 2466, 43238, 1983, 52881, 2398, 45449, 50289, 52872 or 26908 polypeptide, e.g., which functions as an agonist (mimetics) or as an antagonist. Variants of the 18636, 2466, 43238, 1983, 52881, 2398, 45449, 50289, 52872 or 26908 proteins can be generated by mutagenesis, e.g., discrete point mutation, the insertion or deletion of sequences or the truncation of a 18636, 2466, 43238, 1983, 52881, 2398, 45449, 50289, 52872 or 26908 protein. An agonist of the 18636, 2466, 43238, 1983, 52881, 2398, 45449, 50289, 52872 or 26908 proteins can retain substantially the same, or a subset, of the biological activities of the naturally occurring form of a 18636, 2466, 43238, 1983, 52881, 2398, 45449, 50289, 52872 or 26908 protein. An antagonist of a 18636, 2466, 43238, 1983, 52881, 2398, 45449, 50289, 52872 or 26908 protein can inhibit one or more of the activities of the naturally occurring form of the 18636, 2466, 43238, 1983, 52881, 2398, 45449, 50289, 52872 or 26908 protein by, for example, competitively modulating a 18636, 2466, 43238, 1983, 52881, 2398, 45449, 50289, 52872 or 26908-mediated activity of a 18636, 2466, 43238, 1983, 52881, 2398, 45449, 50289, 52872 or 26908 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 18636, 2466, 43238, 1983, 52881, 2398, 45449, 50289, 52872 or 26908 protein.

Variants of a 18636, 2466, 43238, 1983, 52881, 2398, 45449, 50289, 52872 or 26908 protein can be identified by screening combinatorial libraries of mutants, e.g., truncation mutants, of a 18636, 2466, 43238, 1983, 52881, 2398, 45449, 50289, 52872 or 26908 protein for agonist or antagonist activity.

Libraries of fragments e.g., N terminal, C terminal, or internal fragments, of a 18636, 2466, 43238, 1983, 52881, 2398, 45449, 50289, 52872 or 26908 protein coding sequence can be used to generate a variegated population of fragments for screening and subsequent selection of variants of a 18636, 2466, 43238, 1983, 52881, 2398, 45449, 50289, 52872 or 26908 protein.

Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Recursive ensemble mutagenesis (REM), a new technique which enhances the frequency of functional mutants in the libraries, can be used in combination with the screening assays to identify 18636, 2466, 43238, 1983, 52881, 2398, 45449, 50289, 52872 or 26908 variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated 18636, 2466, 43238, 1983, 52881, 2398, 45449, 50289, 52872 or 26908 library. For example, a library of expression vectors can be transfected into a cell line, e.g., a cell line, which ordinarily responds to 18636, 2466, 43238, 1983, 52881, 2398, 45449, 50289, 52872 or 26908 in a substrate-dependent manner. The transfected cells are then contacted with 18636, 2466, 43238, 1983, 52881, 2398, 45449, 50289, 52872 or 26908 and the effect of the expression of the mutant on signaling by the 18636, 2466, 43238, 1983, 52881, 2398, 45449, 50289, 52872 or 26908 substrate can be detected, e.g., by measuring GPCR activity, or other activity. Plasmid DNA can then be recovered from the cells which score for inhibition, or alternatively, potentiation of signaling by the 18636, 2466, 43238, 1983, 52881, 2398, 45449, 50289, 52872 or 26908 substrate, and the individual clones further characterized.

In another aspect, the invention features a method of making a 18636, 2466, 43238, 1983, 52881, 2398, 45449, 50289, 52872 or 26908 polypeptide, e.g., a peptide having a non-wild type activity, e.g., an antagonist, agonist, or super agonist of a naturally occurring 18636, 2466, 43238, 1983, 52881, 2398, 45449, 50289, 52872 or 26908 polypeptide, e.g., a naturally occurring 18636, 2466, 43238, 1983, 52881, 2398, 45449, 50289, 52872 or 26908 polypeptide. The method includes altering the sequence of a 18636, 2466, 43238, 1983, 52881, 2398, 45449, 50289, 52872 or 26908 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 18636, 2466, 43238, 1983, 52881, 2398, 45449, 50289, 52872 or 26908 polypeptide a biological activity of a naturally occurring 18636, 2466, 43238, 1983, 52881, 2398, 45449, 50289, 52872 or 26908 polypeptide. The method includes altering the sequence, e.g., by substitution or deletion of one or more residues, of a 18636, 2466, 43238, 1983, 52881, 2398, 45449, 50289, 52872 or 26908 polypeptide, e.g., altering the sequence of a non-conserved region, or a domain or residue described herein, and testing the altered polypeptide for the desired activity.

### Anti-18636, 2466, 43238, 1983, 52881, 2398, 45449, 50289, 52872 or 26908 Antibodies

In another aspect, the invention provides an anti-18636, 2466, 43238, 1983, 52881, 2398, 45449, 50289, 52872 or 26908 antibody. The term “antibody” as used herein refers to an immunoglobulin molecule or immunologically active portion thereof, i.e., an antigen-binding portion. Examples of immunologically active portions of immunoglobulin molecules include scFV and dcFV fragments, Fab and F(ab′)₂ fragments which can be generated by treating the antibody with an enzyme such as papain or pepsin, respectively.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length 18636, 2466, 43238, 1983, 52881, 2398, 45449, 50289, 52872 or 26908 protein or, antigenic peptide fragment of 18636, 2466, 43238, 1983, 52881, 2398, 45449, 50289, 52872 or 26908 can be used as an immunogen or can be used to identify anti-18636, 2466, 43238, 1983, 52881, 2398, 45449, 50289, 52872 or 26908 antibodies made with other immunogens, e.g., cells, membrane preparations, and the like. The antigenic peptide of 18636, 2466, 43238, 1983, 52881, 2398, 45449, 50289, 52872 or 26908 should include at least 8 amino acid residues of the amino acid sequence shown in SEQ ID NO:2, 30, 70, 78, 81, 84, 87, 90, 93 or 104 and encompasses an epitope of 18636, 2466, 43238, 1983, 52881, 2398, 45449, 50289, 52872 or 26908. Preferably, the antigenic peptide includes at least 10 amino acid residues, more preferably at least 15 amino acid residues, even more preferably at least 20 amino acid residues, and most preferably at least 30 amino acid residues.

Fragments of 18636, 2466, 43238, 1983, 52881, 2398, 45449, 50289, 52872 or 26908 which include hydrophilic regions of SEQ ID NO:2, 30, 70, 78, 81, 84, 87, 90, 93 or 104 can be used to make, e.g., used as immunogens or used to characterize the specificity of an antibody, antibodies against hydrophilic regions of the 18636, 2466, 43238, 1983, 52881, 2398, 45449, 50289, 52872 or 26908 protein. Similarly, fragments of 18636, 2466, 43238, 1983, 52881, 2398, 45449, 50289, 52872 or 26908 which include hydrophobic regions of SEQ ID NO:2, 30, 70, 78, 81, 84, 87, 90, 93 or 104 can be used to make an antibody against a hydrophobic region of the 18636, 2466, 43238, 1983, 52881, 2398, 45449, 50289, 52872 or 26908 protein; fragments of 18636, 2466, 43238, 1983, 52881, 2398, 45449, 50289, 52872 or 26908 which include residues within extra cellular domain(s) of SEQ ID NO:2, 30, 70, 78, 81, 84, 87, 90, 93 or 104 can be used to make an antibody against an extracellular or non-cytoplasmic region of the 18636, 2466, 43238, 1983, 52881, 2398, 45449, 50289, 52872 or 26908 protein; fragments of 18636, 2466, 43238, 1983, 52881, 2398, 45449, 50289, 52872 or 26908 which include residues within intracellular regions of SEQ ID NO:2, 30, 70, 78, 81, 84, 87, 90, 93 or 104 can be used to make an antibody against an intracellular region of the 18636, 2466, 43238, 1983, 52881, 2398, 45449, 50289, 52872 or 26908 protein; a fragment of 18636, 2466, 43238, 1983, 52881, 2398, 45449, 50289, 52872 or 26908 which include residues within identified or conserved domains of SEQ ID NO:2, 30, 70, 78, 81, 84, 87, 90, 93 or 104 can be used to make an antibody against the identified or conserved domain of the 18636, 2466, 43238, 1983, 52881, 2398, 45449, 50289, 52872 or 26908 protein.

Antibodies reactive with, or specific or selective for, any of these regions, or other regions or domains described herein are provided.

Preferred epitopes encompassed by the antigenic peptide are regions of 18636, 2466, 43238, 1983, 52881, 2398, 45449, 50289, 52872 or 26908 located on the surface of the protein, e.g., hydrophilic regions, as well as regions with high antigenicity. For example, an Emini surface probability analysis of the human 18636, 2466, 43238, 1983, 52881, 2398, 45449, 50289, 52872 or 26908 protein sequence can be used to indicate the regions that have a particularly high probability of being localized to the surface of the 18636, 2466, 43238, 1983, 52881, 2398, 45449, 50289, 52872 or 26908 protein and are thus likely to constitute surface residues useful for targeting antibody production.

In a preferred embodiment the antibody can bind to the extracellular portion of the 18636, 2466, 43238, 1983, 52881, 2398, 45449, 50289, 52872 or 26908 protein, e.g., it can bind to a whole cell which expresses the 18636, 2466, 43238, 1983, 52881, 2398, 45449, 50289, 52872 or 26908 protein. In another embodiment, the antibody binds an intracellular portion of the 18636, 2466, 43238, 1983, 52881, 2398, 45449, 50289, 52872 or 26908 protein.

In a preferred embodiment the antibody binds an epitope on any domain or region on 18636, 2466, 43238, 1983, 52881, 2398, 45449, 50289, 52872 or 26908 proteins described herein.

Additionally, chimeric, humanized, and completely human antibodies are also within the scope of the invention. Chimeric, humanized, but most preferably, completely human antibodies are desirable for applications which include repeated administration, e.g., therapeutic treatment of human patients, and some diagnostic applications.

Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559).

A humanized or complementarity determining region (CDR)-grafted antibody will have at least one or two, but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a 18636, 2466, 43238, 1983, 52881, 2398, 45449, 50289, 52872 or 26908 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, (1987) From Genes to Clones (Verlagsgesellschaft, Weinheim, Germany). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison (1985) Science 229:1202-1207, by Oi et al. (1986) BioTechniques 4:214, and by Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 18636, 2466, 43238, 1983, 52881, 2398, 45449, 50289, 52872 or 26908 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. (1986) Nature 321:552-525; Verhoeyan et al. (1988) Science 239:1534; Beidler et al. (1988) J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

The anti-18636, 2466, 43238, 1983, 52881, 2398, 45449, 50289, 52872 or 26908 antibody can be a single chain antibody. A single-chain antibody (scFV) can be engineered as described in, for example, Colcher et al. (1999) Ann. N Y Acad. Sci. 880:263-80; and Reiter (1996) Clin. Cancer Res. 2:245-52. The single chain antibody can be dimerized or multimerized to generate multivalent antibodies having specificities for different epitopes of the same target 18636, 2466, 43238, 1983, 52881, 2398, 45449, 50289, 52872 or 26908 protein.

In a preferred embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

Radioactive ions include, but are not limited to iodine, yttrium and praseodymium.

The conjugates of the invention can be used for modifying a given biological response, the therapeutic moiety is not to be construed as limited to classical chemical therapeutic agents. For example, the therapeutic moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

An anti-18636, 2466, 43238, 1983, 52881, 2398, 45449, 50289, 52872 or 26908 antibody (e.g., monoclonal antibody) can be used to isolate 18636, 2466, 43238, 1983, 52881, 2398, 45449, 50289, 52872 or 26908 by standard techniques, such as affinity chromatography or immunoprecipitation. Moreover, an anti-18636, 2466, 43238, 1983, 52881, 2398, 45449, 50289, 52872 or 26908 antibody can be used to detect 18636, 2466, 43238, 1983, 52881, 2398, 45449, 50289, 52872 or 26908 protein (e.g., in a cellular lysate or cell supernatant) in order to evaluate the abundance and pattern of expression of the protein. Anti-18636, 2466, 43238, 1983, 52881, 2398, 45449, 50289, 52872 or 26908 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In preferred embodiments, an antibody can be made by immunizing with a purified 18636, 2466, 43238, 1983, 52881, 2398, 45449, 50289, 52872 or 26908 antigen, or a fragment thereof, e.g., a fragment described herein, a membrane associated antigen, tissues, e.g., crude tissue preparations, whole cells, preferably living cells, lysed cells, or cell fractions, e.g., membrane fractions.

Antibodies which bind only a native 18636, 2466, 43238, 1983, 52881, 2398, 45449, 50289, 52872 or 26908 protein, only denatured or otherwise non-native 18636, 2466, 43238, 1983, 52881, 2398, 45449, 50289, 52872 or 26908 protein, or which bind both, are within the invention. Antibodies with linear or conformational epitopes are within the invention. Conformational epitopes sometimes can be identified by identifying antibodies which bind to native but not denatured 18636, 2466, 43238, 1983, 52881, 2398, 45449, 50289, 52872 or 26908 protein.

### Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 18636, 2466, 43238, 1983, 52881, 2398, 45449, 50289, 52872 or 26908 nucleic acid in a form suitable for expression of the nucleic acid in a host cell.

Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 18636, 2466, 43238, 1983, 52881, 2398, 45449, 50289, 52872 or 26908 proteins, mutant forms of 18636, 2466, 43238, 1983, 52881, 2398, 45449, 50289, 52872 or 26908 proteins, fusion proteins, and the like).

The recombinant expression vectors of the invention can be designed for expression of 18636, 2466, 43238, 1983, 52881, 2398, 45449, 50289, 52872 or 26908 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 18636, 2466, 43238, 1983, 52881, 2398, 45449, 50289, 52872 or 26908 activity assays, (e.g., direct assays or competitive assays described in detail below), or to generate antibodies specific or selective for 18636, 2466, 43238, 1983, 52881, 2398, 45449, 50289, 52872 or 26908 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 18636, 2466, 43238, 1983, 52881, 2398, 45449, 50289, 52872 or 26908 expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub et al., (1986) Reviews—Trends in Genetics 1:1.

Another aspect the invention provides a host cell which includes a nucleic acid molecule described herein, e.g., a 18636, 2466, 43238, 1983, 52881, 2398, 45449, 50289, 52872 or 26908 nucleic acid molecule within a recombinant expression vector or a 18636, 2466, 43238, 1983, 52881, 2398, 45449, 50289, 52872 or 26908 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications can occur in succeeding generations due to either mutation or environmental influences, such progeny may not, in fact, be identical to the parent cell, but are still included within the scope of the term as used herein.

A host cell can be any prokaryotic or eukaryotic cell. For example, a 18636, 2466, 43238, 1983, 52881, 2398, 45449, 50289, 52872 or 26908 protein can be expressed in bacterial cells such as E. coli, insect cells, yeast or mammalian cells (such as Chinese hamster ovary (CHO) cells or CV-1 origin, SV-40 (COS) cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a 18636, 2466, 43238, 1983, 52881, 2398, 45449, 50289, 52872 or 26908 protein. Accordingly, the invention further provides methods for producing a 18636, 2466, 43238, 1983, 52881, 2398, 45449, 50289, 52872 or 26908 protein using the host cells of the invention. In one embodiment, the method includes culturing the host cell of the invention (into which a recombinant expression vector encoding a 18636, 2466, 43238, 1983, 52881, 2398, 45449, 50289, 52872 or 26908 protein has been introduced) in a suitable medium such that a 18636, 2466, 43238, 1983, 52881, 2398, 45449, 50289, 52872 or 26908 protein is produced. In another embodiment, the method further includes isolating a 18636, 2466, 43238, 1983, 52881, 2398, 45449, 50289, 52872 or 26908 protein from the medium or the host cell.

In another aspect, the invention features, a cell or purified preparation of cells which include a 18636, 2466, 43238, 1983, 52881, 2398, 45449, 50289, 52872 or 26908 transgene, or which otherwise misexpress 18636, 2466, 43238, 1983, 52881, 2398, 45449, 50289, 52872 or 26908. The cell preparation can consist of human or non-human cells, e.g., rodent cells, e.g., mouse or rat cells, rabbit cells, or pig cells. In preferred embodiments, the cell or cells include a 18636, 2466, 43238, 1983, 52881, 2398, 45449, 50289, 52872 or 26908 transgene, e.g., a heterologous form of a 18636, 2466, 43238, 1983, 52881, 2398, 45449, 50289, 52872 or 26908, e.g., a gene derived from humans (in the case of a non-human cell). The 18636, 2466, 43238, 1983, 52881, 2398, 45449, 50289, 52872 or 26908 transgene can be misexpressed, e.g., overexpressed or underexpressed. In other preferred embodiments, the cell or cells include a gene which misexpresses an endogenous 18636, 2466, 43238, 1983, 52881, 2398, 45449, 50289, 52872 or 26908, e.g., a gene the expression of which is disrupted, e.g., a knockout. Such cells can serve as a model for studying disorders which are related to mutated or misexpressed 18636, 2466, 43238, 1983, 52881, 2398, 45449, 50289, 52872 or 26908 alleles or for use in drug screening.

In another aspect, the invention features, a human cell, e.g., a hematopoietic stem cell, transformed with nucleic acid which encodes a subject 18636, 2466, 43238, 1983, 52881, 2398, 45449, 50289, 52872 or 26908 polypeptide.

Also provided are cells, preferably human cells, e.g., human hematopoietic or fibroblast cells, in which an endogenous 18636, 2466, 43238, 1983, 52881, 2398, 45449, 50289, 52872 or 26908 is under the control of a regulatory sequence that does not normally control the expression of the endogenous 18636, 2466, 43238, 1983, 52881, 2398, 45449, 50289, 52872 or 26908 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 18636, 2466, 43238, 1983, 52881, 2398, 45449, 50289, 52872 or 26908 gene. For example, an endogenous 18636, 2466, 43238, 1983, 52881, 2398, 45449, 50289, 52872 or 26908 gene which is “transcriptionally silent,” e.g., not normally expressed, or expressed only at very low levels, can be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

### Transgenic Animals

The invention provides non-human transgenic animals. Such animals are useful for studying the function and/or activity of a 18636, 2466, 43238, 1983, 52881, 2398, 45449, 50289, 52872 or 26908 protein and for identifying and/or evaluating modulators of 18636, 2466, 43238, 1983, 52881, 2398, 45449, 50289, 52872 or 26908 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 18636, 2466, 43238, 1983, 52881, 2398, 45449, 50289, 52872 or 26908 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 18636, 2466, 43238, 1983, 52881, 2398, 45449, 50289, 52872 or 26908 protein to particular cells. A transgenic founder animal can be identified based upon the presence of a 18636, 2466, 43238, 1983, 52881, 2398, 45449, 50289, 52872 or 26908 transgene in its genome and/or expression of 18636, 2466, 43238, 1983, 52881, 2398, 45449, 50289, 52872 or 26908 mRNA in tissues or cells of the animals. A transgenic founder animal can then be used to breed additional animals carrying the transgene. Moreover, transgenic animals carrying a transgene encoding a 18636, 2466, 43238, 1983, 52881, 2398, 45449, 50289, 52872 or 26908 protein can further be bred to other transgenic animals carrying other transgenes.

18636, 2466, 43238, 1983, 52881, 2398, 45449, 50289, 52872 or 26908 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

### Uses

The nucleic acid molecules, proteins, protein homologs, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a 18636, 2466, 43238, 1983, 52881, 2398, 45449, 50289, 52872 or 26908 protein (e.g., via a recombinant expression vector in a host cell in gene therapy applications), to detect a 18636, 2466, 43238, 1983, 52881, 2398, 45449, 50289, 52872 or 26908 mRNA (e.g., in a biological sample) or a genetic alteration in a 18636, 2466, 43238, 1983, 52881, 2398, 45449, 50289, 52872 or 26908 gene, and to modulate 18636, 2466, 43238, 1983, 52881, 2398, 45449, 50289, 52872 or 26908 activity, as described further below. The 18636, 2466, 43238, 1983, 52881, 2398, 45449, 50289, 52872 or 26908 proteins can be used to treat disorders characterized by insufficient or excessive production of a 18636, 2466, 43238, 1983, 52881, 2398, 45449, 50289, 52872 or 26908 substrate or production of 18636, 2466, 43238, 1983, 52881, 2398, 45449, 50289, 52872 or 26908 inhibitors. In addition, the 18636, 2466, 43238, 1983, 52881, 2398, 45449, 50289, 52872 or 26908 proteins can be used to screen for naturally occurring 18636, 2466, 43238, 1983, 52881, 2398, 45449, 50289, 52872 or 26908 substrates, to screen for drugs or compounds which modulate 18636, 2466, 43238, 1983, 52881, 2398, 45449, 50289, 52872 or 26908 activity, as well as to treat disorders characterized by insufficient or excessive production of 18636, 2466, 43238, 1983, 52881, 2398, 45449, 50289, 52872 or 26908 protein or production of 18636, 2466, 43238, 1983, 52881, 2398, 45449, 50289, 52872 or 26908 protein forms which have decreased, aberrant or unwanted activity compared to 18636, 2466, 43238, 1983, 52881, 2398, 45449, 50289, 52872 or 26908 wild type protein (e.g., aberrant or deficient GPCR activity, or other activity). Moreover, the anti-18636, 2466, 43238, 1983, 52881, 2398, 45449, 50289, 52872 or 26908 antibodies of the invention can be used to detect and isolate 18636, 2466, 43238, 1983, 52881, 2398, 45449, 50289, 52872 or 26908 proteins, regulate the bioavailability of 18636, 2466, 43238, 1983, 52881, 2398, 45449, 50289, 52872 or 26908 proteins, and modulate 18636, 2466, 43238, 1983, 52881, 2398, 45449, 50289, 52872 or 26908 activity.

A method of evaluating a compound for the ability to interact with, e.g., bind, a subject 18636, 2466, 43238, 1983, 52881, 2398, 45449, 50289, 52872 or 26908 polypeptide is provided. The method includes: contacting the compound with the subject 18636, 2466, 43238, 1983, 52881, 2398, 45449, 50289, 52872 or 26908 polypeptide; and evaluating ability of the compound to interact with, e.g., to bind or form a complex with the subject 18636, 2466, 43238, 1983, 52881, 2398, 45449, 50289, 52872 or 26908 polypeptide. This method can be performed in vitro, e.g., in a cell free system, or in vivo, e.g., in a two-hybrid interaction trap assay. This method can be used to identify naturally occurring molecules which interact with subject 18636, 2466, 43238, 1983, 52881, 2398, 45449, 50289, 52872 or 26908 polypeptide. It can also be used to find natural or synthetic inhibitors of subject 18636, 2466, 43238, 1983, 52881, 2398, 45449, 50289, 52872 or 26908 polypeptide. Screening methods are discussed in more detail below.

### Screening Assays:

The invention provides methods (also referred to herein as “screening assays”) for identifying modulators, i.e., candidate or test compounds or agents (e.g., proteins, peptides, peptidomimetics, peptoids, small molecules or other drugs) which bind to 18636, 2466, 43238, 1983, 52881, 2398, 45449, 50289, 52872 or 26908 proteins, have a stimulatory or inhibitory effect on, for example, 18636, 2466, 43238, 1983, 52881, 2398, 45449, 50289, 52872 or 26908 expression or 18636, 2466, 43238, 1983, 52881, 2398, 45449, 50289, 52872 or 26908 activity, or have a stimulatory or inhibitory effect on, for example, the expression or activity of a 18636, 2466, 43238, 1983, 52881, 2398, 45449, 50289, 52872 or 26908 substrate. Compounds thus identified can be used to modulate the activity of target gene products (e.g., 18636, 2466, 43238, 1983, 52881, 2398, 45449, 50289, 52872 or 26908 genes) in a therapeutic protocol, to elaborate the biological function of the target gene product, or to identify compounds that disrupt normal target gene interactions.

In one embodiment, the invention provides assays for screening candidate or test compounds which are substrates of a 18636, 2466, 43238, 1983, 52881, 2398, 45449, 50289, 52872 or 26908 protein or polypeptide or a biologically active portion thereof. In another embodiment, the invention provides assays for screening candidate or test compounds which bind to or modulate the activity of a 18636, 2466, 43238, 1983, 52881, 2398, 45449, 50289, 52872 or 26908 protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909-13; Erb et al. (1994) Proc. Natl. Acad. Sci. USA 91:11422-426; Zuckermann et al. (1994). J. Med. Chem. 37:2678-85; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233-51.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a 18636, 2466, 43238, 1983, 52881, 2398, 45449, 50289, 52872 or 26908 protein or biologically active portion thereof is contacted with a test compound, and the ability of the test compound to modulate 18636, 2466, 43238, 1983, 52881, 2398, 45449, 50289, 52872 or 26908 activity is determined. Determining the ability of the test compound to modulate 18636, 2466, 43238, 1983, 52881, 2398, 45449, 50289, 52872 or 26908 activity can be accomplished by monitoring, for example, GPCR activity, or other activity. The cell, for example, can be of mammalian origin, e.g., human.

The ability of the test compound to modulate 18636, 2466, 43238, 1983, 52881, 2398, 45449, 50289, 52872 or 26908 binding to a compound, e.g., a 18636, 2466, 43238, 1983, 52881, 2398, 45449, 50289, 52872 or 26908 substrate, or to bind to 18636, 2466, 43238, 1983, 52881, 2398, 45449, 50289, 52872 or 26908 can also be evaluated. This can be accomplished, for example, by coupling the compound, e.g., the substrate, with a radioisotope or enzymatic label such that binding of the compound, e.g., the substrate, to 18636, 2466, 43238, 1983, 52881, 2398, 45449, 50289, 52872 or 26908 can be determined by detecting the labeled compound, e.g., substrate, in a complex. Alternatively, 18636, 2466, 43238, 1983, 52881, 2398, 45449, 50289, 52872 or 26908 could be coupled with a radioisotope or enzymatic label to monitor the ability of a test compound to modulate 18636, 2466, 43238, 1983, 52881, 2398, 45449, 50289, 52872 or 26908 binding to a 18636, 2466, 43238, 1983, 52881, 2398, 45449, 50289, 52872 or 26908 substrate in a complex. For example, compounds (e.g., 18636, 2466, 43238, 1983, 52881, 2398, 45449, 50289, 52872 or 26908 substrates) can be labeled with ¹²⁵I, ¹⁴C, ³⁵S or ³H., either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 18636, 2466, 43238, 1983, 52881, 2398, 45449, 50289, 52872 or 26908 substrate) to interact with 18636, 2466, 43238, 1983, 52881, 2398, 45449, 50289, 52872 or 26908 with or without the labeling of any of the interactants can be evaluated. For example, a microphysiometer can be used to detect the interaction of a compound with 18636, 2466, 43238, 1983, 52881, 2398, 45449, 50289, 52872 or 26908 without the labeling of either the compound or the 18636, 2466, 43238, 1983, 52881, 2398, 45449, 50289, 52872 or 26908. McConnell et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 18636, 2466, 43238, 1983, 52881, 2398, 45449, 50289, 52872 or 26908.

In yet another embodiment, a cell-free assay is provided in which a 18636, 2466, 43238, 1983, 52881, 2398, 45449, 50289, 52872 or 26908 protein or biologically active portion thereof is contacted with a test compound and the ability of the test compound to bind to the 18636, 2466, 43238, 1983, 52881, 2398, 45449, 50289, 52872 or 26908 protein or biologically active portion thereof is evaluated. Preferred biologically active portions of the 18636, 2466, 43238, 1983, 52881, 2398, 45449, 50289, 52872 or 26908 proteins to be used in assays of the present invention include fragments which participate in interactions with non-18636, 2466, 43238, 1983, 52881, 2398, 45449, 50289, 52872 or 26908 molecules, e.g., fragments with high surface probability scores.

Soluble and/or membrane-bound forms of isolated proteins (e.g., 18636, 2466, 43238, 1983, 52881, 2398, 45449, 50289, 52872 or 26908 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 18636, 2466, 43238, 1983, 52881, 2398, 45449, 50289, 52872 or 26908 protein to bind to a target molecule can be accomplished using real-time Biomolecular Interaction Analysis (BIA) (see, e.g., Sjolander and Urbaniczky (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 18636, 2466, 43238, 1983, 52881, 2398, 45449, 50289, 52872 or 26908, an anti-18636, 2466, 43238, 1983, 52881, 2398, 45449, 50289, 52872 or 26908 antibody or its target molecule to facilitate separation of complexed from uncomplexed forms of one or both of the proteins, as well as to accommodate automation of the assay. Binding of a test compound to a 18636, 2466, 43238, 1983, 52881, 2398, 45449, 50289, 52872 or 26908 protein, or interaction of a 18636, 2466, 43238, 1983, 52881, 2398, 45449, 50289, 52872 or 26908 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/18636, 2466, 43238, 1983, 52881, 2398, 45449, 50289, 52872 or 26908 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 18636, 2466, 43238, 1983, 52881, 2398, 45449, 50289, 52872 or 26908 protein, and the mixture incubated under conditions conducive to complex formation (e.g., at physiological conditions for salt and pH).

Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 18636, 2466, 43238, 1983, 52881, 2398, 45449, 50289, 52872 or 26908 binding or activity determined using standard techniques.

Other techniques for immobilizing either a 18636, 2466, 43238, 1983, 52881, 2398, 45449, 50289, 52872 or 26908 protein or a target molecule on matrices include using conjugation of biotin and streptavidin. Biotinylated 18636, 2466, 43238, 1983, 52881, 2398, 45449, 50289, 52872 or 26908 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific or selective for the immobilized component (the antibody, in turn, can be directly labeled or indirectly labeled with, e.g., a labeled anti-Ig antibody).

In one embodiment, this assay is performed utilizing antibodies reactive with 18636, 2466, 43238, 1983, 52881, 2398, 45449, 50289, 52872 or 26908 protein or target molecules but which do not interfere with binding of the 18636, 2466, 43238, 1983, 52881, 2398, 45449, 50289, 52872 or 26908 protein to its target molecule. Such antibodies can be derivatized to the wells of the plate, and unbound target or 18636, 2466, 43238, 1983, 52881, 2398, 45449, 50289, 52872 or 26908 protein trapped in the wells by antibody conjugation. Methods for detecting such complexes, in addition to those described above for the GST-immobilized complexes, include immunodetection of complexes using antibodies reactive with the 18636, 2466, 43238, 1983, 52881, 2398, 45449, 50289, 52872 or 26908 protein or target molecule, as well as enzyme-linked assays which rely on detecting an enzymatic activity associated with the 18636, 2466, 43238, 1983, 52881, 2398, 45449, 50289, 52872 or 26908 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas and Minton (1993) Trends Biochem Sci 18:284-7); chromatography (gel filtration chromatography, ion-exchange chromatography); electrophoresis (see, e.g., Ausubel et al., eds. (1999) Current Protocols in Molecular Biology, J. Wiley, New York.); and immunoprecipitation (see, for example, Ausubel et al., eds. (1999) Current Protocols in Molecular Biology, J. Wiley, New York). Such resins and chromatographic techniques are known to one skilled in the art (see, e.g., Heegaard (1998) J Mol Recognit 11: 141-8; Hage and Tweed (1997) J Chromatogr B Biomed Sci Appl. 699:499-525). Further, fluorescence energy transfer can also be conveniently utilized, as described herein, to detect binding without further purification of the complex from solution.

In a preferred embodiment, the assay includes contacting the 18636, 2466, 43238, 1983, 52881, 2398, 45449, 50289, 52872 or 26908 protein or biologically active portion thereof with a known compound which binds 18636, 2466, 43238, 1983, 52881, 2398, 45449, 50289, 52872 or 26908 to form an assay mixture, contacting the assay mixture with a test compound, and determining the ability of the test compound to interact with a 18636, 2466, 43238, 1983, 52881, 2398, 45449, 50289, 52872 or 26908 protein, wherein determining the ability of the test compound to interact with a 18636, 2466, 43238, 1983, 52881, 2398, 45449, 50289, 52872 or 26908 protein includes determining the ability of the test compound to preferentially bind to 18636, 2466, 43238, 1983, 52881, 2398, 45449, 50289, 52872 or 26908 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the 18636, 2466, 43238, 1983, 52881, 2398, 45449, 50289, 52872 or 26908 genes herein identified. In an alternative embodiment, the invention provides methods for determining the ability of the test compound to modulate the activity of a 18636, 2466, 43238, 1983, 52881, 2398, 45449, 50289, 52872 or 26908 protein through modulation of the activity of a downstream effector of a 18636, 2466, 43238, 1983, 52881, 2398, 45449, 50289, 52872 or 26908 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner.

Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific or selective for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific or selective for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific or selective for one of the binding components to anchor any complexes formed in solution, and a labeled antibody specific or selective for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 18636, 2466, 43238, 1983, 52881, 2398, 45449, 50289, 52872 or 26908 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 18636, 2466, 43238, 1983, 52881, 2398, 45449, 50289, 52872 or 26908 (“18636, 2466, 43238, 1983, 52881, 2398, 45449, 50289, 52872 or 26908-binding proteins” or “18636, 2466, 43238, 1983, 52881, 2398, 45449, 50289, 52872 or 26908-bp”) and are involved in 18636, 2466, 43238, 1983, 52881, 2398, 45449, 50289, 52872 or 26908 activity. Such 18636, 2466, 43238, 1983, 52881, 2398, 45449, 50289, 52872 or 26908-bps can be activators or inhibitors of signals by the 18636, 2466, 43238, 1983, 52881, 2398, 45449, 50289, 52872 or 26908 proteins or 18636, 2466, 43238, 1983, 52881, 2398, 45449, 50289, 52872 or 26908 targets as, for example, downstream elements of a 18636, 2466, 43238, 1983, 52881, 2398, 45449, 50289, 52872 or 26908-mediated signaling pathway.

The two-hybrid system is based on the modular nature of most transcription factors, which consist of separable DNA-binding and activation domains. Briefly, the assay utilizes two different DNA constructs. In one construct, the gene that codes for a 18636, 2466, 43238, 1983, 52881, 2398, 45449, 50289, 52872 or 26908 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 18636, 2466, 43238, 1983, 52881, 2398, 45449, 50289, 52872 or 26908 protein can be the fused to the activator domain.) If the “bait” and the “prey” proteins are able to interact, in vivo, forming a 18636, 2466, 43238, 1983, 52881, 2398, 45449, 50289, 52872 or 26908-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 18636, 2466, 43238, 1983, 52881, 2398, 45449, 50289, 52872 or 26908 protein.

In another embodiment, modulators of 18636, 2466, 43238, 1983, 52881, 2398, 45449, 50289, 52872 or 26908 expression are identified. For example, a cell or cell free mixture is contacted with a candidate compound and the expression of 18636, 2466, 43238, 1983, 52881, 2398, 45449, 50289, 52872 or 26908 mRNA or protein evaluated relative to the level of expression of 18636, 2466, 43238, 1983, 52881, 2398, 45449, 50289, 52872 or 26908 mRNA or protein in the absence of the candidate compound. When expression of 18636, 2466, 43238, 1983, 52881, 2398, 45449, 50289, 52872 or 26908 mRNA or protein is greater in the presence of the candidate compound than in its absence, the candidate compound is identified as a stimulator of 18636, 2466, 43238, 1983, 52881, 2398, 45449, 50289, 52872 or 26908 mRNA or protein expression. Alternatively, when expression of 18636, 2466, 43238, 1983, 52881, 2398, 45449, 50289, 52872 or 26908 mRNA or protein is less (statistically significantly less) in the presence of the candidate compound than in its absence, the candidate compound is identified as an inhibitor of 18636, 2466, 43238, 1983, 52881, 2398, 45449, 50289, 52872 or 26908 mRNA or protein expression. The level of 18636, 2466, 43238, 1983, 52881, 2398, 45449, 50289, 52872 or 26908 mRNA or protein expression can be determined by methods described herein for detecting 18636, 2466, 43238, 1983, 52881, 2398, 45449, 50289, 52872 or 26908 mRNA or protein.

In another aspect, the invention pertains to a combination of two or more of the assays described herein. For example, a modulating agent can be identified using a cell-based or a cell free assay, and the ability of the agent to modulate the activity of a 18636, 2466, 43238, 1983, 52881, 2398, 45449, 50289, 52872 or 26908 protein can be confirmed in vivo, e.g., in an animal such as an animal model for aberrant or deficient GPCR activity.

This invention further pertains to novel agents identified by the above-described screening assays. Accordingly, it is within the scope of this invention to further use an agent identified as described herein (e.g., a 18636, 2466, 43238, 1983, 52881, 2398, 45449, 50289, 52872 or 26908 modulating agent, an antisense 18636, 2466, 43238, 1983, 52881, 2398, 45449, 50289, 52872 or 26908 nucleic acid molecule, a 18636, 2466, 43238, 1983, 52881, 2398, 45449, 50289, 52872 or 26908-specific antibody, or a 18636, 2466, 43238, 1983, 52881, 2398, 45449, 50289, 52872 or 26908-binding partner) in an appropriate animal model to determine the efficacy, toxicity, side effects, or mechanism of action, of treatment with such an agent. Furthermore, novel agents identified by the above-described screening assays can be used for treatments as described herein.

### Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 18636, 2466, 43238, 1983, 52881, 2398, 45449, 50289, 52872 or 26908 with a disease; (ii) identify an individual from a minute biological sample (tissue typing); and (iii) aid in forensic identification of a biological sample. These applications are described in the subsections below.

### Chromosome Mapping

The 18636, 2466, 43238, 1983, 52881, 2398, 45449, 50289, 52872 or 26908 nucleotide sequences or portions thereof can be used to map the location of the 18636, 2466, 43238, 1983, 52881, 2398, 45449, 50289, 52872 or 26908 genes on a chromosome. This process is called chromosome mapping. Chromosome mapping is useful in correlating the 18636, 2466, 43238, 1983, 52881, 2398, 45449, 50289, 52872 or 26908 sequences with genes associated with disease.

Briefly, 18636, 2466, 43238, 1983, 52881, 2398, 45449, 50289, 52872 or 26908 genes can be mapped to chromosomes by preparing PCR primers (preferably 15-25 bp in length) from the 18636, 2466, 43238, 1983, 52881, 2398, 45449, 50289, 52872 or 26908 nucleotide sequences. These primers can then be used for PCR screening of somatic cell hybrids containing individual human chromosomes. Only those hybrids containing the human gene corresponding to the 18636, 2466, 43238, 1983, 52881, 2398, 45449, 50289, 52872 or 26908 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio et al. (1983) Science 220:919-924).

Other mapping strategies e.g., in situ hybridization (described in Fan et al. (1990) Proc. Natl. Acad. Sci. USA, 87:6223-27), pre-screening with labeled flow-sorted chromosomes, and pre-selection by hybridization to chromosome specific cDNA libraries can be used to map 18636, 2466, 43238, 1983, 52881, 2398, 45449, 50289, 52872 or 26908 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al. (1988) Human Chromosomes: A Manual of Basic Techniques, Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland et al. (1987) Nature, 325:783-787.

Moreover, differences in the DNA sequences between individuals affected and unaffected with a disease associated with the 18636, 2466, 43238, 1983, 52881, 2398, 45449, 50289, 52872 or 26908 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

18636, 2466, 43238, 1983, 52881, 2398, 45449, 50289, 52872 or 26908 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 18636, 2466, 43238, 1983, 52881, 2398, 45449, 50289, 52872 or 26908 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 29, 69, 77, 80, 83, 86, 89, 92 or 103 can provide positive individual identification with a panel of perhaps 10 to 1,000 primers which each yield a noncoding amplified sequence of 100 bases. If predicted coding sequences, such as those in SEQ ID NO:3, 31, 71, 79, 82, 85, 88, 91, 94 or 105 are used, a more appropriate number of primers for positive individual identification would be 500-2,000.

If a panel of reagents from 18636, 2466, 43238, 1983, 52881, 2398, 45449, 50289, 52872 or 26908 nucleotide sequences described herein is used to generate a unique identification database for an individual, those same reagents can later be used to identify tissue from that individual. Using the unique identification database, positive identification of the individual, living or dead, can be made from extremely small tissue samples.

### Use of Partial 18636, 2466, 43238, 1983, 52881, 2398, 45449, 50289, 52872 or 26908 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1, 29, 69, 77, 80, 83, 86, 89, 92 or 103 (e.g., fragments derived from the noncoding regions of SEQ ID NO:1, 29, 69, 77, 80, 83, 86, 89, 92 or 103 having a length of at least 20 bases, preferably at least 30 bases) are particularly appropriate for this use.

The 18636, 2466, 43238, 1983, 52881, 2398, 45449, 50289, 52872 or 26908 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such 18636, 2466, 43238, 1983, 52881, 2398, 45449, 50289, 52872 or 26908 probes can be used to identify tissue by species and/or by organ type.

In a similar fashion, these reagents, e.g., 18636, 2466, 43238, 1983, 52881, 2398, 45449, 50289, 52872 or 26908 primers or probes can be used to screen tissue culture for contamination (i.e. screen for the presence of a mixture of different types of cells in a culture).

### Predictive Medicine

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 18636, 2466, 43238, 1983, 52881, 2398, 45449, 50289, 52872 or 26908.

Such disorders include, e.g., a disorder associated with the misexpression of 18636, 2466, 43238, 1983, 52881, 2398, 45449, 50289, 52872 or 26908 gene; a cellular proliferative and/or differentiative disorder, brain, blood vessel, platelet, kidney or renal, hematopoeitic, prostate, testicular, skin, eye or opthalmological, hormonal, bone metabolism, immune e.g., inflammatory, cardiovascular, endothelial cell, liver, viral, pain, metabolic, neurological or CNS, hematological, intestinal, respiratory, fibrotic or angiogenic disorder.

The method includes one or more of the following: detecting, in a tissue of the subject, the presence or absence of a mutation which affects the expression of the 18636, 2466, 43238, 1983, 52881, 2398, 45449, 50289, 52872 or 26908 gene, or detecting the presence or absence of a mutation in a region which controls the expression of the gene, e.g., a mutation in the 5′ control region; detecting, in a tissue of the subject, the presence or absence of a mutation which alters the structure of the 18636, 2466, 43238, 1983, 52881, 2398, 45449, 50289, 52872 or 26908 gene; detecting, in a tissue of the subject, the misexpression of the 18636, 2466, 43238, 1983, 52881, 2398, 45449, 50289, 52872 or 26908 gene, at the mRNA level, e.g., detecting a non-wild type level of an mRNA; or detecting, in a tissue of the subject, the misexpression of the gene, at the protein level, e.g., detecting a non-wild type level of a 18636, 2466, 43238, 1983, 52881, 2398, 45449, 50289, 52872 or 26908 polypeptide.

In preferred embodiments the method includes: ascertaining the existence of at least one of: a deletion of one or more nucleotides from the 18636, 2466, 43238, 1983, 52881, 2398, 45449, 50289, 52872 or 26908 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:1, 29, 69, 77, 80, 83, 86, 89, 92 or 103, or naturally occurring mutants thereof or 5′ or 3′ flanking sequences naturally associated with the 18636, 2466, 43238, 1983, 52881, 2398, 45449, 50289, 52872 or 26908 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 18636, 2466, 43238, 1983, 52881, 2398, 45449, 50289, 52872 or 26908 gene; the presence of a non-wild type splicing pattern of a messenger RNA transcript of the gene; or a non-wild type level of 18636, 2466, 43238, 1983, 52881, 2398, 45449, 50289, 52872 or 26908.

Methods of the invention can be used prenatally or to determine if a subject's offspring will be at risk for a disorder.

In preferred embodiments the method includes determining the structure of a 18636, 2466, 43238, 1983, 52881, 2398, 45449, 50289, 52872 or 26908 gene, an abnormal structure being indicative of risk for the disorder.

In preferred embodiments the method includes contacting a sample from the subject with an antibody to the 18636, 2466, 43238, 1983, 52881, 2398, 45449, 50289, 52872 or 26908 protein or a nucleic acid, which hybridizes specifically with the gene. These and other embodiments are discussed below.

### Diagnostic and Prognostic Assays

The presence, level, or absence of 18636, 2466, 43238, 1983, 52881, 2398, 45449, 50289, 52872 or 26908 protein or nucleic acid in a biological sample can be evaluated by obtaining a biological sample from a test subject and contacting the biological sample with a compound or an agent capable of detecting 18636, 2466, 43238, 1983, 52881, 2398, 45449, 50289, 52872 or 26908 protein or nucleic acid (e.g., mRNA, genomic DNA) that encodes 18636, 2466, 43238, 1983, 52881, 2398, 45449, 50289, 52872 or 26908 protein such that the presence of 18636, 2466, 43238, 1983, 52881, 2398, 45449, 50289, 52872 or 26908 protein or nucleic acid is detected in the biological sample. The term “biological sample” includes tissues, cells and biological fluids isolated from a subject, as well as tissues, cells and fluids present within a subject. A preferred biological sample is serum. The level of expression of the 18636, 2466, 43238, 1983, 52881, 2398, 45449, 50289, 52872 or 26908 gene can be measured in a number of ways, including, but not limited to: measuring the mRNA encoded by the 18636, 2466, 43238, 1983, 52881, 2398, 45449, 50289, 52872 or 26908 genes; measuring the amount of protein encoded by the 18636, 2466, 43238, 1983, 52881, 2398, 45449, 50289, 52872 or 26908 genes; or measuring the activity of the protein encoded by the 18636, 2466, 43238, 1983, 52881, 2398, 45449, 50289, 52872 or 26908 genes.

The level of mRNA corresponding to the 18636, 2466, 43238, 1983, 52881, 2398, 45449, 50289, 52872 or 26908 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 18636, 2466, 43238, 1983, 52881, 2398, 45449, 50289, 52872 or 26908 nucleic acid, such as the nucleic acid of SEQ ID NO:1, 29, 69, 77, 80, 83, 86, 89, 92 or 103, or a portion thereof, such as an oligonucleotide of at least 7, 15, 30, 50, 100, 250 or 500 nucleotides in length and sufficient to specifically hybridize under stringent conditions to 18636, 2466, 43238, 1983, 52881, 2398, 45449, 50289, 52872 or 26908 mRNA or genomic DNA. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the 18636, 2466, 43238, 1983, 52881, 2398, 45449, 50289, 52872 or 26908 genes.

The level of mRNA in a sample that is encoded by one of 18636, 2466, 43238, 1983, 52881, 2398, 45449, 50289, 52872 or 26908 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 18636, 2466, 43238, 1983, 52881, 2398, 45449, 50289, 52872 or 26908 gene being analyzed.

In another embodiment, the methods further contacting a control sample with a compound or agent capable of detecting 18636, 2466, 43238, 1983, 52881, 2398, 45449, 50289, 52872 or 26908 mRNA, or genomic DNA, and comparing the presence of 18636, 2466, 43238, 1983, 52881, 2398, 45449, 50289, 52872 or 26908 mRNA or genomic DNA in the control sample with the presence of 18636, 2466, 43238, 1983, 52881, 2398, 45449, 50289, 52872 or 26908 mRNA or genomic DNA in the test sample.

A variety of methods can be used to determine the level of protein encoded by 18636, 2466, 43238, 1983, 52881, 2398, 45449, 50289, 52872 or 26908. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 18636, 2466, 43238, 1983, 52881, 2398, 45449, 50289, 52872 or 26908 protein in a biological sample in vitro as well as in vivo. In vitro techniques for detection of 18636, 2466, 43238, 1983, 52881, 2398, 45449, 50289, 52872 or 26908 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 18636, 2466, 43238, 1983, 52881, 2398, 45449, 50289, 52872 or 26908 protein include introducing into a subject a labeled anti-18636, 2466, 43238, 1983, 52881, 2398, 45449, 50289, 52872 or 26908 antibody. For example, the antibody can be labeled with a radioactive marker whose presence and location in a subject can be detected by standard imaging techniques.

In another embodiment, the methods further include contacting the control sample with a compound or agent capable of detecting 18636, 2466, 43238, 1983, 52881, 2398, 45449, 50289, 52872 or 26908 protein, and comparing the presence of 18636, 2466, 43238, 1983, 52881, 2398, 45449, 50289, 52872 or 26908 protein in the control sample with the presence of 18636, 2466, 43238, 1983, 52881, 2398, 45449, 50289, 52872 or 26908 protein in the test sample.

The invention also includes kits for detecting the presence of 18636, 2466, 43238, 1983, 52881, 2398, 45449, 50289, 52872 or 26908 in a biological sample. For example, the kit can include a compound or agent capable of detecting 18636, 2466, 43238, 1983, 52881, 2398, 45449, 50289, 52872 or 26908 protein or mRNA in a biological sample; and a standard. The compound or agent can be packaged in a suitable container. The kit can further comprise instructions for using the kit to detect 18636, 2466, 43238, 1983, 52881, 2398, 45449, 50289, 52872 or 26908 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 18636, 2466, 43238, 1983, 52881, 2398, 45449, 50289, 52872 or 26908 expression or activity. As used herein, the term “unwanted” includes an unwanted phenomenon involved in a biological response such as pain or deregulated cell proliferation.

In one embodiment, a disease or disorder associated with aberrant or unwanted 18636, 2466, 43238, 1983, 52881, 2398, 45449, 50289, 52872 or 26908 expression or activity is identified. A test sample is obtained from a subject and 18636, 2466, 43238, 1983, 52881, 2398, 45449, 50289, 52872 or 26908 protein or nucleic acid (e.g., mRNA or genomic DNA) is evaluated, wherein the level, e.g., the presence or absence, of 18636, 2466, 43238, 1983, 52881, 2398, 45449, 50289, 52872 or 26908 protein or nucleic acid is diagnostic for a subject having or at risk of developing a disease or disorder associated with aberrant or unwanted 18636, 2466, 43238, 1983, 52881, 2398, 45449, 50289, 52872 or 26908 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 18636, 2466, 43238, 1983, 52881, 2398, 45449, 50289, 52872 or 26908 expression or activity. For example, such methods can be used to determine whether a subject can be effectively treated with an agent for a cellular proliferative and/or differentiative disorder, brain, blood vessel, platelet, kidney or renal, hematopoeitic, prostate, testicular, skin, eye or opthalmological, hormonal, bone metabolism, immune e.g., inflammatory, cardiovascular, endothelial cell, liver, viral, pain, metabolic, neurological or CNS, hematological, intestinal, respiratory, fibrotic or angiogenic disorder.

The methods of the invention can also be used to detect genetic alterations in a 18636, 2466, 43238, 1983, 52881, 2398, 45449, 50289, 52872 or 26908 gene, thereby determining if a subject with the altered gene is at risk for a disorder characterized by misregulation in 18636, 2466, 43238, 1983, 52881, 2398, 45449, 50289, 52872 or 26908 protein activity or nucleic acid expression, such as a cellular proliferative and/or differentiative disorder, brain, blood vessel, platelet, kidney or renal, hematopoeitic, prostate, testicular, skin, eye or opthalmological, hormonal, bone metabolism, immune e.g., inflammatory, cardiovascular, endothelial cell, liver, viral, pain, metabolic, neurological or CNS, hematological, intestinal, respiratory, fibrotic or angiogenic disorder.

In preferred embodiments, the methods include detecting, in a sample from the subject, the presence or absence of a genetic alteration characterized by at least one of an alteration affecting the integrity of a gene encoding a 18636, 2466, 43238, 1983, 52881, 2398, 45449, 50289, 52872 or 26908-protein, or the mis-expression of the 18636, 2466, 43238, 1983, 52881, 2398, 45449, 50289, 52872 or 26908 gene. For example, such genetic alterations can be detected by ascertaining the existence of at least one of 1) a deletion of one or more nucleotides from a 18636, 2466, 43238, 1983, 52881, 2398, 45449, 50289, 52872 or 26908 gene; 2) an addition of one or more nucleotides to a 18636, 2466, 43238, 1983, 52881, 2398, 45449, 50289, 52872 or 26908 gene; 3) a substitution of one or more nucleotides of a 18636, 2466, 43238, 1983, 52881, 2398, 45449, 50289, 52872 or 26908 gene, 4) a chromosomal rearrangement of a 18636, 2466, 43238, 1983, 52881, 2398, 45449, 50289, 52872 or 26908 gene; 5) an alteration in the level of a messenger RNA transcript of a 18636, 2466, 43238, 1983, 52881, 2398, 45449, 50289, 52872 or 26908 gene, 6) aberrant modification of a 18636, 2466, 43238, 1983, 52881, 2398, 45449, 50289, 52872 or 26908 gene, such as of the methylation pattern of the genomic DNA, 7) the presence of a non-wild type splicing pattern of a messenger RNA transcript of a 18636, 2466, 43238, 1983, 52881, 2398, 45449, 50289, 52872 or 26908 gene, 8) a non-wild type level of a 18636, 2466, 43238, 1983, 52881, 2398, 45449, 50289, 52872 or 26908-protein, 9) allelic loss of a 18636, 2466, 43238, 1983, 52881, 2398, 45449, 50289, 52872 or 26908 gene, and 10) inappropriate post-translational modification of a 18636, 2466, 43238, 1983, 52881, 2398, 45449, 50289, 52872 or 26908-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 18636, 2466, 43238, 1983, 52881, 2398, 45449, 50289, 52872 or 26908-gene. This method can include the steps of collecting a sample of cells from a subject, isolating nucleic acid (e.g., genomic, mRNA or both) from the sample, contacting the nucleic acid sample with one or more primers which specifically hybridize to a 18636, 2466, 43238, 1983, 52881, 2398, 45449, 50289, 52872 or 26908 gene under conditions such that hybridization and amplification of the 18636, 2466, 43238, 1983, 52881, 2398, 45449, 50289, 52872 or 26908 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 18636, 2466, 43238, 1983, 52881, 2398, 45449, 50289, 52872 or 26908 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 18636, 2466, 43238, 1983, 52881, 2398, 45449, 50289, 52872 or 26908 can be identified by hybridizing a sample and control nucleic acids, e.g., DNA or RNA, two dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin et al. (1996) Human Mutation 7: 244-255; Kozal et al. (1996) Nature Medicine 2: 753-759). For example, genetic mutations in 18636, 2466, 43238, 1983, 52881, 2398, 45449, 50289, 52872 or 26908 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 18636, 2466, 43238, 1983, 52881, 2398, 45449, 50289, 52872 or 26908 gene and detect mutations by comparing the sequence of the sample 18636, 2466, 43238, 1983, 52881, 2398, 45449, 50289, 52872 or 26908 with the corresponding wild-type (control) sequence. Automated sequencing procedures can be utilized when performing the diagnostic assays (Naeve et al. (1995) Biotechniques 19:448-53), including sequencing by mass spectrometry.

Other methods for detecting mutations in the 18636, 2466, 43238, 1983, 52881, 2398, 45449, 50289, 52872 or 26908 gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 18636, 2466, 43238, 1983, 52881, 2398, 45449, 50289, 52872 or 26908 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 18636, 2466, 43238, 1983, 52881, 2398, 45449, 50289, 52872 or 26908 genes. For example, single strand conformation polymorphism (SSCP) can be used to detect differences in electrophoretic mobility between mutant and wild type nucleic acids (Orita et al. (1989) Proc Natl. Acad. Sci. USA: 86:2766, see also Cotton (1993) Mutat. Res. 285:125-144; and Hayashi (1992) Genet. Anal. Tech. Appl. 9:73-79). Single-stranded DNA fragments of sample and control 18636, 2466, 43238, 1983, 52881, 2398, 45449, 50289, 52872 or 26908 nucleic acids will be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective-primer extension (Saiki et al. (1986) Nature 324:163); Saiki et al. (1989) Proc. Natl. Acad. Sci. USA 86:6230).

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification can also be performed using Taq ligase for amplification (Barany (1991) Proc. Natl. Acad. Sci USA 88:189-93). In such cases, ligation will occur only if there is a perfect match at the 3′ end of the 5′ sequence making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which can be conveniently used, e.g., in clinical settings to diagnose patients exhibiting symptoms or family history of a disease or illness involving a 18636, 2466, 43238, 1983, 52881, 2398, 45449, 50289, 52872 or 26908 gene.

### Use of 18636, 2466, 43238, 1983, 52881, 2398, 45449, 50289, 52872 or 26908 Molecules as Surrogate Markers

The 18636, 2466, 43238, 1983, 52881, 2398, 45449, 50289, 52872 or 26908 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 18636, 2466, 43238, 1983, 52881, 2398, 45449, 50289, 52872 or 26908 molecules of the invention can be detected, and can be correlated with one or more biological states in vivo. For example, the 18636, 2466, 43238, 1983, 52881, 2398, 45449, 50289, 52872 or 26908 molecules of the invention can serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers can serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease can be made using cholesterol levels as a surrogate marker, and an analysis of HIV infection can be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 18636, 2466, 43238, 1983, 52881, 2398, 45449, 50289, 52872 or 26908 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker can be indicative of the concentration of the drug in a biological tissue, in that the marker is either expressed or transcribed or not expressed or transcribed in that tissue in relationship to the level of the drug. In this fashion, the distribution or uptake of the drug can be monitored by the pharmacodynamic marker. Similarly, the presence or quantity of the pharmacodynamic marker can be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug can be sufficient to activate multiple rounds of marker (e.g., a 18636, 2466, 43238, 1983, 52881, 2398, 45449, 50289, 52872 or 26908 marker) transcription or expression, the amplified marker can be in a quantity which is more readily detectable than the drug itself. Also, the marker can be more easily detected due to the nature of the marker itself; for example, using the methods described herein, anti-18636, 2466, 43238, 1983, 52881, 2398, 45449, 50289, 52872 or 26908 antibodies can be employed in an immune-based detection system for a 18636, 2466, 43238, 1983, 52881, 2398, 45449, 50289, 52872 or 26908 protein marker, or 18636, 2466, 43238, 1983, 52881, 2398, 45449, 50289, 52872 or 26908-specific radiolabeled probes can be used to detect a 18636, 2466, 43238, 1983, 52881, 2398, 45449, 50289, 52872 or 26908 mRNA marker. Furthermore, the use of a pharmacodynamic marker can offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Phamm. 56 Suppl. 3: S16-S20.

The 18636, 2466, 43238, 1983, 52881, 2398, 45449, 50289, 52872 or 26908 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, can be selected. For example, based on the presence or quantity of RNA, or protein (e.g., 18636, 2466, 43238, 1983, 52881, 2398, 45449, 50289, 52872 or 26908 protein or RNA) for specific tumor markers in a subject, a drug or course of treatment can be selected that is optimized for the treatment of the specific tumor likely to be present in the subject. Similarly, the presence or absence of a specific sequence mutation in 18636, 2466, 43238, 1983, 52881, 2398, 45449, 50289, 52872 or 26908 dNA can correlate with a 18636, 2466, 43238, 1983, 52881, 2398, 45449, 50289, 52872 or 26908 drug response. The use of pharmacogenomic markers therefore permits the application of the most appropriate treatment for each subject without having to administer the therapy.

### Pharmaceutical Compositions

The nucleic acid and polypeptides, fragments thereof, as well as anti-18636, 2466, 43238, 1983, 52881, 2398, 45449, 50289, 52872 or 26908 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody, unconjugated or conjugated as described herein, can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent can, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### Methods of Treatment:

The present invention provides for both prophylactic and therapeutic methods of treating a subject at risk of (or susceptible to) a disorder or having a disorder associated with aberrant or unwanted 18636, 2466, 43238, 1983, 52881, 2398, 45449, 50289, 52872 or 26908 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments can be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 18636, 2466, 43238, 1983, 52881, 2398, 45449, 50289, 52872 or 26908 molecules of the present invention or 18636, 2466, 43238, 1983, 52881, 2398, 45449, 50289, 52872 or 26908 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 18636, 2466, 43238, 1983, 52881, 2398, 45449, 50289, 52872 or 26908 expression or activity, by administering to the subject a 18636, 2466, 43238, 1983, 52881, 2398, 45449, 50289, 52872 or 26908 or an agent which modulates 18636, 2466, 43238, 1983, 52881, 2398, 45449, 50289, 52872 or 26908 expression or at least one 18636, 2466, 43238, 1983, 52881, 2398, 45449, 50289, 52872 or 26908 activity. Subjects at risk for a disease which is caused or contributed to by aberrant or unwanted 18636, 2466, 43238, 1983, 52881, 2398, 45449, 50289, 52872 or 26908 expression or activity can be identified by, for example, any or a combination of diagnostic or prognostic assays as described herein. Administration of a prophylactic agent can occur prior to the manifestation of symptoms characteristic of the 18636, 2466, 43238, 1983, 52881, 2398, 45449, 50289, 52872 or 26908 aberrance, such that a disease or disorder is prevented or, alternatively, delayed in its progression. Depending on the type of 18636, 2466, 43238, 1983, 52881, 2398, 45449, 50289, 52872 or 26908 aberrance, for example, a 18636, 2466, 43238, 1983, 52881, 2398, 45449, 50289, 52872 or 26908, 18636, 2466, 43238, 1983, 52881, 2398, 45449, 50289, 52872 or 26908 agonist or 18636, 2466, 43238, 1983, 52881, 2398, 45449, 50289, 52872 or 26908 antagonist agent can be used for treating the subject. The appropriate agent can be determined based on screening assays described herein.

It is possible that some 18636, 2466, 43238, 1983, 52881, 2398, 45449, 50289, 52872 or 26908 disorders can be caused, at least in part, by an abnormal level of gene product, or by the presence of a gene product exhibiting abnormal activity. As such, the reduction in the level and/or activity of such gene products would bring about the amelioration of disorder symptoms.

The 18636, 2466, 43238, 1983, 52881, 2398, 45449, 50289, 52872 or 26908 molecules can act as novel diagnostic targets and therapeutic agents for controlling one or more of a cellular proliferative and/or differentiative disorder, brain, blood vessel, platelet, kidney or renal, hematopoeitic, prostate, testicular, skin, eye or opthalmological, hormonal, bone metabolism, immune e.g., inflammatory, cardiovascular, endothelial cell, liver, viral, pain, metabolic, neurological or CNS, hematological, intestinal, respiratory, fibrotic or angiogenic disorder, all of which are described above.

As discussed, successful treatment of 18636, 2466, 43238, 1983, 52881, 2398, 45449, 50289, 52872 or 26908 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 18636, 2466, 43238, 1983, 52881, 2398, 45449, 50289, 52872 or 26908 disorders. Such molecules can include, but are not limited to peptides, phosphopeptides, small organic or inorganic molecules, or antibodies (including, for example, polyclonal, monoclonal, humanized, human, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules can be utilized in treating or preventing a disease characterized by 18636, 2466, 43238, 1983, 52881, 2398, 45449, 50289, 52872 or 26908 expression is through the use of aptamer molecules specific for 18636, 2466, 43238, 1983, 52881, 2398, 45449, 50289, 52872 or 26908 protein. Aptamers are nucleic acid molecules having a tertiary structure which permits them to specifically or selectively bind to protein ligands (see, e.g., Osborne et al. (1997) Curr. Opin. Chem. Biol. 1: 5-9; and Patel (1997) Curr Opin Chem Biol 1:32-46). Since nucleic acid molecules can in many cases be more conveniently introduced into target cells than therapeutic protein molecules can be, aptamers offer a method by which 18636, 2466, 43238, 1983, 52881, 2398, 45449, 50289, 52872 or 26908 protein activity can be specifically decreased without the introduction of drugs or other molecules which can have pluripotent effects.

Antibodies can be generated that are both specific for target gene product and that reduce target gene product activity. Such antibodies can, therefore, by administered in instances whereby negative modulatory techniques are appropriate for the treatment of 18636, 2466, 43238, 1983, 52881, 2398, 45449, 50289, 52872 or 26908 disorders. For a description of antibodies, see the Antibody section above.

In circumstances wherein injection of an animal or a human subject with a 18636, 2466, 43238, 1983, 52881, 2398, 45449, 50289, 52872 or 26908 protein or epitope for stimulating antibody production is harmful to the subject, it is possible to generate an immune response against 18636, 2466, 43238, 1983, 52881, 2398, 45449, 50289, 52872 or 26908 through the use of anti-idiotypic antibodies (see, for example, Herlyn (1999) Ann Med 31:66-78; and Bhattacharya-Chatterjee and Foon (1998) Cancer Treat Res. 94:51-68). If an anti-idiotypic antibody is introduced into a mammal or human subject, it should stimulate the production of anti-anti-idiotypic antibodies, which should be specific to the 18636, 2466, 43238, 1983, 52881, 2398, 45449, 50289, 52872 or 26908 protein.

Vaccines directed to a disease characterized by 18636, 2466, 43238, 1983, 52881, 2398, 45449, 50289, 52872 or 26908 expression can also be generated in this fashion.

In instances where the target antigen is intracellular and whole antibodies are used, internalizing antibodies can be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 18636, 2466, 43238, 1983, 52881, 2398, 45449, 50289, 52872 or 26908 disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays can utilize antibody mimics and/or “biosensors” that have been created through molecular imprinting techniques. The compound which is able to modulate 18636, 2466, 43238, 1983, 52881, 2398, 45449, 50289, 52872 or 26908 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al (1996) Current Opinion in Biotechnology 7:89-94 and in Shea (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis et al (1993) Nature 361:645-647. Through the use of isotope-labeling, the “free” concentration of compound which modulates the expression or activity of 18636, 2466, 43238, 1983, 52881, 2398, 45449, 50289, 52872 or 26908 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz et al (1995) Analytical Chemistry 67:2142-2144.

Another aspect of the invention pertains to methods of modulating 18636, 2466, 43238, 1983, 52881, 2398, 45449, 50289, 52872 or 26908 expression or activity for therapeutic purposes. Accordingly, in an exemplary embodiment, the modulatory method of the invention involves contacting a cell with a 18636, 2466, 43238, 1983, 52881, 2398, 45449, 50289, 52872 or 26908 or agent that modulates one or more of the activities of 18636, 2466, 43238, 1983, 52881, 2398, 45449, 50289, 52872 or 26908 protein activity associated with the cell. An agent that modulates 18636, 2466, 43238, 1983, 52881, 2398, 45449, 50289, 52872 or 26908 protein activity can be an agent as described herein, such as a nucleic acid or a protein, a naturally-occurring target molecule of a 18636, 2466, 43238, 1983, 52881, 2398, 45449, 50289, 52872 or 26908 protein (e.g., a 18636, 2466, 43238, 1983, 52881, 2398, 45449, 50289, 52872 or 26908 substrate or receptor), a 18636, 2466, 43238, 1983, 52881, 2398, 45449, 50289, 52872 or 26908 antibody, a 18636, 2466, 43238, 1983, 52881, 2398, 45449, 50289, 52872 or 26908 agonist or antagonist, a peptidomimetic of a 18636, 2466, 43238, 1983, 52881, 2398, 45449, 50289, 52872 or 26908 agonist or antagonist, or other small molecule.

In one embodiment, the agent stimulates one or 18636, 2466, 43238, 1983, 52881, 2398, 45449, 50289, 52872 or 26908 activities. Examples of such stimulatory agents include active 18636, 2466, 43238, 1983, 52881, 2398, 45449, 50289, 52872 or 26908 protein and a nucleic acid molecule encoding 18636, 2466, 43238, 1983, 52881, 2398, 45449, 50289, 52872 or 26908. In another embodiment, the agent inhibits one or more 18636, 2466, 43238, 1983, 52881, 2398, 45449, 50289, 52872 or 26908 activities. Examples of such inhibitory agents include antisense 18636, 2466, 43238, 1983, 52881, 2398, 45449, 50289, 52872 or 26908 nucleic acid molecules, anti-18636, 2466, 43238, 1983, 52881, 2398, 45449, 50289, 52872 or 26908 antibodies, and 18636, 2466, 43238, 1983, 52881, 2398, 45449, 50289, 52872 or 26908 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 18636, 2466, 43238, 1983, 52881, 2398, 45449, 50289, 52872 or 26908 protein or nucleic acid molecule. In one embodiment, the method involves administering an agent (e.g., an agent identified by a screening assay described herein), or combination of agents that modulates (e.g., up regulates or down regulates) 18636, 2466, 43238, 1983, 52881, 2398, 45449, 50289, 52872 or 26908 expression or activity. In another embodiment, the method involves administering a 18636, 2466, 43238, 1983, 52881, 2398, 45449, 50289, 52872 or 26908 protein or nucleic acid molecule as therapy to compensate for reduced, aberrant, or unwanted 18636, 2466, 43238, 1983, 52881, 2398, 45449, 50289, 52872 or 26908 expression or activity.

Stimulation of 18636, 2466, 43238, 1983, 52881, 2398, 45449, 50289, 52872 or 26908 activity is desirable in situations in which 18636, 2466, 43238, 1983, 52881, 2398, 45449, 50289, 52872 or 26908 is abnormally downregulated and/or in which increased 18636, 2466, 43238, 1983, 52881, 2398, 45449, 50289, 52872 or 26908 activity is likely to have a beneficial effect. For example, stimulation of 18636, 2466, 43238, 1983, 52881, 2398, 45449, 50289, 52872 or 26908 activity is desirable in situations in which a 18636, 2466, 43238, 1983, 52881, 2398, 45449, 50289, 52872 or 26908 is downregulated and/or in which increased 18636, 2466, 43238, 1983, 52881, 2398, 45449, 50289, 52872 or 26908 activity is likely to have a beneficial effect. Likewise, inhibition of 18636, 2466, 43238, 1983, 52881, 2398, 45449, 50289, 52872 or 26908 activity is desirable in situations in which 18636, 2466, 43238, 1983, 52881, 2398, 45449, 50289, 52872 or 26908 is abnormally upregulated and/or in which decreased 18636, 2466, 43238, 1983, 52881, 2398, 45449, 50289, 52872 or 26908 activity is likely to have a beneficial effect.

### Pharmacogenomics

The 18636, 2466, 43238, 1983, 52881, 2398, 45449, 50289, 52872 or 26908 molecules of the present invention, as well as agents, or modulators which have a stimulatory or inhibitory effect on 18636, 2466, 43238, 1983, 52881, 2398, 45449, 50289, 52872 or 26908 activity (e.g., 18636, 2466, 43238, 1983, 52881, 2398, 45449, 50289, 52872 or 26908 gene expression) as identified by a screening assay described herein can be administered to individuals to treat (prophylactically or therapeutically) 18636, 2466, 43238, 1983, 52881, 2398, 45449, 50289, 52872 or 26908-associated disorders (e.g., aberrant or deficient GPCR activity) associated with aberrant or unwanted 18636, 2466, 43238, 1983, 52881, 2398, 45449, 50289, 52872 or 26908 activity.

In conjunction with such treatment, pharmacogenomics (i.e., the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a 18636, 2466, 43238, 1983, 52881, 2398, 45449, 50289, 52872 or 26908 molecule or 18636, 2466, 43238, 1983, 52881, 2398, 45449, 50289, 52872 or 26908 modulator as well as tailoring the dosage and/or therapeutic regimen of treatment with a 18636, 2466, 43238, 1983, 52881, 2398, 45449, 50289, 52872 or 26908 molecule or 18636, 2466, 43238, 1983, 52881, 2398, 45449, 50289, 52872 or 26908 modulator.

Pharmacogenomics deals with clinically significant hereditary variations in the response to drugs due to altered drug disposition and abnormal action in affected persons. See, for example, Eichelbaum et al. (1996) Clin. Exp. Pharmacol. Physiol. 23:983-985 and Linder et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP can occur once per every 1000 bases of DNA. A SNP can be involved in a disease process, however, the vast majority can not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that can be common among such genetically similar individuals.

Alternatively, a method termed the “candidate gene approach”, can be utilized to identify genes that predict drug response. According to this method, if a gene that encodes a drug's target is known (e.g., a 18636, 2466, 43238, 1983, 52881, 2398, 45449, 50289, 52872 or 26908 protein of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a 18636, 2466, 43238, 1983, 52881, 2398, 45449, 50289, 52872 or 26908 molecule or 18636, 2466, 43238, 1983, 52881, 2398, 45449, 50289, 52872 or 26908 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 18636, 2466, 43238, 1983, 52881, 2398, 45449, 50289, 52872 or 26908 molecule or 18636, 2466, 43238, 1983, 52881, 2398, 45449, 50289, 52872 or 26908 modulator, such as a modulator identified by one of the exemplary screening assays described herein.

The present invention further provides methods for identifying new agents, or combinations, that are based on identifying agents that modulate the activity of one or more of the gene products encoded by one or more of the 18636, 2466, 43238, 1983, 52881, 2398, 45449, 50289, 52872 or 26908 genes of the present invention, wherein these products can be associated with resistance of the cells to a therapeutic agent. Specifically, the activity of the proteins encoded by the 18636, 2466, 43238, 1983, 52881, 2398, 45449, 50289, 52872 or 26908 genes of the present invention can be used as a basis for identifying agents for overcoming agent resistance. By blocking the activity of one or more of the resistance proteins, target cells, e.g., human cells, will become sensitive to treatment with an agent to which the unmodified target cells were resistant.

Monitoring the influence of agents (e.g., drugs) on the expression or activity of a 18636, 2466, 43238, 1983, 52881, 2398, 45449, 50289, 52872 or 26908 protein can be applied in clinical trials. For example, the effectiveness of an agent determined by a screening assay as described herein to increase 18636, 2466, 43238, 1983, 52881, 2398, 45449, 50289, 52872 or 26908 gene expression, protein levels, or upregulate 18636, 2466, 43238, 1983, 52881, 2398, 45449, 50289, 52872 or 26908 activity, can be monitored in clinical trials of subjects exhibiting decreased 18636, 2466, 43238, 1983, 52881, 2398, 45449, 50289, 52872 or 26908 gene expression, protein levels, or downregulated 18636, 2466, 43238, 1983, 52881, 2398, 45449, 50289, 52872 or 26908 activity. Alternatively, the effectiveness of an agent determined by a screening assay to decrease 18636, 2466, 43238, 1983, 52881, 2398, 45449, 50289, 52872 or 26908 gene expression, protein levels, or downregulate 18636, 2466, 43238, 1983, 52881, 2398, 45449, 50289, 52872 or 26908 activity, can be monitored in clinical trials of subjects exhibiting increased 18636, 2466, 43238, 1983, 52881, 2398, 45449, 50289, 52872 or 26908 gene expression, protein levels, or upregulated 18636, 2466, 43238, 1983, 52881, 2398, 45449, 50289, 52872 or 26908 activity. In such clinical trials, the expression or activity of a 18636, 2466, 43238, 1983, 52881, 2398, 45449, 50289, 52872 or 26908 gene, and preferably, other genes that have been implicated in, for example, a GPCR-associated or another 18636, 2466, 43238, 1983, 52881, 2398, 45449, 50289, 52872 or 26908-associated disorder can be used as a “read out” or markers of the phenotype of a particular cell.

### Other Embodiments

In another aspect, the invention features a method of analyzing a plurality of capture probes. The method is useful, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence, wherein the capture probes are from a cell or subject which expresses 18636, 2466, 43238, 1983, 52881, 2398, 45449, 50289, 52872 or 26908 or from a cell or subject in which a 18636, 2466, 43238, 1983, 52881, 2398, 45449, 50289, 52872 or 26908 mediated response has been elicited; contacting the array with a 18636, 2466, 43238, 1983, 52881, 2398, 45449, 50289, 52872 or 26908 nucleic acid (preferably purified), a 18636, 2466, 43238, 1983, 52881, 2398, 45449, 50289, 52872 or 26908 polypeptide (preferably purified), or an anti-18636, 2466, 43238, 1983, 52881, 2398, 45449, 50289, 52872 or 26908 antibody, and thereby evaluating the plurality of capture probes. Binding, e.g., in the case of a nucleic acid, hybridization with a capture probe at an address of the plurality, is detected, e.g., by a signal generated from a label attached to the 18636, 2466, 43238, 1983, 52881, 2398, 45449, 50289, 52872 or 26908 nucleic acid, polypeptide, or antibody.

The capture probes can be a set of nucleic acids from a selected sample, e.g., a sample of nucleic acids derived from a control or non-stimulated tissue or cell.

The method can include contacting the 18636, 2466, 43238, 1983, 52881, 2398, 45449, 50289, 52872 or 26908 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of 18636, 2466, 43238, 1983, 52881, 2398, 45449, 50289, 52872 or 26908. Such methods can be used to diagnose a subject, e.g., to evaluate risk for a disease or disorder, to evaluate suitability of a selected treatment for a subject, to evaluate whether a subject has a disease or disorder.

The method can be used to detect SNPs, as described above.

In another aspect, the invention features, a method of analyzing 18636, 2466, 43238, 1983, 52881, 2398, 45449, 50289, 52872 or 26908, e.g., analyzing structure, function, or relatedness to other nucleic acid or amino acid sequences. The method includes: providing a 18636, 2466, 43238, 1983, 52881, 2398, 45449, 50289, 52872 or 26908 nucleic acid or amino acid sequence; comparing the 18636, 2466, 43238, 1983, 52881, 2398, 45449, 50289, 52872 or 26908 sequence with one or more preferably a plurality of sequences from a collection of sequences, e.g., a nucleic acid or protein sequence database; to thereby analyze 18636, 2466, 43238, 1983, 52881, 2398, 45449, 50289, 52872 or 26908.

The method can include evaluating the sequence identity between a 18636, 2466, 43238, 1983, 52881, 2398, 45449, 50289, 52872 or 26908 sequence and a database sequence. The method can be performed by accessing the database at a second site, e.g., over the internet. Preferred databases include GenBank™ and SwissProt.

In another aspect, the invention features, a set of oligonucleotides, useful, e.g., for identifying SNP's, or identifying specific alleles of 18636, 2466, 43238, 1983, 52881, 2398, 45449, 50289, 52872 or 26908. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with differential labels, such that an oligonucleotide which hybridizes to one allele provides a signal that is distinguishable from an oligonucleotides which hybridizes to a second allele.

The sequences of 18636, 2466, 43238, 1983, 52881, 2398, 45449, 50289, 52872 or 26908 molecules are provided in a variety of mediums to facilitate use thereof. A sequence can be provided as a manufacture, other than an isolated nucleic acid or amino acid molecule, which contains a 18636, 2466, 43238, 1983, 52881, 2398, 45449, 50289, 52872 or 26908 molecule. Such a manufacture can provide a nucleotide or amino acid sequence, e.g., an open reading frame, in a form which allows examination of the manufacture using means not directly applicable to examining the nucleotide or amino acid sequences, or a subset thereof, as they exist in nature or in purified form.

A 18636, 2466, 43238, 1983, 52881, 2398, 45449, 50289, 52872 or 26908 nucleotide or amino acid sequence can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as compact disc and CD-ROM; electrical storage media such as RAM, ROM, EPROM, EEPROM, and the like; and general hard disks and hybrids of these categories such as magnetic/optical storage media. The medium is adapted or configured for having thereon 18636, 2466, 43238, 1983, 52881, 2398, 45449, 50289, 52872 or 26908 sequence information of the present invention.

As used herein, the term “electronic apparatus” is intended to include any suitable computing or processing apparatus of other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as personal digital assistants (PDAs), cellular phones, pagers, and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the 18636, 2466, 43238, 1983, 52881, 2398, 45449, 50289, 52872 or 26908 sequence information.

A variety of data storage structures are available to a skilled artisan for creating a computer readable medium having recorded thereon a 18636, 2466, 43238, 1983, 52881, 2398, 45449, 50289, 52872 or 26908 nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

By providing the 18636, 2466, 43238, 1983, 52881, 2398, 45449, 50289, 52872 or 26908 nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif.

The present invention therefore provides a medium for holding instructions for performing a method for determining whether a subject has a GPCR-associated or another 18636, 2466, 43238, 1983, 52881, 2398, 45449, 50289, 52872 or 26908-associated disease or disorder or a pre-disposition to a GPCR-associated or another 18636, 2466, 43238, 1983, 52881, 2398, 45449, 50289, 52872 or 26908-associated disease or disorder, wherein the method comprises the steps of determining 18636, 2466, 43238, 1983, 52881, 2398, 45449, 50289, 52872 or 26908 sequence information associated with the subject and based on the 18636, 2466, 43238, 1983, 52881, 2398, 45449, 50289, 52872 or 26908 sequence information, determining whether the subject has a GPCR-associated or another 18636, 2466, 43238, 1983, 52881, 2398, 45449, 50289, 52872 or 26908-associated disease or disorder and/or recommending a particular treatment for the disease, disorder, or pre-disease condition.

The present invention further provides in an electronic system and/or in a network, a method for determining whether a subject has a GPCR-associated or another 18636, 2466, 43238, 1983, 52881, 2398, 45449, 50289, 52872 or 26908-associated disease or disorder or a pre-disposition to a disease associated with 18636, 2466, 43238, 1983, 52881, 2398, 45449, 50289, 52872 or 26908, wherein the method comprises the steps of determining 18636, 2466, 43238, 1983, 52881, 2398, 45449, 50289, 52872 or 26908 sequence information associated with the subject, and based on the 18636, 2466, 43238, 1983, 52881, 2398, 45449, 50289, 52872 or 26908 sequence information, determining whether the subject has a GPCR-associated or another 18636, 2466, 43238, 1983, 52881, 2398, 45449, 50289, 52872 or 26908-associated disease or disorder or a pre-disposition to a GPCR-associated or another 18636, 2466, 43238, 1983, 52881, 2398, 45449, 50289, 52872 or 26908-associated disease or disorder, and/or recommending a particular treatment for the disease, disorder, or pre-disease condition. The method may further comprise the step of receiving phenotypic information associated with the subject and/or acquiring from a network phenotypic information associated with the subject.

The present invention also provides in a network, a method for determining whether a subject has a GPCR-associated or another 18636, 2466, 43238, 1983, 52881, 2398, 45449, 50289, 52872 or 26908-associated disease or disorder or a pre-disposition to a GPCR-associated or another 18636, 2466, 43238, 1983, 52881, 2398, 45449, 50289, 52872 or 26908-associated disease or disorder, said method comprising the steps of receiving 18636, 2466, 43238, 1983, 52881, 2398, 45449, 50289, 52872 or 26908 sequence information from the subject and/or information related thereto, receiving phenotypic information associated with the subject, acquiring information from the network corresponding to 18636, 2466, 43238, 1983, 52881, 2398, 45449, 50289, 52872 or 26908 and/or corresponding to a GPCR-associated or another 18636, 2466, 43238, 1983, 52881, 2398, 45449, 50289, 52872 or 26908-associated disease or disorder, and based on one or more of the phenotypic information, the 18636, 2466, 43238, 1983, 52881, 2398, 45449, 50289, 52872 or 26908 information (e.g., sequence information and/or information related thereto), and the acquired information, determining whether the subject has a GPCR-associated or another 18636, 2466, 43238, 1983, 52881, 2398, 45449, 50289, 52872 or 26908-associated disease or disorder or a pre-disposition to a GPCR-associated or another 18636, 2466, 43238, 1983, 52881, 2398, 45449, 50289, 52872 or 26908-associated disease or disorder. The method may further comprise the step of recommending a particular treatment for the disease, disorder, or pre-disease condition.

The present invention also provides a business method for determining whether a subject has a GPCR-associated or another 18636, 2466, 43238, 1983, 52881, 2398, 45449, 50289, 52872 or 26908-associated disease or disorder or a pre-disposition to a GPCR-associated or another 18636, 2466, 43238, 1983, 52881, 2398, 45449, 50289, 52872 or 26908-associated disease or disorder, said method comprising the steps of receiving information related to 18636, 2466, 43238, 1983, 52881, 2398, 45449, 50289, 52872 or 26908 (e.g., sequence information and/or information related thereto), receiving phenotypic information associated with the subject, acquiring information from the network related to 18636, 2466, 43238, 1983, 52881, 2398, 45449, 50289, 52872 or 26908 and/or related to a GPCR-associated or another 18636, 2466, 43238, 1983, 52881, 2398, 45449, 50289, 52872 or 26908-associated disease or disorder, and based on one or more of the phenotypic information, the 18636, 2466, 43238, 1983, 52881, 2398, 45449, 50289, 52872 or 26908 information, and the acquired information, determining whether the subject has a GPCR-associated or another 18636, 2466, 43238, 1983, 52881, 2398, 45449, 50289, 52872 or 26908-associated disease or disorder or a pre-disposition to a GPCR-associated or another 18636, 2466, 43238, 1983, 52881, 2398, 45449, 50289, 52872 or 26908-associated disease or disorder. The method may further comprise the step of recommending a particular treatment for the disease, disorder, or pre-disease condition.

The invention also includes an array comprising a 18636, 2466, 43238, 1983, 52881, 2398, 45449, 50289, 52872 or 26908 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be 18636, 2466, 43238, 1983, 52881, 2398, 45449, 50289, 52872 or 26908. This allows a profile to be developed showing a battery of genes specifically expressed in one or more tissues.

In addition to such qualitative information, the invention allows the quantitation of gene expression. Thus, not only tissue specificity, but also the level of expression of a battery of genes in the tissue if ascertainable. Thus, genes can be grouped on the basis of their tissue expression per se and level of expression in that tissue. This is useful, for example, in ascertaining the relationship of gene expression in that tissue. Thus, one tissue can be perturbed and the effect on gene expression in a second tissue can be determined. In this context, the effect of one cell type on another cell type in response to a biological stimulus can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a GPCR-associated or another 18636, 2466, 43238, 1983, 52881, 2398, 45449, 50289, 52872 or 26908-associated disease or disorder, progression of GPCR-associated or another 18636, 2466, 43238, 1983, 52881, 2398, 45449, 50289, 52872 or 26908-associated disease or disorder, and processes, such a cellular transformation associated with the GPCR-associated or another 18636, 2466, 43238, 1983, 52881, 2398, 45449, 50289, 52872 or 26908-associated disease or disorder.

The array is also useful for ascertaining the effect of the expression of a gene on the expression of other genes in the same cell or in different cells (e.g., ascertaining the effect of 18636, 2466, 43238, 1983, 52881, 2398, 45449, 50289, 52872 or 26908 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including 18636, 2466, 43238, 1983, 52881, 2398, 45449, 50289, 52872 or 26908) that could serve as a molecular target for diagnosis or therapeutic intervention.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 18636, 2466, 43238, 1983, 52881, 2398, 45449, 50289, 52872 or 26908 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features a method of analyzing a sequence. The method includes: providing a 18636, 2466, 43238, 1983, 52881, 2398, 45449, 50289, 52872 or 26908 sequence, or record, in computer readable form; comparing a second sequence to the 18636, 2466, 43238, 1983, 52881, 2398, 45449, 50289, 52872 or 26908 sequence; thereby analyzing a sequence. Comparison can include comparing to sequences for sequence identity or determining if one sequence is included within the other, e.g., determining if the 18636, 2466, 43238, 1983, 52881, 2398, 45449, 50289, 52872 or 26908 sequence includes a sequence being compared. In a preferred embodiment the 18636, 2466, 43238, 1983, 52881, 2398, 45449, 50289, 52872 or 26908 or second sequence is stored on a first computer, e.g., at a first site and the comparison is performed, read, or recorded on a second computer, e.g., at a second site. E.g., the 18636, 2466, 43238, 1983, 52881, 2398, 45449, 50289, 52872 or 26908 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

### EXEMPLIFICATION

### Example 1

### Tissue Distribution of 18636, 2466, 43238, 1983, 52881, 2398, 45449, 50289, 52872 or 26908 mRNA

Northern blot hybridizations with various RNA samples can be performed under standard conditions and washed under stringent conditions, i.e., 0.2×SSC at 65° C. A DNA probe corresponding to all or a portion of the 18636, 2466, 43238, 1983, 52881, 2398, 45449, 50289, 52872 or 26908 cDNA (SEQ ID NO:1, 3, 29, 31, 69, 71, 77, 79, 80, 82, 83, 85, 86, 88, 89, 91, 92, 94, 103 or 105) or 18636, 2466, 43238, 1983, 52881, 2398, 45449, 50289, 52872 or 26908 cDNA can be used. The DNA was radioactively labeled with ³²P dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 2

### Recombinant Expression of 18636, 2466, 43238, 1983, 52881, 2398, 45449, 50289, 52872 or 26908 in Bacterial Cells

In this example, 18636, 2466, 43238, 1983, 52881, 2398, 45449, 50289, 52872 or 26908 is expressed as a recombinant glutathione-S-transferase (GST) fusion polypeptide in E. coli and the fusion polypeptide is isolated and characterized. Specifically, 18636, 2466, 43238, 1983, 52881, 2398, 45449, 50289, 52872 or 26908 is fused to GST and this fusion polypeptide is expressed in E. coli, e.g., strain PEB199. Expression of the GST-18636, -2466, -43238, -1983, -52881, -2398, -45449, -50289, -52872 or -26908 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant 18636, 2466, 43238, 1983, 52881, 2398, 45449, 50289, 52872 or 26908 Protein in COS Cells

To express the 18636, 2466, 43238, 1983, 52881, 2398, 45449, 50289, 52872 or 26908 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 18636, 2466, 43238, 1983, 52881, 2398, 45449, 50289, 52872 or 26908 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 18636, 2466, 43238, 1983, 52881, 2398, 45449, 50289, 52872 or 26908 dNA sequence is amplified by PCR using two primers. The 5′ primer contains the restriction site of interest followed by approximately twenty nucleotides of the 18636, 2466, 43238, 1983, 52881, 2398, 45449, 50289, 52872 or 26908 coding sequence starting from the initiation codon; the 3′ end sequence contains complementary sequences to the other restriction site of interest, a translation stop codon, the HA tag or FLAG tag and the last 20 nucleotides of the 18636, 2466, 43238, 1983, 52881, 2398, 45449, 50289, 52872 or 26908 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 18636, 2466, 43238, 1983, 52881, 2398, 45449, 50289, 52872 or 26908 gene is inserted in the correct orientation. The ligation mixture is transformed into E. coli cells (strains HB101, DHSα, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 18636-, 2466-, 43238-, 1983-, 52881-, 2398-, 45449-, 50289-, 52872- or 26908-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 18636, 2466, 43238, 1983, 52881, 2398, 45449, 50289, 52872 or 26908 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 18636, 2466, 43238, 1983, 52881, 2398, 45449, 50289, 52872 or 26908 coding sequence is cloned directly into the polylinker of the pcDNA/Amp vector using the appropriate restriction sites. The resulting plasmid is transfected into COS cells in the manner described above, and the expression of the 18636, 2466, 43238, 1983, 52881, 2398, 45449, 50289, 52872 or 26908 polypeptide is detected by radiolabelling and immunoprecipitation using a 18636, 2466, 43238, 1983, 52881, 2398, 45449, 50289, 52872 or 26908 specific monoclonal antibody.

### Example 4

### TaqMan Analysis of 18636, 2466, 43238, 1983, 52881, 2398, 45449, 50289, 52872 or 26908

Human 18636, 2466, 43238, 1983, 52881, 2398, 45449, 50289, 52872 or 26908 expression was measured by TaqMan® quantitative PCR (Perkin Elmer Applied Biosystems) in cDNA prepared from a variety of normal and diseased (e.g., cancerous) human tissues or cell lines.

Probes were designed by PrimerExpress software (PE Biosystems) based on the sequence of the human 18636, 2466, 43238, 1983, 52881, 2398, 45449, 50289, 52872 or 26908 gene. Each human 18636, 2466, 43238, 1983, 52881, 2398, 45449, 50289, 52872 or 26908 gene probe was labeled using FAM (6-carboxyfluorescein), and the 12-microglobulin reference probe was labeled with a different fluorescent dye, VIC. The differential labeling of the target gene and internal reference gene thus enabled measurement in same well. Forward and reverse primers and the probes for both β2-microglobulin and target gene were added to the TaqMan® Universal PCR Master Mix (PE Applied Biosystems). Although the final concentration of primer and probe could vary, each was internally consistent within a given experiment. A typical experiment contained 200 nM of forward and reverse primers plus 100 nM probe for β-2 microglobulin and 600 nM forward and reverse primers plus 200 nM probe for the target gene. TaqMan matrix experiments were carried out on an ABI PRISM 7700 Sequence Detection System (PE Applied Biosystems). The thermal cycler conditions were as follows: hold for 2 min at 50° C. and 10 min at 95° C., followed by two-step PCR for 40 cycles of 95° C. for 15 sec followed by 60° C. for 1 min.

The following method was used to quantitatively calculate human 18636, 2466, 43238, 1983, 52881, 2398, 45449, 50289, 52872 or 26908 gene expression in the various tissues relative to β-2 microglobulin expression in the same tissue. The threshold cycle (Ct) value is defined as the cycle at which a statistically significant increase in fluorescence is detected. A lower Ct value is indicative of a higher mRNA concentration. The Ct value of the human 18636, 2466, 43238, 1983, 52881, 2398, 45449, 50289, 52872 or 26908 gene is normalized by subtracting the Ct value of the β-2 microglobulin gene to obtain a _{Δ}Ct value using the following formula: _{Δ}Ct=Ctₛₐₘₚₗₑ−Ct_{β-2 microglobulin}. Expression is then calibrated against a cDNA sample showing a comparatively low level of expression of the human 18636, 2466, 43238, 1983, 52881, 2398, 45449, 50289, 52872 or 26908 gene. The _{Δ}Ct value for the calibrator sample is then subtracted from _{Δ}Ct for each tissue sample according to the following formula: _{ΔΔ}Ct=_{Δ}Ct-ₛₐₘₚₗₑ−_{Δ}Ct-_{calibrator}. Relative expression is then calculated using the arithmetic formula given by 2^{-ΔΔCt}.

### Example 5

### In Situ Hybridization of 18636, 2466, 43238, 1983, 52881, 2398, 45449, 50289, 52872 or 26908

The following describes the tissue distribution of 18636, 2466, 43238, 1983, 52881, 2398, 45449, 50289, 52872 or 26908 mRNA, as may be determined by in situ hybridization analysis using oligonucleotide probes based on the human G2RF sequence.

For in situ analysis, various tissues, e.g. tissues obtained from brain, are first frozen on dry ice. Ten-micrometer-thick sections of the tissues are postfixed with 4% formaldehyde in DEPC treated 1× phosphate-buffered saline at room temperature for 10 minutes before being rinsed twice in DEPC 1× phosphate-buffered saline and once in 0.1 M triethanolamine-HCl (pH 8.0). Following incubation in 0.25% acetic anhydride-0.1 M triethanolamine-HCl for 10 minutes, sections are rinsed in DEPC 2×SSC (1×SSC is 0.15M NaCl plus 0.015M sodium citrate). Tissue is then dehydrated through a series of ethanol washes, incubated in 100% chloroform for 5 minutes, and then rinsed in 100% ethanol for 1 minute and 95% ethanol for 1 minute and allowed to air dry.

Hybridizations are performed with ³⁵S-radiolabeled (5×10⁷ cpm/ml) cRNA probes. Probes are incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides are washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides are then rinsed with 2×SSC at room temperature, washed with 2×SSC at 50° C. for 1 hour, washed with 0.2×SSC at 55° C. for 1 hour, and 0.2×SSC at 60° C. for 1 hour. Sections are then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

The contents of all references, patents and published patent applications cited throughout this application are incorporated herein by reference.

### EQUIVALENTS

Those skilled in the art will recognize, or be able to ascertain using no more than routine experimentation, many equivalents to the specific embodiments of the invention described herein.

What is claimed is:

1. An isolated 18636, 2466, 43238, 1983, 52881, 2398, 45449, 50289, 52872 or 26908 nucleic acid molecule selected from the group consisting of:
a) a nucleic acid molecule comprising a nucleotide sequence which is at least 60% identical to the nucleotide sequence of SEQ ID NO:1, 3, 29, 31, 69, 71, 77, 79, 80, 82, 83, 85, 86, 88, 89, 91, 92, 94, 103 or 105;
b) a nucleic acid molecule comprising a fragment of at least 15 nucleotides of the nucleotide sequence of SEQ ID NO:1, 3, 29, 31, 69, 71, 77, 79, 80, 82, 83, 85, 86, 88, 89, 91, 92, 94, 103 or 105;
c) a nucleic acid molecule which encodes a polypeptide comprising the amino acid sequence of SEQ ID NO:2, 30, 70, 78, 81, 84, 87, 90, 93 or 104;
d) a nucleic acid molecule which encodes a fragment of a polypeptide comprising the amino acid sequence of SEQ ID NO:2, 30, 70, 78, 81, 84, 87, 90, 93 or 104, wherein the fragment comprises at least 15 contiguous amino acids of SEQ ID NO:2, 30, 70, 78, 81, 84, 87, 90, 93 or 104;
e) a nucleic acid molecule which encodes a naturally occurring allelic variant of a polypeptide comprising the amino acid sequence of SEQ ID NO:2, 30, 70, 78, 81, 84, 87, 90, 93 or 104, wherein the nucleic acid molecule hybridizes to a nucleic acid molecule comprising SEQ ID NO:1, 3, 29, 31, 69, 71, 77, 79, 80, 82, 83, 85, 86, 88, 89, 91, 92, 94, 103 or 105, or a complement thereof, under stringent conditions;
f) a nucleic acid molecule comprising the nucleotide sequence of SEQ ID NO:1, 3, 29, 31, 69, 71, 77, 79, 80, 82, 83, 85, 86, 88, 89, 91, 92, 94, 103 or 105; and
g) a nucleic acid molecule which encodes a polypeptide comprising the amino acid sequence of SEQ ID NO:2, 30, 70, 78, 81, 84, 87, 90, 93 or 104.

2. The isolated nucleic acid molecule of claim 1, which is the nucleotide sequence SEQ ID NO:1, 29, 69, 77, 80, 83, 86, 89, 92 or 103.

3. A host cell which contains the nucleic acid molecule of claim 1.

4. An isolated 18636, 2466, 43238, 1983, 52881, 2398, 45449, 50289, 52872 or 26908 polypeptide selected from the group consisting of:
a) a polypeptide which is encoded by a nucleic acid molecule comprising a nucleotide sequence which is at least 60% identical to a nucleic acid comprising the nucleotide sequence of SEQ ID NO:1, 3, 29, 31, 69, 71, 77, 79, 80, 82, 83, 85, 86, 88, 89, 91, 92, 94, 103 or 105, or a complement thereof;
b) a naturally occurring allelic variant of a polypeptide comprising the amino acid sequence of SEQ ID NO:2, 30, 70, 78, 81, 84, 87, 90, 93 or 104, wherein the polypeptide is encoded by a nucleic acid molecule which hybridizes to a nucleic acid molecule comprising SEQ ID NO:1, 3, 29, 31, 69, 71, 77, 79, 80, 82, 83, 85, 86, 88, 89, 91, 92, 94, 103 or 105, or a complement thereof under stringent conditions;
c) a fragment of a polypeptide comprising the amino acid sequence of SEQ ID NO:2, 30, 70, 78, 81, 84, 87, 90, 93 or 104, wherein the fragment comprises at least 15 contiguous amino acids of SEQ ID NO:2, 30, 70, 78, 81, 84, 87, 90, 93 or 104; and
d) the amino acid sequence of SEQ ID NO:2, 30, 70, 78, 81, 84, 87, 90, 93 or 104.

5. An antibody which selectively binds to a polypeptide of claim 4.

6. The polypeptide of claim 4, further comprising heterologous amino acid sequences.

7. A method for producing a polypeptide selected from the group consisting of:
a) a polypeptide comprising the amino acid sequence of SEQ ID NO:2, 30, 70, 78, 81, 84, 87, 90, 93 or 104;
b) a polypeptide comprising a fragment of the amino acid sequence of SEQ ID NO:2, 30, 70, 78, 81, 84, 87, 90, 93 or 104, wherein the fragment comprises at least 15 contiguous amino acids of SEQ ID NO:2, 30, 70, 78, 81, 84, 87, 90, 93 or 104;
c) a naturally occurring allelic variant of a polypeptide comprising the amino acid sequence of SEQ ID NO:2, 30, 70, 78, 81, 84, 87, 90, 93 or 104, wherein the polypeptide is encoded by a nucleic acid molecule which hybridizes to a nucleic acid molecule comprising SEQ ID NO:1, 3, 29, 31, 69, 71, 77, 79, 80, 82, 83, 85, 86, 88, 89, 91, 92, 94, 103 or 105; and
d) the amino acid sequence of SEQ ID NO:2, 30, 70, 78, 81, 84, 87, 90, 93 or 104;
comprising culturing the host cell of claim 3 under conditions in which the nucleic acid molecule is expressed.

8. A method for detecting the presence of a nucleic acid molecule of claim 1 or a polypeptide encoded by the nucleic acid molecule in a sample, comprising:
a) contacting the sample with a compound which selectively hybridizes to the nucleic acid molecule of claim 1 or binds to the polypeptide encoded by the nucleic acid molecule; and
b) determining whether the compound hybridizes to the nucleic acid or binds to the polypeptide in the sample.

9. A kit comprising a compound which selectively hybridizes to a nucleic acid molecule of claim 1 or binds to a polypeptide encoded by the nucleic acid molecule and instructions for use.

10. A method for identifying a compound which binds to a polypeptide or modulates the activity of the polypeptide of claim 4 comprising the steps of:
a) contacting a polypeptide, or a cell expressing a polypeptide of claim 4 with a test compound; and
b) determining whether the polypeptide binds to the test compound or determining the effect of the test compound on the activity of the polypeptide.

11. A method for modulating the activity of a polypeptide of claim 4 comprising contacting the polypeptide or a cell expressing the polypeptide with a compound which binds to the polypeptide in a sufficient concentration to modulate the activity of the polypeptide.

12. A method for identifying a compound capable of treating a disorder characterized by aberrant 18636, 2466, 43238, 1983, 52881, 2398, 45449, 50289, 52872 or 26908 activity, comprising assaying the ability of the compound to modulate 18636, 2466, 43238, 1983, 52881, 2398, 45449, 50289, 52872 or 26908 nucleic acid expression or 18636, 2466, 43238, 1983, 52881, 2398, 45449, 50289, 52872 or 26908 polypeptide activity, thereby identifying a compound capable of treating a disorder characterized by aberrant 18636, 2466, 43238, 1983, 52881, 2398, 45449, 50289, 52872 or 26908 activity.

13. A method of identifying a nucleic acid molecule associated with a disorder characterized by aberrant 18636, 2466, 43238, 1983, 52881, 2398, 45449, 50289, 52872 or 26908 activity, comprising:
a) contacting a sample from a subject with a disorder characterized by aberrant 18636, 2466, 43238, 1983, 52881, 2398, 45449, 50289, 52872 or 26908 activity, comprising nucleic acid molecules with a hybridization probe comprising at least 25 contiguous nucleotides of SEQ ID NO:1, 29, 69, 77, 80, 83, 86, 89, 92 or 103 defined in claim 2; and
b) detecting the presence of a nucleic acid molecule in the sample that hybridizes to the probe, thereby identifying a nucleic acid molecule associated with a disorder characterized by aberrant 18636, 2466, 43238, 1983, 52881, 2398, 45449, 50289, 52872 or 26908 activity.

14. A method of identifying a polypeptide associated with a disorder characterized by aberrant 18636, 2466, 43238, 1983, 52881, 2398, 45449, 50289, 52872 or 26908 activity, comprising:
a) contacting a sample comprising polypeptides with a 18636, 2466, 43238, 1983, 52881, 2398, 45449, 50289, 52872 or 26908 polypeptide defined in claim 4; and
b) detecting the presence of a polypeptide in the sample that binds to the 18636, 2466, 43238, 1983, 52881, 2398, 45449, 50289, 52872 or 26908 binding partner, thereby identifying the polypeptide associated with a disorder characterized by aberrant 18636, 2466, 43238, 1983, 52881, 2398, 45449, 50289, 52872 or 26908 activity.

15. A method of identifying a subject having a disorder characterized by aberrant 18636, 2466, 43238, 1983, 52881, 2398, 45449, 50289, 52872 or 26908 activity, comprising:
a) contacting a sample obtained from the subject comprising nucleic acid molecules with a hybridization probe comprising at least 25 contiguous nucleotides of SEQ ID NO: 1, 29, 69, 77, 80, 83, 86, 89, 92 or 103 defined in claim 2; and
b) detecting the presence of a nucleic acid molecule in the sample that hybridizes to the probe, thereby identifying a subject having a disorder characterized by aberrant 18636, 2466, 43238, 1983, 52881, 2398, 45449, 50289, 52872 or 26908 activity.

16. A method for treating a subject having a disorder characterized by aberrant 18636, 2466, 43238, 1983, 52881, 2398, 45449, 50289, 52872 or 26908 activity, or a subject at risk of developing a disorder characterized by aberrant 18636, 2466, 43238, 1983, 52881, 2398, 45449, 50289, 52872 or 26908 activity, comprising administering to the subject a 18636, 2466, 43238, 1983, 52881, 2398, 45449, 50289, 52872 or 26908 modulator of the nucleic acid molecule defined in claim 1 or the polypeptide encoded by the nucleic acid molecule or contacting a cell with a 18636, 2466, 43238, 1983, 52881, 2398, 45449, 50289, 52872 or 26908 modulator.

17. The method defined in claim 16 wherein said disorder is a cellular proliferative and/or differentiative disorder, brain disorder, blood vessel disorder, platelet disorder, kidney or renal disorder, hematopoeitic disorder, prostate disorder, testicular disorder, skin disorder, eye or opthalmological disorder, hormonal disorder, disorder associated with bone metabolism, immune e.g., inflammatory disorder, cardiovascular disorder, endothelial cell disorder, liver disorder, viral diseases, pain, metabolic disorder, neurological or CNS disorder, hematological disorder, intestinal disorder, respiratory disorder, fibrotic disorder or angiogenic disorder.

18. The method of claim 16, wherein the 18636, 2466, 43238, 1983, 52881, 2398, 45449, 50289, 52872 or 26908 modulator is
a) a small molecule;
b) peptide;
c) phosphopeptide;
d) anti-18636, 2466, 43238, 1983, 52881, 2398, 45449, 50289, 52872 or 26908 antibody;
e) a 18636, 2466, 43238, 1983, 52881, 2398, 45449, 50289, 52872 or 26908 polypeptide comprising the amino acid sequence of SEQ ID NO:2, 30, 70, 78, 81, 84, 87, 90, 93 or 104, or a fragment thereof;
f) a 18636, 2466, 43238, 1983, 52881, 2398, 45449, 50289, 52872 or 26908 polypeptide comprising an amino acid sequence which is at least 90 percent identical to the amino acid sequence of SEQ ID NO:2, 30, 70, 78, 81, 84, 87, 90, 93 or 104, wherein the percent identity is calculated using the ALIGN program for comparing amino acid sequences, a PAM120 weight residue table, a gap length penalty of 12, and a gap penalty of 4; or
g) an isolated naturally occurring allelic variant of a polypeptide consisting of the amino acid sequence of SEQ ID NO:2, 30, 70, 78, 81, 84, 87, 90, 93 or 104, wherein the polypeptide is encoded by a nucleic acid molecule which hybridizes to a complement of a nucleic acid molecule consisting of SEQ ID NO:1, 29, 69, 77, 80, 83, 86, 89, 92 or 103 at 6×SSC at 45° C., followed by one or more washes in 0.2×SSC, 0.1% SDS at 65° C.

19. The method of claim 16, wherein the 18636, 2466, 43238, 1983, 52881, 2398, 45449, 50289, 52872 or 26908 modulator is
a) an antisense 18636, 2466, 43238, 1983, 52881, 2398, 45449, 50289, 52872 or 26908 nucleic acid molecule;
b) is a ribozyme;
c) the nucleotide sequence of SEQ ID NO:1, 29, 69, 77, 80, 83, 86, 89, 92 or 103 or a fragment thereof;
d) a nucleic acid molecule encoding a polypeptide comprising an amino acid sequence which is at least 90 percent identical to the amino acid sequence of SEQ ID NO:2, 30, 70, 78, 81, 84, 87, 90, 93 or 104, wherein the percent identity is calculated using the ALIGN program for comparing amino acid sequences, a PAM120 weight residue table, a gap length penalty of 12, and a gap penalty of 4;
e) a nucleic acid molecule encoding a naturally occurring allelic variant of a polypeptide comprising the amino acid sequence of SEQ ID NO:2, 30, 70, 78, 81, 84, 87, 90, 93 or 104, wherein the nucleic acid molecule which hybridizes to a complement of a nucleic acid molecule consisting of SEQ ID NO:1, 29, 69, 77, 80, 83, 86, 89, 92 or 103 at 6×SSC at 45° C., followed by one or more washes in 0.2×SSC, 0.1% SDS at 65° C.; or
f) a gene therapy vector.


SEQUENCE LISTING (110 sequences)

SEQ ID NO: 1 - DNA, 1797 bp, Homo sapiens
  CDS (437)...(1507)
gaccagcctggccaacatgtaaaaccctgtctctactaaaaaaacagaaaaattagccaggtgtggtgcatgtgcctgtagttccagctacttgggaggctgaggcaggaaaattgcttgaccttggaggtggaggttgcagtgagccgagatcataccattgcactacagcctgggtgacagagagagcaagactctgtgtcaaaaaaaaaaaaaaaaaaaaaggtaaaaaaattagccaggcatggtggtaagtgcctgtagtcccagctactccaggaggctgaggggggaggatcccttgagcctaggaggtggaggctgcagtgatctgtgattgtacctttgcactccagcctgggtgacagagcatgacgctgtctctaaaagaataataataataagcattccataaaaattatggaatggagaaatcatgaatggggtctcggaggggaccagaggctgcagtgacaggcaacctggggtcctgacacgtgatcgctcttgttccaggaagatgaactcttccggatgcctgtctgaggaggtggggtccctccgcccactgactgtggttatcctgtctgcgtccattgtcgtcggagtgctgggcaatgggctggtgctgtggatgactgtcttccgtatggcacgcacggtctccaccgtctgcttcttccacctggcccttgccgatttcatgctctcactgtctctgcccattgccatgtactatattgtctccaggcagtggctcctcggagagtgggcctgcaaactctacatcacctttgtgttcctcagctactttgccagtaactgcctccttgtcttcatctctgtggaccgttgcatctctgtcctctaccccgtctgggccctgaaccaccgcactgtgcagcgggcgagctggctggcctttggggtgtggctcctggccgccgccttgtgctctgcgcacctgaaattccggacaaccagaaaatggaatggctgtacgcactgctacttggcgttcaactctgacaatgagactgcccagatttggattgaaggggtcgtggagggacacattatagggaccattggccacttcctgctgggcttcctggggcccttagcaatcataggcacctgcgcccacctcatccgggccaagctcttgcgggagggctgggtccatgccaaccggcccaagaggctgctgctggtgctggtgagcgctttctttatcttctggtccccgtttaacgtggtgctgttggtccatctgtggcgacgggtgatgctcaaggaaatctaccacccccggatgctgctcatcctccaggctagctttgccttgggctgtgtcaacagcagcctcaaccccttcctctacgtcttcgttggcagagatttccaagaaaagtttttccagtctttgacttctgccctggcgagggcgtttggagaggaggagtttctgtcatcctgtccccgtggcaacgccccccgggaatgatggaagacttcagctggaagctggaagccgtccttcttagtttgcttgtggcctcttaccttgactggcttctaaaaccctgccaaatcctgcctcttctttcaggaagtcttccagcaacccctcatctaaagctctgtggtagagacagctacttactccccagtgtccattcttccccgaccccccccttttttttttgagatagagtcttgctctgtcaccaggctggactgcagtggcgcgatctcggctcactgcaacctctgactttcctggttcaagcaatt

SEQ ID NO: 2 - protein, 356 aa, Homo sapiens
MNGVSEGTRGCSDRQPGVLTRDRSCSRKMNSSGCLSEEVGSLRPLTVVILSASIVVGVLGNGLVLWMTVFRMARTVSTVCFFHLALADFMLSLSLPIAMYYIVSRQWLLGEWACKLYITFVFLSYFASNCLLVFISVDRCISVLYPVWALNHRTVQRASWLAFGVWLLAAALCSAHLKFRTTRKWNGCTHCYLAFNSDNETAQIWIEGVVEGHIIGTIGHFLLGFLGPLAIIGTCAHLIRAKLLREGWVHANRPKRLLLVLVSAFFIFWSPFNVVLLVHLWRRVMLKEIYHPRMLLILQASFALGCVNSSLNPFLYVFVGRDFQEKFFQSLTSALARAFGEEEFLSSCPRGNAPRE

SEQ ID NO: 3 - DNA, 1071 bp, Homo sapiens
  CDS (1)...(1071)
atgaatggggtctcggaggggaccagaggctgcagtgacaggcaacctggggtcctgacacgtgatcgctcttgttccaggaagatgaactcttccggatgcctgtctgaggaggtggggtccctccgcccactgactgtggttatcctgtctgcgtccattgtcgtcggagtgctgggcaatgggctggtgctgtggatgactgtcttccgtatggcacgcacggtctccaccgtctgcttcttccacctggcccttgccgatttcatgctctcactgtctctgcccattgccatgtactatattgtctccaggcagtggctcctcggagagtgggcctgcaaactctacatcacctttgtgttcctcagctactttgccagtaactgcctccttgtcttcatctctgtggaccgttgcatctctgtcctctaccccgtctgggccctgaaccaccgcactgtgcagcgggcgagctggctggcctttggggtgtggctcctggccgccgccttgtgctctgcgcacctgaaattccggacaaccagaaaatggaatggctgtacgcactgctacttggcgttcaactctgacaatgagactgcccagatttggattgaaggggtcgtggagggacacattatagggaccattggccacttcctgctgggcttcctggggcccttagcaatcataggcacctgcgcccacctcatccgggccaagctcttgcgggagggctgggtccatgccaaccggcccaagaggctgctgctggtgctggtgagcgctttctttatcttctggtccccgtttaacgtggtgctgttggtccatctgtggcgacgggtgatgctcaaggaaatctaccacccccggatgctgctcatcctccaggctagctttgccttgggctgtgtcaacagcagcctcaaccccttcctctacgtcttcgttggcagagatttccaagaaaagtttttccagtctttgacttctgccctggcgagggcgtttggagaggaggagtttctgtcatcctgtccccgtggcaacgccccccgggaatga

SEQ ID NO: 4 - protein, 342 aa, Homo sapiens
QPGVLTRDRSCSRKMNSSGCLSEEVGSLRPLTVVILSASIVVGVLGNGLVLWMTVFRMARTVSTVCFFHLALADFMLSLSLPIAMYYIVSRQWLLGEWACKLYITFVFLSYFASNCLLVFISVDRCISVLYPVWALNHRTVQRASWLAFGVWLLAAALCSAHLKFRTTRKWNGCTHCYLAFNSDNETAQIWIEGVVEGHIIGTIGHFLLGFLGPLAIIGTCAHLIRAKLLREGWVHANRPKRLLLVLVSAFFIFWSPFNVVLLVHLWRRVMLKEIYHPRMLLILQASFALGCVNSSLNPFLYVFVGRDFQEKFFQSLTSALARAFGEEEFLSSCPRGNAPRE

SEQ ID NO: 5 - protein, 278 aa, Artificial Sequence
  Amino acid consensus sequence
GNLLVILVILRTKKLRTPTNIFILNLAVADLLFLLTLPPWALYYLVGGSEDWPFGSALCKLVTALDVVNMYASILLLTAISIDRYLAIVHPLRYRRRRTSPRRAKVVILLVWVLALLLSLPPLLFSWVKTVEEGNGTLNVNVTVCLIDFPEESTASVSTWLRSYXXXXXXXXXXVLLSTLVGFLLPLLVILVCYTRILRTLRXXXXXXXXKAAKTLLVVVVVFVLCWLPYFIVLLLDTLCXLSIIMSSTCELERVLPTALLVTLWLAYVNSCLNPIIY

SEQ ID NO: 6 - protein, 78 aa, Artificial Sequence
  Amino acid consensus sequence
HLWRRVMLKEIYHPRMLLILQASFALGCVNSSLNPFLYVFVGRDFQEKFFQSLTSALARAFGEEEFLSSCPRGNAPRE

SEQ ID NO: 7 - protein, 73 aa, Artificial Sequence
  Amino acid consensus sequence
MNGVSEGTRGCSDRQPGVLTRDRSCSRKMNSSGCLSEEVGSLRPLTVVILSASIVVGVLGNGLVLWMTVFRMA

SEQ ID NO: 8 - protein, 42 aa, Artificial Sequence
  Amino acid consensus sequence
TTRKWNGCTHCYLAFNSDNETAQIWIEGVVEGHIIGTIGHFL

SEQ ID NO: 9 - protein, 146 aa, Artificial Sequence
  Amino acid consensus sequence
RTVTMNYFLLNLAIADLLVAILTLPFMIVYSLTNFYSQWYFWISFGSCLCKLYSFLYVLSTVQRASWLAFGVWLLAAALCSAHLMYASIFTLTVISIDRYLAICHPMMSYKRRLHYTTIMTKRRAVLVIAVVWVLSILISLPPLIF

SEQ ID NO: 10 - protein, 126 aa, Artificial Sequence
  Amino acid consensus sequence
GFVVPFLIVVASYADISRRLRVRRXFHRRRRTGRLVVIIILAFAAFWLPYHVVDLVERVLAGTLDQSKQQLRNARKVCIALAFLSSSVNPLLYACAGGGLLRSAGVGFVAKLLEATGSEAFSTRRG

SEQ ID NO: 11 - protein, 75 aa, Artificial Sequence
  Amino acid consensus sequence
IYTIMMFILGFILPLLIIVICYSMIIRTLMRKRSPSRQQRSRNKKKRRKRAVRMIIVVVVVFFICWLPYHIVLLL

SEQ ID NO: 12 - protein, 125 aa, Artificial Sequence
  Amino acid consensus sequence
LLTFVLPLVVTLFCYCRISRRLRRPPHVGRARRNSLRIIFAIESTFVGSWLPFSALRAVFHLARLGALPXLPCPLLLALRWGLTIATCLAFVNSCANPLIYLLLDRSFRARALDGACGRTGRLAR

SEQ ID NO: 13 - protein, 80 aa, Artificial Sequence
  Amino acid consensus sequence
MTVADLLMCIVYMKTRPWLSHFNLWLCHPYYVIIWTCQMCSXCLNLVWLNVDKLIYIQFPLHYYQIVNRKRLLWITAATW

SEQ ID NO: 14 - protein, 30 aa, Artificial Sequence
  Amino acid consensus sequence
LYAMNIALVTFLKITRGSCLGVSLNPYVYL

SEQ ID NO: 15 - protein, 126 aa, Artificial Sequence
  Amino acid consensus sequence
FFTFAGMSFYCFLXLPIKMGMLTGHXWDLPRDYCAILMYIDDFSFYFSSWSLTFMAIERINHFCYSTPKLNENTXKALKKMCFPIVWVTSGFQALQMLNNYKKTQMNNEDDYCFLAFERSGHDMWL

SEQ ID NO: 16 - protein, 81 aa, Artificial Sequence
  Amino acid consensus sequence
RLYKDVTHAFIINLALCHFVQCALVLPVSLMVMLIQNWIFGQFLCXFFLPMLQVSENRNVTYKAFTLQKLTTAQSYIYPIW

SEQ ID NO: 17 - protein, 356 aa, Homo sapiens; identical to SEQ ID NO: 2

SEQ ID NO: 18 - protein, 4 aa, Artificial Sequence
  N-glycosylation site
NSSG

SEQ ID NO: 19 - protein, 4 aa, Artificial Sequence
  N-glycosylation site
NETA

SEQ ID NO: 20 - protein, 4 aa, Artificial Sequence
  N-glycosylation site
NSSL

SEQ ID NO: 21 - protein, 4 aa, Artificial Sequence
  Casein kinase II phosphorylation site
SDNE

SEQ ID NO: 22 - protein, 6 aa, Artificial Sequence
  N-myristoylation site
GVSEGT

SEQ ID NO: 23 - protein, 6 aa, Artificial Sequence
  N-myristoylation site
GVLGNG

SEQ ID NO: 24 - protein, 6 aa, Artificial Sequence
  N-myristoylation site
GCTHCY

SEQ ID NO: 25 - protein, 6 aa, Artificial Sequence
  N-myristoylation site
GVVEGH

SEQ ID NO: 26 - protein, 6 aa, Artificial Sequence
  N-myristoylation site
GCVNSS

SEQ ID NO: 27 - protein, 17 aa, Artificial Sequence
  G protein coupled receptor signature sequence
ASNCLLVFISVDRCISV

SEQ ID NO: 28 - protein, 17 aa, Artificial Sequence
  G protein coupled receptor signature sequence
XXXXXXXXXXXXRXXXX

SEQ ID NO: 29 - DNA, 1801 bp, Homo sapiens
  CDS (53)...(1537)
ggccgccgcccccggtgcgggatgaggagatccgcggccgccactgggccccatggaggagccgccgccgccccgcccaccagcgagcatggccttactgggcagccagcactccggcgccccctccgcggccggcccacctggcgggacttcctccgcggccacggcggccgtgctctccttcagcaccgtggcgaccgcggcgctggggaacctgagcgacgcaagcggaggcggcacagctgccgctcccggtggcggcggccttggcgggtccggggcagcgcgggaggcgggggcggcggtgaggcggccgctaggcccggaggcggcgccgctgctgtcgcacggagctgcagtggcggcccaggcgctcgtcctcctgctcatcttcctgctgtctagccttggcaactgcgcggtgatgggggtgattgtgaagcaccggcagctccgcaccgtcaccaacgccttcatcctgtcgctgtccctatcggatctgctcacggcgctgctctgcctgcccgccgccttcctggacctcttcactccgcccgggggttcggcgcctgccgccgccgcggggccctggcgcggcttctgcgccgccagccgcttcttcagctcgtgcttcggcatcgtgtccacgctcagcgtggcgctcatctcgttggaccgttactgcgctatcgtgcggccgccgcgggagaagatcggccgccgccgcgcgctgcagctgctggcgggcgcctggctgacggccctgggcttctccttgccctgggagctgctcggggcgccccgggaactcgcggcggcgcagagcttccacggctgcctctaccggacctccccggaccccgcgcagctgggcgcggccttcagcgtggggctggtggtggcctgctacctgctgcccttcctgctcatgtgcttctgccactaccacatctgcaagacggtgcgcctgtcggacgtgcgcgtgcggccggtgaacacctacgcgcgcgtgctgcgcttcttcagcgaggtgcgcacggccaccaccgtcctcatcatgatcgtcttcgtcatctgctgctgggggccctactgcttcctggtgctgctggccgccgcccggcaggcccagaccatgcaggccccctcgctcctcagcgtggtggccgtctggctgacctgggccaatggggccatcaaccctgtcatctacgccatccgcaatcccaacatttcgatgctcctagggcgcaaccgcgaggagggctaccggactaggaatgtggacgctttcctgcccagccagggcccgggtctgcaagccagaagccgcagtcgccttcgaaaccgctatgccaaccggctgggggcctgcaacaggatgtcctcttccaacccggccagcggagtggcaggggacgtggccatgtgggcccgcaaaaatccagttgtacttttctgccgagagggaccaccagagccggtgacggcagtgaccaaacagcctaaatccgaagctggggataccagcctctaagacggttggaatggccagcttatgaaggcaaatttccactcgcattatttaatgatggaagattctgggggagagttgtggatttcataaagccaaacatttaaagctagagacgggggaggcttaccactttccccaaacaacataaaagacaatgtcccttcttcaaaagtgccaaaaggaatgtaaaatgcaaaaattaaaacaatcttaaaccmcawaammaarmawwrwraamwrwaarwrccrramrygrsrmraata

SEQ ID NO: 30 - protein, 494 aa, Homo sapiens
MEEPPPPRPPASMALLGSQHSGAPSAAGPPGGTSSAATAAVLSFSTVATAALGNLSDASGGGTAAAPGGGGLGGSGAAREAGAAVRRPLGPEAAPLLSHGAAVAAQALVLLLIFLLSSLGNCAVMGVIVKHRQLRTVTNAFILSLSLSDLLTALLCLPAAFLDLFTPPGGSAPAAAAGPWRGFCAASRFFSSCFGIVSTLSVALISLDRYCAIVRPPREKIGRRRALQLLAGAWLTALGFSLPWELLGAPRELAAAQSFHGCLYRTSPDPAQLGAAFSVGLVVACYLLPFLLMCFCHYHICKTVRLSDVRVRPVNTYARVLRFFSEVRTATTVLIMIVFVICCWGPYCFLVLLAAARQAQTMQAPSLLSVVAVWLTWANGAINPVIYAIRNPNISMLLGRNREEGYRTRNVDAFLPSQGPGLQARSRSRLRNRYANRLGACNRMSSSNPASGVAGDVAMWARKNPVVLFCREGPPEPVTAVTKQPKSEAGDTSL

SEQ ID NO: 31 - DNA, 1485 bp, Homo sapiens
  CDS (1)...(1485)
atggaggagccgccgccgccccgcccaccagcgagcatggccttactgggcagccagcactccggcgccccctccgcggccggcccacctggcgggacttcctccgcggccacggcggccgtgctctccttcagcaccgtggcgaccgcggcgctggggaacctgagcgacgcaagcggaggcggcacagctgccgctcccggtggcggcggccttggcgggtccggggcagcgcgggaggcgggggcggcggtgaggcggccgctaggcccggaggcggcgccgctgctgtcgcacggagctgcagtggcggcccaggcgctcgtcctcctgctcatcttcctgctgtctagccttggcaactgcgcggtgatgggggtgattgtgaagcaccggcagctccgcaccgtcaccaacgccttcatcctgtcgctgtccctatcggatctgctcacggcgctgctctgcctgcccgccgccttcctggacctcttcactccgcccgggggttcggcgcctgccgccgccgcggggccctggcgcggcttctgcgccgccagccgcttcttcagctcgtgcttcggcatcgtgtccacgctcagcgtggcgctcatctcgttggaccgttactgcgctatcgtgcggccgccgcgggagaagatcggccgccgccgcgcgctgcagctgctggcgggcgcctggctgacggccctgggcttctccttgccctgggagctgctcggggcgccccgggaactcgcggcggcgcagagcttccacggctgcctctaccggacctccccggaccccgcgcagctgggcgcggccttcagcgtggggctggtggtggcctgctacctgctgcccttcctgctcatgtgcttctgccactaccacatctgcaagacggtgcgcctgtcggacgtgcgcgtgcggccggtgaacacctacgcgcgcgtgctgcgcttcttcagcgaggtgcgcacggccaccaccgtcctcatcatgatcgtcttcgtcatctgctgctgggggccctactgcttcctggtgctgctggccgccgcccggcaggcccagaccatgcaggccccctcgctcctcagcgtggtggccgtctggctgacctgggccaatggggccatcaaccctgtcatctacgccatccgcaatcccaacatttcgatgctcctagggcgcaaccgcgaggagggctaccggactaggaatgtggacgctttcctgcccagccagggcccgggtctgcaagccagaagccgcagtcgccttcgaaaccgctatgccaaccggctgggggcctgcaacaggatgtcctcttccaacccggccagcggagtggcaggggacgtggccatgtgggcccgcaaaaatccagttgtacttttctgccgagagggaccaccagagccggtgacggcagtgaccaaacagcctaaatccgaagctggggataccagcctctaa

SEQ ID NO: 32 - protein, 441 aa, Homo sapiens
NLSDASGGGTAAAPGGGGLGGSGAAREAGAAVRRPLGPEAAPLLSHGAAVAAQALVLLLIFLLSSLGNCAVMGVIVKHRQLRTVTNAFILSLSLSDLLTALLCLPAAFLDLFTPPGGSAPAAAAGPWRGFCAASRFFSSCFGIVSTLSVALISLDRYCAIVRPPREKIGRRRALQLLAGAWLTALGFSLPWELLGAPRELAAAQSFHGCLYRTSPDPAQLGAAFSVGLVVACYLLPFLLMCFCHYHICKTVRLSDVRVRPVNTYARVLRFFSEVRTATTVLIMIVFVICCWGPYCFLVLLAAARQAQTMQAPSLLSVVAVWLTWANGAINPVIYAIRNPNISMLLGRNREEGYRTRNVDAFLPSQGPGLQARSRSRLRNRYANRLGACNRMSSSNPASGVAGDVAMWARKNPVVLFCREGPPEPVTAVTKQPKSEAGDTSL

SEQ ID NO: 33 - protein, 293 aa, Artificial Sequence
  Amino acid consensus sequence
GNLLVILVILRTKKLRTPTNIFILNLAVADLLFLLTLPPWALYYLVGGSEDWPFGSXXXXXXXXXXALCKLVTALDVVNMYASILLLTAISIDRYLAIVHPLRYRRRRTSPRRAKVVILLVWVLALLLSLPPLLFSWVKTVEEGNGTLNXVNVTVCLIDFPEESTASVSTWLRSYVLLSTLVGFLLPLLVILVCYTRILRTLRXXXXXXXXXXXXXXXXXXXXXXKAAKTLLVVVVVFVLCWLPYFIVLLLDTLCXLSIIMSSTCELERVLPTALLVTLWLAYVNSCLNPIIY

SEQ ID NO: 34 - protein, 318 aa, Artificial Sequence
  Amino acid consensus sequence
MALLGSQHSGAPSAAGPPGGTSSAATAAVLSFSTVATAALGNLSDASGGGTAAAPGGGGLGGSGAAREAGAAVRRPLGPEAAPLLSHGAAVAAQALVLLLIFLLSSLGNCAVMGVIVKHRQLRTVTNAFILSLSLSDLLTALLCLPAAFLDLFTPPGGSAPALPAGPWRGFCRPSRFFSSCFGIVYAQRGAHLVGPLLRYRXXXRPPREKIGRRRALQLLAGAWLTALGFSLPWELLGAPRELAAGQSFHGCLYRTSPDPAQLGGPFSVGLVVACYLLPFLLICFCHYHICKTVRLSDVRVRPVNTYARVLRSSARCA

SEQ ID NO: 35 - protein, 86 aa, Artificial Sequence
  Amino acid consensus sequence
NAFQGVVYSANQCKALITILVVLGAFMVTWGPYXMVVIASEALWGKSSVSPSLXETWATWLSFASAVCHPLIYGLWNKTVRKELLG

SEQ ID NO: 36 - protein, 27 aa, Artificial Sequence
  Amino acid consensus sequence
CALFPFLVMLVCYGFIFRVARVKARKV

SEQ ID NO: 37 - protein, 159 aa, Artificial Sequence
  Amino acid consensus sequence
MSSLRHRLSNAXSSLFKYREESRAARISILVVVMFVVSXYLPFGLLVLLQSRLSAANFGGSSQLAIFMILLXXANLSXSPFIFAYRNKRVRRGVKRLFGLDSSSGLQXRNCSSSVKTNGTAGPAASGAQLQXRNSSKLSQYSSNSCKYLTPQSSLVSQV

SEQ ID NO: 38 - protein, 122 aa, Artificial Sequence
  Amino acid consensus sequence
LGALAVLLVVGDTWCAVTDPLRYHSRISGVKTWIFIALTWVVGILFGALSAFRVLDFEADALFSRQRRLAVTYFNISSTNSIFGVVYASVYFIVIILLPFGFVCGMYWRIFSEARGNGLRMR

SEQ ID NO: 39 - protein, 125 aa, Artificial Sequence
  Amino acid consensus sequence
VSLLSHCLVALNRYLLITRAPATYQVLYQRRHTAGMLALSWALALGLVLLLPPWXXXXAPKPGAEPPQVHXXXYXXXXXPALLAAXXXXGALLAXXXXQTALLLHCYLGIVRRVRVSVKRVSVLN

SEQ ID NO: 40 - protein, 21 aa, Artificial Sequence
  Amino acid consensus sequence
ASWLLCCALSALNPLLYTWRN

SEQ ID NO: 41 - protein, 22 aa, Artificial Sequence
  Amino acid consensus sequence
NGMVIYLVSSFRKLQTTSNAFI

SEQ ID NO: 42 - protein, 61 aa, Artificial Sequence
  Amino acid consensus sequence
VWVSLASGFSLPVPWGVQAASWLLCCALSALNPLLYTWRNEEFRXRSVRSVLPGVGDAAAA

SEQ ID NO: 43 - DNA, 650 bp, Homo sapiens
ggccgccgcccccggtgcgggatgaggagatccgcggccgccactgggccccatggaggagccgccgccgccccgcccaccagcgagcatggccttactgggcagccagcactccggcgccccctccgcggccggcccacctggcgggacttcctcagcggccacggcggccgtgctctccttcagcaccgtggcgaccgcggcgctggggaacctgagcgacgcaagcggaggcggcacagctgccgctcccggtggcggcggccttggcgggtccggggcagcgcgggaggcgggggcggcggtgaggcggccgctaggcccggaggcggcgccgctgctgtcgcacggagctgcagtggcggcccaggcgctcgtcctcctgctcatcttcctgctgtctagccttggcaactgcgcggtgatgggggtgattgtgaagcaccggcagctccgcaccgtcaccaacgccttcatcctgtcgctgtccctatcggatctgctcacggcgctgctctgcctgcccgccgccttcctggacctcttcactccgcccgggggttcggcgcctgcgctgcccgcggggccctggcgcggcttctgccggccaagccgcttcttcagctcgtgcttcggcatcgtgtacgctc

SEQ ID NO: 44 - DNA, 424 bp, Homo sapiens
ttcttcagcgaggtgcgcacggccaccaccgtcctcatcatgatcgtcttcgtcatctgctgctgggggccctactgcttcctggtgctgctggccgccgcccggcaggcccagaccatgcaggccccctcgctcctcagcgtggtggccgtctggctgacctgggccaatggggccatcaaccctgtcatctacgccatccgcaatcccaacatttcgatgctcctagggcgcaaccgcgaggagggctaccggactaggaatgtggacgctttcctgcccagccagggcccgggtctgcaagccagaagccgcagtcgccttcgaaaccgctatgccaaccggctgggggcctgcaacaggatgtcctcttccaacccggccagcggagtggcaggggacgtggccatgtgggccgcaaaaa

SEQ ID NO: 45 - DNA, 325 bp, Homo sapiens
cggccgccgcgggagaagatcggccgccgccgcgcgctgcagctgctggcgggcgcctggctgacggccctgggcttctccttgccctgggagctgctcggggcgccccgggaactcgcggcgggccagagcttccacggctgcctctaccggacctccccggaccccgcgcagctgggcggccccttcagcgtggggctggtggtggcctgctacctgctgcccttcctgctcatctgcttctgccactaccacatctgcaagacggtgcgcctgtcggacgtgcgcgtgcggccggtgaacacctacgcgcgcgtgctgcgtt

SEQ ID NO: 46 - DNA, 51 bp, Homo sapiens
gtgtacgctcagcgtggcgctcatctcgttggaccgttactgcgctatcgt

SEQ ID NO: 47 - DNA, 38 bp, Homo sapiens
gggccgcaaaaatccagttgtacttttctgccgagagg

SEQ ID NO: 48 - DNA, 19 bp, Homo sapiens
ggaccaccagagccggtga

SEQ ID NO: 49 - protein, 22 aa, Artificial Sequence
  Leucine zipper pattern
LSLSLSDLLTALLCLPAAFLDL

SEQ ID NO: 50 - protein, 17 aa, Artificial Sequence
  G protein coupled receptor signature sequence
VSTLSVALISLDRYCAI

SEQ ID NO: 51 - protein, 4 aa, Artificial Sequence
  Casein kinase II phosphorylation site
SLSD

SEQ ID NO: 52 - protein, 4 aa, Artificial Sequence
  Casein kinase II phosphorylation site
TSPD

SEQ ID NO: 53 - protein, 4 aa, Artificial Sequence
  Glycosaminoglycan attachment site
SGGG

SEQ ID NO: 54 - protein, 7 aa, Artificial Sequence
  Tyrosine kinase phosphorylated site
RNREEGY

SEQ ID NO: 55 - protein, 4 aa, Artificial Sequence
  Amidation site
IGRR

SEQ ID NO: 56 - protein, 4 aa, Artificial Sequence
  ASN N-glycosylation site
NLSD

SEQ ID NO: 57 - protein, 4 aa, Artificial Sequence
  ASN N-glycosylation site
NISM

SEQ ID NO: 58 - protein, 6 aa, Artificial Sequence
  N-myristoylation site
GSQHSG

SEQ ID NO: 59 - protein, 6 aa, Artificial Sequence
  N-myristoylation site
GGTSSA

SEQ ID NO: 60 - protein, 6 aa, Artificial Sequence
  N-myristoylation site
GGGTAA

SEQ ID NO: 61 - protein, 6 aa, Artificial Sequence
  N-myristoylation site
GGGLGG

SEQ ID NO: 62 - protein, 6 aa, Artificial Sequence
  N-myristoylation site
GAAVAA

SEQ ID NO: 63 - protein, 6 aa, Artificial Sequence
  N-myristoylation site
GSAPAA

SEQ ID NO: 64 - protein, 6 aa, Artificial Sequence
  N-myristoylation site
GIVSTL

SEQ ID NO: 65 - protein, 6 aa, Artificial Sequence
  N-myristoylation site
GAWLTA

SEQ ID NO: 66 - protein, 6 aa, Artificial Sequence
  N-myristoylation site
GAAFSV

SEQ ID NO: 67 - protein, 6 aa, Artificial Sequence
  N-myristoylation site
GLVVAC

SEQ ID NO: 68 - protein, 17 aa, Artificial Sequence
  G protein coupled receptor signature sequence
XXXXXXXXXXXXRXXXX

SEQ ID NO: 69 - DNA, 1970 bp, Homo sapiens
  CDS (166)...(1104)
cccacgcgtccggcgggtccaggagggcccctctcccgcgaccgccgaccacgatgagagcgtgaagaccctctcgaaaggaagggctctgctctacacactggtgtctcctgcggaagggcagctgggcacgccttccagaccgagttacagcagaaagttttcatggagaaatggaatcacacttcaaatgatttcattttgttgggtctgcttcccccaaatcaaactggaatatttctcttgtgccttatcatcctcatattctttctggcctcggtgggtaactcggccatgattcacctcatccacgtggatcctcgtctccacacaccgatgtactttcttctcagccagctctcccttatggacctgatgtacatctccaccaccgtccccaagatggcgtacaacttcctgtccggccagaaaggcatctccttcctgggatgtggtgtgcaaagcttcttcttcctgaccatggcgtgttctgaaggcttactcctgacctccatggcctacgaccgttatttggccatctgccactctctctattatcctatccgcatgagtaaaatgatgtgtgtgaagatgattggaggctcttggacactggggtccatcaactccttggcacacacagtctttgcccttcatattccctactgcaggtctagggctattgaccatttcttctgcgatgtcccagccatgttgcttcttgcctgtacagatacttgggtctatgaatatatggtttttgtaagtacaagcctctttctccttttccctttcattggcatcacttcttcctgtggccgagtcctatttgctgtctatcatatgcactcaaaggaggggagaaaaaaggccttcaccaccatttcaacacatttaactgtagtgatcttttactatgcaccttttgtctacacctatcttcggcccaggaatctccgctcaccagctgaagacaagatcctggcagtcttctacaccatccttacccccatgctcaatcccattatctacagcctgaggaataaggaagtcctgggggctatgaggagagtgtttgggatattctctttcctgaaagaataatcatggccatccccactccctttgtatttcctctttccaagttgattccaacacgctagagcagggttgtccaatagaaatacaacataatttaaaattttctaataggtacatttaagcagtcaaataaatttaaataatatatttaattcaaaacaatgttataattaatattaatattaacaatattgatgttaattacatagcatactatttcaataaattatatgcaatatattatagtacatgtttgtacatatattactattaacgtgataatgtttattcttgtatattgacatagaatttcttcatgtaataacacaaatttgttaatgttgccttttactctgtttgcattctaagtcttcaaagtcttgtgtttgttttatagagtgcagcgcagcttggacaaaacacatttcaagtgcccagtagtcagtgtytatagtgttggacagcacattyttagagcatccccaatcaatagtttcagaagttatatatgcatgtgtatgtgcatgtatggtgkatacaaacatatttttgttatatacaatattgctgatgamctgaaaattaacaagtaaaatgcacatgctaaggtagtgtttatgtgtttctttatctgttggctcatttttagcatatgcacatttattgtaattaacatttgaagtccagtttatcctaactcctttaataaaactattcagggagagcttatctgacttatgattttgctgatttcataaagatactgagataaaataacttgaatatgaatataaaagaacccacatttatgtatacataaaaattaataaaatctttatataaataactttaaaaaaaaaaaaaaaagg

SEQ ID NO: 70 - protein, 312 aa, Homo sapiens
MEKWNHTSNDFILLGLLPPNQTGIFLLCLIILIFFLASVGNSAMIHLIHVDPRLHTPMYFLLSQLSLMDLMYISTTVPKMAYNFLSGQKGISFLGCGVQSFFFLTMACSEGLLLTSMAYDRYLAICHSLYYPIRMSKMMCVKMIGGSWTLGSINSLAHTVFALHIPYCRSRAIDHFFCDVPAMLLLACTDTWVYEYMVFVSTSLFLLFPFIGITSSCGRVLFAVYHMHSKEGRKKAFTTISTHLTVVIFYYAPFVYTYLRPRNLRSPAEDKILAVFYTILTPMLNPIIYSLRNKEVLGAMRRVFGIFSFLKE

SEQ ID NO: 71 - DNA, 939 bp, Homo sapiens
  CDS (1)...(939)
atggagaaatggaatcacacttcaaatgatttcattttgttgggtctgcttcccccaaatcaaactggaatatttctcttgtgccttatcatcctcatattctttctggcctcggtgggtaactcggccatgattcacctcatccacgtggatcctcgtctccacacaccgatgtactttcttctcagccagctctcccttatggacctgatgtacatctccaccaccgtccccaagatggcgtacaacttcctgtccggccagaaaggcatctccttcctgggatgtggtgtgcaaagcttcttcttcctgaccatggcgtgttctgaaggcttactcctgacctccatggcctacgaccgttatttggccatctgccactctctctattatcctatccgcatgagtaaaatgatgtgtgtgaagatgattggaggctcttggacactggggtccatcaactccttggcacacacagtctttgcccttcatattccctactgcaggtctagggctattgaccatttcttctgcgatgtcccagccatgttgcttcttgcctgtacagatacttgggtctatgaatatatggtttttgtaagtacaagcctctttctccttttccctttcattggcatcacttcttcctgtggccgagtcctatttgctgtctatcatatgcactcaaaggaggggagaaaaaaggccttcaccaccatttcaacacatttaactgtagtgatcttttactatgcaccttttgtctacacctatcttcggcccaggaatctccgctcaccagctgaagacaagatcctggcagtcttctacaccatccttacccccatgctcaatcccattatctacagcctgaggaataaggaagtcctgggggctatgaggagagtgtttgggatattctctttcctgaaagaataa

SEQ ID NO: 72 - protein, 259 aa, Artificial Sequence
  Amino acid consensus sequence
GNLLVILVILRTKKLRTPTNIFILNLAVADLLFLLTLPPWALYYLVGGSEDWPFGSALCKLVTALDVVNMYASILLLTAISIDRYLAIVHPLRYRRRRTSPRRAKVVILLVWVLALLLSLPPLLFSWVKTVEEGNGTLNVNVTVCLIDFPEESTASVSTWLRSYVLLSTLVGFLLPLLVILVCYTRILRTLRKAAKTLLVVVVVFVLCWLPYFIVLLLDTLCLSIIMSSTCELERVLPTALLVTLWLAYVNSCLNPIIY

SEQ ID NO: 73 - protein, 61 aa, Artificial Sequence
  Amino acid consensus sequence
TVVSLFYGTAIFVYLRPSSSHSTDKDKVVSVFYTVVTPMLNPFIYSLRNKDVKGALKRLLG

SEQ ID NO: 74 - protein, 80 aa, Artificial Sequence
  Amino acid consensus sequence
LSFCGSNVINHFFCDISPLLKLACSDTSVNELVIFVIAVLILLVPFLLILISYVHIISAILKIPSAEGRYKAFSTCASHL

SEQ ID NO: 75 - protein, 117 aa, Artificial Sequence
  Amino acid consensus sequence
KLRTPTNIFLLNLAIADLLVAILTLPFTLVNYLYSLMSGHWVFGSILCKIYSFLDVISMYASIFTLTAISIDRYLAIICPPHPMYYSQRMMTKRRAMLVIVVVWILSLLISLPPLIF

SEQ ID NO: 76 - protein, 63 aa, Artificial Sequence
  Amino acid consensus sequence
VVLAFYVPLIGLSVVHRFGNSLDPIVHVLMGDVYLLLPPVINPIIYGAKTKQIRTRVLAMFKI

SEQ ID NO: 77 - DNA, 3127 bp, Homo sapiens
  CDS (171)...(2243)
aattcccgggtcgacccacgcgtccgggaaagtgtccggaggaagccggcgcgtcacggaggacggatccgggaccctgccgtgcccgcccagcggcgcccggtcccgggtccacagccgccctcactccgmcgcgctctccgccaccgccaccactgcggccaccgccaatgaaacgcctcccgctcctagtggttttttccactttgttgaattgttcctatactcaaaattgcaccaagacaccttgtctcccaaatgcaaaatgtgaaatacgcaatggaattgaagcctgctattgcaacatgggattttcaggaaatggtgtcacaatttgtgaagatgataatgaatgtggaaatttaactcagtcctgtggcgaaaatgctaattgcactaacacagaaggaagttattattgtatgtgtgtacctggcttcagatccagcagtaaccaagacaggtttatcactaatgatggaaccgtctgtatagaaaatgtgaatgcaaactgccatttagataatgtctgtatagctgcaaatattaataaaactttaacaaaaatcagatccataaaagaacctgtggctttgctacaagaagtctatagaaattctgtgacagatctttcaccaacagatataattacatatatagaaatattagctgaatcatcttcattactaggttacaagaacaacactatctcagccaaggacaccctttctaactcaactcttactgaatttgtaaaaaccgtgaataattttgttcaaagggatacatttgtagtttgggacaagttatctgtgaatcataggagaacacatcttacaaaactcatgcacactgttgaacaagctactttaaggatatcccagagcttccaaaagaccacagagtttgatacaaattcaacggatatagctctcaaagttttcttttttgattcatataacatgaaacatattcatcctcatatgaatatggatggagactacataaatatatttccaaagagaaaagctgcatatgattcaaatggcaatgttgcagttgcatttttatattataagagtattggtcctttgctttcatcatctgacaacttcttattgaaacctcaaaattatgataattctgaagaggaggaaagagtcatatcttcagtaatttcagtctcaatgagctcaaacccacccacattatatgaacttgaaaaaataacatttacattaagtcatcgaaaggtcacagataggtataggagtctatgtgcattttggaattactcacctgataccatgaatggcagctggtcttcagagggctgtgagctgacatactcaaatgagacccacacctcatgccgctgtaatcacctgacacattttgcaattttgatgtcctctggtccttccattggtattaaagattataatattcttacaaggatcactcaactaggaataattatttcactgatttgtcttgccatatgcatttttaccttctggttcttcagtgaaattcaaagcaccaggacaacaattcacaaaaatctttgctgtagcctatttcttgctgaacttgtttttcttgttgggatcaatacaaatactaataagctcttctgttcaatcattgccggactgctacactacttctttttagctgcttttgcatggatgtgcattgaaggcatacatctctatctcattgttgtgggtgtcatctacaacaagggatttttgcacaagaatttttatatctttggctatctaagcccagccgtggtagttggattttcggcagcactaggatacagatattatggcacaaccaaagtatgttggcttagcaccgaaaacaactttatttggagttttataggaccagcatgcctaatcattcttgttaatctcttggcttttggagtcatcatatacaaagtttttcgtcacactgcagggttgaaaccagaagttagttgctttgagaacataaggtcttgtgcaagaggagccctcgctcttctgttccttctcggcaccacctggatctttggggttctccatgttgtgcacgcatcagtggttacagcttacctcttcacagtcagcaatgctttccaggggatgttcatttttttattcctgtgtgttttatctagaaagattcaagaagaatattacagattgttcaaaaatgtcccctgttgttttggatgtttaaggtaaacatagagaatggtggataattacaactgcacaaaaataaaaattccaagctgtggatgaccaatgtataaaaatgactcatcaaattatccaattattaactactagacaaaaagtattttaaatcagtttttctgtttatgctataggaactgtagataataaggtaaaattatgtatcatatagatatactatgtttttctatgtgaaatagttctgtcaaaaatagtattgcagatatttggaaagtaattggtttctcaggagtgatatcactgcacccaaggaaagattttctttctaacacgagaagtatatgaatgtcctgaaggaaaccactggcttgatatttctgtgactcgtgttgcctttgaaactagtcccctaccacctcggtaatgagctccattacagaaagtggaacataagagaatgaaggggcagaatatcaaacagtgaaaagggaatgataagatgtattttgaatgaactgttttttctgtagactagctgagaaattgttgacataaaataaagaattgaagaaacacattttaccattttgtgaattgttctgaacttaaatgtccactaaaacaacttagacttctgtttgctaaatctgtttctttttctaatattctaaaaaaaaaaaaaaggtttacctccacaaattgaaaaaaaaaaagtgaaaaaaatctgtttctaaggttagactgagatatatactatttccttacttatttcacagattgtgactttggatagttaatcagtaaaatataaatgtgtcaagatataatattgtttatacctatcaatgtaaaaacagtgtaataaagctgaagtattctattaaaaaaaaaaaaaaaaaaaaaaaaaa

SEQ ID NO: 78 - protein, 690 aa, Homo sapiens [sequence omitted]

SEQ ID NO: 79 - DNA, 2073 bp, Homo sapiens
  CDS (1)...(2073)
atgaaacgcctcccgctcctagtggttttttccactttgttgaattgttcctatactcaaaattgcaccaagacaccttgtctcccaaatgcaaaatgtgaaatacgcaatggaattgaagcctgctattgcaacatgggattttcaggaaatggtgtcacaatttgtgaagatgataatgaatgtggaaatttaactcagtcctgtggcgaaaatgctaattgcactaacacagaaggaagttattattgtatgtgtgtacctggcttcagatccagcagtaaccaagacaggtttatcactaatgatggaaccgtctgtatagaaaatgtgaatgcaaactgccatttagataatgtctgtatagctgcaaatattaataaaactttaacaaaaatcagatccataaaagaacctgtggctttgctacaagaagtctatagaaattctgtgacagatctttcaccaacagatataattacatatatagaaatattagctgaatcatcttcattactaggttacaagaacaacactatctcagccaaggacaccctttctaactcaactcttactgaatttgtaaaaaccgtgaataattttgttcaaagggatacatttgtagtttgggacaagttatctgtgaatcataggagaacacatcttacaaaactcatgcacactgttgaacaagctactttaaggatatcccagagcttccaaaagaccacagagtttgatacaaattcaacggatatagctctcaaagttttcttttttgattcatataacatgaaacatattcatcctcatatgaatatggatggagactacataaatatatttccaaagagaaaagctgcatatgattcaaatggcaatgttgcagttgcatttttatattataagagtattggtcctttgctttcatcatctgacaacttcttattgaaacctcaaaattatgataattctgaagaggaggaaagagtcatatcttcagtaatttcagtctcaatgagctcaaacccacccacattatatgaacttgaaaaaataacatttacattaagtcatcgaaaggtcacagataggtataggagtctatgtgcattttggaattactcacctgataccatgaatggcagctggtcttcagagggctgtgagctgacatactcaaatgagacccacacctcatgccgctgtaatcacctgacacattttgcaattttgatgtcctctggtccttccattggtattaaagattataatattcttacaaggatcactcaactaggaataattatttcactgatttgtcttgccatatgcatttttaccttctggttcttcagtgaaattcaaagcaccaggacaacaattcacaaaaatctttgctgtagcctatttcttgctgaacttgtttttcttgttgggatcaatacaaatactaataagctcttctgttcaatcattgccggactgctacactacttctttttagctgcttttgcatggatgtgcattgaaggcatacatctctatctcattgttgtgggtgtcatctacaacaagggatttttgcacaagaatttttatatctttggctatctaagcccagccgtggtagttggattttcggcagcactaggatacagatattatggcacaaccaaagtatgttggcttagcaccgaaaacaactttatttggagttttataggaccagcatgcctaatcattcttgttaatctcttggcttttggagtcatcatatacaaagtttttcgtcacactgcagggttgaaaccagaagttagttgctttgagaacataaggtcttgtgcaagaggagccctcgctcttctgttccttctcggcaccacctggatctttggggttctccatgttgtgcacgcatcagtggttacagcttacctcttcacagtcagcaatgctttccaggggatgttcatttttttattcctgtgtgttttatctagaaagattcaagaagaatattacagattgttcaaaaatgtcccctgttgttttggatgtttaaggtaa

SEQ ID NO: 80 - DNA, 4238 bp, Homo sapiens
  CDS (403)...(2232)
  misc_feature (1)...(4238): n = A,T,C or G
attcccgggtcgaacccacgcgtccgcccacgcgtccgcccacgcgtccggctgagcgaagcgcggcggcggcggcggcgcctaggggagggaggggcggcggggccgagcccacctagcggagcgcgccggccgccggtggccgccgccagcatgccccggcccgcgggccgctccgccgccagccacccccgcggccctcggcggcctgcgctcggcccgggggcgcgggaaccgcagccggagccggaggcgggagcagcgagccggagccccgggcgctcgaatgcaggatgctcgtggtccccagcatccttgagccaccaggagtgagggctgctgctccctgagacctggctccaaggaggatgccacagccgcctgccagctccggtctgcaccatgagtgatgagcggcggctgcctggcagtgcagtgggctggctggtatgtgggggcctctccctgctggccaatgcctggggcatcctcagcgttggcgccaagcagaagaagtggaagcccttggagttcctgctgtgtacgctcgcggccacccacatgctaaatgtggccgtgcccatcgccacctactccgtggtgcagctgcggcggcagcgccccgacttcgagtggaatgagggtctctgcaaggtcttcgtgtccaccttctacaccctcaccctggccacctgtttctctgtcacctccctctcctaccaccgcatgtggatggtctgctggcctgtcaactaccggctgagcaatgccaagaagcaggcggtgcacacagtcatgggtatctggatggtgtccttcatcctgtcggccctgcctgccgttggctggcacgacaccagcgagcgcttctacacccatggctgccgcttcatcgtggctgagatcggcctgggctttggcgtctgcttcctgctgctggtgggcggcagcgtggccatgggcgtgatctgcacagccatcgccctcttccagacgctggccgtgcaggtggggcgccaggccgaccaccgcgccttcaccgtgcccaccatcgtggtggaggacgcgcagggcaagcggcgctcctccatcgatggctcggagcccgccaaaacctctctgcagaccacgggcctcgtgaccaccatagtcttcatctacgactgcctcatgggcttccctgtgctggtggtgagcttcagcagcctgcgggccgacgcctcagcgccctggatggcactctgcgtgctgtggtgctccgtggcccaggccctgctgctgcctgtgttcctctgggcctgcgaccgctaccgggctgacctcaaagctgtccgggagaagtgcatggccctcatggccaacgacgaggagtcagacgatgagaccagcctggaaggtggcatctccccggacctggtgttggagcgctccctggactatggctatggaggtgattttgtggccctagataggatggccaagtatgagatctccgccctggaggggggcctgccccagctctacccactgcggcccttgcaggaggacaagatgcaatacctgcaggtcccgcccacgcggcgcttctcccacgacgatgcggacgtgtgggccgccgtcccgctgcccgccttcctgccgcgctggggctccggcgaggacctggccgccctggcgcacctggtgctgcctgccgggcccgagcggcgccgcgccagcctcctggccttcgcggaggacgcaccaccgtcccgcgcgcgccgccgctcggccgagagcctgctgtcgctgcggccctcggccctggatagcggcccgcggggagcccgcgactcgccccccggcagcccgcgccgccgccccgggcccggcccccgctccgcctcggcctcgctgctgcccgacgccttcgccctgaccgccttcgagtgcgagccacaggccctgcgccgcccgcccgggcccttccccgctgcgcccgccgcccccgacggcgcagatcccggagaggccccgacgcccccaagcagcgcccagcggagcccagggccacgcccctctgcgcactcgcacgccggctctctgcgccccggcctgagcgcgtcgtggggcgagcccggggggctgcgcgcggcgggcggcggcggcagcaccagcagcttcctgagttccccctccgagtcctcgggctacgccacgctgcactcggactcgctgggctccgcgtcctaggaccgccggcgcctccccacggacgccaggcaggccaggccgctctccggggccgcagcaccaaagacgcccgcctcccccccgcgcgcagacatgcgccacccctcccaggggtgagggggcngttggcctcagcgtttgtcttccggctcctcccagctggccttgtcccaggggcgacggctgccccggacgactgcgctgggcaccgcatgtcccgggccgagtgaggtcgggcctggggagctgagtgacatcccaagcttgggcttgggtagtgagtgacatgtgcacacgtccagctgcgccatcaccagccctgcgcaacaggacgtcgggagcagggaacctgagacagggccactgcgggatcggacaaagccccgctttggagaggctgagctggagccattggcctccccaggggctttccacccacactgcaccataaccgccacacccttcggggggggggagggtacagaagggtctctaagcacaggggtgttcagagcccgaacaagctttgatcaggtttccctgcttccgacctgtcctgcctcagtttccccatctgtgatgagcaggtgaccacgttaactctcagggctgtttgaagtctcttggttgtaggccctcgccactgagtggcccaggtgtgagaggtagtctagagccctctgcggcctttgtggaggtccgttctcagcatgtggcctggtgtggctccctaggcctgaggcctcccactctcagtgccccctgcccccttgggaacccacactccaccccagctaagcacaacactgtggaccaccaccaatggcacctgagccaccttgaccaccattagtgtccccaccctcattactctgccgtcgttgcctgtccccaccacatcatctccctccatggctcccaccatgacgctggcaggtgcaagcggagagcacgagggcagcagggcctggccttccgtgtccctgcgtcatccccagcctcactccccacccgtgcaggcctggccaggaagggatcctggcaggggcttccaggttctcagctcaaggcctggtcccgggcaggcgtccaacccctgggagcaatgtatttctttgccttccatcctgggcagaccccttacaggcctgggcattgccatgggccctgggtcttcccaggctaaggagaaccaggaacagctataaccttgagctagtgaaatagggtagatgangaaggctgtctcctccagacccctacccctacacagtggccccacaatatgaagacctggggtaattccaaggtgagcatagagcctgcctgtgcccagttccttctggccctcaggtggccaagcccatctcttcatccttcagatagggtcccactcccagaagaagctgctggggtgggggtgggaggctgcctaagcctgtctgtgcttcagaggcccctccagtccctggctgtggggtaactgggggtatgagctgtggccacaggtgagcaaggcagggaactgcaatccagccctggccgcgggaggggccatctctggccaatgctgctgtgccttcaaggactgacaagttacgtaggggcagaggtcgccagctagccagtgtctcctccatctggggggcgtctgtccacttgtcaccttaggttttcactcatttgtcaccttggggttttgctctgtgtgtttcatatccaacggcaatacttgcagggggacagagtcctctaaatactccaatcctgcggtttttacaaacataaagggggaramcccaartggargaccctkggccyggarctccytcccaaactttgtccagcatccagcctgttccctgggctcactggggagggagttgtcttcatagcacacccagagccagggatccctttgtagttttttgacaacggagcatttctcttctgtacaggacccaataaaaacttccttatgaaaaaaaaaaaaaaagggcggccgc

SEQ ID NO: 81 - protein, 609 aa, Homo sapiens
MSDERRLPGSAVGWLVCGGLSLLANAWGILSVGAKQKKWKPLEFLLCTLAATHMLNVAVPIATYSVVQLRRQRPDFEWNEGLCKVFVSTFYTLTLATCFSVTSLSYHRMWMVCWPVNYRLSNAKKQAVHTVMGIWMVSFILSALPAVGWHDTSERFYTHGCRFIVAEIGLGFGVCFLLLVGGSVAMGVICTAIALFQTLAVQVGRQADHRAFTVPTIVVEDAQGKRRSSIDGSEPAKTSLQTTGLVTTIVFIYDCLMGFPVLVVSFSSLRADASAPWMALCVLWCSVAQALLLPVFLWACDRYRADLKAVREKCMALMANDEESDDETSLEGGISPDLVLERSLDYGYGGDFVALDRMAKYEISALEGGLPQLYPLRPLQEDKMQYLQVPPTRRFSHDDADVWAAVPLPAFLPRWGSGEDLAALAHLVLPAGPERRRASLLAFAEDAPPSRARRRSAESLLSLRPSALDSGPRGARDSPPGSPRRRPGPGPRSASASLLPDAFALTAFECEPQALRRPPGPFPAAPAAPDGADPGEAPTPPSSAQRSPGPRPSAHSHAGSLRPGLSASWGEPGGLRAAGGGGSTSSFLSSPSESSGYATLHSDSLGSAS

SEQ ID NO: 82 - DNA, 1830 bp, Homo sapiens
  CDS (1)...(1830)
atgagtgatgagcggcggctgcctggcagtgcagtgggctggctggtatgtgggggcctctccctgctggccaatgcctggggcatcctcagcgttggcgccaagcagaagaagtggaagcccttggagttcctgctgtgtacgctcgcggccacccacatgctaaatgtggccgtgcccatcgccacctactccgtggtgcagctgcggcggcagcgccccgacttcgagtggaatgagggtctctgcaaggtcttcgtgtccaccttctacaccctcaccctggccacctgtttctctgtcacctccctctcctaccaccgcatgtggatggtctgctggcctgtcaactaccggctgagcaatgccaagaagcaggcggtgcacacagtcatgggtatctggatggtgtccttcatcctgtcggccctgcctgccgttggctggcacgacaccagcgagcgcttctacacccatggctgccgcttcatcgtggctgagatcggcctgggctttggcgtctgcttcctgctgctggtgggcggcagcgtggccatgggcgtgatctgcacagccatcgccctcttccagacgctggccgtgcaggtggggcgccaggccgaccaccgcgccttcaccgtgcccaccatcgtggtggaggacgcgcagggcaagcggcgctcctccatcgatggctcggagcccgccaaaacctctctgcagaccacgggcctcgtgaccaccatagtcttcatctacgactgcctcatgggcttccctgtgctggtggtgagcttcagcagcctgcgggccgacgcctcagcgccctggatggcactctgcgtgctgtggtgctccgtggcccaggccctgctgctgcctgtgttcctctgggcctgcgaccgctaccgggctgacctcaaagctgtccgggagaagtgcatggccctcatggccaacgacgaggagtcagacgatgagaccagcctggaaggtggcatctccccggacctggtgttggagcgctccctggactatggctatggaggtgattttgtggccctagataggatggccaagtatgagatctccgccctggaggggggcctgccccagctctacccactgcggcccttgcaggaggacaagatgcaatacctgcaggtcccgcccacgcggcgcttctcccacgacgatgcggacgtgtgggccgccgtcccgctgcccgccttcctgccgcgctggggctccggcgaggacctggccgccctggcgcacctggtgctgcctgccgggcccgagcggcgccgcgccagcctcctggccttcgcggaggacgcaccaccgtcccgcgcgcgccgccgctcggccgagagcctgctgtcgctgcggccctcggccctggatagcggcccgcggggagcccgcgactcgccccccggcagcccgcgccgccgccccgggcccggcccccgctccgcctcggcctcgctgctgcccgacgccttcgccctgaccgccttcgagtgcgagccacaggccctgcgccgcccgcccgggcccttccccgctgcgcccgccgcccccgacggcgcagatcccggagaggccccgacgcccccaagcagcgcccagcggagcccagggccacgcccctctgcgcactcgcacgccggctctctgcgccccggcctgagcgcgtcgtggggcgagcccggggggctgcgcgcggcgggcggcggcggcagcaccagcagcttcctgagttccccctccgagtcctcgggctacgccacgctgcactcggactcgctgggctccgcgtcctag

SEQ ID NO: 83 - DNA, 1113 bp, Homo sapiens
  CDS (1)...(1053)
atggctttggaacagaaccagtcaacagattattattatgaggaaaatgaaatgaatggcacttatgactacagtcaatatgaactgatctgtatcaaagaagatgtcagagaatttgcaaaagttttcctccctgtattcctcacaatagttttcgtcattggacttgcaggcaattccatggtagtggcaatttatgcctattacaagaaacagagaaccaaaacagatgtgtacatcctgaatttggctgtagcagatttactccttctattcactctgcctttttgggctgttaatgcagttcatgggtgggttttagggaaaataatgtgcaaaataacttcagccttgtacacactaaactttgtctctggaatgcagtttctggcttgtatcagcatagacagatatgtggcagtaactaaagtccccagccaatcaggagtgggaaaaccatgctggatcatctgtttctgtgtctggatggctgccatcttgctgagcataccccagctggttttttatacagtaaatgacaatgctaggtgcattcccattttcccccgctacctaggaacatcaatgaaagcattgattcaaatgctagagatctgcattggatttgtagtaccctttcttattatgggggtgtgctactttatcacagcaaggacactcatgaagatgccaaacattaaaatatctcgacccctaaaagttctgctcacagtcgttatagttttcattgtcactcaactgccttataacattgtcaagttctgccgagccatagacatcatctactccctgatcaccagctgcaacatgagcaaacgcatggacatcgccatccaagtcacagaaagcatcgcactctttcacagctgcctcaacccaatcctttatgtttttatgggagcatctttcaaaaactacgttatgaaagtggccaagaaatatgggtcctggagaagacagagacaaagtgtggaggagtttccttttgattctgagggtcctacagagccaaccagtacttttagcatttaaaggtaaaactgctctgccttttgcttggatacatatgaatgatgctttcccctcaaataa

SEQ ID NO: 84 - protein, 350 aa, Homo sapiens
MALEQNQSTDYYYEENEMNGTYDYSQYELICIKEDVREFAKVFLPVFLTIVFVIGLAGNSMVVAIYAYYKKQRTKTDVYILNLAVADLLLLFTLPFWAVNAVHGWVLGKIMCKITSALYTLNFVSGMQFLACISIDRYVAVTKVPSQSGVGKPCWIICFCVWMAAILLSIPQLVFYTVNDNARCIPIFPRYLGTSMKALIQMLEICIGFVVPFLIMGVCYFITARTLMKMPNIKISRPLKVLLTVVIVFIVTQLPYNIVKFCRAIDIIYSLITSCNMSKRMDIAIQVTESIALFHSCLNPILYVFMGASFKNYVMKVAKKYGSWRRQRQSVEEFPFDSEGPTEPTSTFSI

SEQ ID NO: 85 - DNA, 1053 bp, Homo sapiens
  CDS (1)...(1053)
atggctttggaacagaaccagtcaacagattattattatgaggaaaatgaaatgaatggcacttatgactacagtcaatatgaactgatctgtatcaaagaagatgtcagagaatttgcaaaagttttcctccctgtattcctcacaatagttttcgtcattggacttgcaggcaattccatggtagtggcaatttatgcctattacaagaaacagagaaccaaaacagatgtgtacatcctgaatttggctgtagcagatttactccttctattcactctgcctttttgggctgttaatgcagttcatgggtgggttttagggaaaataatgtgcaaaataacttcagccttgtacacactaaactttgtctctggaatgcagtttctggcttgtatcagcatagacagatatgtggcagtaactaaagtccccagccaatcaggagtgggaaaaccatgctggatcatctgtttctgtgtctggatggctgccatcttgctgagcataccccagctggttttttatacagtaaatgacaatgctaggtgcattcccattttcccccgctacctaggaacatcaatgaaagcattgattcaaatgctagagatctgcattggatttgtagtaccctttcttattatgggggtgtgctactttatcacagcaaggacactcatgaagatgccaaacattaaaatatctcgacccctaaaagttctgctcacagtcgttatagttttcattgtcactcaactgccttataacattgtcaagttctgccgagccatagacatcatctactccctgatcaccagctgcaacatgagcaaacgcatggacatcgccatccaagtcacagaaagcatcgcactctttcacagctgcctcaacccaatcctttatgtttttatgggagcatctttcaaaaactacgttatgaaagtggccaagaaatatgggtcctggagaagacagagacaaagtgtggaggagtttccttttgattctgagggtcctacagagccaaccagtacttttagcatttaa

SEQ ID NO: 86 - DNA, 1109 bp, Homo sapiens
  CDS (326)...(997)
ccatgcgtccacgcgtccgggaagaggaaagaggtgccctgctgatgatatatgtaatcatgacttagtgacatatcacaagcataggaagcttctggttcagtggtcctcaaatgcaactcacaaaatatcatctggggaagttctgaattaagtcgttgagagtgttacctgggcatcaggatatttaaaatgagaatctacagttgtaggcaagccgttcaccatcatctcttgcttttgtcaccgctgggtgtttggctggatcggctgccgctggtatggatgggctggatttttcyttggctgtggaagccttatcaccatgactgctgtcagcctggatcgatatttgaaaatctgctatttatcttatcacgcctacatctgcctggcagccatctgggcctatgcttccttctggaccaccatgcccttggtaggtctgggggactacgtacctgagcccttcggaacctcgtgcaccctggactggtggctggcccaggcctcggtagggggccaggttttcatcctgaacatcctcttcttctgcctcttgctcccaacggctgtgatcgtgttctcctacgtaaagatcattgccaaggttaagtcctcttccaaagaagtagctcatttcgacagtcggatccatagcagccatgtgctggaaatgaaactgacaaaggtagcgatgttgatttgtgctggattcctgattgcctggattccttatgcagtggtgtctgtgtggtcagcttttggaaggccagactccattcccatacagctctctgtggtgccaaccctacttgcaaaatctgcagcgatgtacaatcccatcatttaccaagttattgattacaaatttgcctgttgccaaactggtggtttgaaagcaaccaagaagaagtctctggaaggcttcaggctgcacaccgtaaccacagtcaggaagtcttctgctgtgctggaaattcatgaagaggtatgaagatggatacagcatcactatggacactcgtattcacttatttgcctcttcactgctgtaaacatttgattgtggccccaaaaaaaaaaaaaaaaaaaaaaaaaattgcggc

SEQ ID NO: 87 - protein, 223 aa, Homo sapiens
MTAVSLDRYLKICYLSYHAYICLAAIWAYASFWTTMPLVGLGDYVPEPFGTSCTLDWWLAQASVGGQVFILNILFFCLLLPTAVIVFSYVKIIAKVKSSSKEVAHFDSRIHSSHVLEMKLTKVAMLICAGFLIAWIPYAVVSVWSAFGRPDSIPIQLSVVPTLLAKSAAMYNPIIYQVIDYKFACCQTGGLKATKKKSLEGFRLHTVTTVRKSSAVLEIHEEV

SEQ ID NO: 88 - DNA, 672 bp, Homo sapiens
  CDS (1)...(672)
atgactgctgtcagcctggatcgatatttgaaaatctgctatttatcttatcacgcctacatctgcctggcagccatctgggcctatgcttccttctggaccaccatgcccttggtaggtctgggggactacgtacctgagcccttcggaacctcgtgcaccctggactggtggctggcccaggcctcggtagggggccaggttttcatcctgaacatcctcttcttctgcctcttgctcccaacggctgtgatcgtgttctcctacgtaaagatcattgccaaggttaagtcctcttccaaagaagtagctcatttcgacagtcggatccatagcagccatgtgctggaaatgaaactgacaaaggtagcgatgttgatttgtgctggattcctgattgcctggattccttatgcagtggtgtctgtgtggtcagcttttggaaggccagactccattcccatacagctctctgtggtgccaaccctacttgcaaaatctgcagcgatgtacaatcccatcatttaccaagttattgattacaaatttgcctgttgccaaactggtggtttgaaagcaaccaagaagaagtctctggaaggcttcaggctgcacaccgtaaccacagtcaggaagtcttctgctgtgctggaaattcatgaagaggtatga

SEQ ID NO: 89 - DNA, 3489 bp, Homo sapiens
  CDS (52)...(2610)
  misc_feature (1)...(3489): n = A,T,C or G
ttactatagggagtcgacccactgcgtccgctgttggaagttgcctctgccatgctgggccctgctgtcctgggcctcagcctctgggctctcctgcaccctgggacgggggccccattgtgcctgtcacagcaacttaggatgaagggggactacgtgctgggggggctgttccccctgggcgaggccgaggaggctggcctccgcagccggacacggcccagcagccctgtgtgcaccaggttctcctcaaacggcctgctctgggcactggccatgaaaatggccgtggaggagatcaacaacaagtcggatctgctgcccgggctgcgcctgggctacgacctctttgatacgtgctcggagcctgtggtggccatgaagcccagcctcatgttcctggccaaggcaggcagccgcgacatcgccgcctactgcaactacacgcagtaccagccccgtgtgctggctgtcatcgggccccactcgtcagagctcgccatggtcaccggcaagttcttcagcttcttcctcatgccccaggtcagctacggtgctagcatggagctgctgagcgcccgggagaccttcccctccttcttccgcaccgtgcccagcgaccgtgtgcagctgacggccgccgcggagctgctgcaggagttcggctggaactgggtggccgccctgggcagcgacgacgagtacggccggcagggcctgagcatcttctcggccctggccgcggcacgcggcatctgcatcgcgcacgagggcctggtgccgctgccccgtgccgatgactcgcggctggggaaggtgcaggacgtcctgcaccaggtgaaccagagcagcgtgcaggtggtgctgctgttcgcctccgtgcacgccgcccacgccctcttcaactacagcatcagcagcaggctctcgcccaaggtgtgggtggccagcgaggcctggctgacctctgacctggtcatggggctgcccggcatggcccagatgggcacggtgcttggcttcctccagaggggtgcccagctgcacgagttcccccagtacgtgaagacgcacctggccctggccaccgacccggccttctgctctgccctgggcgagagggagcagggtctggaggaggacgtggtgggccagcgctgcccgcagtgtgactgcatcacgctgcagaacgtgagcgcagggctaaatcaccaccagacgttctctgtctacgcagctgtgtatagcgtggcccaggccctgcacaacactcttcagtgcaacgcctcaggctgccccgcgcaggaccccgtgaagccctggcagctcctggagaacatgtacaacctgaccttccacgtgggcgggctgccgctgcggttcgacagcagcggaaacgtggacatggagtacgacctgaagctgtgggtgtggcagggctcagtgcccaggctccacgacgtgggcaggttcaacggcagcctcaggacagagcgcctgaagatccgctggcacacgtctgacaaccagaagcccgtgtcccgatgctcgcggcagtgccaggagggccaggtgcgccgggtcaaggggttccactcctgctgctacgactgtgtggactgcgaggcgggcagctaccggcaaaacccagacgacatcgcctgcaccttttgtggccaggatgagtggtccccggagcgaagcacacgctgcttccgccgcaggtctcggttcctggcatggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtgacccaaccctgtgatctcagccccggtgaacccagacttagctgcgatcccccccaagccagcaatgacccgtgtctcgctacagagaccctcccgctctaggttctgaccccaggttgtctcctgaccctgaccccacagtgagccctaggcctggagcacgtggacacccctgtgaccatctgggccccagagccaagctgtgtccctgtccctctgtgcccagaccaggcctgcccaggtaacccagacccactgttctggaaagaggcccggagggctcccagggtacccgcaacccacaccgtgagctcaggaaaaggacgcagggaggccccggccagatggctggaagcccaaatcaggccctgccgacctgaccatgtcccaccagggcccccatcctgcaccctgccaggcaccacagcagtgggaggccaggtgggggcacacaggcatatgcccagggcagagcccgccgaggtgggggtggcacccagcttcctactctgccccttgcccagtgggtagacagcatcatgactgtcaccagtaccagggacagagcccaggtggggtgggggcggggtccagcaccacggccagcaccgaccaccaggaccccggagccagcaccatggacagaaaactgcccaccaggatctgacgccagcacgccgccaggcccacacagggtctccggtcagagtcccagggtcagctcccagcagggcctaggggaggctggaccagctccctgtgcctcattccaaggcagcccagccggagagaaggggcacaggccncacatctgtcccataaaattaaacgctttttagtgtttnaaatannnnnaaaagggtttngggcgcccgctaaa

SEQ ID NO: 90 - protein, 852 aa, Homo sapiens
MLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELLQEFGWNWVAALGSDDEYGRQGLSIFSALAAARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

SEQ ID NO: 91 - DNA, 2559 bp, Homo sapiens
  CDS (1)...(2559)
atgctgggccctgctgtcctgggcctcagcctctgggctctcctgcaccctgggacgggggccccattgtgcctgtcacagcaacttaggatgaagggggactacgtgctgggggggctgttccccctgggcgaggccgaggaggctggcctccgcagccggacacggcccagcagccctgtgtgcaccaggttctcctcaaacggcctgctctgggcactggccatgaaaatggccgtggaggagatcaacaacaagtcggatctgctgcccgggctgcgcctgggctacgacctctttgatacgtgctcggagcctgtggtggccatgaagcccagcctcatgttcctggccaaggcaggcagccgcgacatcgccgcctactgcaactacacgcagtaccagccccgtgtgctggctgtcatcgggccccactcgtcagagctcgccatggtcaccggcaagttcttcagcttcttcctcatgccccaggtcagctacggtgctagcatggagctgctgagcgcccgggagaccttcccctccttcttccgcaccgtgcccagcgaccgtgtgcagctgacggccgccgcggagctgctgcaggagttcggctggaactgggtggccgccctgggcagcgacgacgagtacggccggcagggcctgagcatcttctcggccctggccgcggcacgcggcatctgcatcgcgcacgagggcctggtgccgctgccccgtgccgatgactcgcggctggggaaggtgcaggacgtcctgcaccaggtgaaccagagcagcgtgcaggtggtgctgctgttcgcctccgtgcacgccgcccacgccctcttcaactacagcatcagcagcaggctctcgcccaaggtgtgggtggccagcgaggcctggctgacctctgacctggtcatggggctgcccggcatggcccagatgggcacggtgcttggcttcctccagaggggtgcccagctgcacgagttcccccagtacgtgaagacgcacctggccctggccaccgacccggccttctgctctgccctgggcgagagggagcagggtctggaggaggacgtggtgggccagcgctgcccgcagtgtgactgcatcacgctgcagaacgtgagcgcagggctaaatcaccaccagacgttctctgtctacgcagctgtgtatagcgtggcccaggccctgcacaacactcttcagtgcaacgcctcaggctgccccgcgcaggaccccgtgaagccctggcagctcctggagaacatgtacaacctgaccttccacgtgggcgggctgccgctgcggttcgacagcagcggaaacgtggacatggagtacgacctgaagctgtgggtgtggcagggctcagtgcccaggctccacgacgtgggcaggttcaacggcagcctcaggacagagcgcctgaagatccgctggcacacgtctgacaaccagaagcccgtgtcccgatgctcgcggcagtgccaggagggccaggtgcgccgggtcaaggggttccactcctgctgctacgactgtgtggactgcgaggcgggcagctaccggcaaaacccagacgacatcgcctgcaccttttgtggccaggatgagtggtccccggagcgaagcacacgctgcttccgccgcaggtctcggttcctggcatggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtga

SEQ ID NO: 92 - DNA, 1609 bp, Homo sapiens
  CDS (176)...(1372)
gtcgaccacgcgtccggagcccagcacagctgccctctggaccctgcggaccccagcccgagccccttcctgagttccacaggcgcagcccccgggcggtcgggcggaggggtccccggggcggtgccagggcgcaatcctggagggcggccgggaggaggaggtgcgcgcggccatgcacaccgtggctacgtccgggcccaacgcgtcctggggggcaccggccaacgcctccggctgcccgggctgtggcgccaacgcctcggacggcccagtcccttcgccgcgggccgtggacgcctggctcgtgccgctcttcttcgcggcgctgatgctgctgggcctggtggggaactcgctggtcatctacgtcatctgccgccacaagccgatgcggaccgtgaccaacttctacatcgccaacctggcggccacggacgtgaccttcctcctgtgctgcgtccccttcacggccctgctgtacccgctgcccggctgggtgctgggcgacttcatgtgcaagttcgtcaactacatccagcaggtctcggtgcaggccacgtgtgccactctgaccgccatgagtgtggaccgctggtacgtgacggtgttcccgttgcgcgccctgcaccgccgcacgccccgcctggcgctggctgtcagcctcagcatctgggtaggctctgcggcggtgtctgcgccggtgctcgccctgcaccgcctgtcacccgggccgcgcgcctactgcagtgaggccttccccagccgcgccctggagcgcgccttcgcactgtacaacctgctggcgctgtacctgctgccgctgctcgccacctgcgcctgctatgcggccatgctgcgccacctgggccgggtcgccgtgcgccccgcgcccgccgatagcgccctgcaggggcaggtgctggcagagcgcgcaggcgccgtgcgggccaaggtctcgcggctggtggcggccgtggtcctgctcttcgccgcctgctggggccccatccagctgttcctggtgctgcaggcgctgggccccgcgggctcctggcacccacgcagctacgccgcctacgcgcttaagacctgggctcactgcatgtcctacagcaactccgcgctgaacccgctgctctacgccttcctgggctcgcacttccgacaggccttccgccgcgtctgcccctgcgcgccgcgccgcccccgccgcccccgccggcccggaccctcggaccccgcagccccacacgcggagctgcaccgcctggggtcccacccggcccccgccagggcgcagaagccagggagcagtgggctggccgcgcgcgggctgtgcgtcctgggggaggacaacgcccctctctgagcggacccggtgggaatccgagcggctccctcgggagcggggactgctggaacagcggctattcttctgttattagtatttttcttactgtccaagatcaactgtggaaatattttggtctcttgtgacgttcggtgcagtttcgttgtgaagtttgctattgatattgaaattatgacttctgtgtttcctgaaattaaacatgtgtcaacaaaaaaaaaaaaaaaaaaagggcgg

SEQ ID NO: 93 - protein, 398 aa, Homo sapiens
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELHRLGSHPAPARAQKPGSSGLAARGLCVLGEDNAPL

SEQ ID NO: 94 - DNA, 1197 bp, Homo sapiens
  CDS (1)...(1197)
atgcacaccgtggctacgtccgggcccaacgcgtcctggggggcaccggccaacgcctccggctgcccgggctgtggcgccaacgcctcggacggcccagtcccttcgccgcgggccgtggacgcctggctcgtgccgctcttcttcgcggcgctgatgctgctgggcctggtggggaactcgctggtcatctacgtcatctgccgccacaagccgatgcggaccgtgaccaacttctacatcgccaacctggcggccacggacgtgaccttcctcctgtgctgcgtccccttcacggccctgctgtacccgctgcccggctgggtgctgggcgacttcatgtgcaagttcgtcaactacatccagcaggtctcggtgcaggccacgtgtgccactctgaccgccatgagtgtggaccgctggtacgtgacggtgttcccgttgcgcgccctgcaccgccgcacgccccgcctggcgctggctgtcagcctcagcatctgggtaggctctgcggcggtgtctgcgccggtgctcgccctgcaccgcctgtcacccgggccgcgcgcctactgcagtgaggccttccccagccgcgccctggagcgcgccttcgcactgtacaacctgctggcgctgtacctgctgccgctgctcgccacctgcgcctgctatgcggccatgctgcgccacctgggccgggtcgccgtgcgccccgcgcccgccgatagcgccctgcaggggcaggtgctggcagagcgcgcaggcgccgtgcgggccaaggtctcgcggctggtggcggccgtggtcctgctcttcgccgcctgctggggccccatccagctgttcctggtgctgcaggcgctgggccccgcgggctcctggcacccacgcagctacgccgcctacgcgcttaagacctgggctcactgcatgtcctacagcaactccgcgctgaacccgctgctctacgccttcctgggctcgcacttccgacaggccttccgccgcgtctgcccctgcgcgccgcgccgcccccgccgcccccgccggcccggaccctcggaccccgcagccccacacgcggagctgcaccgcctggggtcccacccggcccccgccagggcgcagaagccagggagcagtgggctggccgcgcgcgggctgtgcgtcctgggggaggacaacgcccctctctga

SEQ ID NO: 95 - protein, 268 aa, Artificial Sequence
  Amino acid consensus sequence
ALLLKVIYTVGYSLSLVALLLAIFIFLLFRRLHCTRNYIHLNLFISFILRALLFLIGDAVLQNSVGPESLHCSNQVGCKVVAVFLHYFFLANFFWMLVEGLYLYTLLVVEVFFSERKRLRWYILIGWGVPAVFVVVWAIVRLIKPKGYGEDDGCLWLSNEDNTGFWWIIKGPVLLAILVNFILFINILRILVTKLRESNTGESDQYRLVKSTLVLLPLLGITWILFLFAPENDARGISSVVFLYLFLILNSFQGFFVALLYCFLNGEV

SEQ ID NO: 96 - protein, 45 aa, Artificial Sequence
  Amino acid consensus sequence
CAPNNPCSNGGTCVNTPGGSSDNFGGYTCECPPGDYYLSYTGKRC

SEQ ID NO: 97 - protein, 54 aa, Artificial Sequence
  Amino acid consensus sequence
SNPICVFWDESELSLGVWSTDRGCELVETSKPSHTTCSCNHLTSFAVLMDVSPN

SEQ ID NO: 98 - protein, 77 aa, Artificial Sequence
  Amino acid consensus sequence
SALCKLVTALDVVNMYASILLLTAISIDRYLAIVHPLRYRRRRTSPRRAKVVILLVWVLALLLSLPPLLFSWVKTVE

SEQ ID NO: 99 - protein, 259 aa, Artificial Sequence; identical to SEQ ID NO: 72
  Amino acid consensus sequence

SEQ ID NO: 100 - protein, 183 aa, Artificial Sequence
  Amino acid consensus sequence
LTAISIDRYLAIVHPLRYRRRRTSPRRAKVVILLVWVLALLLSLPPLLFSWVKTVEEGNGTLNVNVTVCLIDFPEESTASVSTWLRSYVLLSTLVGFLLPLLVILVCYTRILRTLRKAAKTLLVVVVVFVLCWLPYFIVLLLDTLCLSIIMSSTCELERVLPTALLVTLWLAYVNSCLNPIIY

SEQ ID NO: 101 - protein, 464 aa, Artificial Sequence
  Amino acid consensus sequence
GLINEAVRGILRLEAMLGAFDRINADPALLPGLALGLAILDINSLRTVALEQSFTFVYALLIKSECDCSSVRCAAVDLALEHGVAKKMIVGVIGPSCSSSAIQVANLASLLNIPMISYASTAPELSDKDTRYPTLSRTIPSDAKLGEAMVDNNILKHFNWNRYVSLVYSDGDYGEEGCEALKEALREAGGICIALSVKIGEFDRADEEEFDQLLRELKRRKPEARVVVMCGHSALLGGETLRELLEAALRLGLTGEDYVFISDDLFNKSLPAEPGYEEVAPGAITIELANASMLRFAYYFVLVLTLNNPRNPWFLEFWKENFICALQDFETNKSNFRRKCTGVERITALLEPYEVEGKAGFVYDAVYLYAHALHNMTLALGGDCGLCPAMKWVDGEKLVQHLRNVTFEGVTGPPVTFDENNGDRPGDYVLLDTQNTETGQLKVTLSGMYPITFTYDGVGKWTEP

SEQ ID NO: 102 - protein, 175 aa, Artificial Sequence
  Amino acid consensus sequence
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX

SEQ ID NO: 103 - DNA, 1829 bp, Hom sapiens
  CDS (142)...(1404)
cccgtctcatccttttcgctcctttccccgccgcatacaccgggtatccgagtgcctcagagaaccgaaggtggtgtgcggggctgcagggcacgacttcaagcggtcctcagctccgcactagggggcacgggcaacagcatggacaccaagcgctgcttcgccaatcgcttcgatgactaccagggcagcctgctggcgggccagtgtgaggaggcggtggcgcccttggtcaccgccaccatcgagcgcatcctccaggagcttcccccactcgggggcggcgcggaggcccgaggggcgacggcggaggctagcgcctgccagggggggctttatggcggcgtggccggagtggcgtatatgctctaccacgtctcgcagagcccgcttttcgccacggcccgggaacgctacctgcgctcggctaagcgcctcatcgacgcgtgcgcccgcgctgaggagtggggcgaaccggacgccgacacccgcgccgccttcctgctcgggggcgcgggcgtgtacgccgtggccacgctcgtataccacgccctgggccggtccgactacgtgcagccgctgggcaagttccgggctctgtgtaccgtctgcgcgccggtctccttcctggagtgcggctccgacgagctgttcgtgggccgcgcgggttacctgtgtgccgcgctggtgctcaagcagaaactcgcccaggaggtgctgactccagcacagatcaagtcaatttgtcaggcaattctggactctgggaagcagtatgccataaagaagaggaaaccattccccctgatgtattcttactatggaaccgaatacttgggggcagctcacggcttgtcgtctattcttcagatgcttctttcttaccatgagcatctcaagccctcagatcgggaattggtatggcagagcgtggactttctcatggaacaggaacaaaactgcaactggccacctgagctcggcgagaccatcgagagagagaatgagctggtgcactggtgccatggcgctccaggaattgcctatctgtttgccaaagcttatctggtttccaagaaaccgcagtacctggacacatgtattcggtgtggggaactcacatggcagaaaggcctgctaaagaaggggcctgggatttgccatggagtagccggcagtgcctatgtcttcctgctgctgtaccggctcacgggaaactctaaatacatctaccgagctcaaaggtttgctcaattcttatttaccgaggaattcaaggccggttctcgggtccttgaaagtatatacagcttgtatgaaggcttctctgggacagtgtgctttctgattgacctgctgcagcccaatcaggctgaattcccactcttcagcgtctttgtttagaaggctctatcttccactgtggccctgcagagatcccctgagccaagccgaggcagtttccacataagccacattcaatggtatcgcaaccatgagccttaacattgccatcagaaggaaggaatcaggcaggtgaaggcaacatgatgccagatttgagaaaggatctgcaaaataaagataccacaattcatcttaaaactgcagagatttaatgtgtcagggaatagatgtgaaacaagggatcataggaaaaggggaaagagaaatgatctgtttttcagttatgacatagaaaaccaaactgcaagtgtagactatgacaaaaaatacactaatacctttgcaatctgaatgagaatttgaccatttgtgtgtgccctctacccttaaattcagaaataaagacaataaaaaattaaaac

SEQ ID NO: 104 - protein, 420 aa, Hom sapiens
MDTKRCFANRFDDYQGSLLAGQCEEAVAPLVTATIERILQELPPLGGGAEARGATAEASACQGGLYGGVAGVAYMLYHVSQSPLFATARERYLRSAKRLIDACARAEEWGEPDADTRAAFLLGGAGVYAVATLVYHALGRSDYVQPLGKFRALCTVCAPVSFLECGSDELFVGRAGYLCAALVLKQKLAQEVLTPAQIKSICQAILDSGKQYAIKKRKPFPLMYSYYGTEYLGAAHGLSSILQMLLSYHEHLKPSDRELVWQSVDFLMEQEQNCNWPPELGETIERENELVHWCHGAPGIAYLFAKAYLVSKKPQYLDTCIRCGELTWQKGLLKKGPGICHGVAGSAYVFLLLYRLTGNSKYIYRAQRFAQFLFTEEFKAGSRVLESIYSLYEGFSGTVCFLIDLLQPNQAEFPLFSVFV

SEQ ID NO: 105 - DNA, 1263 bp, Homo sapiens
  CDS (1)...(1263)
atggacaccaagcgctgcttcgccaatcgcttcgatgactaccagggcagcctgctggcgggccagtgtgaggaggcggtggcgcccttggtcaccgccaccatcgagcgcatcctccaggagcttcccccactcgggggcggcgcggaggcccgaggggcgacggcggaggctagcgcctgccagggggggctttatggcggcgtggccggagtggcgtatatgctctaccacgtctcgcagagcccgcttttcgccacggcccgggaacgctacctgcgctcggctaagcgcctcatcgacgcgtgcgcccgcgctgaggagtggggcgaaccggacgccgacacccgcgccgccttcctgctcgggggcgcgggcgtgtacgccgtggccacgctcgtataccacgccctgggccggtccgactacgtgcagccgctgggcaagttccgggctctgtgtaccgtctgcgcgccggtctccttcctggagtgcggctccgacgagctgttcgtgggccgcgcgggttacctgtgtgccgcgctggtgctcaagcagaaactcgcccaggaggtgctgactccagcacagatcaagtcaatttgtcaggcaattctggactctgggaagcagtatgccataaagaagaggaaaccattccccctgatgtattcttactatggaaccgaatacttgggggcagctcacggcttgtcgtctattcttcagatgcttctttcttaccatgagcatctcaagccctcagatcgggaattggtatggcagagcgtggactttctcatggaacaggaacaaaactgcaactggccacctgagctcggcgagaccatcgagagagagaatgagctggtgcactggtgccatggcgctccaggaattgcctatctgtttgccaaagcttatctggtttccaagaaaccgcagtacctggacacatgtattcggtgtggggaactcacatggcagaaaggcctgctaaagaaggggcctgggatttgccatggagtagccggcagtgcctatgtcttcctgctgctgtaccggctcacgggaaactctaaatacatctaccgagctcaaaggtttgctcaattcttatttaccgaggaattcaaggccggttctcgggtccttgaaagtatatacagcttgtatgaaggcttctctgggacagtgtgctttctgattgacctgctgcagcccaatcaggctgaattcccactcttcagcgtctttgtttag

SEQ ID NO: 106 - protein, 216 aa, Artificial Sequence
  Amino acid consensus sequence
CSSILMKNLISKDKGIGWKPPWQETPLTGAAHGLAGIMYALLHSYLKTDDEKYLKIVKSTLDYERSNRFPQSGNWPDSREGNKRDKLGPDVHWCHGAPGVAITLLKAYKVNFSKEEEYKEEAEECADVVWKKGLLKKNYGLCHGVAGNAYVFLSLYRLTNNSKYEYRAKKFAEWLLNYGQKLGWETGMNEPDRPYSLFEGVAGIAYGLLRYISPSE

SEQ ID NO: 107 - protein, 127 aa, Artificial Sequence
  Amino acid consensus sequence
LAGYYYFLKRLSKVWNKEISCEDMQYNNLKHWDDLIQEDETYDFIGGYAGVLVVLTNLYILFDEPNYLEIALKCSSILMKNLISKDKGIGWKPPWQETPLTGAAHGLAGIMYALLHSYLKTDDEKYL

SEQ ID NO: 108 - protein, 26 aa, Artificial Sequence
  Amino acid consensus sequence
FIGGYAGVLVVLTNLYILFDEPNYLE

SEQ ID NO: 109 - protein, 148 aa, Artificial Sequence
  Amino acid consensus sequence
TGDQTYLQLCLEYVKACADLANLSSRHVTFLCGDAGPCAVGAVIYHKLGDEEEADDCLTRFLHLNKSSELPEPHEYGGFGMPNELLYGRAGYLWACLFLNKYIGEETIPDDHIQEICEAIITSGRSLSRENNKTSKCPLMYQWHGKRY

SEQ ID NO: 110 - protein, 22 aa, Artificial Sequence
  Amino acid consensus sequence
LXXXXXXLXXXXXXLXXXXXXL